(19) United States
(12) Patent Application Publication
(10) Pub. No.: US 2026/0188490 A1
(43) Pub. Date: Jul. 2, 2026

(54) CELL-FREE DNA METHYLATION TEST FOR BREAST CANCER

(71) Applicant: UNIVERSITY OF SOUTHERN CALIFORNIA, Los Angeles, CA (US)

(72) Inventors: Bodour SALHIA, Los Angeles, CA (US); David BUCKLEY, Los Angeles, CA (US)

(73) Assignee: UNIVERSITY OF SOUTHERN CALIFORNIA, Los Angeles, CA (US)

(21) Appl. No.: 19/131,602

(22) PCT Filed: Nov. 22, 2023
(86) PCT No.: PCT/US2023/081012

§ 371 Date: May 21, 2025

Related U.S. Application Data
(60) Provisional application No. 63/384,731, filed on Nov. 22, 2022.

(51) Int. Cl. G16H 50/20; C12Q 1/6886; G16B 20/20; G16B 30/00; G16B 40/30; G16H 20/10; G16H 20/40; G16H 50/70

(52) U.S. Cl. CPC G16H 50/20; C12Q 1/6886; G16B 20/20; G16B 30/00; G16B 40/30; G16H 20/10; G16H 20/40; G16H 50/70; C12Q 2600/154

(57) ABSTRACT

The disclosure provides for assays and methods of using the same for determining whether a subject has Minimum Residual Disease (MRD) including the steps: training a machine learning model to develop an MRD signature, determining a methylation pattern of target regions of a cell-free deoxyribonucleic acid (cfDNA) sample obtained from the subject; applying the MRD signature to the methylation pattern of the target regions of the cfDNA obtained from the subject; and determining that the subject has or does not have the MRD based on the MRD signature.

### RELATED APPLICATIONS

This application claims priority under 35 U.S.C. § 119 (e) to U.S. Provisional Patent Application No. 63/384,731, filed Nov. 22, 2022, which is incorporated herein by reference.

### STATEMENT REGARDING FEDERAL FUNDING

This invention was made with government support under grant no. CA201352, awarded by the National Institutes of Health. The government has certain rights in the invention.

### BACKGROUND OF THE INVENTION

Despite recent improvements in breast cancer (BC) screening, diagnosis, and treatment, there are patients who still develop metastasis and succumb to their disease. The 5-year overall survival (OS) of patients with metastatic breast cancer is less than 25%. The risk of metastasis increases with tumor size, lymph node involvement, lack of estrogen receptor (ER) expression, over-expression of human epidermal growth factor receptor 2 (HER2), and higher histopathological differentiation (grade).

Clinical tests based on molecular profiles have improved our ability to stratify patients based on recurrence risk. The most widely used multigene predictive classifiers is the 21-gene Oncotype Dx signature (Exact Sciences, USA), and others include the 70-gene MammaPrint signature (Agendia, Netherlands), the 76-gene Rotterdam signature and the PAM50 intrinsic classifier (NanoString, USA). Although these tests are based on large sets of specifically selected genes, none can precisely predict disease recurrence. Additionally, these tests reflect a snapshot of each individual cancer at the time of biopsy and are not intended to be used to monitor changes in cancer molecular profiles over time. All these tests are recommended for ER positive tumors but are not approved for higher risk BC subtypes such as HER2 positive and triple negative breast cancer (TNBC). An alternate strategy for stratifying patients as high risk for systemic recurrence is response to neoadjuvant therapy. However meta-analyses have not demonstrated correlation of pathologic complete response to treatment with disease free survival (DFS) or OS.

Metastatic breast cancer (MBC) is an incurable disease affecting 10-15% of breast cancer patients. MBC arises from disseminated cells from the primary tumor mass before treatment and/or minimal residual disease remaining after therapy. If these cells persist after systemic chemotherapy (either adjuvant or neoadjuvant) they can lead to a recurrence several months or even years after primary treatment. Historically, the only method to detect a recurrence is discovery of a local recurrence or a metastatic nodule. A full body CT scan may be indicated in high-risk patients, but for most MBC patients the first indicator of recurrence is symptoms caused by organ damage due to local metastatic growth. Such metastases are often well established and difficult to treat even with high dose chemotherapy and surgical intervention. More recently, Personalized, tumor-informed circulating tumor (ct) DNA molecular residual disease (MRD) testing for breast cancer to inform critical decisions for care. The Signatera™ Residual Disease Test is a custom-built blood test for people who have been diagnosed with breast cancer or other solid tumors. Signatera™ can detect molecular residual disease (MRD) in the form of circulating tumor DNA. However, this test uses tumor-informed mutation data and its sensitivity and specificity remain limited.

Accordingly, there is a need for new methods of diagnosing MBC and MRD that is more sensitive and has higher specificity than previous methods. The present disclosure satisfies these needs.

### SUMMARY

Disclosed herein are tools and assays specifically designed to detect evidence of MRD prior to clinical recurrence using cost-effective, minimally invasive techniques that can be repeatedly applied following end of care or possibly even at diagnosis and during treatment. Embodiments of the disclosure may be used to identify and measure methylation patterns in cell-free (cf) DNA to develop an MRD signature. This signature would identify patients at highest risk of recurrence.

Utilizing cfDNA to monitor MRD has emerged as a promising blood-based biomarker strategy. Evidence for MRD is considered a prognostic marker to identify individuals at high risk of recurrence. cfDNA is an excellent substrate to analyze for MRD monitoring as it 1) contains a wealth of information from multiple tissue types 2) is minimally invasive to the patient, requiring only a standard veinous blood draw, and 3) is easily repeatable over time. Furthermore, cfDNA may give a more accurate representation of the primary tissue, as traditional biopsy can be biased by subclones and tumor heterogeneity.

Accordingly, the disclosure provides for panel assays and methods of using the same. In some embodiments, a method for determining whether a subject has Minimum Residual Disease (MRD) comprising steps: a) training a machine learning model to develop an MRD signature, wherein the machine learning program is trained using target regions from cancerous samples and corresponding target regions from non-cancerous samples, wherein the MRD signature is based on a comparison of a methylation pattern of target regions of the cancerous samples compared to a methylation pattern of corresponding target regions of the non-cancerous samples; b) determining a methylation pattern of target regions of a cell-free deoxyribonucleic acid (cfDNA) sample obtained from the subject; c) applying the MRD signature to the methylation pattern of the target regions of the cfDNA obtained from the subject; and d) determining that the subject has or does not have the MRD based on the MRD signature.

These and other features and advantages of this invention will be more fully understood from the following detailed description of the invention taken together with the accompanying claims. It is noted that the scope of the claims is defined by the recitations therein and not by the specific discussion of features and advantages set forth in the present description.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIG. 1. Example of CpG methylation states in a hypothetical genomic region. Filled black dot represents a methyl group, empty dot represents an absent methyl group.

FIG. 2. Coverage and beta value evaluated by FLAME and BSSEQ.

FIG. 3. Synthetic methylation fragments as tabulated by FLAME compared to expected counts.

FIG. 4. CSFs detected from in silico spike in of MBC cfDNA into healthy cfDNA.

FIG. 5A-B. WGBS reveals MBC methylation profiles differs from DFS and Healthy. A) Heat scatterplots show percent methylation form pair-wise comparisons of three study groups. Numbers in the upper right corner denote Pearson's correlation coefficients. The histograms on the diagonal are frequency of percent methylation per CpG for each pool. MBC demonstrates a shift to the left compared to DFS and Healthy indicating genome wide hypomethylation. B) Principal component analysis (PCA of the methylation profiles of each cfDNA pool. Samples closer to each other in clustering or PCA are similar in methylation profiles. (See, for example, Legendre et al. Clin Epigenetics 2015 Sep. 16; 7 (1): 100. doi: 10.1186/s13148-015-0135-8).

FIG. 6. Receiver operating characteristic (ROC) curve of random forest classifier model performance in a training set of 30 samples shows high sensitivity and specificity at classifying MBC from healthy patients using cfDNA. Area under the curve (AUC) is annotated.

FIG. 7. Evidence for MRD in cfDNA collected post-neoadjuvant therapy and postoperative (color). Each plot is subdivided by patient outcome: DFS (disease free survivor), REC (recurred), and NDF (never disease free). A) Probability score evaluated by the RF model (FIG. 3) shows little change between timepoints, and minimal difference between samples. B) Number of cancer specific fragments (CSFs) per sample shows large decrease in DFS, increase in both recurrent samples, and slight increase in the never disease-free sample.

### DETAILED DESCRIPTION

### Definitions

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14^{th} Edition, by R. J. Lewis, John Wiley & Sons, New York, N.Y., 2001 or Singleton, et al., Dictionary of Microbiology and Molecular Biology, 2d ed., John Wiley and Sons, New York (1994), and Hale & Markham, The Harper Collins Dictionary of Biology. Harper Perennial, N.Y. (1991). General laboratory techniques (DNA extraction, RNA extraction, cloning, cell culturing, etc.) are known in the art and described, for example, in Molecular Cloning: A Laboratory Manual, J. Sambrook et al., 4th edition, Cold Spring Harbor Laboratory Press, 2012.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

Wherever the term “comprising” is used herein, options are contemplated wherein the terms “consisting of” or “consisting essentially of” are used instead. As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the aspect element. As used herein, “consisting essentially of” does not exclude materials or steps that do not materially affect the basic and novel characteristics of the aspect. In each instance herein any of the terms “comprising”, “consisting essentially of” and “consisting of” may be replaced with either of the other two terms. The disclosure illustratively described herein may be suitably practiced in the absence of any element or elements, limitation, or limitations not specifically disclosed herein.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrases “one or more” and “at least one” are readily understood by one of skill in the art, particularly when read in context of its usage. For example, the phrase can mean one, two, three, four, five, six, ten, 100, or any upper limit approximately 10, 100, or 1000 times higher than a recited lower limit. For example, one or more substituents on a phenyl ring refers to one to five substituents on the ring.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements. When values are expressed as approximations, by use of the antecedent “about,” it will be understood that the particular value without the modifier “about” also forms a further aspect.

The terms “about” and “approximately” are used interchangeably. Both terms can refer to a variation of ±5%, ±10%, ±20%, or ±25% of the value specified. For example, “about 50” percent can in some embodiments carry a variation from 45 to 55 percent, or as otherwise defined by a particular claim. For integer ranges, the term “about” can include one or two integers greater than and/or less than a recited integer at each end of the range. Unless indicated otherwise herein, the terms “about” and “approximately” are intended to include values, e.g., weight percentages, proximate to the recited range that are equivalent in terms of the functionality of the individual ingredient, composition, or embodiment. The terms “about” and “approximately” can also modify the endpoints of a recited range as discussed above in this paragraph.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. It is therefore understood that each unit between two particular units are also disclosed. For example, if 10 to 15 is disclosed, then 11, 12, 13, and 14 are also disclosed, individually, and as part of a range. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents. It will be further understood that the endpoints of each of the ranges are significant both in relation to the other endpoint, and independently of the other endpoint.

This disclosure provides ranges, limits, and deviations to variables such as volume, mass, percentages, ratios, etc. It is understood by an ordinary person skilled in the art that a range, such as “number 1” to “number 2”, implies a continuous range of numbers that includes the whole numbers and fractional numbers. For example, 1 to 10 means 1, 2, 3, 4, 5, . . . 9, 10. It also means 1.0, 1.1, 1.2. 1.3, . . . , 9.8, 9.9, 10.0, and also means 1.01, 1.02, 1.03, and so on. If the variable disclosed is a number less than “number10”, it implies a continuous range that includes whole numbers and fractional numbers less than number10, as discussed above. Similarly, if the variable disclosed is a number greater than “number10”, it implies a continuous range that includes whole numbers and fractional numbers greater than number10. These ranges can be modified by the term “about”, whose meaning has been described above.

The term “substantially” as used herein, is a broad term and is used in its ordinary sense, including, without limitation, being largely but not necessarily wholly that which is specified. For example, the term could refer to a numerical value that may not be 100% the full numerical value. The full numerical value may be less by about 1%, about 2%, about 3%, about 4%, about 5%, about 6%, about 7%, about 8%, about 9%, about 10%, about 15%, or about 20%.

As used herein, the term “a portion of” or “a portion thereof” means consecutive nucleotides of the sequence of said particular region. A portion according to the invention can comprise or consist of at least 15 or 20 consecutive nucleotides, preferably at least 100, 200, 300, 500 or 700 consecutive nucleotides, and more preferably at least 1, 2, 3, 4 or 5 consecutive kb of said particular region. For example, a portion can comprise or consist of 1, 2, 3, 4, 5, 6, 7, 8, 9, 10, 11, 12, 13, 14, 15 consecutive kb of said particular region.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

An “effective amount” refers to an amount effective to treat a disease, disorder, and/or condition, or to bring about a recited effect. For example, an effective amount can be an amount effective to reduce the progression or severity of the condition or symptoms being treated. Determination of a therapeutically effective amount is well within the capacity of persons skilled in the art. The term “effective amount” is intended to include an amount of a compound described herein, or an amount of a combination of compounds described herein, e.g., that is effective to treat or prevent a disease or disorder, or to treat the symptoms of the disease or disorder, in a host. Thus, an “effective amount” generally means an amount that provides the desired effect.

Alternatively, the terms “effective amount” or “therapeutically effective amount,” as used herein, refer to a sufficient amount of an agent or a composition or combination of compositions being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case may be determined using techniques, such as a dose escalation study. The dose could be administered in one or more administrations. However, the precise determination of what would be considered an effective dose may be based on factors individual to each patient, including, but not limited to, the patient's age, size, type or extent of disease, stage of the disease, route of administration of the compositions, the type or extent of supplemental therapy used, ongoing disease process and type of treatment desired (e.g., aggressive vs. conventional treatment).

The terms “treating”, “treat” and “treatment” include (i) preventing a disease, pathologic or medical condition from occurring (e.g., prophylaxis); (ii) inhibiting the disease, pathologic or medical condition or arresting its development; (iii) relieving the disease, pathologic or medical condition; and/or (iv) diminishing symptoms associated with the disease, pathologic or medical condition. Thus, the terms “treat”, “treatment”, and “treating” can extend to prophylaxis and can include prevent, prevention, preventing, lowering, stopping, or reversing the progression or severity of the condition or symptoms being treated. As such, the term “treatment” can include medical, therapeutic, and/or prophylactic administration, as appropriate.

As used herein, “subject” or “patient” means an individual having symptoms of, or at risk for, a disease or other malignancy. A patient may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. In one embodiment of the methods provided herein, the mammal is a human.

As used herein, the terms “providing”, “administering,” “introducing,” are used interchangeably herein and refer to the placement of a compound of the disclosure into a subject by a method or route that results in at least partial localization of the compound to a desired site. The compound can be administered by any appropriate route that results in delivery to a desired location in the subject.

The terms “inhibit”, “inhibiting”, and “inhibition” refer to the slowing, halting, or reversing the growth or progression of a disease, infection, condition, or group of cells. The inhibition can be greater than about 20%, 40%, 60%, 80%, 90%, 95%, or 99%, for example, compared to the growth or progression that occurs in the absence of the treatment or contacting.

The term “amplicon” refers to nucleic acid products resulting from the amplification of a target nucleic acid sequence. Amplification is often performed by PCR. Amplicons can range in size from 20 base pairs to 15000 base pairs in the case of long-range PCR but are more commonly 100-1000 base pairs for bisulfite-treated DNA used for methylation analysis.

The term “amplification” refers to an increase in the number of copies of a nucleic acid molecule. The resulting amplification products are called “amplicons.” Amplification of a nucleic acid molecule (such as a DNA or RNA molecule) refers to use of a technique that increases the number of copies of a nucleic acid molecule in a sample. An example of amplification is the polymerase chain reaction (PCR), in which a sample is contacted with a pair of oligonucleotide primers under conditions that allow for the hybridization of the primers to a nucleic acid template in the sample. The product of amplification can be characterized by such techniques as electrophoresis, restriction endonuclease cleavage patterns, oligonucleotide hybridization or ligation, and/or nucleic acid sequencing. In some embodiments, the methods provided herein can include a step of producing an amplified nucleic acid under isothermal or thermal variable conditions.

The term “biological sample” refers to a sample obtained from an individual. As used herein, biological samples include all clinical samples containing genomic DNA (such as cell-free genomic DNA) useful for cancer diagnosis and prognosis, including, but not limited to, cells, tissues, and bodily fluids, such as: blood, derivatives and fractions of blood (such as serum or plasma), buccal epithelium, saliva, urine, stools, bronchial aspirates, sputum, biopsy (such as tumor biopsy), and CVS samples. A “biological sample” obtained or derived from an individual includes any such sample that has been processed in any suitable manner (for example, processed to isolate genomic DNA for bisulfite treatment) after being obtained from the individual.

The term “bisulfite treatment” refers to the treatment of DNA with bisulfite or a salt thereof, such as sodium bisulfite (NaHSO₃). Bisulfite reacts readily with the 5,6-double bond of cytosine, but poorly with methylated cytosine. Cytosine reacts with the bisulfite ion to form a sulfonated cytosine reaction intermediate which is susceptible to deamination, giving rise to a sulfonated uracil. The sulfonate group can be removed under alkaline conditions, resulting in the formation of uracil. Uracil is recognized as a thymine by polymerases and amplification will result in an adenine-thymine base pair instead of a cytosine-guanine base pair.

The term “cancer” refers to a biological condition in which a malignant tumor or other neoplasm has undergone characteristic anaplasia with loss of differentiation, increased rate of growth, invasion of surrounding tissue, and which is capable of metastasis. A neoplasm is a new and abnormal growth, particularly a new growth of tissue or cells in which the growth is uncontrolled and progressive. A tumor is an example of a neoplasm. Non-limiting examples of types of cancer include lung cancer, stomach cancer, colon cancer, breast cancer, uterine cancer, bladder, head and neck, kidney, liver, ovarian, pancreas, prostate, and rectum cancer.

The terms “polynucleotide” and “nucleic acid” are used interchangeably and mean at least two or more ribo- or deoxy-ribo nucleic acid base pairs (nucleotide) linked which are through a phosphoester bond or equivalent. The nucleic acid includes polynucleotide and polynucleoside. The nucleic acid includes a single molecule, a double molecule, a triple molecule, a circular molecule, or a linear molecule. Examples of the nucleic acid include RNA, DNA, cDNA, a genomic nucleic acid, a naturally existing nucleic acid, and a non-natural nucleic acid such as a synthetic nucleic acid but are not limited. Short nucleic acids and polynucleotides (e.g., 10 to 20, 20 to 30, 30 to 50, 50 to 100 nucleotides) are commonly called “oligonucleotides” or “probes” of single-stranded or double-stranded DNA.

The term “DNA (deoxyribonucleic acid)” refers to a long chain polymer which comprises the genetic material of most living organisms. The repeating units in DNA polymers are four different nucleotides, each of which comprises one of the four bases, adenine, guanine, cytosine, and thymine bound to a deoxyribose sugar to which a phosphate group is attached. Triplets of nucleotides (referred to as codons) code for each amino acid in a polypeptide, or for a stop signal. The term codon is also used for the corresponding (and complementary) sequences of three nucleotides in the mRNA into which the DNA sequence is transcribed.

The term “cell-free DNA” refers to DNA which is no longer fully contained within an intact cell, for example DNA found in plasma or serum.

The term “target nucleic acid molecule” refers to a nucleic acid molecule whose detection, amplification, quantitation, qualitative detection, or a combination thereof, is intended. The nucleic acid molecule need not be in a purified form. Various other nucleic acid molecules can also be present with the target nucleic acid molecule. For example, the target nucleic acid molecule can be a specific nucleic acid molecule of which the amplification and/or evaluation of methylation status is intended. Purification or isolation of the target nucleic acid molecule, if needed, can be conducted by methods known to those in the art, such as by using a commercially available purification kit or the like.

The term “methylation level” refers to the state of methylation (methylated or not methylated) of the cytosine nucleotide of one or more CpG sites within a genomic sequence.

The term “CpG Site” refers to a di-nucleotide DNA sequence comprising a cytosine followed by a guanine in the 5′ to 3′ direction. The cytosine nucleotides of CpG sites in genomic DNA are the target of intracellular methyltransferases and can have a methylation status of methylated or not methylated. Reference to “methylated CpG site” or similar language refers to a CpG site in genomic DNA having a 5-methylcytosine nucleotide.

As used herein, “sequence identity” or “identity” in the context of two nucleic acid or polypeptide sequences makes reference to a specified percentage of residues in the two sequences that are the same when aligned for maximum correspondence over a specified comparison window, as measured by sequence comparison algorithms or by visual inspection. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

The term “substantial identity” in the context of a peptide indicates that a peptide comprises a sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, or 94%, or even 95%, 96%, 97%, 98% or 99%, sequence identity to the reference sequence over a specified comparison window. In certain embodiments, optimal alignment is conducted using the homology alignment algorithm of Needleman and Wunsch (Needleman and Wunsch, JMB, 48, 443 (1970)). An indication that two peptide sequences are substantially identical is that one peptide is immunologically reactive with antibodies raised against the second peptide. Thus, a peptide is substantially identical to a second peptide, for example, where the two peptides differ only by a conservative substitution. Thus, embodiment of the invention also provides nucleic acid molecules and peptides that are substantially identical to the nucleic acid molecules and peptides presented herein.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

The term “primer” as used herein refers to a short polynucleotide that hybridizes to a target polynucleotide sequence and serves as the starting point for synthesis of new polynucleotides.

The term “multiplex” refers to the use of more than one pair of primers intended to amplify multiple target gene segments simultaneously within a single tube. In this manner, all the primers may be contained within one tube to which a sample is introduced or positioned. All desired influenza virus and control gene segments are then amplified via the plurality of forward and reverse primers within the tube.

The term “complement” as used herein means the complementary sequence to a nucleic acid according to standard Watson/Crick base pairing rules. A complement sequence can also be a sequence of RNA complementary to the DNA sequence or its complement sequence and can also be a cDNA. The term “substantially complementary” as used herein means that two sequences hybridize under stringent hybridization conditions. The skilled artisan will understand that substantially complementary sequences need not hybridize along their entire length. In particular, substantially complementary sequences comprise a contiguous sequence of bases that do not hybridize to a target or marker sequence, positioned 3′ or 5′ to a contiguous sequence of bases that hybridize under stringent hybridization conditions to a target or marker sequence.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PC reaction, or the enzymatic cleavage of a polynucleotide by a ribozyme.

Examples of stringent hybridization conditions include incubation temperatures of about 25° C. to about 37° C.; hybridization buffer concentrations of about 6×SSC to about 10×SSC; formamide concentrations of about 0% to about 25%; and wash solutions from about 4×SSC to about 8×SSC. Examples of moderate hybridization conditions include incubation temperatures of about 40° C. to about 50° C.; buffer concentrations of about 9×SSC to about 2×SSC; formamide concentrations of about 30% to about 50%; and wash solutions of about 5×SSC to about 2×SSC. Examples of high stringency conditions include incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

As used herein, the term “reference genome” refers to any particular known, sequenced or characterized genome, whether partial or complete, of any organism or virus that may be used to reference identified sequences from a subject. Exemplary reference genomes used for human subjects as well as many other organisms are provided in the on-line genome browser hosted by the National Center for Biotechnology Information (“NCBI”) or the University of California, Santa Cruz (UCSC). A “genome” refers to the complete genetic information of an organism or virus, expressed in nucleic acid sequences. As used herein, a reference sequence or reference genome often is an assembled or partially assembled genomic sequence from an individual or multiple individuals. In some embodiments, a reference genome is an assembled or partially assembled genomic sequence from one or more human individuals. The reference genome can be viewed as a representative example of a species' set of genes. In some embodiments, a reference genome comprises sequences assigned to chromosomes. One exemplary human reference genome is GRCh37 (UCSC equivalent: hg19).

As used herein, the term “normal reference standard” intends a control level, degree, or range of DNA methylation at a particular genomic region or gene in a sample that is not associated with cancer. The term “normal reference cutoff value” refers to a control threshold level of DNA methylation at a particular genomic region or gene or a differential methylation value (DMV). In some embodiments, DNA methylation levels enriched above the normal reference cutoff value are associated with having or developing cancer. In some embodiments, DNA methylation levels at or below the normal reference cutoff value are associated with not having or developing cancer.

“Detecting” as used herein refers to determining the presence and/or degree of methylation in a nucleic acid of interest in a sample. Detection does not require the method to provide 100% sensitivity and/or 100% specificity.

“RT-PCR” refers to reverse transcription polymerase chain reaction and is used to detect specific RNA, in this case specific gene segments of the influenza virus genome, such as by reverse transcribing the RNA of interest into its DNA complement through the use of reverse transcriptase. The newly synthesized cDNA can be amplified using traditional PCR. In an aspect, the RT-PCR provided herein is by a one-step approach, wherein the entire reaction from cDNA synthesis to PCR amplification occurs in a single tube. Alternatively, the process described herein is compatible with a two-step reaction requires that the reverse transcriptase reaction and PCR amplification be performed in separate tubes. Real-Time PCR: Current Technology and Applications, Logan, Edwards, and Saunders eds., Caister Academic Press, 2009; Bustin A-Z of Quantitative PCR (IUL Biotechnology, No. 5).

As used here, a “fragment” of DNA refers to a piece of cell-free DNA that is about 10 bp, about 20 bp, about 30 bp, about 40 bp, about 50 bp, about 60 bp, about 70 bp, about 80 bp, about 90 bp, about 100 bp, about 110 bp, about 120 bp, about 130 bp, about 140 bp, about 150 bp, about 160 bp, about 170 bp, about 180 bp, about 190 bp, about 200 bp, about 210 bp, about 220 bp, about 230 bp, 240 bp, about 250 bp, about 260 bp, about 270 bp, 280 bp, about 290 bp, about 300 bp, about 310 bp, about 320 bp, about 330 bp, about 340 bp, about 350 bp, about 360 bp, about 370 bp, about 380 bp, about 390 bp, or about 400 bp in length. Typically, DNA fragments are about 100 bp to about 200 bp, about 120 bp to about 180 bp, or about 140 bp to about 160 bp.

The term “neoadjuvant treatment” refers to treatment (such as chemotherapy or hormone therapy) administered before primary cancer treatment (such as surgery) to enhance the outcome of primary treatment.

The term “chemotherapy” refers to the treatment of cancer with an antitumor or chemotherapeutic agent as part of a standardized regimen. Chemotherapy may be given with a curative intent or it may aim to prolong life or to palliate symptoms. It may be used in conjunction with other cancer treatments, such as radiation therapy or surgery.

The term “methylation” refers to the addition of a methyl group to the 5′ carbon of the cytosine base in a deoxyribonucleic acid sequence of CpG within a genome.

The term “neighboring CpG site” refers to the collection of CpG sites within a genomic feature or over a short genetic distance. The genomic feature may be a promoter, an enhancer, an exon, an intron, a 5′-untranslated region (UTR), a 3′-UTR, a gene body, a stem cell associated region, a CpG island, a CpG shelf, a CpG shore, a LINE, a SINE, or an LTR. The short genetic distance may be 10 bp, 11 bp, 12 bp, 13 bp, 14 bp, 15 bp, 16 bp, 17 bp, 18 bp, 19 bp, 20 bp, 21 bp, 22 bp, 23 bp, 24 bp, 25 bp, 26 bp, 27 bp, 28 bp, 29 bp, 30 bp, 31 bp, 32 bp, 33 bp, 34 bp, 35 bp, 36 bp, 37 bp, 38 bp, 39 bp, 40 bp, 41 bp, 42 bp, 43 bp, 44 bp, 45 bp, 46 bp, 47 bp, 48 bp, 49 bp, 50 bp, 51 bp, 52 bp, 53 bp, 54 bp, 55 bp, 56 bp, 57 bp, 58 bp, 59 bp, 60 bp, 61 bp, 62 bp, 63 bp, 64 bp, 65 bp, 66 bp, 67 bp, 68 bp, 69 bp, 70 bp, 71 bp, 72 bp, 73 bp, 74 bp, 75 bp, 76 bp, 77 bp, 78 bp, 79 bp, 80 bp, 81 bp, 82 bp, 83 bp, 84 bp, 85 bp, 86 bp, 87 bp, 88 bp, 89 bp, 90 bp, 91 bp, 92 bp, 93 bp, 94 bp, 95 bp, 96 bp, 97 bp, 98 bp, 99 bp, 100 bp, 250 bp, 500 bp, 750 bp or 1,000 bp. Optionally, neighboring CpG sites occur within a sequencing read.

The term “Minimal Residual Disease” or “MRD” refers to cancer cells (e.g., breast cancer cells) remaining after treatment that cannot be detected using the scans or tests to identify a remission state (i.e., cancer free). Treatment of any cancer listed herein may result in MRD.

The term “fragment assessment regions” or “FAR” refers to a set of coordinates within a DMR call (target region) with n CpGs within 1 base pairs of each other where 1 is less than the expected fragment length (typically 160 bp).

### Embodiments of the Invention

The disclosure provides for panel assays and various methods for detecting differences in methylation patterns of a target region of cfDNA. The differences in methylation patterns of the target regions of the sample can indicate, for example, the presence or absence of breast cancer, the severity of the breast cancer, a susceptibility to breast cancer, recurrence or susceptibility to recurrence of breast cancer, the presence or absence of minimal residual disease (MRD), and susceptibility to MRD. The methylation pattern of the target region of cfDNA in a sample may be analyzed using a trained machine learning algorithm that is trained using target regions of cfDNA of cancerous samples such as metastatic breast cancer and non-cancerous control samples to develop and MRD signature used to detect MRD in a subject.

### Statement of Embodiments

Statement 1. A method for determining whether a subject has Minimum Residual Disease (MRD) comprising steps: a) training a machine learning program to develop an MRD signature, wherein the machine learning program is trained using a plurality of target regions from cancerous samples and a plurality of corresponding target regions from non-cancerous samples, wherein the MRD signature is based on a comparison of a methylation pattern of a plurality of target regions of the cancerous samples compared to a methylation pattern of a plurality of corresponding target regions of the non-cancerous samples; b) determining a methylation pattern of a plurality of target regions of a cell-free deoxyribonucleic acid (cfDNA) sample obtained from the subject; c) applying the MRD signature to the methylation pattern of the plurality of target regions of the cfDNA obtained from the subject; and d) determining that the subject has or does not have the MRD based the MRD signature.

Statement 2. The method of statement 1 wherein the plurality of target regions in the cfDNA sample from the subject are identical to the plurality of target genomic regions of both the cancerous sample and the non-cancerous samples used to develop the MRD signature.

Statement 3. The method of statement 1 or 2 wherein the methylation pattern of the plurality of target regions is determined using one or more of post whole genome library hybrid probe capture, enzymatic treatment, bisulfite amplicon sequencing (BSAS), bisulfite treatment of DNA, methylation sensitive polymerase chain reaction, and bisulfite conversion combined with bisulfite restriction analysis.

Statement 4. The method of any one of statements 1-3 wherein the methylation pattern of each of the plurality of target regions is determined using a hybrid probe capture method.

Statement 5. The method of any one of statements 1˜4 wherein the hybrid probe capture method comprises using one or more hybrid capture probes comprising ribonucleic acid or deoxyribonucleic acid.

Statement 6. The method any one of statements 1-5 wherein each of the one or more hybrid capture probes further comprises an affinity tag selected from the group consisting of biotin and streptavidin.

Statement 7. The method of any one of statements 1-6 wherein the plurality of target regions from cancerous samples and from non-cancerous samples comprises about 60% to at about 70% of the target regions of Table 1.

Statement 8. The method any one of statements 1-7 wherein the plurality of target regions comprises about 70% to about 80% of the target regions of Table 1.

Statement 9. The method any one of statements 1-8 wherein the plurality of target regions comprises about 80% to about 90% of the target regions of Table 1.

Statement 10. The method any one of statements 1-9 wherein the plurality of target regions comprises greater than about 95% of the target regions of Table 1.

Statement 11. The method any one of statements 1-10 wherein the cfDNA sample is extracted from whole blood, plasma, serum, or urine.

Statement 12. The method any one of statements 1-11 further comprising steps: e) combining adjacent CpGs of each of the plurality of target regions into contiguous n through m number of CpG blocks wherein n is at least 1 and m is less than a length of a corresponding target region; f) removing any target region having less than the n number of CpG blocks and greater than the m number of CpG blocks; and g) filtering the target regions remaining after step f) using a k-means clustering function based on adjacent CpGs to provide one or more fragment assessment regions (FAR).

Statement 13. The method of any one of statements 1-12 further comprising tabulating a methylation state of each FAR according to the steps of: h) identifying all or substantially all possible methylation patterns of CpGs in the FAR; i) selecting all sequence reads that overlap the FAR; j) extracting the methylation states of each of the CpGs in the sequence read that spans the FAR; k) counting each distinct methylation pattern in the FAR to provide a count of methylation states; and 1) outputting a result of steps h)-k), wherein the output comprises one or more of the FAR location, the methylation pattern of the FAR, and the count of the FAR.

Statement 14. The method any one of statements 1-13 further comprising merging each of the counts of the FAR; normalizing the counts of the FAR based on sequence depth; and identifying a FAR that is differentially expressed between the cfDNA sample of the subject and the cancerous samples and the non-cancerous samples.

Statement 15. The method any one of statements 1-14 comprising using the trained machine learning program to determine whether the subject is likely to have or develop metastatic breast cancer, breast cancer recurrence, or both metastatic breast cancer and breast cancer recurrence.

Statement 16. The method any one of statements 1-15 wherein the machine learning program comprises one or more of a RandomForest, a support vector machine (SVM), a neural network, Generalized Linear Model (GLM), Gradient Boosted Model (GBM), Extreme Gradient Boosting (XGB), and a deep learning algorithm.

Statement 17. The method of any one of statements 1-16 wherein the cancerous samples and the non-cancerous samples comprise one or more of breast cancer samples, known metastatic breast cancer samples, breast cancer recurrence samples, samples from a subject that has completed a cancer treatment regimen, and samples from subjects with no evidence of disease using standard of care treatment.

Statement 18. The method of any one of statements 1-17 further comprising treating the subject having the MRD, wherein the treatment comprises one or more of radiation therapy, surgery to remove the cancer, and administering a therapeutic agent to the patient, thereby treating the MRD.

Generally, embodiments of the disclosure comprise the steps of bisulfite conversion of the nucleic acids from a cfDNA sample of a subject using, for example, Whole Genome Bisulfite Sequencing (WGBS) or hybrid probe capture; next generation sequencing the converted and enriched nucleic acids; collecting the methylation data from the targeted regions (e.g., the target regions listed in Table 1); and using a trained machine learning algorithm to determine, for example, the presence or absence of breast cancer, the severity of breast cancer, the histological subtype of breast cancer, or the susceptibility to breast cancer.

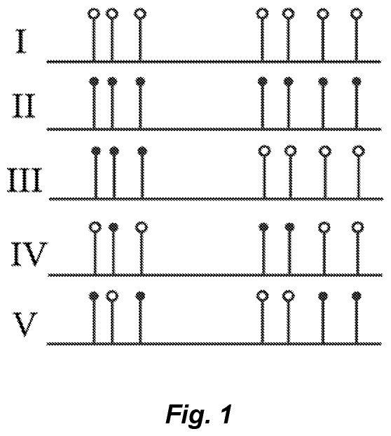

In some embodiments, the methylation data may be used to develop a cancer signature, such as a minimal residual disease (MRD), breast cancer recurrence, or MBC signature indicating the presence of, for example, MRD in a patient or to identify patients at high risk of cancer recurrence or developing MRD. Certain embodiments may be used to detect evidence of MRD prior to clinical recurrence where the non-invasive methods may be easily repeated following the conclusion of a primary treatment regimen. In one embodiment, a method of determining the presence of MRD comprises analyzing methylation patterns of certain target regions of cfDNA. Typically, a beta value, which is a ratio of methylated CpGs at a given locus to the total number of CpGs at the same locus, may be used to develop a differentially methylated region score, or “DMR” score, that my used to determine, for example, the presence or absence of a cancer, or the presence or absence of MRD, or a likelihood of developing MRD, or the likelihood of a cancer recurrence based on a comparison of the DMR value of a test subject compared to the DMR value of a health subject or a control value. Because the source of the test sample is cfDNA, which may be derived from multiple tissue sources, the derived beta value represents an average methylation state across multiple tissues of origin. In contrast, methylation pattern analysis, or fragment level approach, tabulates all possible methylation states for adjacent CpGs, thereby retaining the context of each CpG island. By way of illustration, in FIG. 1, fragments IV and V show a mean beta value across the last 4 CpGs of 0.5 (half the CpGs are methylated), yielding a Δβ value of 0. However, the fragments have completely opposite methylation patterns suggesting separate tissues of origin where one of the fragments may be tumor derived. Thus, this Fragment level methylation pattern analysis allows Boolean (binary) feature classification—that is, evaluating whether or not cancer specific fragments (CSFs) of DNA are present in a given cfDNA sample. This approach may be more sensitive in low tumor burden situations, such as MRD.

In one embodiment, a method of analyzing a methylation pattern of a certain target region comprises the steps of CpG clustering, methylation tabulation, and fragment analysis. The CpG clustering step comprises combining neighboring CpGs into discrete blocks with n to m CpGs where n and m are user-specified. Preferably, these blocks are of a length that is less than the fragment length. A sequence read must span these CpGs to evaluate methylation patterns of CpGs within a selected block. More specifically, the CPG clustering step functions in two stages: first, combine closely adjacent CpGs into contiguous regions and any region with less than n CpGs is removed but regions containing between n and m CpGs and are less than the maximum length are retained. Next, all other regions are recursively split until the user set constraints are met or the region is found to be unsuitable. Sub-division of regions may be performed using k-means clustering based on nearest adjacent CpGs. Target regions that remain may be referred to as ‘fragment assessment regions (FAR).

Generally, methylation tabulation may be performed after the FARs have been selected to count all possible methylation states in each fragment. For example, methylation tabulation may comprise two files as input: a bedGraph listing the genomic coordinates and number of CpGs per FAR, and a bam file containing mapped sequence reads. The bam files may then be filtered to retain only the sequence reads that overlap a FAR. This is done to speed up runtime and reduce the memory footprint of subsequent steps. For each sequence read, the genomic coordinates, mapping information, and the methylation states are recorded in a custom data structure. More specifically, methylation tabulation has the following steps (a) identification of all possible methylation patterns, given the number of CpGs in the FAR, (b) selection of all reads that overlap the FAR, (c) extraction of the methylation states of each CpG in the read that spans the FAR, and (d) counting each distinct methylation pattern in the fragment. If no reads overlap a FAR, all values are assigned as NA. This produces an output in the form of a table with a row detailing, inter alia, the FAR location, the methylation pattern, and a count or value of the methylation pattern.

In the fragment analysis step, fragment counts may be merged from multiple samples, normalizes based on sequencing depth, and examined for fragments that are differentially expressed between groups. Additionally, the output may support data visualization functions, and export functions for further analysis in packages such as SAS, SPSS and Microsoft Excel.

In some embodiments, the biological sample containing the cfDNA that may be examined for methylation patterns is collected from a patient having, for example, a tumor or a mass or is suspected of having a tumor or mass. In some embodiments, the biological sample containing the cfDNA that may be examined for methylation patterns is collected from a patient, for example, after completing a cancer treatment regimen, and may be suspected of having or having MRD. In some embodiment, the biological sample containing the cfDNA may be collected from a patient previously diagnosed as having a cancer, and/or is now diagnosed as being in remission. In some embodiments, the biological sample containing the cfDNA may be collected from a patient that has completed a partial or full regimen of cancer treatment. Preferably, the biological sample is collected through a standard biopsy or a liquid biopsy. The cfDNA may be collected from whole blood, plasma, serum, or urine. In some embodiments, an amount of sample, such as whole blood, may include an amount of about 50 μL to about 5 mL, about 100 μL to about 5 mL, about 150 μL to about 5 mL, about 200 μL to about 5 mL, about 250 μL to about 5 mL, about 300 μL to about 5 mL, about 350 μL to about 5 mL, about 400 μL to about 5 mL, about 450 μL to about 5 mL, about 500 μL to about 5 mL, about 550 μL to about 5 mL, about 600 μL to about 5 mL, about 700 μL to about 5 mL, about 750 μL to about 5 mL, about 800 μL to about 5 mL, about 850 μL to about 5 mL, about 900 μL to about 5 mL, about 950 μL to about 5 mL, about 1 mL to about 5 mL, about 1.5 mL to about 5 mL, about 2 mL to about 5 mL, about 2.5 mL to about 5 mL, or about 3 mL to about 5 mL. In another embodiment, an amount of sample, such as whole blood, may include an amount of about 5 mL to about 10 mL.

Isolation and extraction of cfDNA may be performed through collection of bodily fluids using a variety of techniques. In some cases, collection may comprise aspiration of a bodily fluid from a subject using a syringe. In other cases, collection may comprise pipetting or direct collection of fluid into a collecting vessel.

After collection of bodily fluid, cfDNA may be isolated and extracted using a variety of techniques known to a person of ordinary skill in the art. In some cases, cell-free nucleic acid may be isolated, extracted and prepared using commercially available kits such as the Thermofisher MagMax cfDNA Kit or Qiagen Qiamp® Circulating Nucleic Acid Kit protocol. In other examples, Qiagen Qubit™ dsDNA HS Assay kit protocol, Agilent™ DNA 1000 kit, or TruSeq™ Sequencing Library Preparation; Low-Throughput (LT) protocol, Roche KAPA Hyper Prep Kit, Swift Biosciences Methyl-Seq Library Prep Kit, Nugen Ultra-low Methyl-Seq Kit.

Alternatively, cfDNA may be extracted and isolated by from bodily fluids through a partitioning step in which cfDNAs, as found in solution, are separated from cells and other non-soluble components of the bodily fluid. Partitioning may include, but is not limited to, techniques such as centrifugation or filtration. In other cases, cells may not be partitioned from cfDNA first, but rather lysed. For instance, the genomic DNA of intact cells may be partitioned through selective precipitation.

In some embodiments, the method used to determine the methylation pattern of the one or more target nucleic acids includes methylation sequencing. For example, the methylation pattern of CpG sites within the target regions listed in Table 1 may be detected using DNA methylation sequencing. DNA methylation sequencing can involve, for example, treating DNA from a sample with bisulfite to convert unmethylated cytosine to uracil followed by amplification (such as PCR amplification) of a target nucleic acid within the treated genomic DNA, and sequencing of the resulting amplicon. Sequencing produces nucleotide reads that may be aligned to a genomic reference sequence that may be used to quantitate methylation levels of all the CpGs within an amplicon. Cytosines in non-CpG context may be used to track bisulfite conversion efficiency for each individual sample. The procedure is both time and cost-effective, as multiple samples may be sequenced in parallel using a 96 well plate and generates reproducible measurements of methylation when assayed in independent experiments.

Nucleic acid molecules may be subjected to conditions sufficient to convert unmethylated cytosines in the nucleic acid molecules to uracils (e.g., subsequent to extraction from a sample). For example, to detect DNA methylation, certain embodiments provide for first converting the DNA to be analyzed so that the unmethylated cytosine is converted to uracil. In one embodiment, a chemical reagent that selectively modifies either the methylated or non-methylated form of CpG dinucleotide motifs may be used. Suitable chemical reagents include hydrazine and bisulphite ions and the like. Preferably, isolated DNA is treated with sodium bisulfite (NaHSO₃) which converts unmethylated cytosine to uracil, while methylated cytosines are maintained. Without wishing to be bound by a theory, it is understood that sodium bisulfite reacts readily with the 5,6-double bond of cytosine, but poorly with methylated cytosine. Cytosine reacts with the bisulfite ion to form a sulfonated cytosine reaction intermediate that is susceptible to deamination, giving rise to a sulfonated uracil. The sulfonated group can be removed under alkaline conditions, resulting in the formation of uracil. The nucleotide conversion results in a change in the sequence of the original DNA. It is general knowledge that the resulting uracil has the base pairing behavior of thymine, which differs from cytosine base pairing behavior. To that end, uracil is recognized as a thymine by DNA polymerase. Therefore, after PCR or sequencing, the resultant product contains cytosine only at the position where 5-methylcytosine occurs in the starting template DNA. This makes the discrimination between unmethylated and methylated cytosine possible.

Nucleic acid molecules may also be subjected to further processing including other derivatization processes (e.g., to incorporate, modify, and/or delete one or more sequences, tags, or labels). In some cases, functional sequences (e.g., sequencing adapters, flow cell adapters, sequencing primers, etc.) may be added to nucleic acid molecules to facilitate nucleic acid sequencing. Accordingly, derivatives of nucleic acid molecules from a sample may comprise processed nucleic acid molecules including bisulfite-modified nucleic acid molecules, reverse-transcribed nucleic acid molecules, tagged nucleic acid molecules, barcoded nucleic acid molecules, and other modified nucleic acid molecules.

In some embodiments, methylation pattern of a target region may be determined using one or more of hybrid probe capture (Buckley et al., NAR Genom Bioinform. 2022 Dec. 31; 4(4):lqac099. doi: 10.1093/nargab/lqac099), targeted bisulfite amplicon sequencing, bisulfite DNA treatment, WGBS, bisulfite conversion combined with bisulfite restriction analysis (COBRA), bisulfite PCR, bisulfite modification, bisulfite pyrosequencing, methylated CpG island amplification, CpG binding column based isolation of CpG islands, CpG island arrays with differential methylation hybridization, high performance liquid chromatography, DNA methyltransferase assay, methylation sensitive PCR, cloning differentially methylated sequences, methylation detection following restriction, restriction landmark genomic scanning, methylation sensitive restriction fingerprinting, or Southern blot analysis.

In some embodiments, the one or more hybrid capture probes that hybridize to the plurality of target regions, wherein each of the plurality of the target regions comprise an uracil at each position corresponding to an unmethylated cytosine in the DNA molecule, and wherein each of the one or more hybrid capture probes are complementary to one or more of the plurality of target regions. In some embodiments, the one or more hybrid capture probes that hybridize to the plurality of target regions, wherein each of the plurality of the target regions comprise a thymine at each position corresponding to an unmethylated cytosine in the DNA molecule.

In some embodiments, the one or more hybrid capture probes is configured to hybridize to: a) a nucleotide sequence of the plurality of target regions comprising uracil at each position corresponding to a cytosine of a CpG site of the nucleic acid molecule; b) a nucleotide sequence of the plurality of target regions comprising uracil at each position corresponding to a cytosine of a CpG site of the nucleic acid molecule; or c) a nucleotide sequence of the plurality of target regions comprising cytosine at each position corresponding to a cytosine of a CpG site of the nucleic acid molecule.

In one embodiment, the method used to determine the methylation level of the one or more target regions in cfDNA is WGBS (Cokus, et al. 2008. Nature, 452 (7184): 215-219; Lister, et al. 2009. Nature, 462 (7271): 315-322; Harris, et al. 2010. Nat Biotechnol, 28 (10): 1097-1105).

Other methods to assay the methylation status of CpG sites can also be used. Numerous DNA methylation detection methods are known in the art, including but not limited to hybrid probe capture (REF), methylation-specific enzyme digestion (Singer-Sam et al., Nucleic Acids Res. 18 (3): 687, 1990; Taylor et al., Leukemia 15 (4): 583-9, 2001), methylation-specific PCR (MSP or MSPCR) (Herman et al., Proc Natl Acad Sci USA 93 (18): 9821-6, 1996), methylation-sensitive single nucleotide primer extension (MS-SnuPE) (Gonzalgo et al., Nucleic Acids Res. 25(12): 2529-31, 1997), restriction landmark genomic scanning (RLGS) (Kawai, Mol Cell Biol. 14 (11): 7421-7, 1994; Akama, et al., Cancer Res. 57 (15): 3294-9, 1997), and differential methylation hybridization (DMH) (Huang et al., Hum Mol Genet. 8 (3): 459-70, 1999). In some embodiments, the methylation levels may be determined using one or more DNA methylation sequencing assays with or without bisulfite treatment of DNA.

In one embodiment, Reduced Representation Bisulfite Sequencing (RRBS) is used to measure methylation levels of a target region. Generally, RRBS begins with the treatment of nucleic acid with bisulfite to convert all unmethylated cytosines into uracil, followed by restriction enzyme digestion (for example, by an enzyme that recognizes a site that includes a CG sequence such as MspI) and complete fragment sequencing after coupling with an adapter ligand. The selection of the restriction enzyme enriches the fragments of the dense regions in CpG, reducing the number of redundant sequences that can map multiple positions of the gene during the analysis. Therefore, RRBS reduces the sample complexity of the nucleic acid sample by selecting a subset (e.g., by size selection using preparative gel electrophoresis) of restriction fragments for sequencing. In opposition to the sequencing of the complete genome with bisulfite, each fragment produced by restriction enzyme digestion contains information on DNA methylation for at least one CpG dinucleotide. Therefore, RRBS enriches the sample in promoters, CpG islands, and other genomic characteristics with a high frequency of restriction enzyme cleavage sites in these regions and, thus, provides an assay to assess the methylation status of one or more genomic loci.

A typical protocol for RRBS comprises the steps of digesting a sample of nucleic acid with a restriction enzyme such as Mspl, filling with projections and A-tails, ligating adapters, conversion with bisulfite, and PCR. See, for example, Gu et al. (2010), Nat Methods 7:133-6; Meissner et al (2005), Nucleic Acids Res. 33:5868-77.

In some embodiments, identifying, for example, the presence and/or severity of a cancer, such as metastatic breast cancer, identifying breast cancer recurrence, identifying susceptibility to breast cancer recurrence, identifying MRD, identifying susceptibility to MRD, or identifying MRD after the subject has concluded a cancer treatment regimen in a subject may comprise using hybrid capture probes configured to selectively enrich nucleic acid molecules (e.g., DNA or RNA molecules) or sequences thereof. Such probes may be pull-down probes (e.g., bait sets). Selectively enriched nucleic acid molecules or sequences thereof may correspond to one or more target regions in the methylation profile of the data set. The presence of particular sequences, modifications (e.g., methylation states), deletions, additions, single nucleotide polymorphisms, copy number variations, or other features in the selectively enriched nucleic acid molecules or sequences thereof may be indicative of, for example, a presence and/or severity of a breast cancer the presence or absence of MRD, or susceptibility to MRD, or the presence of absence of MRD or susceptibility to developing MRD during or after a cancer treatment regimen (e.g., adjuvant or neoadjuvant treatment). The probes may be selective (i.e., complementary to the target regions) for a subset of certain target regions of Table 1 in the cfDNA sample and/or for differentially methylated regions (e.g., CpG sites, CpA, sites, CpT sites, and/or CpC sites). The probes may be configured to selectively enrich nucleic acid molecules (e.g., DNA or RNA molecules) or sequences thereof corresponding to a plurality of target nucleic acid of target genomic sequences, such as the subset of the one or more genomic regions in the cell-free biological sample and/or differentially methylated regions (e.g., CpG sites, CpA, sites, CpT sites, and/or CpC sites). The probes may be nucleic acid molecules (e.g., DNA or RNA molecules) having sequence complementarity with target nucleic acid sequences. These nucleic acid molecules may be primers or enrichment sequences. The assaying of the nucleic acid molecules of the sample (e.g., cell-free biological sample) using probes that are selected for target nucleic acid sequences may comprise use of array hybridization, polymerase chain reaction (PCR), or nucleic acid sequencing (e.g., DNA sequencing or RNA sequencing). The number of target nucleic acid sequences selectively enriched using such a scheme may comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 50, at least 100, at least 150, at least 200, at least 300, at least 500, at least 1000, at least 2000, at least 3000, at least 4000, at least 5000, or more than 5000 different target nucleic acid sequences of the target genomic regions. Use of such probes for enrichment of target nucleic acids may be termed “hybrid capture”. Use of such hybrid capture probes may take place prior to or after bisulfite conversion (if applicable). Examples of target nucleic acid sequences include those associated with the target regions included in Table 1.

In some embodiments, cfDNA samples may be collected from plasma samples in a subject having or suspected of having a breast cancer, recurrence of breast cancer, MBC, or MRD. The extracted cfDNAs are contacted with a bisulfite compound to undergo bisulfite conversion. A library may then be prepared from the bisulfite converted nucleic acids. A portion of the library may then be hybridized with various capture probes in which the capture probes are complementary to one or more DNA strands of a target region or complementary to the target sequence in which the CpG islands and the like are modified because of bisulfite conversion.

Nonlimiting examples of methods for preparing the library include using a transposome-mediated protocol with dual indexing, and/or a kit (e.g., TruSeq Methyl Capture EPIC Library Prep Kit, Illumina, CA, USA, Kapa Hyper Prep Kit (Kapa Biosystems). Adapters such as TruSeq DNA LT adapters (Illumina) can be used for indexing. Sequencing is performed on the library using a sequencer platform (e.g., MiSeq, HiSeq, Illumina Roche KAPA Hyper Prep Kit, Swift Biosciences Methyl-Seq Library Prep Kit, Nugen Ultra-low Methyl-Seq Kit).

Preferably, the capture probe is an DNA probe or an RNA probe that is complementary to at least a portion of a nucleic acid sequence of a target genomic region or complementary to at least a portion of a nucleic acid sequence of a target genomic region that is modified because of bisulfite conversion. In some embodiments, several capture probes may be used that overlap one or more portions of each target genomic region (i.e., tiling). In this way, numerous capture probes may be used to saturate a target genomic region to ensure enrichment of that target genomic region. Capture probes may be designed using publicly available software or purchased commercially.

Generally, the target strand can be the “positive” strand (e.g., the strand transcribed into mRNA, and subsequently translated into a protein) or the complementary “negative” strand. In some embodiments, an assay panel includes sets of two probes, one probe targeting the positive strand and the other probe targeting the negative strand of a target genomic region.

In some embodiments, a capture probe may be tagged with an affinity tag such as biotin, streptavidin, digitonin or other tags that are known in the art. After hybridization to target genomic region, the biotinylated capture probes may be “pulled-down” from the library using streptavidin beads or other streptavidin coated surface, thus causing enrichment of the targeted genomic region. In other embodiments, the probes may be immobilized on an assay panel comprising, for example, a solid surface such as a glass microarray slide. In some embodiments, exemplary assay panels comprise at least 1,000, 2,000, 2,500, 5,000, 10,000, 12,000, 15,000, 20,000, 25,000, 30,000, 35,000, or 40,000 hybrid capture probes complementary to a target region disclosed in Table 1. In some embodiments, the assay panels comprise about 100, about 200, about 300, about 400, about 500, about 600, about 700, about 800, about 900, about 1000, about 1,500, about 2,000, about 2,500, about 3,000, about 3,500, about 4,000, about 4,500, about 5,000, about 5,500, about 6,00, about 6,500, about 7,000, about 7,500, about 8,000, about 8,500, about 9,000, about 9,500, or about 10,000 pairs of hybrid capture probes complementary to a target region disclosed in Table 1. In some embodiments, each of the hybrid capture probes on the assay panel comprises less than 300, 250, 200, or 150 nucleotides. In some embodiments, each of the probes on the panel comprises 100-150 nucleotides.

The enriched target genomic region then may be sequenced using next generation sequencing techniques, such as pyrosequencing, single-molecule real-time sequencing, sequencing by synthesis, sequencing by ligation (SOLID sequencing), and nanopore sequencing.

Nucleic acid molecules (e.g., extracted cfDNA) or derivatives thereof may be subjected to sequencing to provide a plurality of sequencing reads. Sequencing reads may be aligned with and/or analyzed with regard to a reference genome. Based at least in part on sequencing reads, an absolute amount or relative amount of nucleic acid molecules (including an absolute or relative level of methylation within said molecules) corresponding to one or more genomic regions may be measured. Alternatively, sequencing reads may not be used to determine an amount or relative amount of nucleic acid molecules. A data set comprising a genomic profile (e.g., methylation profile) of one or more genomic regions of a sample may be generated based at least in part on sequencing reads. Sequencing reads may be processed to identify methylation patterns of the target regions of the cfDNA in a sample.

Sequence identification may be performed by sequencing, array hybridization (e.g., Affymetrix), or nucleic acid amplification (e.g., PCR), for example. Sequencing may be performed by any suitable sequencing methods, such as massively parallel sequencing (MPS), paired-end sequencing, high-throughput sequencing, next-generation sequencing (NGS), shotgun sequencing, single-molecule sequencing, nanopore sequencing, nanopore sequencing with direct detection or inference of methylation status, semiconductor sequencing, pyrosequencing, sequencing-by-synthesis (SBS), sequencing-by-ligation, sequencing-by hybridization, and RNA-Seq (Illumina).

Sequencing and/or preparing a nucleic acid sample for sequencing may comprise performing one or more nucleic acid reactions such as one or more nucleic acid amplification processes (e.g., of DNA or RNA molecules). Nucleic acid amplification may comprise, for example, reverse transcription, primer extension, asymmetric amplification, rolling circle amplification, ligase chain reaction, polymerase chain reaction (PCR), and multiple displacement amplification. Examples of PCR methods include digital PCR (dPCR), emulsion PCR (ePCR), quantitative PCR (qPCR), real-time PCR (RT-PCR), hot start PCR, multiplex PCR, asymmetric PCR, nested PCR, and assembly PCR. A suitable number of rounds of nucleic acid amplification (e.g., PCR, such as qPCR, RT-PCR, dPCR, etc.) may be performed to sufficiently amplify an initial amount of nucleic acid molecule (e.g., DNA molecule) or derivative thereof to a desired input quantity for subsequent sequencing. In some cases, the PCR may be used for global amplification of nucleic acid molecules. This may comprise using adapter sequences that may be first ligated to different molecules followed by PCR amplification using universal primers. PCR may be performed using any of a number of commercial kits, e.g., provided by Life Technologies, Affymetrix, Promega, Qiagen, etc. In other cases, only certain target nucleic acids within a population of nucleic acids may be amplified. Specific primers, possibly in conjunction with adapter ligation, may be used to selectively amplify certain targets for downstream sequencing. In some cases, nested primers may be used to target specific genomic regions. Nucleic acid amplification may comprise targeted amplification of one or more genetic loci, genomic regions, cfDNA target regions, or differentially methylated regions (e.g., CpG sites, CpA, sites, CpT sites, and/or CpC sites), and in particular, the target regions listed in Table 1 (below). In some cases, nucleic acid amplification is performed after bisulfite conversion. Such a procedure may be termed targeted bisulfite amplicon sequencing (TBAS). Nucleic acid amplification may comprise the use of one or more primers, probes, enzymes (e.g., polymerases), buffers, and deoxyribonucleotides.

Nucleic acid amplification may be isothermal or may comprise thermal cycling. Thermal cycling may involve changing a temperature associated with various processes of nucleic acid amplification including, for example, initialization, denaturation, annealing, and extension. Sequencing may comprise use of simultaneous reverse transcription (RT) and PCR, such as a OneStep RT-PCR kit protocol by Qiagen, NEB, Thermo Fisher Scientific, or Bio-Rad.

Nucleic acid molecules (e.g., DNA or RNA molecules) or derivatives thereof may be labeled or tagged, e.g., with identifiable tags, to allow for multiplexing of a plurality of samples. For example, every nucleic acid molecule or derivative thereof associated with a given sample or subject may be tagged or labeled (e.g., with a barcode such as a nucleic acid barcode sequence or a fluorescent label). Nucleic acid molecules or derivatives thereof associated with other samples or subjects may be tagged or labels with different tags or labels such that nucleic acid molecules or derivatives thereof may be associated with the sample or subject from which they derive. Such tagging or labeling also facilitates multiplexing such that nucleic acid molecules or derivatives thereof from multiple samples and/or subjects may be analyzed (e.g., sequenced) at the same time. Any number of samples may be multiplexed. For example, a multiplexed reaction may contain nucleic acid molecules or derivatives thereof from at least about 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, or more than 100 initial samples. Such samples may be derived from the same or different subjects. For example, a plurality of samples may be tagged with sample barcodes (e.g., nucleic acid barcode sequences) such that each nucleic acid molecule (e.g., DNA molecule) or derivative thereof may be traced back to the sample (and/or the subject) from which the nucleic acid molecule originated. Sample barcodes may permit samples from multiple subjects to be differentiated from one another, which may permit sequences in such samples to be identified simultaneously, such as in a pool. Tags, labels, and/or barcodes may be attached to nucleic acid molecules or derivatives thereof by ligation, primer extension, nucleic acid amplification, or another process. In some cases, nucleic acid molecules or derivatives thereof of a particular sample may be tagged, labeled, or barcoded with different tags, labels, or barcodes (e.g., unique molecular identifiers) such that different nucleic acid molecules or derivatives thereof deriving from the same sample may be differentially tagged, labeled, or barcoded. In some cases, nucleic acid molecules or derivatives thereof from a given sample may be labeled with both different labels and identical labels, such that each nucleic acid molecule or derivative thereof associated with the sample includes both a unique label and a shared label.

After subjecting the nucleic acid molecules or derivatives thereof to sequencing, suitable bioinformatics processes may be performed on the sequence reads to generate the data set comprising the methylation pattern of one or more target regions of the cfDNA sample. For example, sequence reads may be aligned to one or more reference genomes (e.g., a human genome). The aligned sequence reads may be quantified at one or more genomic loci or target regions to generate the data set comprising the methylation pattern profile of one or more target regions of the cell-free biological sample. Quantification of sequences may be expressed as un-normalized or normalized values.

In some embodiments, alignment of bisulfite converted DNA is performed using a software program such as Bismark (Krueger et al. (2011) Bioinformatics, 27 (11): 157171). Bismark performs both read mapping and methylation calling in a single step and its output discriminates between cytosines in CpG, CHG and CHH contexts. Bismark is released under the GNU GPLv3+ license. The source code is freely available at bioinformatics.bbsrc.ac.uk/projects/bismark/. In some embodiments, differential methylation is calculated for specific loci/regions using, for example, one or more publicly available programs to analyze and/or determine methylation levels or a target polynucleotide region. In some embodiments, the method used to analyze and/or determine methylation levels of a target polynucleotide region include Metilene (Juhling et al., Genome Res., 2016; 26 (2): 256-262) or GenomeStudio Software available online from Illumina, Inc. Other methods of determining differentially methylated target polynucleotide regions are described in Hovestadt et al., 2014; Nature, 510 (7506), 537-541.

In some embodiments, the target genomic regions that are examined to determine the presence or absence of breast cancer in a subject comprise at least 5%, at least 10%, at least 15%, at least 20%, at least 25%, at least 30%, at least 40%, at least 45%, at least 50%, at least 55%, at least 60%, at least 65%, at least 70%, at least 75%, at least 80%, a least 85%, at least 90%, at least 95%, at least 96%, at least 97%, at least 98%, at least 99%, or 100% of the target regions listed in Table 1.

In some embodiments, the target regions that are examined to determine the severity of breast cancer (i.e., stage I, stage II, stage III, or stage IV cancer) subject comprise at least 5%, at least 10%, at least 15%, at least 20%, at least 25%, at least 30%, at least 40%, at least 45%, at least 50%, at least 55%, at least 60%, at least 65%, at least 70%, at least 75%, at least 80%, a least 85%, at least 90%, at least 95%, at least 96%, at least 97%, at least 98%, at least 99%, or 100% of the target regions listed in Table 1.

Some embodiments may be used to determine the presence of MBC, breast cancer recurrence, and/or Minimum residual disease (MRD), which is the name given to small numbers of cancer cells that remain in the person during treatment, or after treatment when the patient is in remission or thought to be in remission. It is the major cause of relapse in cancer.

Target genomic regions that are examined to determine the presence of MBC in a subject comprise at least 5%, at least 10%, at least 15%, at least 20%, at least 25%, at least 30%, at least 40%, at least 45%, at least 50%, at least 55%, at least 60%, at least 65%, at least 70%, at least 75%, at least 80%, a least 85%, at least 90%, at least 95%, at least 96%, at least 97%, at least 98%, at least 99%, or 100% of the target regions listed in Table 1.

Target genomic regions that are examined to determine breast cancer recurrence in a subject comprise at least 5%, at least 10%, at least 15%, at least 20%, at least 25%, at least 30%, at least 40%, at least 45%, at least 50%, at least 55%, at least 60%, at least 65%, at least 70%, at least 75%, at least 80%, a least 85%, at least 90%, at least 95%, at least 96%, at least 97%, at least 98%, at least 99%, or 100% of the target regions listed in Table 1.

Target genomic regions that are examined to determine the susceptibility of a subject to breast cancer recurrence at the time of diagnosis comprise at least 5%, at least 10%, at least 15%, at least 20%, at least 25%, at least 30%, at least 40%, at least 45%, at least 50%, at least 55%, at least 60%, at least 65%, at least 70%, at least 75%, at least 80%, a least 85%, at least 90%, at least 95%, at least 96%, at least 97%, at least 98%, at least 99%, or 100% of the target regions listed in Table 1.

Target genomic regions that are examined to determine the presence of MRD in a subject comprise at least 5%, at least 10%, at least 15%, at least 20%, at least 25%, at least 30%, at least 40%, at least 45%, at least 50%, at least 55%, at least 60%, at least 65%, at least 70%, at least 75%, at least 80%, a least 85%, at least 90%, at least 95%, at least 96%, at least 97%, at least 98%, at least 99%, or 100% of the target regions listed in Table 1.

Target genomic regions that are examined to determine the susceptibility to MRD in a subject comprise at least 5%, at least 10%, at least 15%, at least 20%, at least 25%, at least 30%, at least 40%, at least 45%, at least 50%, at least 55%, at least 60%, at least 65%, at least 70%, at least 75%, at least 80%, a least 85%, at least 90%, at least 95%, at least 96%, at least 97%, at least 98%, at least 99%, or 100% of the target regions listed in Table 1.

Target genomic regions that are examined to determine the presence or absence, or the susceptibility to MRD in a subject undergoing a cancer treatment regimen comprise at least 5%, at least 10%, at least 15%, at least 20%, at least 25%, at least 30%, at least 40%, at least 45%, at least 50%, at least 55%, at least 60%, at least 65%, at least 70%, at least 75%, at least 80%, a least 85%, at least 90%, at least 95%, at least 96%, at least 97%, at least 98%, at least 99%, or 100% of the target regions listed in Table 1.

Target genomic regions that are examined to determine the presence or absence, or the susceptibility to MRD in a subject after completion of a cancer treatment regimen comprise at least 5%, at least 10%, at least 15%, at least 20%, at least 25%, at least 30%, at least 40%, at least 45%, at least 50%, at least 55%, at least 60%, at least 65%, at least 70%, at least 75%, at least 80%, a least 85%, at least 90%, at least 95%, at least 96%, at least 97%, at least 98%, at least 99%, or 100% of the target regions listed in Table 1.

In some embodiments, target genomic regions that are examined to determine, for example, the presence of MBC, the presence of or susceptibility to MRD, or the presence of or susceptibility to breast cancer recurrence in a subject may comprise about 20% to about 30%, about 30% to about 40%, about 40% to about 50%, about 50% to about 60%, about 60% to about 70%, about 70% to about 80%, about 80% to about 90%, about 95%, about 96%, about 97%, about 98%, about 99%, or about 100% of the target regions listed in Table 1.

In some embodiments, target genomic regions that are examined to determine for example, the presence of MBC, the presence of or susceptibility to MRD, or the presence of or susceptibility of breast cancer recurrence in a subject may comprise about 50, about 100, about 150, about 200, about 250, about 300, about 350, about 400, about 450, about 500, about 550, about 600, about 650, about 700, about 750, about 800 about 850, about 900, about 950, about 1000, about 1050, about 1100, about 1150, about 1200, about 1250, about 1300, about 1350, about 1400, or about 1450, about 1500, about 1550, or about 1564 of the target regions listed in Table 1.

TABLE 1
Exemplary target regions analyzed for methylation patterns.
Target regions correspond to chromosomes, start, and stop
positions corresponding to the human reference genome
GRCh37 (UCSC version hg19; www.genome.ucsc.edu).

| chr | start | end |
|---|---|---|
| C1 | 10194 | 11229 |
| C1 | 564100 | 565425 |
| C1 | 566667 | 568070 |
| C1 | 568581 | 569772 |
| C1 | 714358 | 715580 |
| C1 | 823591 | 825860 |
| C1 | 843973 | 845617 |
| C1 | 844665 | 845757 |
| C1 | 867163 | 868225 |
| C1 | 868868 | 870121 |
| C1 | 870941 | 872090 |
| C1 | 912133 | 913268 |
| C1 | 942791 | 943894 |
| C1 | 1002238 | 1003301 |
| C1 | 1011666 | 1012847 |
| C1 | 1012176 | 1013357 |
| C1 | 1015217 | 1016828 |
| C1 | 1052410 | 1053511 |
| C1 | 1068247 | 1069383 |
| C1 | 1098573 | 1099950 |
| C1 | 1099374 | 1100562 |
| C1 | 1108936 | 1110195 |
| C1 | 1147797 | 1148914 |
| C1 | 1166866 | 1167912 |
| C1 | 1223768 | 1224863 |
| C1 | 1226688 | 1227744 |
| C1 | 1228846 | 1229909 |
| C1 | 1267350 | 1268581 |
| C1 | 1267644 | 1268796 |
| C1 | 1268216 | 1269394 |
| C1 | 1272731 | 1273943 |
| C1 | 1289965 | 1291019 |
| C1 | 1309012 | 1310137 |
| C1 | 1341136 | 1342248 |
| C1 | 1355352 | 1356466 |
| C1 | 1361924 | 1363076 |
| C1 | 1365678 | 1366734 |
| C1 | 1371877 | 1373012 |
| C1 | 1374304 | 1375618 |
| C1 | 1474820 | 1476060 |
| C1 | 1475567 | 1476648 |
| C1 | 1522695 | 1523916 |
| C1 | 1541491 | 1542589 |
| C1 | 1564399 | 1565563 |
| C1 | 1564771 | 1566009 |
| C1 | 1607286 | 1608588 |
| C1 | 1607633 | 1608818 |
| C1 | 1609008 | 1610212 |
| C1 | 1642780 | 1643938 |
| C1 | 1643108 | 1644197 |
| C1 | 1654334 | 1655606 |
| C1 | 1689110 | 1690236 |
| C1 | 1695085 | 1696323 |
| C1 | 1696192 | 1697293 |
| C1 | 1710350 | 1711681 |
| C1 | 1850395 | 1851488 |
| C1 | 1956696 | 1957782 |
| C1 | 2043189 | 2044778 |
| C1 | 2053010 | 2055721 |
| C1 | 2054758 | 2055830 |
| C1 | 2054947 | 2056094 |
| C1 | 2055163 | 2056328 |
| C1 | 2064159 | 2065258 |
| C1 | 2064556 | 2065608 |
| C1 | 2079713 | 2080994 |
| C1 | 2081815 | 2083028 |
| C1 | 2120478 | 2121815 |
| C1 | 2138392 | 2139453 |
| C1 | 2163089 | 2164359 |
| C1 | 2200175 | 2201380 |
| C1 | 2221475 | 2222847 |
| C1 | 2231793 | 2233358 |
| C1 | 2243667 | 2244920 |
| C1 | 2344910 | 2346159 |
| C1 | 2401440 | 2402551 |
| C1 | 2508660 | 2509773 |
| C1 | 2523221 | 2524766 |
| C1 | 2538234 | 2539515 |
| C1 | 2591384 | 2592796 |
| C1 | 2603151 | 2604621 |
| C1 | 2605221 | 2606863 |
| C1 | 2754582 | 2755642 |
| C1 | 2771254 | 2772414 |
| C1 | 2799115 | 2800195 |
| C1 | 2822206 | 2823322 |
| C1 | 2827425 | 2828930 |
| C1 | 2849944 | 2851558 |
| C1 | 2937975 | 2939691 |
| C1 | 2978073 | 2979413 |
| C1 | 2979145 | 2980344 |
| C1 | 2979526 | 2981012 |
| C1 | 2999208 | 3000464 |
| C1 | 3025461 | 3026885 |
| C1 | 3036307 | 3037397 |
| C1 | 3037774 | 3038911 |
| C1 | 3051301 | 3052691 |
| C1 | 3087949 | 3089135 |
| C1 | 3110619 | 3112409 |
| C1 | 3148105 | 3149319 |
| C1 | 3157696 | 3158877 |
| C1 | 3182399 | 3183536 |
| C1 | 3194987 | 3196070 |
| C1 | 3203568 | 3204725 |
| C1 | 3203773 | 3204952 |
| C1 | 3208295 | 3209434 |
| C1 | 3229316 | 3230380 |
| C1 | 3251414 | 3252910 |
| C1 | 3320688 | 3322166 |
| C1 | 3327701 | 3329433 |
| C1 | 3375759 | 3376832 |
| C1 | 3446331 | 3447416 |
| C1 | 3613125 | 3614328 |
| C1 | 3797003 | 3798072 |
| C1 | 4152051 | 4153109 |
| C1 | 4270713 | 4271905 |
| C1 | 4559662 | 4560881 |
| C1 | 4944905 | 4945983 |
| C1 | 5337958 | 5338973 |
| C1 | 5393752 | 5395173 |
| C1 | 5652699 | 5653738 |
| C1 | 5773089 | 5774325 |
| C1 | 6067004 | 6068127 |
| C1 | 6185975 | 6187174 |
| C1 | 6204717 | 6205775 |
| C1 | 6268321 | 6269382 |
| C1 | 6514122 | 6515171 |
| C1 | 6525846 | 6527034 |
| C1 | 6673043 | 6674111 |
| C1 | 7552358 | 7553430 |
| C1 | 7565426 | 7566704 |
| C1 | 7617529 | 7618577 |
| C1 | 7876809 | 7878057 |
| C1 | 7886747 | 7887965 |
| C1 | 8276696 | 8277858 |
| C1 | 8276890 | 8277961 |
| C1 | 8930635 | 8932066 |
| C1 | 8935988 | 8937314 |
| C1 | 9377433 | 9378521 |
| C1 | 9869406 | 9870422 |
| C1 | 9887311 | 9888452 |
| C1 | 10088673 | 10089829 |
| C1 | 10698494 | 10699607 |
| C1 | 10948597 | 10949700 |
| C1 | 11071326 | 11072498 |
| C1 | 11118164 | 11119438 |
| C1 | 11309704 | 11310802 |
| C1 | 11399339 | 11400369 |
| C1 | 11561057 | 11562159 |
| C1 | 11561213 | 11562268 |
| C1 | 11795563 | 11796631 |
| C1 | 11836169 | 11837208 |
| C1 | 11956188 | 11957709 |
| C1 | 12187498 | 12188546 |
| C1 | 12694045 | 12695126 |
| C1 | 13012090 | 13013776 |
| C1 | 13038661 | 13040410 |
| C1 | 13147659 | 13149362 |
| C1 | 13207667 | 13209341 |
| C1 | 13212681 | 13214418 |
| C1 | 13340067 | 13341258 |
| C1 | 13522875 | 13524169 |
| C1 | 15475565 | 15477105 |
| C1 | 15928879 | 15930121 |
| C1 | 15945154 | 15946309 |
| C1 | 16162979 | 16164091 |
| C1 | 16163123 | 16164520 |
| C1 | 16278432 | 16279450 |
| C1 | 16472387 | 16473591 |
| C1 | 16533257 | 16534615 |
| C1 | 16552941 | 16554022 |
| C1 | 16860948 | 16862020 |
| C1 | 16861659 | 16862699 |
| C1 | 16874189 | 16875334 |
| C1 | 16974032 | 16975180 |
| C1 | 17012090 | 17013196 |
| C1 | 17018361 | 17019527 |
| C1 | 17022561 | 17023622 |
| C1 | 17025787 | 17026985 |
| C1 | 17033327 | 17034385 |
| C1 | 17086058 | 17087158 |
| C1 | 17091915 | 17093482 |
| C1 | 17198621 | 17199666 |
| C1 | 17199442 | 17200515 |
| C1 | 17230037 | 17231114 |
| C1 | 17264942 | 17266052 |
| C1 | 18901842 | 18903245 |
| C1 | 19110130 | 19111478 |
| C1 | 19111263 | 19112295 |
| C1 | 19121254 | 19122275 |
| C1 | 19156611 | 19157774 |
| C1 | 19192496 | 19193530 |
| C1 | 19407137 | 19408477 |
| C1 | 19599915 | 19601347 |
| C1 | 19651428 | 19652664 |
| C1 | 19664950 | 19665994 |
| C1 | 19669940 | 19670960 |
| C1 | 20569099 | 20570347 |
| C1 | 21008803 | 21010028 |
| C1 | 21141836 | 21143060 |
| C1 | 21142125 | 21143208 |
| C1 | 21348724 | 21350414 |
| C1 | 21659457 | 21660655 |
| C1 | 21724033 | 21725143 |
| C1 | 21995373 | 21996432 |
| C1 | 22097677 | 22099308 |
| C1 | 22149609 | 22150864 |
| C1 | 22203006 | 22204105 |
| C1 | 22224926 | 22225938 |
| C1 | 22420231 | 22421392 |
| C1 | 22480128 | 22481205 |
| C1 | 22755097 | 22756333 |
| C1 | 23110687 | 23112133 |
| C1 | 23224614 | 23225625 |
| C1 | 23235135 | 23236412 |
| C1 | 24152407 | 24153547 |
| C1 | 24274428 | 24275643 |
| C1 | 24357994 | 24359067 |
| C1 | 24626673 | 24627952 |
| C1 | 25070164 | 25071464 |
| C1 | 25240227 | 25241312 |
| C1 | 25257273 | 25258332 |
| C1 | 25257949 | 25259037 |
| C1 | 25593478 | 25594624 |
| C1 | 25694631 | 25695837 |
| C1 | 25714355 | 25715988 |
| C1 | 25757384 | 25758477 |
| C1 | 25809633 | 25811249 |
| C1 | 25889709 | 25890808 |
| C1 | 26076138 | 26077293 |
| C1 | 26439202 | 26440307 |
| C1 | 26463592 | 26465419 |
| C1 | 26503312 | 26504402 |
| C1 | 26527223 | 26528414 |
| C1 | 26621484 | 26622583 |
| C1 | 26871795 | 26872923 |
| C1 | 26921602 | 26922704 |
| C1 | 26948031 | 26949242 |
| C1 | 27144843 | 27146195 |
| C1 | 27189424 | 27190646 |
| C1 | 27247706 | 27248747 |
| C1 | 27390287 | 27391688 |
| C1 | 27675541 | 27676914 |
| C1 | 27683382 | 27684467 |
| C1 | 27686686 | 27687902 |
| C1 | 27960899 | 27962449 |
| C1 | 28180200 | 28181582 |
| C1 | 28180695 | 28182483 |
| C1 | 28573219 | 28574367 |
| C1 | 28577592 | 28578844 |
| C1 | 28641014 | 28642185 |
| C1 | 28844651 | 28845714 |
| C1 | 29075234 | 29076383 |
| C1 | 30027752 | 30029255 |
| C1 | 30239650 | 30240736 |
| C1 | 30463511 | 30464658 |
| C1 | 31190070 | 31191193 |
| C1 | 31505512 | 31506801 |
| C1 | 32052151 | 32053261 |
| C1 | 32390621 | 32391688 |
| C1 | 32409572 | 32410754 |
| C1 | 32409850 | 32411020 |
| C1 | 32479187 | 32480252 |
| C1 | 32720565 | 32721703 |
| C1 | 32807764 | 32808828 |
| C1 | 33439081 | 33440273 |
| C1 | 33516201 | 33517386 |
| C1 | 33517461 | 33518577 |
| C1 | 33517596 | 33518686 |
| C1 | 33517802 | 33518909 |
| C1 | 33815259 | 33816383 |
| C1 | 34160625 | 34161656 |
| C1 | 34890735 | 34891764 |
| C1 | 35358189 | 35359275 |
| C1 | 36039089 | 36040306 |
| C1 | 36039310 | 36040413 |
| C1 | 36042133 | 36043244 |
| C1 | 36643965 | 36645110 |
| C1 | 36708751 | 36710015 |
| C1 | 36785359 | 36787252 |
| C1 | 37773395 | 37774516 |
| C1 | 38259256 | 38260308 |
| C1 | 38461085 | 38462176 |
| C1 | 38461396 | 38462462 |
| C1 | 38747147 | 38748222 |
| C1 | 38941756 | 38942834 |
| C1 | 39136332 | 39137383 |
| C1 | 39281151 | 39282211 |
| C1 | 39542743 | 39543764 |
| C1 | 39699106 | 39700927 |
| C1 | 39980644 | 39981738 |
| C1 | 39981009 | 39982588 |
| C1 | 40024774 | 40025885 |
| C1 | 40098242 | 40099323 |
| C1 | 40149162 | 40150261 |
| C1 | 40235353 | 40236648 |
| C1 | 40282208 | 40283549 |
| C1 | 40387656 | 40388830 |
| C1 | 40682836 | 40683936 |
| C1 | 41222578 | 41224061 |
| C1 | 41349586 | 41350735 |
| C1 | 41406895 | 41407981 |
| C1 | 41908347 | 41909374 |
| C1 | 42610994 | 42612337 |
| C1 | 43424952 | 43426219 |
| C1 | 43472657 | 43473961 |
| C1 | 43585328 | 43586471 |
| C1 | 43814166 | 43815310 |
| C1 | 44031008 | 44032124 |
| C1 | 44448044 | 44449168 |
| C1 | 44478513 | 44479791 |
| C1 | 44685737 | 44686800 |
| C1 | 44688238 | 44689415 |
| C1 | 44703603 | 44704741 |
| C1 | 44759508 | 44760604 |
| C1 | 44879657 | 44880707 |
| C1 | 45082204 | 45083438 |
| C1 | 45082514 | 45083785 |
| C1 | 45452222 | 45453347 |
| C1 | 45572535 | 45573582 |
| C1 | 45688381 | 45689511 |
| C1 | 45962896 | 45964154 |
| C1 | 46214893 | 46216001 |
| C1 | 46465207 | 46466343 |
| C1 | 46705515 | 46706664 |
| C1 | 47005528 | 47007041 |
| C1 | 47696403 | 47697543 |
| C1 | 47700522 | 47701799 |
| C1 | 47798347 | 47799409 |
| C1 | 47998885 | 48000001 |
| C1 | 48847732 | 48848815 |
| C1 | 51840423 | 51841469 |
| C1 | 52067253 | 52068317 |
| C1 | 52176704 | 52177874 |
| C1 | 52384171 | 52385257 |
| C1 | 53741936 | 53743073 |
| C1 | 53742119 | 53743218 |
| C1 | 53924826 | 53925971 |
| C1 | 53935446 | 53936521 |
| C1 | 54518712 | 54519822 |
| C1 | 54561488 | 54562713 |
| C1 | 54821353 | 54822603 |
| C1 | 54845645 | 54846768 |
| C1 | 55026529 | 55028056 |
| C1 | 55246596 | 55247716 |
| C1 | 55521410 | 55522514 |
| C1 | 58843059 | 58844085 |
| C1 | 58843886 | 58844959 |
| C1 | 58844076 | 58845342 |
| C1 | 58933375 | 58934385 |
| C1 | 59089760 | 59090861 |
| C1 | 59279850 | 59280927 |
| C1 | 61519390 | 61520530 |
| C1 | 61620776 | 61621830 |
| C1 | 62106420 | 62107637 |
| C1 | 63233970 | 63234976 |
| C1 | 63477074 | 63478080 |
| C1 | 63785403 | 63786651 |
| C1 | 63795049 | 63796162 |
| C1 | 63832637 | 63833762 |
| C1 | 64360862 | 64361899 |
| C1 | 64936638 | 64937737 |
| C1 | 66029441 | 66030607 |
| C1 | 67599918 | 67601018 |
| C1 | 67772972 | 67774042 |
| C1 | 68510301 | 68511387 |
| C1 | 68515779 | 68516919 |
| C1 | 68516889 | 68518044 |
| C1 | 68606748 | 68608429 |
| C1 | 69078905 | 69079996 |
| C1 | 69283854 | 69284988 |
| C1 | 70330690 | 70332001 |
| C1 | 72353878 | 72355626 |
| C1 | 73101262 | 73102313 |
| C1 | 74912886 | 74915484 |
| C1 | 75589900 | 75591106 |
| C1 | 75590634 | 75591767 |
| C1 | 75590817 | 75592038 |
| C1 | 75841146 | 75842311 |
| C1 | 77980716 | 77981827 |
| C1 | 78511546 | 78512630 |
| C1 | 79214467 | 79215544 |
| C1 | 79316235 | 79317391 |
| C1 | 84971148 | 84972257 |
| C1 | 86206013 | 86207031 |
| C1 | 86295826 | 86297069 |
| C1 | 86967579 | 86968766 |
| C1 | 86968248 | 86970121 |
| C1 | 88618842 | 88619851 |
| C1 | 89592208 | 89593422 |
| C1 | 89593260 | 89594847 |
| C1 | 90211599 | 90213657 |
| C1 | 90217346 | 90218787 |
| C1 | 90684609 | 90685706 |
| C1 | 91624389 | 91625512 |
| C1 | 91832022 | 91833059 |
| C1 | 91852291 | 91853646 |
| C1 | 92865692 | 92866761 |
| C1 | 92945913 | 92947273 |
| C1 | 92948357 | 92949446 |
| C1 | 93297052 | 93298083 |
| C1 | 93635586 | 93636612 |
| C1 | 95249146 | 95250604 |
| C1 | 95689769 | 95691257 |
| C1 | 96494188 | 96495257 |
| C1 | 98261844 | 98264061 |
| C1 | 98510647 | 98511759 |
| C1 | 98978395 | 98979531 |
| C1 | 99260135 | 99261233 |
| C1 | 100230829 | 100231918 |
| C1 | 100512500 | 100513667 |
| C1 | 101146628 | 101148034 |
| C1 | 103007291 | 103008432 |
| C1 | 103095865 | 103098271 |
| C1 | 103573168 | 103574450 |
| C1 | 104067689 | 104068727 |
| C1 | 105267216 | 105268250 |
| C1 | 105317685 | 105319073 |
| C1 | 106623177 | 106624282 |
| C1 | 107479562 | 107480595 |
| C1 | 108117116 | 108118270 |
| C1 | 108742361 | 108743407 |
| C1 | 108751862 | 108752926 |
| C1 | 109202655 | 109203907 |
| C1 | 109394757 | 109396386 |
| C1 | 109515015 | 109516055 |
| C1 | 109632207 | 109633277 |
| C1 | 109643495 | 109644823 |
| C1 | 110211478 | 110212595 |
| C1 | 110229840 | 110231206 |
| C1 | 110330396 | 110331544 |
| C1 | 110430147 | 110431328 |
| C1 | 110612313 | 110613363 |
| C1 | 110626271 | 110627351 |
| C1 | 110672452 | 110673661 |
| C1 | 111216100 | 111217162 |
| C1 | 111771530 | 111772660 |
| C1 | 111991287 | 111992398 |
| C1 | 112057985 | 112059222 |
| C1 | 112631202 | 112632416 |
| C1 | 113315622 | 113316735 |
| C1 | 113441371 | 113442555 |
| C1 | 113612568 | 113613669 |
| C1 | 113932520 | 113933556 |
| C1 | 114650735 | 114651793 |
| C1 | 114834153 | 114835189 |
| C1 | 114837685 | 114838712 |
| C1 | 114888306 | 114889494 |
| C1 | 115160147 | 115161211 |
| C1 | 115568422 | 115569557 |
| C1 | 115690217 | 115691302 |
| C1 | 115796337 | 115797875 |
| C1 | 116740766 | 116741816 |
| C1 | 117133328 | 117134925 |
| C1 | 119394576 | 119395885 |
| C1 | 119534730 | 119535835 |
| C1 | 119549085 | 119550245 |
| C1 | 120684752 | 120686337 |
| C1 | 120905162 | 120906473 |
| C1 | 121102417 | 121104683 |
| C1 | 121142844 | 121144078 |
| C1 | 121382640 | 121383957 |
| C1 | 121407800 | 121409369 |
| C1 | 142890188 | 142891273 |
| C1 | 143131047 | 143132449 |
| C1 | 143542472 | 143543561 |
| C1 | 143663862 | 143665289 |
| C1 | 144480828 | 144482371 |
| C1 | 144533204 | 144534563 |
| C1 | 144567067 | 144568343 |
| C1 | 144960220 | 144961315 |
| C1 | 145429558 | 145430576 |
| C1 | 145496494 | 145497532 |
| C1 | 145562057 | 145563317 |
| C1 | 145562397 | 145563536 |
| C1 | 145667850 | 145668990 |
| C1 | 145945417 | 145946621 |
| C1 | 145979798 | 145980933 |
| C1 | 145991295 | 145992402 |
| C1 | 146009304 | 146010527 |
| C1 | 146126373 | 146127842 |
| C1 | 146367366 | 146368467 |
| C1 | 146367855 | 146368981 |
| C1 | 146521719 | 146522835 |
| C1 | 146543755 | 146545046 |
| C1 | 146548157 | 146551590 |
| C1 | 146551300 | 146552500 |
| C1 | 146554929 | 146556124 |
| C1 | 146556441 | 146557479 |
| C1 | 146649015 | 146650340 |
| C1 | 146873776 | 146875101 |
| C1 | 147142786 | 147144032 |
| C1 | 147532897 | 147534124 |
| C1 | 147544213 | 147546415 |
| C1 | 147550608 | 147551927 |
| C1 | 147551074 | 147552235 |
| C1 | 147718324 | 147719451 |
| C1 | 147775221 | 147776488 |
| C1 | 147781831 | 147782917 |
| C1 | 147789747 | 147791585 |
| C1 | 147800491 | 147802397 |
| C1 | 147814910 | 147815942 |
| C1 | 147833421 | 147834554 |
| C1 | 148192802 | 148193924 |
| C1 | 148517250 | 148518454 |
| C1 | 148598533 | 148600305 |
| C1 | 148854302 | 148855374 |
| C1 | 148854690 | 148855759 |
| C1 | 148864475 | 148865615 |
| C1 | 149055103 | 149057398 |
| C1 | 149137388 | 149138545 |
| C1 | 149161908 | 149163047 |
| C1 | 149193260 | 149194480 |
| C1 | 149194001 | 149195215 |
| C1 | 149211680 | 149212762 |
| C1 | 149230272 | 149231458 |
| C1 | 149286024 | 149287210 |
| C1 | 149286313 | 149287451 |
| C1 | 149287207 | 149288400 |
| C1 | 149288307 | 149289396 |
| C1 | 149293213 | 149294413 |
| C1 | 149376780 | 149378703 |
| C1 | 149398340 | 149399601 |
| C1 | 149408672 | 149410385 |
| C1 | 149453635 | 149455305 |
| C1 | 149510569 | 149512100 |
| C1 | 149604054 | 149605538 |
| C1 | 149669756 | 149670851 |
| C1 | 149670152 | 149671422 |
| C1 | 149671700 | 149673164 |
| C1 | 149672893 | 149674156 |
| C1 | 149678879 | 149680141 |
| C1 | 149683598 | 149684737 |
| C1 | 149718113 | 149719285 |
| C1 | 149718909 | 149720056 |
| C1 | 150186324 | 150187659 |
| C1 | 150700069 | 150701531 |
| C1 | 150947340 | 150948447 |
| C1 | 150949017 | 150950727 |
| C1 | 150971162 | 150972455 |
| C1 | 151043548 | 151044749 |
| C1 | 151103369 | 151104476 |
| C1 | 151104789 | 151105979 |
| C1 | 151168094 | 151169227 |
| C1 | 151169404 | 151170627 |
| C1 | 151251865 | 151254038 |
| C1 | 151300126 | 151301209 |
| C1 | 151572403 | 151573514 |
| C1 | 151572942 | 151574060 |
| C1 | 151963862 | 151965440 |
| C1 | 152161151 | 152162315 |
| C1 | 152168756 | 152170072 |
| C1 | 152282409 | 152283966 |
| C1 | 152390228 | 152391339 |
| C1 | 152853520 | 152854621 |
| C1 | 153232779 | 153234136 |
| C1 | 153474883 | 153475916 |
| C1 | 153581586 | 153583566 |
| C1 | 153607374 | 153608655 |
| C1 | 153965264 | 153966324 |
| C1 | 154106870 | 154108195 |
| C1 | 154155869 | 154157265 |
| C1 | 154419712 | 154421096 |
| C1 | 154430099 | 154431294 |
| C1 | 154474785 | 154475880 |
| C1 | 154570237 | 154571275 |
| C1 | 154617162 | 154618588 |
| C1 | 154719854 | 154721556 |
| C1 | 154756643 | 154757674 |
| C1 | 154780886 | 154782162 |
| C1 | 154839201 | 154840410 |
| C1 | 154841749 | 154843247 |
| C1 | 154933471 | 154934527 |
| C1 | 154989506 | 154990565 |
| C1 | 155097990 | 155099086 |
| C1 | 155111343 | 155112495 |
| C1 | 155137904 | 155140265 |
| C1 | 155243307 | 155244708 |
| C1 | 155290107 | 155291259 |
| C1 | 155290379 | 155291501 |
| C1 | 155659131 | 155660317 |
| C1 | 155970450 | 155971984 |
| C1 | 155975946 | 155976970 |
| C1 | 156045705 | 156047402 |
| C1 | 156123749 | 156124960 |
| C1 | 156148489 | 156150224 |
| C1 | 156185738 | 156186883 |
| C1 | 156185972 | 156187140 |
| C1 | 156253648 | 156254670 |
| C1 | 156357261 | 156358413 |
| C1 | 156389918 | 156391113 |
| C1 | 156404907 | 156405996 |
| C1 | 156405637 | 156406847 |
| C1 | 156431534 | 156433128 |
| C1 | 156450133 | 156451170 |
| C1 | 156527126 | 156528357 |
| C1 | 156611829 | 156612913 |
| C1 | 156615998 | 156617445 |
| C1 | 156731286 | 156732292 |
| C1 | 156813950 | 156815070 |
| C1 | 156818022 | 156819506 |
| C1 | 156829527 | 156830961 |
| C1 | 156845787 | 156846914 |
| C1 | 156867530 | 156868887 |
| C1 | 156877352 | 156878642 |
| C1 | 156878067 | 156879148 |
| C1 | 156890082 | 156891380 |
| C1 | 156893040 | 156894175 |
| C1 | 157164117 | 157165443 |
| C1 | 157164498 | 157165747 |
| C1 | 157415665 | 157417094 |
| C1 | 157539972 | 157541477 |
| C1 | 157557199 | 157558221 |
| C1 | 157791441 | 157793454 |
| C1 | 157894862 | 157896067 |
| C1 | 157978425 | 157979496 |
| C1 | 158047607 | 158048858 |
| C1 | 158058710 | 158059954 |
| C1 | 158118991 | 158120203 |
| C1 | 158147098 | 158148237 |
| C1 | 158150121 | 158151389 |
| C1 | 158153866 | 158154903 |
| C1 | 158318543 | 158319689 |
| C1 | 158668216 | 158669267 |
| C1 | 158672313 | 158674181 |
| C1 | 158711610 | 158712777 |
| C1 | 158752751 | 158754620 |
| C1 | 158829731 | 158831129 |
| C1 | 158920064 | 158922138 |
| C1 | 158988636 | 158990040 |
| C1 | 159010152 | 159012384 |
| C1 | 159035254 | 159036848 |
| C1 | 159057018 | 159058052 |
| C1 | 159088166 | 159090048 |
| C1 | 159095659 | 159096870 |
| C1 | 159141406 | 159142780 |
| C1 | 159162529 | 159164422 |
| C1 | 159239956 | 159241786 |
| C1 | 159249988 | 159252369 |
| C1 | 159303683 | 159304871 |
| C1 | 159324719 | 159325924 |
| C1 | 159365427 | 159366474 |
| C1 | 159462204 | 159463257 |
| C1 | 159491060 | 159492238 |
| C1 | 159499448 | 159501556 |
| C1 | 159600730 | 159601771 |
| C1 | 159649535 | 159650696 |
| C1 | 159651732 | 159652986 |
| C1 | 159666830 | 159667850 |
| C1 | 159678414 | 159679576 |
| C1 | 159891417 | 159892697 |
| C1 | 160053848 | 160054900 |
| C1 | 160254891 | 160256011 |
| C1 | 160483659 | 160484696 |
| C1 | 160691932 | 160693326 |
| C1 | 160693379 | 160694596 |
| C1 | 160864189 | 160865597 |
| C1 | 160914786 | 160915914 |
| C1 | 160952116 | 160953463 |
| C1 | 161007561 | 161008698 |
| C1 | 161037026 | 161040375 |
| C1 | 161048900 | 161049960 |
| C1 | 161167605 | 161168828 |
| C1 | 161184370 | 161185686 |
| C1 | 161251440 | 161252689 |
| C1 | 161368946 | 161370025 |
| C1 | 161391675 | 161392777 |
| C1 | 161391873 | 161393005 |
| C1 | 161392642 | 161393701 |
| C1 | 161410969 | 161412085 |
| C1 | 161411692 | 161412822 |
| C1 | 161411876 | 161413016 |
| C1 | 161412408 | 161413490 |
| C1 | 161418741 | 161419816 |
| C1 | 161423767 | 161424823 |
| C1 | 161425940 | 161427062 |
| C1 | 161426344 | 161427775 |
| C1 | 161427168 | 161428609 |
| C1 | 161428151 | 161429318 |
| C1 | 161440837 | 161442244 |
| C1 | 161441355 | 161442432 |
| C1 | 161441466 | 161442493 |
| C1 | 161474245 | 161475339 |
| C1 | 161494484 | 161495593 |
| C1 | 161575679 | 161576821 |
| C1 | 161590715 | 161592185 |
| C1 | 162237488 | 162238504 |
| C1 | 162874252 | 162875308 |
| C1 | 164327900 | 164329035 |
| C1 | 165100305 | 165101315 |
| C1 | 166052365 | 166053599 |
| C1 | 166311774 | 166312875 |
| C1 | 166851197 | 166852257 |
| C1 | 166957951 | 166959183 |
| C1 | 166959119 | 166961309 |
| C1 | 167040849 | 167041969 |
| C1 | 167682978 | 167684066 |
| C1 | 168025049 | 168026175 |
| C1 | 168069616 | 168070661 |
| C1 | 169395991 | 169397410 |
| C1 | 169428892 | 169430325 |
| C1 | 170630027 | 170631213 |
| C1 | 170637117 | 170638270 |
| C1 | 171398061 | 171399079 |
| C1 | 171737047 | 171738080 |
| C1 | 171810109 | 171811290 |
| C1 | 172540748 | 172541967 |
| C1 | 172954275 | 172955917 |
| C1 | 173146145 | 173148191 |
| C1 | 173794525 | 173795779 |
| C1 | 174094618 | 174095676 |
| C1 | 174317241 | 174319126 |
| C1 | 174817564 | 174818647 |
| C1 | 174978357 | 174979515 |
| C1 | 175066309 | 175068069 |
| C1 | 175122330 | 175123741 |
| C1 | 175567877 | 175569062 |
| C1 | 176064055 | 176065127 |
| C1 | 176568784 | 176570172 |
| C1 | 177374530 | 177376099 |
| C1 | 178455562 | 178456772 |
| C1 | 178631203 | 178632228 |
| C1 | 178914606 | 178915940 |
| C1 | 179333735 | 179334993 |
| C1 | 179388055 | 179389142 |
| C1 | 179713288 | 179714451 |
| C1 | 180142552 | 180144069 |
| C1 | 180201904 | 180203103 |
| C1 | 180203607 | 180204771 |
| C1 | 180316344 | 180317953 |
| C1 | 180841158 | 180842324 |
| C1 | 180881874 | 180882961 |
| C1 | 181042343 | 181043716 |
| C1 | 181381953 | 181383119 |
| C1 | 181556181 | 181557506 |
| C1 | 181642743 | 181643960 |
| C1 | 181701309 | 181702558 |
| C1 | 181749202 | 181751068 |
| C1 | 181767204 | 181768448 |
| C1 | 181828742 | 181829868 |
| C1 | 181989899 | 181991181 |
| C1 | 182025613 | 182026983 |
| C1 | 182205801 | 182206829 |
| C1 | 182288994 | 182290072 |
| C1 | 182289079 | 182290132 |
| C1 | 182479132 | 182480290 |
| C1 | 182951611 | 182952788 |
| C1 | 182992203 | 182993378 |
| C1 | 183111323 | 183112333 |
| C1 | 183241363 | 183243015 |
| C1 | 183252572 | 183254204 |
| C1 | 183265910 | 183266931 |
| C1 | 183385313 | 183386927 |
| C1 | 183415829 | 183417068 |
| C1 | 183804906 | 183806358 |
| C1 | 183890623 | 183891959 |
| C1 | 185335455 | 185336593 |
| C1 | 185372964 | 185374216 |
| C1 | 185375916 | 185377013 |
| C1 | 185835181 | 185836559 |
| C1 | 186132712 | 186134126 |
| C1 | 186759847 | 186761466 |
| C1 | 187041225 | 187042323 |
| C1 | 187318121 | 187319271 |
| C1 | 187717185 | 187718281 |
| C1 | 190411659 | 190412967 |
| C1 | 190443709 | 190445613 |
| C1 | 192092597 | 192093991 |
| C1 | 192485366 | 192486871 |
| C1 | 196536951 | 196538098 |
| C1 | 196846659 | 196847918 |
| C1 | 197512847 | 197514650 |
| C1 | 197676763 | 197678552 |
| C1 | 197897646 | 197898850 |
| C1 | 199873251 | 199874387 |
| C1 | 200004206 | 200005369 |
| C1 | 200007201 | 200008386 |
| C1 | 200010839 | 200011989 |
| C1 | 200194666 | 200196425 |
| C1 | 200382401 | 200383679 |
| C1 | 200446084 | 200448065 |
| C1 | 200469846 | 200470893 |
| C1 | 200477687 | 200479518 |
| C1 | 200941116 | 200942230 |
| C1 | 200959975 | 200961996 |
| C1 | 200975742 | 200976782 |
| C1 | 201139950 | 201140981 |
| C1 | 201170028 | 201171078 |
| C1 | 201617527 | 201618649 |
| C1 | 201618277 | 201620316 |
| C1 | 201665278 | 201666793 |
| C1 | 202160562 | 202162137 |
| C1 | 202219884 | 202220951 |
| C1 | 202310062 | 202311201 |
| C1 | 202776180 | 202777264 |
| C1 | 203044682 | 203046089 |
| C1 | 203154039 | 203155053 |
| C1 | 203296045 | 203297123 |
| C1 | 203428905 | 203430258 |
| C1 | 203664700 | 203665755 |
| C1 | 203704818 | 203706052 |
| C1 | 203829302 | 203830543 |
| C1 | 203981740 | 203982810 |
| C1 | 204448246 | 204449867 |
| C1 | 204633230 | 204634644 |
| C1 | 204720126 | 204722055 |
| C1 | 205094111 | 205095271 |
| C1 | 205818240 | 205820110 |
| C1 | 206135194 | 206136578 |
| C1 | 206276073 | 206277204 |
| C1 | 206328376 | 206329736 |
| C1 | 206680738 | 206681855 |
| C1 | 206785717 | 206786888 |
| C1 | 206901468 | 206902718 |
| C1 | 207661425 | 207664322 |
| C1 | 207817876 | 207819319 |
| C1 | 207991122 | 207992193 |
| C1 | 207996502 | 207997608 |
| C1 | 208082378 | 208084132 |
| C1 | 208083413 | 208084627 |
| C1 | 208660424 | 208661482 |
| C1 | 209126923 | 209128184 |
| C1 | 209367246 | 209368385 |
| C1 | 209404585 | 209405659 |
| C1 | 209704633 | 209705651 |
| C1 | 210061849 | 210062961 |
| C1 | 210439043 | 210440618 |
| C1 | 210637050 | 210638738 |
| C1 | 211401951 | 211403366 |
| C1 | 211589517 | 211590756 |
| C1 | 211633499 | 211635405 |
| C1 | 211651869 | 211653319 |
| C1 | 211786895 | 211787990 |
| C1 | 212062420 | 212063496 |
| C1 | 212554965 | 212556042 |
| C1 | 212687608 | 212688900 |
| C1 | 212687942 | 212689137 |
| C1 | 212724064 | 212725104 |
| C1 | 212876624 | 212877671 |
| C1 | 212963546 | 212964969 |
| C1 | 213210830 | 213212602 |
| C1 | 213830400 | 213831487 |
| C1 | 214152755 | 214153795 |
| C1 | 214152944 | 214153966 |
| C1 | 214781500 | 214782561 |
| C1 | 214846013 | 214847288 |
| C1 | 215090055 | 215091162 |
| C1 | 215227398 | 215228442 |
| C1 | 216184852 | 216185974 |
| C1 | 217306885 | 217308149 |
| C1 | 217310133 | 217311163 |
| C1 | 217310406 | 217311523 |
| C1 | 217310979 | 217312357 |
| C1 | 217311753 | 217313273 |
| C1 | 217312541 | 217313666 |
| C1 | 218097812 | 218099165 |
| C1 | 218187613 | 218189076 |
| C1 | 218398649 | 218399743 |
| C1 | 219916097 | 219917109 |
| C1 | 219922849 | 219923972 |
| C1 | 219928307 | 219929362 |
| C1 | 220924174 | 220925203 |
| C1 | 221049681 | 221050897 |
| C1 | 221049899 | 221051186 |
| C1 | 221054055 | 221055470 |
| C1 | 221057308 | 221058962 |
| C1 | 221064198 | 221065534 |
| C1 | 221066433 | 221067843 |
| C1 | 221066948 | 221068113 |
| C1 | 221067447 | 221068598 |
| C1 | 221827379 | 221828441 |
| C1 | 222660439 | 222661609 |
| C1 | 222720644 | 222721661 |
| C1 | 223340875 | 223341940 |
| C1 | 223566354 | 223567556 |
| C1 | 223737793 | 223739457 |
| C1 | 223738941 | 223740108 |
| C1 | 223744628 | 223746457 |
| C1 | 224288176 | 224289312 |
| C1 | 224362769 | 224364108 |
| C1 | 224392371 | 224393493 |
| C1 | 224708148 | 224709236 |
| C1 | 225117559 | 225119120 |
| C1 | 225256156 | 225257343 |
| C1 | 225761530 | 225763098 |
| C1 | 225837696 | 225839377 |
| C1 | 225841521 | 225843051 |
| C1 | 225935495 | 225937336 |
| C1 | 225954913 | 225957066 |
| C1 | 226064696 | 226065954 |
| C1 | 226098690 | 226099803 |
| C1 | 226126613 | 226127760 |
| C1 | 226126977 | 226128023 |
| C1 | 226162701 | 226164105 |
| C1 | 226164868 | 226166820 |
| C1 | 226252871 | 226254180 |
| C1 | 226302411 | 226303529 |
| C1 | 226612801 | 226614148 |
| C1 | 226867223 | 226868407 |
| C1 | 226924068 | 226925183 |
| C1 | 226949560 | 226950741 |
| C1 | 227055060 | 227056064 |
| C1 | 227695335 | 227696482 |
| C1 | 227745530 | 227747746 |
| C1 | 227747942 | 227749223 |
| C1 | 227748268 | 227749417 |
| C1 | 227953805 | 227955189 |
| C1 | 228023606 | 228024882 |
| C1 | 228073943 | 228075093 |
| C1 | 228074878 | 228076410 |
| C1 | 228083282 | 228084683 |
| C1 | 228208709 | 228209971 |
| C1 | 228328750 | 228329976 |
| C1 | 228345435 | 228346593 |
| C1 | 228345927 | 228346990 |
| C1 | 228346034 | 228347129 |
| C1 | 228361600 | 228362733 |
| C1 | 228369522 | 228370701 |
| C1 | 228399919 | 228400998 |
| C1 | 228463708 | 228464846 |
| C1 | 228558379 | 228559764 |
| C1 | 228651902 | 228653081 |
| C1 | 228673142 | 228674431 |
| C1 | 228742702 | 228744000 |
| C1 | 228743815 | 228745008 |
| C1 | 228744835 | 228746144 |
| C1 | 228745384 | 228746464 |
| C1 | 228747094 | 228748458 |
| C1 | 228749852 | 228751831 |
| C1 | 228755100 | 228756352 |
| C1 | 228756019 | 228757289 |
| C1 | 228757461 | 228758816 |
| C1 | 228757962 | 228759080 |
| C1 | 228758769 | 228759825 |
| C1 | 228761641 | 228762778 |
| C1 | 228761827 | 228763013 |
| C1 | 228763230 | 228764490 |
| C1 | 228763507 | 228764665 |
| C1 | 228764066 | 228765170 |
| C1 | 228766097 | 228767168 |
| C1 | 228767733 | 228768797 |
| C1 | 228767835 | 228769174 |
| C1 | 228768233 | 228769451 |
| C1 | 228768677 | 228769878 |
| C1 | 228770279 | 228771415 |
| C1 | 228770481 | 228771664 |
| C1 | 228770961 | 228772219 |
| C1 | 228771392 | 228772664 |
| C1 | 228772860 | 228773921 |
| C1 | 228773076 | 228774226 |
| C1 | 228774932 | 228776457 |
| C1 | 228777360 | 228778615 |
| C1 | 228779821 | 228780842 |
| C1 | 228779924 | 228782888 |
| C1 | 228814532 | 228815632 |
| C1 | 228871663 | 228872796 |
| C1 | 229282507 | 229283810 |
| C1 | 229283064 | 229284246 |
| C1 | 229406577 | 229407651 |
| C1 | 229542354 | 229543550 |
| C1 | 229705715 | 229706852 |
| C1 | 230426829 | 230428249 |
| C1 | 230467751 | 230469264 |
| C1 | 231263427 | 231264571 |
| C1 | 231424699 | 231426273 |
| C1 | 231474688 | 231475846 |
| C1 | 231738491 | 231739728 |
| C1 | 232416986 | 232418055 |
| C1 | 232467450 | 232468550 |
| C1 | 232640864 | 232641925 |
| C1 | 232844740 | 232845954 |
| C1 | 233601133 | 233602585 |
| C1 | 233767120 | 233768171 |
| C1 | 234324208 | 234325214 |
| C1 | 234366811 | 234367944 |
| C1 | 234492169 | 234493269 |
| C1 | 234666760 | 234668050 |
| C1 | 234858414 | 234859507 |
| C1 | 234878999 | 234880434 |
| C1 | 234910929 | 234912043 |
| C1 | 234944326 | 234945613 |
| C1 | 234951724 | 234953367 |
| C1 | 235121487 | 235122701 |
| C1 | 235492952 | 235494765 |
| C1 | 235685559 | 235686774 |
| C1 | 235804840 | 235806028 |
| C1 | 236029763 | 236030843 |
| C1 | 236065054 | 236066109 |
| C1 | 236078651 | 236079971 |
| C1 | 236156374 | 236157658 |
| C1 | 236228015 | 236229104 |
| C1 | 236262316 | 236263657 |
| C1 | 236408141 | 236409449 |
| C1 | 236550112 | 236551241 |
| C1 | 236653206 | 236654262 |
| C1 | 236653900 | 236655153 |
| C1 | 236701859 | 236703510 |
| C1 | 237238218 | 237239509 |
| C1 | 237242153 | 237244321 |
| C1 | 237286491 | 237287617 |
| C1 | 237946592 | 237947935 |
| C1 | 240255176 | 240256288 |
| C1 | 240370591 | 240371861 |
| C1 | 240529602 | 240531384 |
| C1 | 240556295 | 240559120 |
| C1 | 240655727 | 240657008 |
| C1 | 240881197 | 240882207 |
| C1 | 240986155 | 240987451 |
| C1 | 241151588 | 241152624 |
| C1 | 241262941 | 241264132 |
| C1 | 241342940 | 241344131 |
| C1 | 241375955 | 241377846 |
| C1 | 241440016 | 241441276 |
| C1 | 241714496 | 241715836 |
| C1 | 241912116 | 241913547 |
| C1 | 242107458 | 242109172 |
| C1 | 242120581 | 242121775 |
| C1 | 242152829 | 242154514 |
| C1 | 242214084 | 242215230 |
| C1 | 242219922 | 242220977 |
| C1 | 242460482 | 242461632 |
| C1 | 243138786 | 243139920 |
| C1 | 243264474 | 243265545 |
| C1 | 243264603 | 243265751 |
| C1 | 243430318 | 243431553 |
| C1 | 243636139 | 243637832 |
| C1 | 243645691 | 243647001 |
| C1 | 243646075 | 243647235 |
| C1 | 243782060 | 243784021 |
| C1 | 244377076 | 244378384 |
| C1 | 244394778 | 244396027 |
| C1 | 244432105 | 244433311 |
| C1 | 245030614 | 245031936 |
| C1 | 245214147 | 245215793 |
| C1 | 245219850 | 245221069 |
| C1 | 245473454 | 245475007 |
| C1 | 245498935 | 245500765 |
| C1 | 245627819 | 245628942 |
| C1 | 245850865 | 245852387 |
| C1 | 245925204 | 245926657 |
| C1 | 246171414 | 246172863 |
| C1 | 246198099 | 246199441 |
| C1 | 246277582 | 246278804 |
| C1 | 246592663 | 246594412 |
| C1 | 246684783 | 246686140 |
| C1 | 246916447 | 246917621 |
| C1 | 246951715 | 246952838 |
| C1 | 247266110 | 247267304 |
| C1 | 247278647 | 247279823 |
| C1 | 247280726 | 247282716 |
| C1 | 247290474 | 247291672 |
| C1 | 247536659 | 247537785 |
| C1 | 247587605 | 247588949 |
| C1 | 247634003 | 247635073 |
| C1 | 247681041 | 247682334 |
| C1 | 247684550 | 247685751 |
| C1 | 247693536 | 247694729 |
| C1 | 247704725 | 247705761 |
| C1 | 247910308 | 247911453 |
| C1 | 247990284 | 247991314 |
| C1 | 248010195 | 248011287 |
| C1 | 248020132 | 248021256 |
| C1 | 248027414 | 248028715 |
| C1 | 248033443 | 248034606 |
| C1 | 248056657 | 248057894 |
| C1 | 248057011 | 248058216 |
| C1 | 248099845 | 248101247 |
| C1 | 248123037 | 248124056 |
| C1 | 248123816 | 248124836 |
| C1 | 248124349 | 248125358 |
| C1 | 248144703 | 248145863 |
| C1 | 248207401 | 248208530 |
| C1 | 248258553 | 248259976 |
| C1 | 248660418 | 248662281 |
| C1 | 248801282 | 248802546 |
| C1 | 248821087 | 248822429 |
| C1 | 249088584 | 249091373 |
| C2 | 10432 | 11527 |
| C2 | 10606 | 11647 |
| C2 | 10712 | 11794 |
| C2 | 197098 | 198150 |
| C2 | 307468 | 308648 |
| C2 | 308066 | 309211 |
| C2 | 314178 | 315637 |
| C2 | 419723 | 420911 |
| C2 | 426053 | 427112 |
| C2 | 449166 | 450208 |
| C2 | 494784 | 496205 |
| C2 | 496402 | 497693 |
| C2 | 636139 | 637265 |
| C2 | 730560 | 732286 |
| C2 | 731537 | 732748 |
| C2 | 731843 | 733450 |
| C2 | 799336 | 800470 |
| C2 | 831139 | 832270 |
| C2 | 863475 | 865286 |
| C2 | 864462 | 865719 |
| C2 | 923238 | 924601 |
| C2 | 930204 | 931624 |
| C2 | 1102365 | 1103626 |
| C2 | 1203757 | 1204928 |
| C2 | 1218349 | 1219714 |
| C2 | 1219449 | 1220973 |
| C2 | 1221723 | 1223181 |
| C2 | 1222740 | 1224021 |
| C2 | 1273240 | 1274966 |
| C2 | 1480318 | 1481445 |
| C2 | 1552122 | 1553136 |
| C2 | 1724883 | 1726692 |
| C2 | 1770923 | 1771964 |
| C2 | 1800556 | 1801738 |
| C2 | 1800983 | 1802672 |
| C2 | 1816722 | 1817904 |
| C2 | 1890556 | 1891600 |
| C2 | 1946335 | 1947586 |
| C2 | 2146298 | 2147511 |
| C2 | 2519578 | 2520949 |
| C2 | 2712024 | 2713169 |
| C2 | 2875682 | 2876816 |
| C2 | 2897574 | 2899358 |
| C2 | 3451899 | 3453056 |
| C2 | 3522138 | 3523220 |
| C2 | 3632631 | 3633720 |
| C2 | 3637567 | 3638663 |
| C2 | 3642078 | 3643150 |
| C2 | 3642210 | 3643251 |
| C2 | 3652379 | 3653430 |
| C2 | 3652472 | 3653752 |
| C2 | 3685200 | 3686425 |
| C2 | 3690821 | 3691911 |
| C2 | 3833438 | 3834818 |
| C2 | 3842889 | 3844773 |
| C2 | 3952838 | 3954171 |
| C2 | 6392950 | 6394149 |
| C2 | 6461485 | 6462493 |
| C2 | 7058251 | 7059366 |
| C2 | 7815811 | 7816866 |
| C2 | 8447630 | 8448658 |
| C2 | 8494025 | 8495063 |
| C2 | 8596847 | 8597933 |
| C2 | 8616503 | 8617573 |
| C2 | 8683589 | 8684670 |
| C2 | 8723503 | 8724622 |
| C2 | 8784721 | 8785853 |
| C2 | 9035633 | 9036730 |
| C2 | 9374399 | 9375818 |
| C2 | 9451030 | 9452385 |
| C2 | 9470724 | 9471811 |
| C2 | 9784602 | 9785618 |
| C2 | 9878789 | 9880516 |
| C2 | 9896664 | 9897711 |
| C2 | 9920817 | 9922522 |
| C2 | 9948378 | 9949679 |
| C2 | 10146013 | 10147165 |
| C2 | 10184004 | 10185099 |
| C2 | 10231038 | 10232374 |
| C2 | 10544429 | 10545961 |
| C2 | 10600195 | 10601368 |
| C2 | 10633395 | 10634497 |
| C2 | 11052864 | 11053965 |
| C2 | 11621965 | 11623201 |
| C2 | 11796474 | 11797589 |
| C2 | 11809171 | 11810264 |
| C2 | 15400496 | 15401540 |
| C2 | 15925283 | 15926496 |
| C2 | 16080392 | 16081447 |
| C2 | 16250650 | 16251789 |
| C2 | 16407633 | 16409407 |
| C2 | 18135173 | 18136243 |
| C2 | 20395955 | 20397297 |
| C2 | 20865814 | 20866937 |
| C2 | 20868837 | 20870118 |
| C2 | 20869587 | 20871687 |
| C2 | 21266120 | 21267250 |
| C2 | 22277700 | 22278761 |
| C2 | 23746757 | 23747954 |
| C2 | 25354240 | 25355306 |
| C2 | 25354386 | 25355605 |
| C2 | 25383644 | 25385090 |
| C2 | 25438300 | 25439382 |
| C2 | 25438534 | 25439585 |
| C2 | 25896221 | 25897265 |
| C2 | 26044601 | 26046043 |
| C2 | 26139081 | 26140238 |
| C2 | 26407482 | 26408753 |
| C2 | 26464754 | 26466245 |
| C2 | 26465320 | 26466409 |
| C2 | 26950536 | 26951760 |
| C2 | 27037908 | 27039222 |
| C2 | 27271369 | 27272714 |
| C2 | 27300797 | 27302206 |
| C2 | 27398073 | 27399081 |
| C2 | 27433207 | 27434348 |
| C2 | 27530860 | 27531927 |
| C2 | 27581212 | 27582338 |
| C2 | 27581490 | 27582655 |
| C2 | 27664752 | 27665832 |
| C2 | 27664966 | 27666084 |
| C2 | 27763356 | 27764623 |
| C2 | 29167191 | 29168259 |
| C2 | 29179126 | 29180426 |
| C2 | 29584129 | 29585166 |
| C2 | 29770753 | 29772227 |
| C2 | 29918599 | 29919666 |
| C2 | 30612158 | 30613179 |
| C2 | 31215289 | 31216555 |
| C2 | 33094407 | 33095621 |
| C2 | 36569272 | 36570396 |
| C2 | 36955178 | 36956284 |
| C2 | 37571204 | 37572399 |
| C2 | 38300385 | 38301538 |
| C2 | 38630281 | 38631388 |
| C2 | 38952891 | 38954018 |
| C2 | 39470225 | 39471983 |
| C2 | 39471605 | 39473359 |
| C2 | 40240965 | 40242106 |
| C2 | 41941950 | 41943253 |
| C2 | 41973951 | 41975101 |
| C2 | 41974223 | 41975330 |
| C2 | 42193253 | 42194369 |
| C2 | 42213242 | 42214282 |
| C2 | 42719378 | 42720538 |
| C2 | 42772511 | 42773583 |
| C2 | 43019185 | 43020277 |
| C2 | 43188509 | 43190066 |
| C2 | 43245087 | 43246196 |
| C2 | 43290464 | 43291584 |
| C2 | 43327762 | 43328824 |
| C2 | 43437360 | 43438643 |
| C2 | 43524203 | 43525352 |
| C2 | 43524480 | 43526108 |
| C2 | 44288266 | 44289354 |
| C2 | 44462772 | 44464105 |
| C2 | 44467162 | 44468885 |
| C2 | 44497161 | 44498363 |
| C2 | 44995313 | 44996489 |
| C2 | 45231313 | 45232606 |
| C2 | 45396902 | 45398731 |
| C2 | 46479563 | 46480655 |
| C2 | 46923299 | 46924357 |
| C2 | 47183525 | 47184727 |
| C2 | 47226138 | 47227169 |
| C2 | 47421661 | 47422701 |
| C2 | 48008318 | 48009441 |
| C2 | 48008749 | 48010108 |
| C2 | 48132601 | 48133751 |
| C2 | 48781319 | 48782895 |
| C2 | 48782181 | 48783317 |
| C2 | 48910572 | 48911580 |
| C2 | 51268947 | 51270069 |
| C2 | 52573437 | 52574576 |
| C2 | 52632325 | 52633487 |
| C2 | 54086277 | 54087360 |
| C2 | 54086689 | 54087766 |
| C2 | 54256756 | 54258344 |
| C2 | 54693015 | 54694153 |
| C2 | 55100367 | 55102295 |
| C2 | 55657645 | 55658661 |
| C2 | 55921458 | 55922586 |
| C2 | 56149161 | 56150467 |
| C2 | 60934772 | 60935799 |
| C2 | 61991609 | 61992819 |
| C2 | 62116019 | 62117242 |
| C2 | 62421756 | 62422875 |
| C2 | 62443317 | 62444384 |
| C2 | 62664344 | 62665394 |
| C2 | 62797831 | 62798951 |
| C2 | 63283948 | 63285295 |
| C2 | 63285457 | 63286497 |
| C2 | 64690008 | 64691026 |
| C2 | 64863134 | 64864294 |
| C2 | 65217123 | 65218170 |
| C2 | 65571185 | 65572434 |
| C2 | 65827775 | 65828896 |
| C2 | 66067590 | 66068654 |
| C2 | 66666670 | 66667757 |
| C2 | 66667273 | 66668560 |
| C2 | 66671932 | 66673129 |
| C2 | 66673085 | 66674643 |
| C2 | 68133115 | 68134171 |
| C2 | 70078156 | 70079262 |
| C2 | 70159628 | 70160809 |
| C2 | 70175350 | 70176457 |
| C2 | 70634431 | 70636029 |
| C2 | 70751124 | 70752198 |
| C2 | 71131508 | 71132651 |
| C2 | 71191709 | 71192796 |
| C2 | 71643951 | 71645062 |
| C2 | 71692709 | 71693829 |
| C2 | 71969499 | 71970784 |
| C2 | 71995689 | 71997822 |
| C2 | 72078897 | 72079976 |
| C2 | 73146863 | 73147909 |
| C2 | 73495609 | 73496963 |
| C2 | 74645122 | 74646297 |
| C2 | 74730493 | 74731589 |
| C2 | 75022423 | 75023514 |
| C2 | 75022916 | 75024018 |
| C2 | 75092028 | 75093144 |
| C2 | 75210790 | 75211847 |
| C2 | 79337672 | 79339207 |
| C2 | 80741550 | 80742583 |
| C2 | 83254127 | 83255192 |
| C2 | 83317934 | 83319972 |
| C2 | 84103381 | 84104545 |
| C2 | 85241334 | 85242348 |
| C2 | 85834427 | 85835465 |
| C2 | 85914094 | 85915405 |
| C2 | 85992178 | 85993295 |
| C2 | 86180510 | 86181567 |
| C2 | 86425216 | 86426722 |
| C2 | 87015672 | 87016769 |
| C2 | 87016049 | 87017148 |
| C2 | 87140793 | 87141878 |
| C2 | 87140954 | 87141983 |
| C2 | 87575445 | 87576541 |
| C2 | 87575706 | 87576865 |
| C2 | 87756007 | 87757447 |
| C2 | 87830679 | 87832354 |
| C2 | 88123166 | 88124805 |
| C2 | 88124441 | 88126027 |
| C2 | 88295855 | 88296977 |
| C2 | 88302228 | 88303346 |
| C2 | 88703262 | 88704268 |
| C2 | 88833441 | 88834506 |
| C2 | 89547115 | 89548579 |
| C2 | 89910596 | 89912973 |
| C2 | 89982359 | 89983538 |
| C2 | 90015559 | 90016831 |
| C2 | 90197593 | 90199476 |
| C2 | 90284089 | 90285255 |
| C2 | 90383765 | 90385021 |
| C2 | 90389516 | 90390841 |
| C2 | 90391013 | 90392272 |
| C2 | 90412819 | 90413961 |
| C2 | 90440231 | 90441543 |
| C2 | 90447163 | 90448224 |
| C2 | 90447326 | 90448389 |
| C2 | 90447856 | 90448948 |
| C2 | 90448261 | 90449350 |
| C2 | 90449053 | 90450186 |
| C2 | 90526674 | 90527882 |
| C2 | 91594630 | 91596101 |
| C2 | 91606942 | 91608276 |
| C2 | 91607915 | 91609145 |
| C2 | 91612733 | 91613871 |
| C2 | 91634331 | 91635439 |
| C2 | 91635333 | 91636531 |
| C2 | 91701066 | 91702126 |
| C2 | 91715077 | 91716420 |
| C2 | 91749498 | 91750873 |
| C2 | 91750098 | 91751287 |
| C2 | 91750293 | 91751439 |
| C2 | 91910573 | 91911733 |
| C2 | 92023291 | 92024441 |
| C2 | 92203647 | 92204753 |
| C2 | 92260138 | 92261263 |
| C2 | 92261829 | 92262983 |
| C2 | 96314496 | 96315591 |
| C2 | 97127453 | 97128558 |
| C2 | 97127635 | 97128736 |
| C2 | 97215388 | 97216410 |
| C2 | 97215504 | 97216610 |
| C2 | 97426879 | 97428580 |
| C2 | 97471099 | 97472296 |
| C2 | 97504756 | 97505839 |
| C2 | 97527180 | 97528373 |
| C2 | 97759573 | 97760756 |
| C2 | 98062789 | 98064579 |
| C2 | 98611234 | 98612336 |
| C2 | 98697834 | 98698962 |
| C2 | 99386098 | 99387339 |
| C2 | 99438421 | 99439571 |
| C2 | 99439050 | 99440138 |
| C2 | 100306205 | 100307267 |
| C2 | 102857760 | 102858777 |
| C2 | 102984539 | 102985696 |
| C2 | 103977782 | 103978901 |
| C2 | 105275282 | 105276355 |
| C2 | 105827644 | 105828746 |
| C2 | 105853026 | 105854172 |
| C2 | 106002268 | 106003307 |
| C2 | 106483127 | 106484355 |
| C2 | 106540690 | 106541730 |
| C2 | 106747002 | 106748274 |
| C2 | 106760710 | 106762398 |
| C2 | 106810154 | 106811234 |
| C2 | 106958815 | 106960054 |
| C2 | 107082670 | 107083739 |
| C2 | 107093596 | 107095717 |
| C2 | 107410074 | 107411201 |
| C2 | 107832182 | 107833186 |
| C2 | 108363153 | 108364593 |
| C2 | 108428153 | 108429327 |
| C2 | 109591598 | 109592774 |
| C2 | 109873027 | 109874096 |
| C2 | 109947499 | 109948982 |
| C2 | 110077486 | 110078661 |
| C2 | 110968946 | 110970930 |
| C2 | 111180200 | 111181318 |
| C2 | 111189966 | 111191195 |
| C2 | 111333844 | 111334967 |
| C2 | 111876054 | 111877236 |
| C2 | 112896774 | 112898067 |
| C2 | 112917746 | 112918809 |
| C2 | 113189779 | 113190936 |
| C2 | 113190266 | 113191353 |
| C2 | 113341100 | 113342217 |
| C2 | 113447662 | 113448886 |
| C2 | 113992343 | 113993502 |
| C2 | 113992958 | 113994448 |
| C2 | 114035472 | 114036533 |
| C2 | 114257462 | 114258542 |
| C2 | 114360560 | 114361964 |
| C2 | 114410161 | 114411664 |
| C2 | 114736683 | 114737752 |
| C2 | 116669454 | 116670828 |
| C2 | 118045518 | 118046779 |
| C2 | 118406320 | 118407476 |
| C2 | 118553200 | 118554277 |
| C2 | 118561534 | 118563153 |
| C2 | 118617198 | 118618255 |
| C2 | 118627917 | 118630052 |
| C2 | 119392206 | 119393230 |
| C2 | 119531979 | 119533215 |
| C2 | 119912635 | 119913894 |
| C2 | 120048095 | 120049288 |
| C2 | 120933840 | 120935601 |
| C2 | 120979794 | 120980887 |
| C2 | 121076286 | 121077495 |
| C2 | 121103392 | 121104442 |
| C2 | 121236016 | 121237028 |
| C2 | 121243143 | 121244236 |
| C2 | 121245086 | 121246113 |
| C2 | 121695948 | 121696961 |
| C2 | 121894987 | 121896302 |
| C2 | 122078990 | 122080008 |
| C2 | 122650101 | 122651410 |
| C2 | 122659505 | 122660706 |
| C2 | 122809037 | 122810582 |
| C2 | 123106575 | 123108505 |
| C2 | 123972028 | 123973073 |
| C2 | 124707606 | 124708729 |
| C2 | 125353390 | 125355359 |
| C2 | 126055764 | 126057082 |
| C2 | 127002722 | 127003860 |
| C2 | 127838973 | 127840039 |
| C2 | 127954866 | 127956076 |
| C2 | 128372819 | 128374140 |
| C2 | 128381136 | 128382282 |
| C2 | 128395529 | 128396699 |
| C2 | 128452600 | 128453946 |
| C2 | 128544592 | 128545781 |
| C2 | 128933508 | 128934567 |
| C2 | 128948074 | 128949133 |
| C2 | 128970001 | 128971607 |
| C2 | 129017963 | 129019126 |
| C2 | 129076811 | 129078132 |
| C2 | 129175106 | 129176263 |
| C2 | 129230317 | 129231558 |
| C2 | 129250452 | 129251568 |
| C2 | 129658709 | 129660626 |
| C2 | 130344600 | 130345639 |
| C2 | 130793825 | 130796269 |
| C2 | 130901298 | 130902463 |
| C2 | 131045577 | 131047092 |
| C2 | 131084970 | 131086007 |
| C2 | 131349496 | 131350654 |
| C2 | 131411925 | 131414112 |
| C2 | 131594007 | 131595150 |
| C2 | 131798325 | 131799414 |
| C2 | 131851575 | 131852718 |
| C2 | 132020664 | 132021947 |
| C2 | 132056483 | 132057963 |
| C2 | 132057017 | 132058150 |
| C2 | 132109625 | 132111188 |
| C2 | 132152360 | 132153543 |
| C2 | 132200201 | 132201404 |
| C2 | 132202050 | 132203247 |
| C2 | 132217764 | 132218871 |
| C2 | 132403785 | 132405239 |
| C2 | 132481090 | 132482449 |
| C2 | 132510746 | 132511939 |
| C2 | 132558657 | 132559808 |
| C2 | 132559408 | 132560489 |
| C2 | 132559672 | 132560756 |
| C2 | 132586599 | 132587749 |
| C2 | 132586789 | 132587917 |
| C2 | 132587067 | 132588175 |
| C2 | 132587670 | 132588783 |
| C2 | 133011733 | 133013489 |
| C2 | 133025686 | 133026781 |
| C2 | 133030743 | 133031870 |
| C2 | 133038490 | 133039741 |
| C2 | 133402275 | 133403375 |
| C2 | 135215131 | 135216194 |
| C2 | 135413266 | 135414375 |
| C2 | 135808641 | 135809764 |
| C2 | 136782775 | 136783818 |
| C2 | 136833891 | 136834911 |
| C2 | 136921491 | 136922517 |
| C2 | 137496582 | 137497603 |
| C2 | 137522440 | 137523489 |
| C2 | 137995716 | 137996845 |
| C2 | 139538120 | 139539502 |
| C2 | 140168015 | 140169243 |
| C2 | 141172216 | 141173230 |
| C2 | 141585534 | 141586635 |
| C2 | 142790076 | 142791099 |
| C2 | 143783746 | 143784943 |
| C2 | 143979518 | 143980569 |
| C2 | 144871370 | 144872382 |
| C2 | 145281442 | 145282537 |
| C2 | 148600855 | 148602169 |
| C2 | 148846492 | 148847508 |
| C2 | 148847527 | 148848538 |
| C2 | 149477840 | 149478903 |
| C2 | 149683695 | 149684952 |
| C2 | 150176659 | 150177850 |
| C2 | 150263908 | 150265096 |
| C2 | 150392123 | 150393188 |
| C2 | 151398413 | 151399649 |
| C2 | 151858361 | 151859368 |
| C2 | 152144912 | 152146029 |
| C2 | 152890512 | 152891619 |
| C2 | 153245657 | 153246917 |
| C2 | 155314186 | 155315266 |
| C2 | 157110583 | 157111882 |
| C2 | 157177718 | 157178798 |
| C2 | 157178698 | 157179864 |
| C2 | 157178927 | 157180076 |
| C2 | 158299866 | 158301460 |
| C2 | 159313906 | 159315000 |
| C2 | 160149476 | 160151121 |
| C2 | 160150840 | 160151981 |
| C2 | 162093369 | 162094724 |
| C2 | 162197071 | 162198370 |
| C2 | 162827713 | 162828782 |
| C2 | 163882304 | 163883397 |
| C2 | 166141943 | 166143020 |
| C2 | 166593326 | 166594449 |
| C2 | 168027973 | 168029012 |
| C2 | 168149335 | 168150385 |
| C2 | 169312145 | 169313210 |
| C2 | 169542002 | 169543340 |
| C2 | 169966703 | 169967868 |
| C2 | 170184012 | 170185170 |
| C2 | 170184187 | 170185335 |
| C2 | 170431369 | 170432476 |
| C2 | 170551245 | 170552798 |
| C2 | 170624415 | 170625592 |
| C2 | 170696385 | 170697491 |
| C2 | 171006009 | 171007103 |
| C2 | 171006307 | 171007516 |
| C2 | 171572805 | 171574069 |
| C2 | 171573149 | 171574330 |
| C2 | 172050592 | 172051610 |
| C2 | 172944294 | 172945877 |
| C2 | 172963397 | 172965108 |
| C2 | 173099419 | 173100597 |
| C2 | 173118007 | 173119031 |
| C2 | 173179815 | 173181167 |
| C2 | 173378524 | 173379751 |
| C2 | 173538503 | 173539591 |
| C2 | 173539009 | 173540133 |
| C2 | 174912119 | 174913529 |
| C2 | 175111355 | 175112532 |
| C2 | 175201434 | 175202586 |
| C2 | 175274419 | 175275611 |
| C2 | 175351078 | 175352212 |
| C2 | 175435658 | 175436998 |
| C2 | 176170884 | 176171913 |
| C2 | 177035755 | 177036988 |
| C2 | 177036292 | 177037381 |
| C2 | 178077041 | 178078089 |
| C2 | 178498685 | 178499818 |
| C2 | 178811174 | 178812274 |
| C2 | 179440884 | 179441964 |
| C2 | 179849305 | 179850364 |
| C2 | 181737636 | 181738730 |
| C2 | 184249533 | 184250701 |
| C2 | 184921594 | 184922695 |
| C2 | 185192154 | 185193227 |
| C2 | 187867505 | 187868692 |
| C2 | 191242646 | 191243775 |
| C2 | 191262223 | 191263241 |
| C2 | 191512052 | 191513142 |
| C2 | 191625216 | 191626713 |
| C2 | 192606786 | 192607900 |
| C2 | 194082404 | 194083638 |
| C2 | 197033510 | 197034672 |
| C2 | 197770043 | 197771140 |
| C2 | 198254008 | 198257685 |
| C2 | 198257196 | 198258575 |
| C2 | 198260280 | 198261552 |
| C2 | 198262178 | 198263340 |
| C2 | 198262685 | 198263996 |
| C2 | 198263193 | 198264252 |
| C2 | 198264279 | 198265390 |
| C2 | 198264476 | 198265658 |
| C2 | 198264939 | 198266160 |
| C2 | 198265624 | 198266749 |
| C2 | 198265966 | 198267112 |
| C2 | 198266209 | 198267354 |
| C2 | 198266645 | 198267676 |
| C2 | 198266780 | 198268050 |
| C2 | 198267173 | 198268259 |
| C2 | 198267809 | 198268988 |
| C2 | 198269300 | 198270401 |
| C2 | 198269499 | 198270696 |
| C2 | 198272160 | 198273343 |
| C2 | 198272593 | 198273918 |
| C2 | 198273994 | 198275231 |
| C2 | 198280818 | 198282135 |
| C2 | 198282733 | 198285766 |
| C2 | 198285253 | 198286357 |
| C2 | 198288032 | 198289198 |
| C2 | 198299196 | 198300315 |
| C2 | 198786662 | 198787978 |
| C2 | 199123887 | 199124909 |
| C2 | 200333188 | 200334293 |
| C2 | 200422551 | 200423903 |
| C2 | 200468367 | 200469458 |
| C2 | 200523502 | 200524589 |
| C2 | 201460339 | 201461364 |
| C2 | 201720210 | 201721515 |
| C2 | 201729183 | 201730318 |
| C2 | 201752017 | 201753109 |
| C2 | 201980004 | 201981084 |
| C2 | 201988431 | 201989683 |
| C2 | 202073143 | 202074424 |
| C2 | 202148121 | 202149507 |
| C2 | 202765967 | 202767423 |
| C2 | 202797953 | 202799537 |
| C2 | 202900031 | 202901143 |
| C2 | 203399012 | 203400552 |
| C2 | 204649054 | 204650186 |
| C2 | 206118918 | 206120079 |
| C2 | 207022335 | 207023482 |
| C2 | 207115072 | 207116132 |
| C2 | 207118094 | 207119575 |
| C2 | 207264885 | 207265958 |
| C2 | 208631257 | 208632416 |
| C2 | 208631681 | 208632763 |
| C2 | 208795026 | 208796339 |
| C2 | 208976690 | 208977953 |
| C2 | 208988363 | 208989559 |
| C2 | 208988709 | 208989925 |
| C2 | 210009645 | 210010756 |
| C2 | 210073427 | 210074527 |
| C2 | 211234125 | 211235335 |
| C2 | 212669201 | 212670231 |
| C2 | 212753252 | 212754756 |
| C2 | 213697144 | 213698364 |
| C2 | 215699235 | 215700344 |
| C2 | 216143720 | 216145006 |
| C2 | 217091449 | 217092662 |
| C2 | 218620392 | 218621759 |
| C2 | 218842709 | 218844432 |
| C2 | 218898011 | 218899250 |
| C2 | 219117856 | 219119092 |
| C2 | 219232884 | 219233946 |
| C2 | 219246352 | 219247486 |
| C2 | 219710771 | 219712391 |
| C2 | 219737601 | 219738669 |
| C2 | 219738151 | 219739287 |
| C2 | 220071230 | 220072300 |
| C2 | 220312389 | 220313575 |
| C2 | 220378574 | 220379650 |
| C2 | 220796195 | 220797361 |
| C2 | 223621690 | 223622742 |
| C2 | 223681372 | 223683631 |
| C2 | 224702492 | 224703588 |
| C2 | 224901728 | 224902741 |
| C2 | 225466939 | 225468302 |
| C2 | 227833795 | 227835263 |
| C2 | 227859477 | 227860768 |
| C2 | 228189372 | 228190460 |
| C2 | 230044988 | 230046153 |
| C2 | 230940357 | 230941415 |
| C2 | 231324870 | 231325984 |
| C2 | 231854738 | 231855967 |
| C2 | 232347573 | 232348986 |
| C2 | 232348113 | 232349349 |
| C2 | 232446024 | 232447058 |
| C2 | 232486947 | 232487964 |
| C2 | 232571918 | 232573036 |
| C2 | 232577046 | 232578183 |
| C2 | 232826684 | 232827806 |
| C2 | 233216711 | 233217858 |
| C2 | 233251155 | 233252256 |
| C2 | 233273992 | 233275014 |
| C2 | 233740388 | 233741488 |
| C2 | 234239736 | 234240839 |
| C2 | 234330515 | 234331559 |
| C2 | 234846980 | 234848623 |
| C2 | 236402422 | 236403618 |
| C2 | 236458753 | 236459766 |
| C2 | 237028369 | 237029693 |
| C2 | 237122562 | 237123646 |
| C2 | 237122832 | 237123909 |
| C2 | 238423444 | 238424467 |
| C2 | 238480243 | 238481350 |
| C2 | 238584733 | 238585759 |
| C2 | 238599369 | 238600502 |
| C2 | 238814271 | 238815539 |
| C2 | 239129471 | 239130581 |
| C2 | 239326152 | 239327365 |
| C2 | 240017551 | 240018722 |
| C2 | 240113792 | 240114967 |
| C2 | 240144522 | 240145576 |
| C2 | 240181352 | 240182466 |
| C2 | 240529178 | 240530243 |
| C2 | 240530391 | 240531674 |
| C2 | 240659406 | 240660603 |
| C2 | 240815996 | 240817043 |
| C2 | 240877846 | 240878948 |
| C2 | 240884331 | 240885461 |
| C2 | 240899725 | 240900754 |
| C2 | 241459111 | 241460167 |
| C2 | 241518790 | 241520298 |
| C2 | 241562314 | 241563493 |
| C2 | 241622731 | 241623896 |
| C2 | 241771138 | 241772212 |
| C2 | 241846735 | 241848536 |
| C2 | 241849464 | 241850590 |
| C2 | 241900772 | 241901959 |
| C2 | 241905269 | 241906407 |
| C2 | 241930557 | 241931860 |
| C2 | 241975214 | 241976246 |
| C2 | 241975457 | 241976637 |
| C2 | 242034924 | 242036024 |
| C2 | 242294376 | 242295524 |
| C2 | 242508993 | 242510950 |
| C2 | 242742280 | 242743449 |
| C2 | 242755737 | 242756990 |
| C2 | 242789663 | 242790771 |
| C2 | 242832500 | 242833693 |
| C2 | 242843373 | 242845138 |
| C2 | 242844170 | 242845252 |
| C2 | 242988206 | 242989403 |
| C2 | 242988479 | 242989538 |
| C2 | 242988588 | 242989647 |
| C2 | 242988737 | 242989913 |
| C2 | 242988965 | 242990159 |
| C2 | 243152786 | 243153828 |
| C3 | 524121 | 525276 |
| C3 | 524314 | 525609 |
| C3 | 524655 | 525708 |
| C3 | 577400 | 578470 |
| C3 | 850929 | 852332 |
| C3 | 1309529 | 1310708 |
| C3 | 1781637 | 1782769 |
| C3 | 2028604 | 2029758 |
| C3 | 2952824 | 2953908 |
| C3 | 4855552 | 4856760 |
| C3 | 5137088 | 5138401 |
| C3 | 6313580 | 6314873 |
| C3 | 6381724 | 6382813 |
| C3 | 6429466 | 6430881 |
| C3 | 6549033 | 6550124 |
| C3 | 6746419 | 6747481 |
| C3 | 8480570 | 8481695 |
| C3 | 8580285 | 8581362 |
| C3 | 8720232 | 8721391 |
| C3 | 8720631 | 8721866 |
| C3 | 9821572 | 9822936 |
| C3 | 9834236 | 9835369 |
| C3 | 9987437 | 9988811 |
| C3 | 9987845 | 9988906 |
| C3 | 9987969 | 9989046 |
| C3 | 9988266 | 9989543 |
| C3 | 9988751 | 9989935 |
| C3 | 9999030 | 10000084 |
| C3 | 10183632 | 10184771 |
| C3 | 10397175 | 10398472 |
| C3 | 10670157 | 10671503 |
| C3 | 11033768 | 11034821 |
| C3 | 11265577 | 11266700 |
| C3 | 11311820 | 11313493 |
| C3 | 11528234 | 11529420 |
| C3 | 11745966 | 11747141 |
| C3 | 11907891 | 11908931 |
| C3 | 12585860 | 12586869 |
| C3 | 12595173 | 12596317 |
| C3 | 12828447 | 12829592 |
| C3 | 12965582 | 12966782 |
| C3 | 13000810 | 13002391 |
| C3 | 13056902 | 13058721 |
| C3 | 13114181 | 13115303 |
| C3 | 13114659 | 13115773 |
| C3 | 13555111 | 13556165 |
| C3 | 13612561 | 13613604 |
| C3 | 14338782 | 14339911 |
| C3 | 14338922 | 14339987 |
| C3 | 15203689 | 15204793 |
| C3 | 15247562 | 15248589 |
| C3 | 16386603 | 16387956 |
| C3 | 17745762 | 17746861 |
| C3 | 19188165 | 19189410 |
| C3 | 19189152 | 19190349 |
| C3 | 20167043 | 20168197 |
| C3 | 21206312 | 21207801 |
| C3 | 25475422 | 25476455 |
| C3 | 25824370 | 25825416 |
| C3 | 26431333 | 26432474 |
| C3 | 26431506 | 26432687 |
| C3 | 26663049 | 26664126 |
| C3 | 26665168 | 26666664 |
| C3 | 26786393 | 26787496 |
| C3 | 27673901 | 27674919 |
| C3 | 27772230 | 27773334 |
| C3 | 28241589 | 28242645 |
| C3 | 28241750 | 28242780 |
| C3 | 29150403 | 29152505 |
| C3 | 31718869 | 31720005 |
| C3 | 32500932 | 32501981 |
| C3 | 32542048 | 32543175 |
| C3 | 32738530 | 32739566 |
| C3 | 32862693 | 32863775 |
| C3 | 32943617 | 32945283 |
| C3 | 35660133 | 35661856 |
| C3 | 36872804 | 36873815 |
| C3 | 37230589 | 37231715 |
| C3 | 38031606 | 38032958 |
| C3 | 38040132 | 38041301 |
| C3 | 38121498 | 38122558 |
| C3 | 38122032 | 38123467 |
| C3 | 38332203 | 38333720 |
| C3 | 39308855 | 39309936 |
| C3 | 39543307 | 39544499 |
| C3 | 39575437 | 39576489 |
| C3 | 41041533 | 41042785 |
| C3 | 41358098 | 41359212 |
| C3 | 42727303 | 42728401 |
| C3 | 42814170 | 42815380 |
| C3 | 43030822 | 43031913 |
| C3 | 43894427 | 43895517 |
| C3 | 44619392 | 44620425 |
| C3 | 45076826 | 45078219 |
| C3 | 45236660 | 45237738 |
| C3 | 45768002 | 45769129 |
| C3 | 46535801 | 46536922 |
| C3 | 46758767 | 46759844 |
| C3 | 46915440 | 46916613 |
| C3 | 46939776 | 46940924 |
| C3 | 47005179 | 47006428 |
| C3 | 47020693 | 47021756 |
| C3 | 47050419 | 47051544 |
| C3 | 48251805 | 48252887 |
| C3 | 48280331 | 48281417 |
| C3 | 48556804 | 48557950 |
| C3 | 48694023 | 48695246 |
| C3 | 49055338 | 49056432 |
| C3 | 49056434 | 49057704 |
| C3 | 49236418 | 49237488 |
| C3 | 49459054 | 49460250 |
| C3 | 49755651 | 49756722 |
| C3 | 49756153 | 49757279 |
| C3 | 49802795 | 49803848 |
| C3 | 50021548 | 50022594 |
| C3 | 50121892 | 50122986 |
| C3 | 50230559 | 50231615 |
| C3 | 50376445 | 50377845 |
| C3 | 50487696 | 50488761 |
| C3 | 51428308 | 51429407 |
| C3 | 51679899 | 51680935 |
| C3 | 51740625 | 51741912 |
| C3 | 52006845 | 52007997 |
| C3 | 52406716 | 52408073 |
| C3 | 52567916 | 52569229 |
| C3 | 52737843 | 52739193 |
| C3 | 53381822 | 53383125 |
| C3 | 54121488 | 54122577 |
| C3 | 54173736 | 54176114 |
| C3 | 54842886 | 54844003 |
| C3 | 56617628 | 56618704 |
| C3 | 57113066 | 57114154 |
| C3 | 58275130 | 58276299 |
| C3 | 61068559 | 61069624 |
| C3 | 61419578 | 61420685 |
| C3 | 63121772 | 63122937 |
| C3 | 63202419 | 63203492 |
| C3 | 63202705 | 63203875 |
| C3 | 63922898 | 63924368 |
| C3 | 65341590 | 65342951 |
| C3 | 65342144 | 65343384 |
| C3 | 65737105 | 65738163 |
| C3 | 66701530 | 66702608 |
| C3 | 67022319 | 67023375 |
| C3 | 67494812 | 67495981 |
| C3 | 68271424 | 68272468 |
| C3 | 69153202 | 69154473 |
| C3 | 69740334 | 69741461 |
| C3 | 71803480 | 71804637 |
| C3 | 72048598 | 72049671 |
| C3 | 72496265 | 72497423 |
| C3 | 72677302 | 72678354 |
| C3 | 72977556 | 72978823 |
| C3 | 73487520 | 73488592 |
| C3 | 73989296 | 73990437 |
| C3 | 74416496 | 74417548 |
| C3 | 74948864 | 74950219 |
| C3 | 75444508 | 75445596 |
| C3 | 75444760 | 75446129 |
| C3 | 75486454 | 75487652 |
| C3 | 75600722 | 75601944 |
| C3 | 75605624 | 75607536 |
| C3 | 75716460 | 75717606 |
| C3 | 75717848 | 75719052 |
| C3 | 75836724 | 75837893 |
| C3 | 75842233 | 75844303 |
| C3 | 77817911 | 77819061 |
| C3 | 78632111 | 78633281 |
| C3 | 79545134 | 79546194 |
| C3 | 80818988 | 80820096 |
| C3 | 82764009 | 82765080 |
| C3 | 86487065 | 86488145 |
| C3 | 86855166 | 86856174 |
| C3 | 87101314 | 87102398 |
| C3 | 87377708 | 87378729 |
| C3 | 87782336 | 87783424 |
| C3 | 88255290 | 88256417 |
| C3 | 94884911 | 94886042 |
| C3 | 96208652 | 96209751 |
| C3 | 96495118 | 96496287 |
| C3 | 96737188 | 96738261 |
| C3 | 97627604 | 97629165 |
| C3 | 97628699 | 97629868 |
| C3 | 98555090 | 98556198 |
| C3 | 99594263 | 99595482 |
| C3 | 99980459 | 99982062 |
| C3 | 101158223 | 101159725 |
| C3 | 101181495 | 101182557 |
| C3 | 101351399 | 101352816 |
| C3 | 101567846 | 101568905 |
| C3 | 102283458 | 102284593 |
| C3 | 102720735 | 102721807 |
| C3 | 102938791 | 102939827 |
| C3 | 102939021 | 102940118 |
| C3 | 102939159 | 102940504 |
| C3 | 103279959 | 103281550 |
| C3 | 103308452 | 103309461 |
| C3 | 104774365 | 104775454 |
| C3 | 105587108 | 105588139 |
| C3 | 105600867 | 105602335 |
| C3 | 105792667 | 105793759 |
| C3 | 107234512 | 107235563 |
| C3 | 107465148 | 107466187 |
| C3 | 108307886 | 108308993 |
| C3 | 108467907 | 108469413 |
| C3 | 108921331 | 108923341 |
| C3 | 109035342 | 109036602 |
| C3 | 111279288 | 111280734 |
| C3 | 111868587 | 111869673 |
| C3 | 113159910 | 113161031 |
| C3 | 113234213 | 113235516 |
| C3 | 113417433 | 113418674 |
| C3 | 114174046 | 114175053 |
| C3 | 115510450 | 115511618 |
| C3 | 115885238 | 115886424 |
| C3 | 116078275 | 116079789 |
| C3 | 116817828 | 116818864 |
| C3 | 117987886 | 117989007 |
| C3 | 118570392 | 118571541 |
| C3 | 118725395 | 118726568 |
| C3 | 119502403 | 119503432 |
| C3 | 120025008 | 120026143 |
| C3 | 121268807 | 121269818 |
| C3 | 121655240 | 121656807 |
| C3 | 121883605 | 121884726 |
| C3 | 122380197 | 122381260 |
| C3 | 122488095 | 122489184 |
| C3 | 122498563 | 122499706 |
| C3 | 122695166 | 122696225 |
| C3 | 123071694 | 123072718 |
| C3 | 123303419 | 123304490 |
| C3 | 124468359 | 124469462 |
| C3 | 124468469 | 124469634 |
| C3 | 124468856 | 124470023 |
| C3 | 125708592 | 125709650 |
| C3 | 125708738 | 125709984 |
| C3 | 125709006 | 125710727 |
| C3 | 126113163 | 126114335 |
| C3 | 126184529 | 126185659 |
| C3 | 126208559 | 126209591 |
| C3 | 126667928 | 126668974 |
| C3 | 126944965 | 126946537 |
| C3 | 127022670 | 127023892 |
| C3 | 127056497 | 127057818 |
| C3 | 127146975 | 127148037 |
| C3 | 127479923 | 127481208 |
| C3 | 127794154 | 127795418 |
| C3 | 127795020 | 127796141 |
| C3 | 127795272 | 127796359 |
| C3 | 128201536 | 128202841 |
| C3 | 128215065 | 128216273 |
| C3 | 128321902 | 128324226 |
| C3 | 128371828 | 128373071 |
| C3 | 128372776 | 128373837 |
| C3 | 128709807 | 128710941 |
| C3 | 128722198 | 128723414 |
| C3 | 128764757 | 128765926 |
| C3 | 128780458 | 128781559 |
| C3 | 128799809 | 128801377 |
| C3 | 128839896 | 128840978 |
| C3 | 128899887 | 128900988 |
| C3 | 129356882 | 129357916 |
| C3 | 130445650 | 130446758 |
| C3 | 130445788 | 130446819 |
| C3 | 132095354 | 132096534 |
| C3 | 132188859 | 132190012 |
| C3 | 132547812 | 132548823 |
| C3 | 132626085 | 132627245 |
| C3 | 133146914 | 133148088 |
| C3 | 133190578 | 133192188 |
| C3 | 133291857 | 133292952 |
| C3 | 133392854 | 133393999 |
| C3 | 133502202 | 133503313 |
| C3 | 135157738 | 135158916 |
| C3 | 137577704 | 137578714 |
| C3 | 137591216 | 137592357 |
| C3 | 137830819 | 137832182 |
| C3 | 137905924 | 137906970 |
| C3 | 138151719 | 138153671 |
| C3 | 138311465 | 138312709 |
| C3 | 138503500 | 138504684 |
| C3 | 138664260 | 138665319 |
| C3 | 138678786 | 138679892 |
| C3 | 138724356 | 138725419 |
| C3 | 138762621 | 138764120 |
| C3 | 138763335 | 138764481 |
| C3 | 141495476 | 141496925 |
| C3 | 141944316 | 141945394 |
| C3 | 142665733 | 142666844 |
| C3 | 142839422 | 142840513 |
| C3 | 143474473 | 143475535 |
| C3 | 144134767 | 144135826 |
| C3 | 147105399 | 147106576 |
| C3 | 147136531 | 147137873 |
| C3 | 147140726 | 147141773 |
| C3 | 148522766 | 148523784 |
| C3 | 149097617 | 149099449 |
| C3 | 150126264 | 150127339 |
| C3 | 150537562 | 150538783 |
| C3 | 151177783 | 151179460 |
| C3 | 152879443 | 152880496 |
| C3 | 152879815 | 152881156 |
| C3 | 152934888 | 152936464 |
| C3 | 152939321 | 152940388 |
| C3 | 153534371 | 153536107 |
| C3 | 154095677 | 154097357 |
| C3 | 154574154 | 154575259 |
| C3 | 154842292 | 154843562 |
| C3 | 155028364 | 155029826 |
| C3 | 155259455 | 155260566 |
| C3 | 155751712 | 155752829 |
| C3 | 155817886 | 155818898 |
| C3 | 157738037 | 157739633 |
| C3 | 158430480 | 158431885 |
| C3 | 159557254 | 159558389 |
| C3 | 159826767 | 159827940 |
| C3 | 159939806 | 159943113 |
| C3 | 159943323 | 159944605 |
| C3 | 162345762 | 162346793 |
| C3 | 162428067 | 162429255 |
| C3 | 162954379 | 162955552 |
| C3 | 163147494 | 163148530 |
| C3 | 163463705 | 163464832 |
| C3 | 163463835 | 163465695 |
| C3 | 164096783 | 164097930 |
| C3 | 165107181 | 165108244 |
| C3 | 166100862 | 166102238 |
| C3 | 166649439 | 166650614 |
| C3 | 169383329 | 169385500 |
| C3 | 169530317 | 169531835 |
| C3 | 169719789 | 169721081 |
| C3 | 170038708 | 170040005 |
| C3 | 170470210 | 170471247 |
| C3 | 170532200 | 170533892 |
| C3 | 170745529 | 170747015 |
| C3 | 171175438 | 171176517 |
| C3 | 172164873 | 172166017 |
| C3 | 172165367 | 172166824 |
| C3 | 172279668 | 172280853 |
| C3 | 172340974 | 172342772 |
| C3 | 172456324 | 172457410 |
| C3 | 173113065 | 173114120 |
| C3 | 173113261 | 173114371 |
| C3 | 173114961 | 173116082 |
| C3 | 173302252 | 173304096 |
| C3 | 176079792 | 176080844 |
| C3 | 176531631 | 176532913 |
| C3 | 176861261 | 176862973 |
| C3 | 177111793 | 177112927 |
| C3 | 178579361 | 178582131 |
| C3 | 178750296 | 178751325 |
| C3 | 178832586 | 178833620 |
| C3 | 178838598 | 178840240 |
| C3 | 178849480 | 178851124 |
| C3 | 178865402 | 178866507 |
| C3 | 178865811 | 178866891 |
| C3 | 178866815 | 178868004 |
| C3 | 178916038 | 178917467 |
| C3 | 178916978 | 178918187 |
| C3 | 178918578 | 178919828 |
| C3 | 178920832 | 178922077 |
| C3 | 178921791 | 178922876 |
| C3 | 178926883 | 178927988 |
| C3 | 178927474 | 178928626 |
| C3 | 178927719 | 178928853 |
| C3 | 178935498 | 178936622 |
| C3 | 178936484 | 178937565 |
| C3 | 178936839 | 178938023 |
| C3 | 178937237 | 178938340 |
| C3 | 178938274 | 178939445 |
| C3 | 178941369 | 178942475 |
| C3 | 178941988 | 178943109 |
| C3 | 178943250 | 178944328 |
| C3 | 178946560 | 178947730 |
| C3 | 178947292 | 178948409 |
| C3 | 178947513 | 178948664 |
| C3 | 178951382 | 178958381 |
| C3 | 179030576 | 179031844 |
| C3 | 179040857 | 179042082 |
| C3 | 179154650 | 179156393 |
| C3 | 179167782 | 179169513 |
| C3 | 179168790 | 179169832 |
| C3 | 179391170 | 179392477 |
| C3 | 181428299 | 181429376 |
| C3 | 182509662 | 182510814 |
| C3 | 182815327 | 182816615 |
| C3 | 182967611 | 182969022 |
| C3 | 183240369 | 183241383 |
| C3 | 183271827 | 183274581 |
| C3 | 183739848 | 183740926 |
| C3 | 183774760 | 183776017 |
| C3 | 183903341 | 183904389 |
| C3 | 183958828 | 183960150 |
| C3 | 184055851 | 184057153 |
| C3 | 184208526 | 184209787 |
| C3 | 184280107 | 184281957 |
| C3 | 184301392 | 184302566 |
| C3 | 184359653 | 184361207 |
| C3 | 185047316 | 185048468 |
| C3 | 185486851 | 185487863 |
| C3 | 185541999 | 185543157 |
| C3 | 185716692 | 185717847 |
| C3 | 185854792 | 185856123 |
| C3 | 185999536 | 186001159 |
| C3 | 186210918 | 186212311 |
| C3 | 186309470 | 186310707 |
| C3 | 186501464 | 186502490 |
| C3 | 186544512 | 186545669 |
| C3 | 186581921 | 186583058 |
| C3 | 186646355 | 186648284 |
| C3 | 186917178 | 186918454 |
| C3 | 186953129 | 186954385 |
| C3 | 187037986 | 187039917 |
| C3 | 187129513 | 187130610 |
| C3 | 187416148 | 187417225 |
| C3 | 187462172 | 187463225 |
| C3 | 187675943 | 187677168 |
| C3 | 187763330 | 187764393 |
| C3 | 188597597 | 188598618 |
| C3 | 189837756 | 189838871 |
| C3 | 189851246 | 189853643 |
| C3 | 189955112 | 189956203 |
| C3 | 190039667 | 190040873 |
| C3 | 190155901 | 190157436 |
| C3 | 190249011 | 190250999 |
| C3 | 190290034 | 190291278 |
| C3 | 190682475 | 190683549 |
| C3 | 190862081 | 190863291 |
| C3 | 190921237 | 190922500 |
| C3 | 191347700 | 191348838 |
| C3 | 192232112 | 192233196 |
| C3 | 192288510 | 192289891 |
| C3 | 192506362 | 192507399 |
| C3 | 192546062 | 192547267 |
| C3 | 192580161 | 192581182 |
| C3 | 192781327 | 192782476 |
| C3 | 192873143 | 192874714 |
| C3 | 192881809 | 192883224 |
| C3 | 192883675 | 192885873 |
| C3 | 193521086 | 193522092 |
| C3 | 193751934 | 193753042 |
| C3 | 193987719 | 193989140 |
| C3 | 194014439 | 194015561 |
| C3 | 194117616 | 194119299 |
| C3 | 194120753 | 194122212 |
| C3 | 194292746 | 194293797 |
| C3 | 194308429 | 194309503 |
| C3 | 194408129 | 194409234 |
| C3 | 194875997 | 194877254 |
| C3 | 194892198 | 194893774 |
| C3 | 194900350 | 194901649 |
| C3 | 195357136 | 195358196 |
| C3 | 195362463 | 195363706 |
| C3 | 195440232 | 195441975 |
| C3 | 195488384 | 195489737 |
| C3 | 195488758 | 195490090 |
| C3 | 195489094 | 195490233 |
| C3 | 195575988 | 195577269 |
| C3 | 195580620 | 195581771 |
| C3 | 195680911 | 195682002 |
| C3 | 195681865 | 195682986 |
| C3 | 195928878 | 195930035 |
| C3 | 195943026 | 195944137 |
| C3 | 195946503 | 195947579 |
| C3 | 196069304 | 196070483 |
| C3 | 196351418 | 196352636 |
| C3 | 196366641 | 196369999 |
| C3 | 196487455 | 196489244 |
| C3 | 196612345 | 196613825 |
| C3 | 196730250 | 196731373 |
| C3 | 196947747 | 196948844 |
| C3 | 197271324 | 197272878 |
| C3 | 197281968 | 197283027 |
| C3 | 197330599 | 197331830 |
| C3 | 197477814 | 197479021 |
| C3 | 197807273 | 197808502 |
| C3 | 197826108 | 197827311 |
| C3 | 197826408 | 197827709 |
| C3 | 197826858 | 197828611 |
| C3 | 197827934 | 197829477 |
| C3 | 197830082 | 197831209 |
| C3 | 197831576 | 197833077 |
| C3 | 197839851 | 197841099 |
| C3 | 197886733 | 197888055 |
| C3 | 197947261 | 197948396 |
| C4 | 10844 | 11895 |
| C4 | 123593 | 124657 |
| C4 | 639866 | 641011 |
| C4 | 659349 | 660799 |
| C4 | 675126 | 676262 |
| C4 | 704762 | 705848 |
| C4 | 720301 | 721520 |
| C4 | 738470 | 739594 |
| C4 | 775978 | 777205 |
| C4 | 779380 | 780494 |
| C4 | 780511 | 781655 |
| C4 | 880901 | 881913 |
| C4 | 940177 | 941335 |
| C4 | 940538 | 941596 |
| C4 | 941007 | 942069 |
| C4 | 961175 | 962314 |
| C4 | 961563 | 962642 |
| C4 | 1027385 | 1028518 |
| C4 | 1039357 | 1040673 |
| C4 | 1040477 | 1041607 |
| C4 | 1044244 | 1045320 |
| C4 | 1051831 | 1053557 |
| C4 | 1106248 | 1107379 |
| C4 | 1106406 | 1107603 |
| C4 | 1107194 | 1108283 |
| C4 | 1138732 | 1139826 |
| C4 | 1187696 | 1188818 |
| C4 | 1188289 | 1189374 |
| C4 | 1229699 | 1230718 |
| C4 | 1238906 | 1240042 |
| C4 | 1242442 | 1243502 |
| C4 | 1294467 | 1295702 |
| C4 | 1407568 | 1408678 |
| C4 | 1407777 | 1408869 |
| C4 | 1408032 | 1409107 |
| C4 | 1408350 | 1409418 |
| C4 | 1503904 | 1505081 |
| C4 | 1513817 | 1514986 |
| C4 | 1521484 | 1522942 |
| C4 | 1521982 | 1523111 |
| C4 | 1522223 | 1523352 |
| C4 | 1558217 | 1559267 |
| C4 | 1569882 | 1571107 |
| C4 | 1579809 | 1580938 |
| C4 | 1624512 | 1625729 |
| C4 | 1757319 | 1758573 |
| C4 | 1799773 | 1800876 |
| C4 | 1828970 | 1830823 |
| C4 | 2062059 | 2063192 |
| C4 | 2062209 | 2063250 |
| C4 | 2365972 | 2367064 |
| C4 | 2366528 | 2367638 |
| C4 | 2403586 | 2404871 |
| C4 | 2431151 | 2432758 |
| C4 | 2467218 | 2468295 |
| C4 | 2486226 | 2487472 |
| C4 | 2801589 | 2802850 |
| C4 | 3042648 | 3044011 |
| C4 | 3267794 | 3269084 |
| C4 | 3288052 | 3289169 |
| C4 | 3390282 | 3391676 |
| C4 | 3475726 | 3476741 |
| C4 | 3477400 | 3478513 |
| C4 | 3485402 | 3486748 |
| C4 | 3494795 | 3495947 |
| C4 | 3741681 | 3742811 |
| C4 | 4052783 | 4053924 |
| C4 | 4126001 | 4127241 |
| C4 | 4334726 | 4335845 |
| C4 | 4863970 | 4865384 |
| C4 | 5044071 | 5045206 |
| C4 | 5894097 | 5895242 |
| C4 | 6009429 | 6010489 |
| C4 | 6106664 | 6107977 |
| C4 | 6658970 | 6660079 |
| C4 | 6659216 | 6660257 |
| C4 | 6763225 | 6764321 |
| C4 | 6890820 | 6892005 |
| C4 | 6955176 | 6956513 |
| C4 | 7052112 | 7053262 |
| C4 | 7257102 | 7258208 |
| C4 | 7760006 | 7761600 |
| C4 | 7783364 | 7784441 |
| C4 | 8045926 | 8047092 |
| C4 | 8228442 | 8229623 |
| C4 | 8405412 | 8406487 |
| C4 | 8467793 | 8468811 |
| C4 | 9041941 | 9043032 |
| C4 | 9167575 | 9168939 |
| C4 | 9177072 | 9178258 |
| C4 | 9216529 | 9217731 |
| C4 | 9232051 | 9233485 |
| C4 | 9240352 | 9241703 |
| C4 | 9475621 | 9476791 |
| C4 | 9475880 | 9477200 |
| C4 | 9478457 | 9479856 |
| C4 | 10146618 | 10147802 |
| C4 | 15374973 | 15376029 |
| C4 | 15704197 | 15705542 |
| C4 | 15907195 | 15908245 |
| C4 | 15907291 | 15908472 |
| C4 | 16038400 | 16039817 |
| C4 | 16615911 | 16617066 |
| C4 | 16822567 | 16823714 |
| C4 | 17202365 | 17203519 |
| C4 | 17550859 | 17551914 |
| C4 | 17577445 | 17578589 |
| C4 | 17643151 | 17644232 |
| C4 | 18294754 | 18295924 |
| C4 | 19521525 | 19522577 |
| C4 | 20005060 | 20006332 |
| C4 | 20254236 | 20255291 |
| C4 | 23073929 | 23074938 |
| C4 | 24586091 | 24587185 |
| C4 | 24975175 | 24976373 |
| C4 | 25160750 | 25161881 |
| C4 | 25236436 | 25238074 |
| C4 | 25681970 | 25682994 |
| C4 | 25751084 | 25752288 |
| C4 | 28118454 | 28119568 |
| C4 | 30159548 | 30160615 |
| C4 | 30227597 | 30228863 |
| C4 | 31997468 | 31998728 |
| C4 | 31997774 | 31998900 |
| C4 | 34094022 | 34095207 |
| C4 | 34201898 | 34203187 |
| C4 | 35482963 | 35483995 |
| C4 | 35852287 | 35853422 |
| C4 | 38663718 | 38664855 |
| C4 | 39003025 | 39004921 |
| C4 | 39045850 | 39046896 |
| C4 | 40266174 | 40267894 |
| C4 | 40858366 | 40859478 |
| C4 | 40924641 | 40925744 |
| C4 | 41277522 | 41278732 |
| C4 | 41282143 | 41283314 |
| C4 | 41881671 | 41882816 |
| C4 | 42397873 | 42398973 |
| C4 | 42398320 | 42399474 |
| C4 | 43641094 | 43642245 |
| C4 | 46725949 | 46727070 |
| C4 | 47942911 | 47943937 |
| C4 | 48485303 | 48486409 |
| C4 | 48492497 | 48493549 |
| C4 | 48497048 | 48498095 |
| C4 | 49096365 | 49098530 |
| C4 | 49110218 | 49111745 |
| C4 | 49131579 | 49133085 |
| C4 | 49142439 | 49143685 |
| C4 | 49249030 | 49251099 |
| C4 | 49290323 | 49291596 |
| C4 | 49331203 | 49332630 |
| C4 | 52891985 | 52893219 |
| C4 | 52942458 | 52943698 |
| C4 | 54102651 | 54104176 |
| C4 | 54163236 | 54164245 |
| C4 | 54458828 | 54459967 |
| C4 | 54968476 | 54970355 |
| C4 | 54974821 | 54975958 |
| C4 | 55015014 | 55016099 |
| C4 | 55015150 | 55016404 |
| C4 | 55268414 | 55270396 |
| C4 | 55352718 | 55353848 |
| C4 | 55405252 | 55406399 |
| C4 | 55548387 | 55549521 |
| C4 | 55621571 | 55622718 |
| C4 | 55808256 | 55809395 |
| C4 | 56023397 | 56024848 |
| C4 | 57252230 | 57253445 |
| C4 | 57396262 | 57397426 |
| C4 | 57458246 | 57459444 |
| C4 | 57521900 | 57523080 |
| C4 | 57546920 | 57548112 |
| C4 | 57559285 | 57560434 |
| C4 | 57918486 | 57920203 |
| C4 | 58060013 | 58061494 |
| C4 | 58072473 | 58073553 |
| C4 | 60569981 | 60571135 |
| C4 | 63418616 | 63419649 |
| C4 | 64783751 | 64784961 |
| C4 | 65079333 | 65080470 |
| C4 | 65199104 | 65200212 |
| C4 | 66607847 | 66608920 |
| C4 | 67522432 | 67523672 |
| C4 | 68017120 | 68018189 |
| C4 | 69036616 | 69037711 |
| C4 | 69308313 | 69309463 |
| C4 | 69353905 | 69355054 |
| C4 | 69928405 | 69929532 |
| C4 | 69959380 | 69960519 |
| C4 | 71702879 | 71704076 |
| C4 | 71703263 | 71704415 |
| C4 | 72897238 | 72898322 |
| C4 | 73282502 | 73283526 |
| C4 | 75751898 | 75752932 |
| C4 | 76555165 | 76556272 |
| C4 | 76555503 | 76556641 |
| C4 | 77996056 | 77997104 |
| C4 | 79970669 | 79972078 |
| C4 | 80625573 | 80626649 |
| C4 | 80858598 | 80859695 |
| C4 | 81109530 | 81110586 |
| C4 | 81118619 | 81119750 |
| C4 | 81334747 | 81335882 |
| C4 | 83253394 | 83254416 |
| C4 | 83353509 | 83354555 |
| C4 | 84033206 | 84035086 |
| C4 | 84105673 | 84106861 |
| C4 | 84105996 | 84107087 |
| C4 | 85047492 | 85048670 |
| C4 | 85401870 | 85403011 |
| C4 | 85402452 | 85403609 |
| C4 | 86660428 | 86661521 |
| C4 | 86880105 | 86881440 |
| C4 | 86941317 | 86942457 |
| C4 | 87824795 | 87826297 |
| C4 | 88784357 | 88785438 |
| C4 | 89151902 | 89153148 |
| C4 | 89165898 | 89167029 |
| C4 | 89618286 | 89619499 |
| C4 | 89945976 | 89947073 |
| C4 | 91602306 | 91603371 |
| C4 | 93392615 | 93393884 |
| C4 | 95078733 | 95080099 |
| C4 | 96834773 | 96835913 |
| C4 | 98132913 | 98134186 |
| C4 | 99017789 | 99018857 |
| C4 | 99241505 | 99242798 |
| C4 | 99850385 | 99851782 |
| C4 | 100572654 | 100573838 |
| C4 | 103613035 | 103614214 |
| C4 | 108641072 | 108642130 |
| C4 | 109091592 | 109092630 |
| C4 | 109107411 | 109108508 |
| C4 | 109450849 | 109452327 |
| C4 | 110189654 | 110191494 |
| C4 | 110623753 | 110624789 |
| C4 | 111331852 | 111333033 |
| C4 | 111396679 | 111397781 |
| C4 | 111540073 | 111541296 |
| C4 | 114605954 | 114607045 |
| C4 | 117420179 | 117421308 |
| C4 | 117846910 | 117847943 |
| C4 | 119186190 | 119187369 |
| C4 | 119186511 | 119187572 |
| C4 | 119273728 | 119274766 |
| C4 | 119512322 | 119513388 |
| C4 | 119809566 | 119810736 |
| C4 | 119938694 | 119939751 |
| C4 | 120262797 | 120264314 |
| C4 | 120374599 | 120375910 |
| C4 | 121470822 | 121472173 |
| C4 | 121506829 | 121507956 |
| C4 | 122853135 | 122854352 |
| C4 | 125867024 | 125868143 |
| C4 | 128704066 | 128705149 |
| C4 | 131694345 | 131695554 |
| C4 | 132644064 | 132645156 |
| C4 | 132648004 | 132649099 |
| C4 | 132649450 | 132650842 |
| C4 | 132652982 | 132654128 |
| C4 | 132655516 | 132656721 |
| C4 | 132660219 | 132661324 |
| C4 | 132661918 | 132663274 |
| C4 | 132662299 | 132663460 |
| C4 | 132663030 | 132664117 |
| C4 | 132665559 | 132666795 |
| C4 | 132667019 | 132668241 |
| C4 | 132682876 | 132684096 |
| C4 | 132886304 | 132887369 |
| C4 | 132895410 | 132897992 |
| C4 | 133790115 | 133791261 |
| C4 | 133819287 | 133820390 |
| C4 | 134141323 | 134142409 |
| C4 | 137316168 | 137317305 |
| C4 | 138404559 | 138405975 |
| C4 | 138416941 | 138418299 |
| C4 | 140097919 | 140099058 |
| C4 | 140199269 | 140200374 |
| C4 | 140656206 | 140657294 |
| C4 | 141145502 | 141146734 |
| C4 | 141325924 | 141327060 |
| C4 | 142253621 | 142254805 |
| C4 | 142254088 | 142255208 |
| C4 | 144257341 | 144258440 |
| C4 | 144572831 | 144573917 |
| C4 | 144832567 | 144833714 |
| C4 | 144832729 | 144834004 |
| C4 | 145565888 | 145567014 |
| C4 | 147270964 | 147272095 |
| C4 | 148549077 | 148550097 |
| C4 | 148791899 | 148792909 |
| C4 | 149083321 | 149084367 |
| C4 | 150950522 | 150952084 |
| C4 | 151503990 | 151505057 |
| C4 | 151580538 | 151581623 |
| C4 | 153241910 | 153247883 |
| C4 | 153248860 | 153250725 |
| C4 | 153250324 | 153251437 |
| C4 | 153251384 | 153252520 |
| C4 | 153253248 | 153254419 |
| C4 | 153258454 | 153259588 |
| C4 | 153267464 | 153268723 |
| C4 | 153269326 | 153270458 |
| C4 | 153270694 | 153271776 |
| C4 | 153273122 | 153274623 |
| C4 | 153302841 | 153304167 |
| C4 | 153331812 | 153333530 |
| C4 | 153333132 | 153334258 |
| C4 | 153450925 | 153452502 |
| C4 | 153455593 | 153457753 |
| C4 | 154727494 | 154728508 |
| C4 | 155702200 | 155703575 |
| C4 | 159644634 | 159645697 |
| C4 | 161033883 | 161034986 |
| C4 | 161579316 | 161580966 |
| C4 | 161753687 | 161754721 |
| C4 | 162129738 | 162130814 |
| C4 | 162209089 | 162210243 |
| C4 | 162613435 | 162614525 |
| C4 | 165001687 | 165002791 |
| C4 | 166879425 | 166880972 |
| C4 | 167363107 | 167364113 |
| C4 | 167388363 | 167389385 |
| C4 | 167422536 | 167423735 |
| C4 | 167676685 | 167677888 |
| C4 | 167761468 | 167762616 |
| C4 | 168046258 | 168048028 |
| C4 | 169552243 | 169553388 |
| C4 | 172218946 | 172220010 |
| C4 | 172383717 | 172384852 |
| C4 | 174618639 | 174619688 |
| C4 | 174870495 | 174872018 |
| C4 | 174960221 | 174961239 |
| C4 | 176348567 | 176349645 |
| C4 | 176952366 | 176953391 |
| C4 | 177240519 | 177241588 |
| C4 | 177615278 | 177617402 |
| C4 | 181179338 | 181181072 |
| C4 | 181316822 | 181317925 |
| C4 | 181865303 | 181866401 |
| C4 | 181865837 | 181867191 |
| C4 | 182474163 | 182475296 |
| C4 | 183060184 | 183061476 |
| C4 | 183062914 | 183064005 |
| C4 | 183262148 | 183263168 |
| C4 | 183720819 | 183721909 |
| C4 | 183727696 | 183728964 |
| C4 | 183728518 | 183729579 |
| C4 | 183729953 | 183731272 |
| C4 | 183747272 | 183748416 |
| C4 | 184263435 | 184264525 |
| C4 | 184263556 | 184264792 |
| C4 | 184329726 | 184330766 |
| C4 | 184566617 | 184567667 |
| C4 | 184642610 | 184643950 |
| C4 | 185127503 | 185128513 |
| C4 | 185724036 | 185725138 |
| C4 | 185732322 | 185733978 |
| C4 | 185779153 | 185780346 |
| C4 | 185879546 | 185880709 |
| C4 | 186198447 | 186199577 |
| C4 | 187070924 | 187072369 |
| C4 | 187072660 | 187073668 |
| C4 | 187124806 | 187126074 |
| C4 | 187219038 | 187220295 |
| C4 | 187268775 | 187269809 |
| C4 | 187795320 | 187796471 |
| C4 | 187881232 | 187882389 |
| C4 | 187984851 | 187985944 |
| C4 | 187999334 | 188000357 |
| C4 | 188449806 | 188450907 |
| C4 | 188735696 | 188736766 |
| C4 | 188735820 | 188736899 |
| C4 | 188805432 | 188806446 |
| C4 | 188861621 | 188862752 |
| C4 | 188917628 | 188918719 |
| C4 | 189123467 | 189125050 |
| C4 | 189629125 | 189630201 |
| C4 | 189956482 | 189957765 |
| C4 | 189956798 | 189957945 |
| C4 | 189972315 | 189973577 |
| C4 | 190057770 | 190058989 |
| C4 | 190058111 | 190059447 |
| C4 | 190185765 | 190186800 |
| C4 | 190471706 | 190473288 |
| C4 | 190472618 | 190474102 |
| C4 | 190474637 | 190476558 |
| C4 | 190475930 | 190477207 |
| C4 | 190479344 | 190485206 |
| C4 | 190485820 | 190487456 |
| C4 | 190490590 | 190492961 |
| C4 | 190493095 | 190494613 |
| C4 | 190495388 | 190497336 |
| C4 | 190500068 | 190501836 |
| C4 | 190501758 | 190504363 |
| C4 | 190504718 | 190507329 |
| C4 | 190506992 | 190508701 |
| C4 | 190508238 | 190509683 |
| C4 | 190509526 | 190511393 |
| C4 | 190511107 | 190513097 |
| C4 | 190513880 | 190515476 |
| C4 | 190517973 | 190519372 |
| C4 | 190521887 | 190523480 |
| C4 | 190525233 | 190526657 |
| C4 | 190526322 | 190528171 |
| C4 | 190528133 | 190529678 |
| C4 | 190528944 | 190530260 |
| C4 | 190530565 | 190533188 |
| C4 | 190538292 | 190539903 |
| C4 | 190567064 | 190568145 |
| C4 | 190607080 | 190608419 |
| C4 | 190804870 | 190807205 |
| C4 | 190813301 | 190815149 |
| C4 | 190819587 | 190822261 |
| C4 | 190861196 | 190862256 |
| C4 | 190861841 | 190862921 |
| C4 | 190942412 | 190943620 |
| C4 | 190942823 | 190943929 |
| C4 | 190943274 | 190944396 |
| C4 | 190958657 | 190959997 |
| C4 | 190961570 | 190963130 |
| C4 | 190986433 | 190987577 |
| C4 | 190987837 | 190988965 |
| C4 | 190988000 | 190989202 |
| C4 | 190988938 | 190990061 |
| C4 | 190989171 | 190990239 |
| C4 | 190989315 | 190990409 |
| C4 | 190989543 | 190991108 |
| C4 | 190990239 | 190991507 |
| C4 | 190991492 | 190992664 |
| C4 | 190993753 | 190994978 |
| C4 | 190993986 | 190995093 |
| C4 | 190994110 | 190995213 |
| C4 | 191000426 | 191001553 |
| C4 | 191002695 | 191003839 |
| C4 | 191010388 | 191011706 |
| C4 | 191013510 | 191014605 |
| C5 | 164032 | 165497 |
| C5 | 191220 | 192391 |
| C5 | 210910 | 212648 |
| C5 | 211794 | 212942 |
| C5 | 259770 | 260883 |
| C5 | 309402 | 310487 |
| C5 | 391733 | 392864 |
| C5 | 415007 | 416212 |
| C5 | 420835 | 421879 |
| C5 | 428855 | 430128 |
| C5 | 467477 | 469030 |
| C5 | 481645 | 482725 |
| C5 | 483111 | 484401 |
| C5 | 483425 | 484542 |
| C5 | 483825 | 485032 |
| C5 | 505750 | 506923 |
| C5 | 553933 | 555130 |
| C5 | 561764 | 563065 |
| C5 | 562822 | 563953 |
| C5 | 562971 | 564026 |
| C5 | 563167 | 564711 |
| C5 | 563713 | 564800 |
| C5 | 564242 | 565372 |
| C5 | 565019 | 566126 |
| C5 | 664052 | 665271 |
| C5 | 804831 | 805914 |
| C5 | 958214 | 959703 |
| C5 | 1023078 | 1024160 |
| C5 | 1067538 | 1068573 |
| C5 | 1108051 | 1109318 |
| C5 | 1130565 | 1131873 |
| C5 | 1161458 | 1162627 |
| C5 | 1178315 | 1179892 |
| C5 | 1191325 | 1192585 |
| C5 | 1245402 | 1246507 |
| C5 | 1245644 | 1246689 |
| C5 | 1474596 | 1475847 |
| C5 | 1490347 | 1491488 |
| C5 | 1494694 | 1495745 |
| C5 | 1503224 | 1504297 |
| C5 | 1521386 | 1523378 |
| C5 | 1548531 | 1549562 |
| C5 | 1594138 | 1595220 |
| C5 | 1594625 | 1595708 |
| C5 | 1634511 | 1635686 |
| C5 | 1663231 | 1664286 |
| C5 | 1728361 | 1729717 |
| C5 | 1839654 | 1840837 |
| C5 | 1849556 | 1850608 |
| C5 | 1856511 | 1857911 |
| C5 | 1867524 | 1868588 |
| C5 | 1875715 | 1876899 |
| C5 | 1878043 | 1879192 |
| C5 | 1948495 | 1949593 |
| C5 | 1950719 | 1951832 |
| C5 | 1975293 | 1977066 |
| C5 | 2007111 | 2008334 |
| C5 | 2024514 | 2025574 |
| C5 | 2866412 | 2867707 |
| C5 | 2925743 | 2926938 |
| C5 | 3056959 | 3058393 |
| C5 | 3102025 | 3103070 |
| C5 | 3187536 | 3188760 |
| C5 | 3637491 | 3638855 |
| C5 | 3638085 | 3639581 |
| C5 | 3931618 | 3932829 |
| C5 | 4143338 | 4144548 |
| C5 | 4630149 | 4631219 |
| C5 | 5571222 | 5572780 |
| C5 | 5639050 | 5640399 |
| C5 | 5722247 | 5723413 |
| C5 | 5806147 | 5808033 |
| C5 | 6348979 | 6350820 |
| C5 | 6418836 | 6420707 |
| C5 | 6773029 | 6774444 |
| C5 | 6773722 | 6774956 |
| C5 | 6774495 | 6776498 |
| C5 | 6822011 | 6823651 |
| C5 | 6846226 | 6847344 |
| C5 | 7289263 | 7290441 |
| C5 | 7300624 | 7301808 |
| C5 | 8052760 | 8054122 |
| C5 | 8135966 | 8137829 |
| C5 | 8422123 | 8423151 |
| C5 | 8423835 | 8425222 |
| C5 | 8456933 | 8458772 |
| C5 | 8519055 | 8520653 |
| C5 | 8839307 | 8840469 |
| C5 | 8839902 | 8841092 |
| C5 | 9044000 | 9045962 |
| C5 | 9051513 | 9052823 |
| C5 | 9052830 | 9054816 |
| C5 | 9178445 | 9181572 |
| C5 | 9400141 | 9401472 |
| C5 | 9431790 | 9433600 |
| C5 | 9545604 | 9546740 |
| C5 | 9900425 | 9901932 |
| C5 | 9984721 | 9986714 |
| C5 | 10561791 | 10563346 |
| C5 | 10564350 | 10565461 |
| C5 | 10565038 | 10566534 |
| C5 | 10625689 | 10626754 |
| C5 | 10649115 | 10650194 |
| C5 | 10649444 | 10650533 |
| C5 | 10649742 | 10650817 |
| C5 | 10681535 | 10683036 |
| C5 | 10878657 | 10879666 |
| C5 | 12186169 | 12187288 |
| C5 | 12354128 | 12355229 |
| C5 | 12817846 | 12819548 |
| C5 | 13000356 | 13001429 |
| C5 | 13578170 | 13579262 |
| C5 | 13611965 | 13613001 |
| C5 | 13854840 | 13856008 |
| C5 | 14530997 | 14532816 |
| C5 | 14559792 | 14561361 |
| C5 | 14992773 | 14993836 |
| C5 | 15342236 | 15343383 |
| C5 | 16623887 | 16624959 |
| C5 | 17061796 | 17062873 |
| C5 | 17421342 | 17423318 |
| C5 | 17466411 | 17467544 |
| C5 | 17590031 | 17591191 |
| C5 | 17607906 | 17609141 |
| C5 | 17736015 | 17738620 |
| C5 | 17778468 | 17780287 |
| C5 | 18131946 | 18133763 |
| C5 | 18671710 | 18672717 |
| C5 | 19161105 | 19162322 |
| C5 | 19287375 | 19288396 |
| C5 | 21340080 | 21341764 |
| C5 | 21341057 | 21342116 |
| C5 | 21341219 | 21342367 |
| C5 | 22852566 | 22853844 |
| C5 | 23506870 | 23508236 |
| C5 | 23657446 | 23658532 |
| C5 | 23881393 | 23882494 |
| C5 | 25190283 | 25191369 |
| C5 | 25315912 | 25317130 |
| C5 | 27426172 | 27427422 |
| C5 | 27908470 | 27909556 |
| C5 | 28927118 | 28929112 |
| C5 | 29519956 | 29520978 |
| C5 | 29712457 | 29713485 |
| C5 | 30345349 | 30346420 |
| C5 | 30816943 | 30819502 |
| C5 | 30821193 | 30823094 |
| C5 | 31506926 | 31507936 |
| C5 | 31701276 | 31702434 |
| C5 | 31749459 | 31750481 |
| C5 | 31782854 | 31784375 |
| C5 | 31802168 | 31803552 |
| C5 | 32207682 | 32208785 |
| C5 | 32208423 | 32209652 |
| C5 | 32437891 | 32439165 |
| C5 | 32445748 | 32446883 |
| C5 | 32514186 | 32515208 |
| C5 | 32708617 | 32709837 |
| C5 | 32711418 | 32712507 |
| C5 | 33355111 | 33356197 |
| C5 | 33515046 | 33516476 |
| C5 | 33936816 | 33938240 |
| C5 | 34044226 | 34045276 |
| C5 | 34244733 | 34245869 |
| C5 | 37012359 | 37013872 |
| C5 | 37208573 | 37209752 |
| C5 | 37208810 | 37209948 |
| C5 | 37378159 | 37379426 |
| C5 | 38844723 | 38845980 |
| C5 | 39073900 | 39074975 |
| C5 | 39787621 | 39789021 |
| C5 | 40747833 | 40748868 |
| C5 | 42923692 | 42924950 |
| C5 | 42923976 | 42924988 |
| C5 | 42951596 | 42952764 |
| C5 | 42980272 | 42982204 |
| C5 | 42999858 | 43000918 |
| C5 | 43017572 | 43018767 |
| C5 | 43033757 | 43034810 |
| C5 | 43036873 | 43037971 |
| C5 | 43396774 | 43397952 |
| C5 | 43745493 | 43746640 |
| C5 | 52231328 | 52232586 |
| C5 | 53846813 | 53847912 |
| C5 | 53956382 | 53957445 |
| C5 | 54613738 | 54614767 |
| C5 | 55155466 | 55156495 |
| C5 | 55622912 | 55624447 |
| C5 | 57303241 | 57304263 |
| C5 | 57893149 | 57894438 |
| C5 | 58335181 | 58336290 |
| C5 | 60584517 | 60585580 |
| C5 | 62099378 | 62101056 |
| C5 | 62620332 | 62621459 |
| C5 | 62704008 | 62705106 |
| C5 | 63256308 | 63257374 |
| C5 | 63972516 | 63973576 |
| C5 | 64459786 | 64461396 |
| C5 | 65028479 | 65030248 |
| C5 | 67483046 | 67484103 |
| C5 | 68376546 | 68377659 |
| C5 | 68856745 | 68858127 |
| C5 | 70199614 | 70200764 |
| C5 | 70820740 | 70821812 |
| C5 | 70820888 | 70821990 |
| C5 | 71145675 | 71147585 |
| C5 | 72221089 | 72222148 |
| C5 | 72528748 | 72529771 |
| C5 | 72593207 | 72594509 |
| C5 | 72793856 | 72794906 |
| C5 | 73977555 | 73978615 |
| C5 | 74349632 | 74350738 |
| C5 | 74806180 | 74807261 |
| C5 | 76249646 | 76250680 |
| C5 | 76279839 | 76280935 |
| C5 | 76372735 | 76374112 |
| C5 | 76714164 | 76715202 |
| C5 | 76934474 | 76935608 |
| C5 | 77104831 | 77105972 |
| C5 | 77141969 | 77143759 |
| C5 | 77143074 | 77144137 |
| C5 | 77146041 | 77147823 |
| C5 | 78272260 | 78273624 |
| C5 | 78276296 | 78277423 |
| C5 | 78431933 | 78433542 |
| C5 | 79338247 | 79339283 |
| C5 | 79511493 | 79512973 |
| C5 | 79627939 | 79628947 |
| C5 | 80255973 | 80257170 |
| C5 | 81435093 | 81436370 |
| C5 | 81435939 | 81437104 |
| C5 | 82482331 | 82483465 |
| C5 | 82758778 | 82759887 |
| C5 | 84294065 | 84295175 |
| C5 | 84632622 | 84633652 |
| C5 | 84716298 | 84717333 |
| C5 | 87989551 | 87990674 |
| C5 | 92905647 | 92906858 |
| C5 | 92922970 | 92924363 |
| C5 | 92923952 | 92924995 |
| C5 | 94109209 | 94110370 |
| C5 | 95909406 | 95910541 |
| C5 | 96743576 | 96744602 |
| C5 | 98174571 | 98176065 |
| C5 | 100239548 | 100240775 |
| C5 | 100422232 | 100423355 |
| C5 | 100637437 | 100639868 |
| C5 | 101490030 | 101491158 |
| C5 | 102087117 | 102088308 |
| C5 | 105676702 | 105677715 |
| C5 | 105752534 | 105753654 |
| C5 | 107826599 | 107827616 |
| C5 | 108676948 | 108678015 |
| C5 | 109296810 | 109297904 |
| C5 | 110229496 | 110230554 |
| C5 | 110637699 | 110638808 |
| C5 | 111791203 | 111792270 |
| C5 | 114264189 | 114265391 |
| C5 | 114730788 | 114731934 |
| C5 | 114731033 | 114732196 |
| C5 | 115025838 | 115026982 |
| C5 | 115696635 | 115697775 |
| C5 | 116790182 | 116791458 |
| C5 | 119024814 | 119026530 |
| C5 | 122033864 | 122034908 |
| C5 | 122403299 | 122404320 |
| C5 | 124070486 | 124071664 |
| C5 | 126111082 | 126112190 |
| C5 | 126115155 | 126116257 |
| C5 | 126189007 | 126190082 |
| C5 | 126204482 | 126205599 |
| C5 | 126289349 | 126290621 |
| C5 | 126457925 | 126459222 |
| C5 | 126904503 | 126905965 |
| C5 | 127091151 | 127092422 |
| C5 | 127872086 | 127873179 |
| C5 | 130246577 | 130247597 |
| C5 | 130599187 | 130600272 |
| C5 | 131606519 | 131608055 |
| C5 | 131706197 | 131707551 |
| C5 | 132160769 | 132162129 |
| C5 | 133268350 | 133269655 |
| C5 | 134240059 | 134241191 |
| C5 | 134262269 | 134263339 |
| C5 | 134262740 | 134264053 |
| C5 | 134362957 | 134364046 |
| C5 | 134826913 | 134828133 |
| C5 | 135415952 | 135417080 |
| C5 | 136384324 | 136385806 |
| C5 | 136919228 | 136920264 |
| C5 | 137390256 | 137391278 |
| C5 | 137394804 | 137395826 |
| C5 | 138346005 | 138347358 |
| C5 | 138346422 | 138347523 |
| C5 | 138713702 | 138714816 |
| C5 | 138727772 | 138729186 |
| C5 | 139088294 | 139089434 |
| C5 | 139138704 | 139139739 |
| C5 | 139227217 | 139228308 |
| C5 | 139991472 | 139992582 |
| C5 | 140098386 | 140099516 |
| C5 | 140165671 | 140166714 |
| C5 | 140214748 | 140215826 |
| C5 | 140215096 | 140216283 |
| C5 | 140222218 | 140223310 |
| C5 | 140222409 | 140223566 |
| C5 | 140236705 | 140237802 |
| C5 | 140256083 | 140257181 |
| C5 | 140455513 | 140456645 |
| C5 | 140481349 | 140482513 |
| C5 | 140481889 | 140483011 |
| C5 | 140502750 | 140503823 |
| C5 | 140580527 | 140581695 |
| C5 | 140593207 | 140594487 |
| C5 | 140595040 | 140596139 |
| C5 | 140604356 | 140605480 |
| C5 | 140615504 | 140616585 |
| C5 | 140626355 | 140627476 |
| C5 | 140711811 | 140712858 |
| C5 | 140720098 | 140721205 |
| C5 | 140736345 | 140737394 |
| C5 | 140750891 | 140751989 |
| C5 | 140751254 | 140752326 |
| C5 | 140754755 | 140755902 |
| C5 | 140777274 | 140778385 |
| C5 | 140788898 | 140789994 |
| C5 | 140794201 | 140795289 |
| C5 | 140809606 | 140810661 |
| C5 | 140810432 | 140811636 |
| C5 | 140870867 | 140871986 |
| C5 | 140871825 | 140872986 |
| C5 | 140873815 | 140875243 |
| C5 | 141165129 | 141166163 |
| C5 | 141278389 | 141279415 |
| C5 | 142344466 | 142345816 |
| C5 | 143160415 | 143161473 |
| C5 | 143977691 | 143978742 |
| C5 | 143999698 | 144000858 |
| C5 | 145721632 | 145722822 |
| C5 | 146032679 | 146033965 |
| C5 | 146798364 | 146799406 |
| C5 | 146881362 | 146882784 |
| C5 | 147698858 | 147700487 |
| C5 | 147699864 | 147700937 |
| C5 | 150052955 | 150054031 |
| C5 | 150225778 | 150226947 |
| C5 | 150313305 | 150314378 |
| C5 | 150618445 | 150619679 |
| C5 | 150910717 | 150911950 |
| C5 | 151093190 | 151094267 |
| C5 | 151149683 | 151150771 |
| C5 | 151250684 | 151251925 |
| C5 | 151456052 | 151457344 |
| C5 | 152195198 | 152196316 |
| C5 | 152675184 | 152676303 |
| C5 | 152675705 | 152676840 |
| C5 | 152756880 | 152758018 |
| C5 | 153861857 | 153862924 |
| C5 | 154423355 | 154424368 |
| C5 | 154645657 | 154647039 |
| C5 | 156092358 | 156093575 |
| C5 | 156870888 | 156871897 |
| C5 | 157097895 | 157099189 |
| C5 | 157117314 | 157118352 |
| C5 | 158477886 | 158478995 |
| C5 | 158531009 | 158532147 |
| C5 | 159619943 | 159621061 |
| C5 | 159902633 | 159903879 |
| C5 | 160189605 | 160191382 |
| C5 | 161178846 | 161179921 |
| C5 | 162167628 | 162168831 |
| C5 | 165140750 | 165141831 |
| C5 | 165695459 | 165696584 |
| C5 | 166795337 | 166796600 |
| C5 | 167285245 | 167286334 |
| C5 | 167286796 | 167287986 |
| C5 | 167860771 | 167861904 |
| C5 | 167912159 | 167913232 |
| C5 | 169063165 | 169064185 |
| C5 | 169179292 | 169180337 |
| C5 | 169669531 | 169671796 |
| C5 | 169722188 | 169724971 |
| C5 | 169734239 | 169735555 |
| C5 | 170342609 | 170344313 |
| C5 | 170739073 | 170740181 |
| C5 | 170846548 | 170847632 |
| C5 | 171723335 | 171724515 |
| C5 | 172110153 | 172111231 |
| C5 | 172110364 | 172111586 |
| C5 | 172110719 | 172111793 |
| C5 | 172110907 | 172112124 |
| C5 | 172175117 | 172176184 |
| C5 | 172226645 | 172227714 |
| C5 | 174181296 | 174182362 |
| C5 | 175084994 | 175086519 |
| C5 | 175664998 | 175666060 |
| C5 | 175732198 | 175733686 |
| C5 | 175956261 | 175957786 |
| C5 | 175978405 | 175979506 |
| C5 | 176302413 | 176303680 |
| C5 | 176388365 | 176389463 |
| C5 | 176388699 | 176390136 |
| C5 | 176543555 | 176544767 |
| C5 | 176558757 | 176559922 |
| C5 | 176675989 | 176677007 |
| C5 | 176734270 | 176735393 |
| C5 | 176758693 | 176759973 |
| C5 | 176763877 | 176764974 |
| C5 | 176793354 | 176794594 |
| C5 | 176797428 | 176798565 |
| C5 | 176826740 | 176827781 |
| C5 | 176826837 | 176827923 |
| C5 | 176829905 | 176831019 |
| C5 | 176893361 | 176894548 |
| C5 | 176934805 | 176935904 |
| C5 | 176937334 | 176938517 |
| C5 | 177097217 | 177098785 |
| C5 | 177199002 | 177200124 |
| C5 | 177208873 | 177210067 |
| C5 | 177209365 | 177210513 |
| C5 | 177233799 | 177234925 |
| C5 | 177434066 | 177435710 |
| C5 | 177435021 | 177436208 |
| C5 | 177560304 | 177561362 |
| C5 | 177716285 | 177717329 |
| C5 | 177739456 | 177740535 |
| C5 | 177763721 | 177764840 |
| C5 | 177822448 | 177823586 |
| C5 | 178155706 | 178156947 |
| C5 | 178232284 | 178233492 |
| C5 | 178450417 | 178451481 |
| C5 | 178565437 | 178566723 |
| C5 | 178677386 | 178678412 |
| C5 | 178692262 | 178693324 |
| C5 | 178762571 | 178763616 |
| C5 | 178889717 | 178891058 |
| C5 | 178956705 | 178957780 |
| C5 | 178956835 | 178958098 |
| C5 | 178985900 | 178987098 |
| C5 | 178986177 | 178987328 |
| C5 | 179228311 | 179229502 |
| C5 | 179339670 | 179340700 |
| C5 | 179539824 | 179540920 |
| C5 | 179588220 | 179589273 |
| C5 | 179740140 | 179741601 |
| C5 | 180045290 | 180046409 |
| C5 | 180045432 | 180046504 |
| C5 | 180045653 | 180046892 |
| C5 | 180048130 | 180049247 |
| C5 | 180085660 | 180086716 |
| C5 | 180100695 | 180101846 |
| C5 | 180334875 | 180335935 |
| C5 | 180430201 | 180431996 |
| C5 | 180609585 | 180610914 |
| C5 | 180609998 | 180611130 |
| C5 | 180611187 | 180612372 |
| C5 | 180621956 | 180623066 |
| C5 | 180645203 | 180646320 |
| C6 | 290842 | 292780 |
| C6 | 291855 | 292993 |
| C6 | 292011 | 293104 |
| C6 | 511730 | 512819 |
| C6 | 683566 | 684624 |
| C6 | 867319 | 868337 |
| C6 | 1317241 | 1318431 |
| C6 | 1555495 | 1556549 |
| C6 | 1605178 | 1606324 |
| C6 | 1614882 | 1615981 |
| C6 | 1615073 | 1616180 |
| C6 | 2146334 | 2147417 |
| C6 | 2762384 | 2763580 |
| C6 | 2903012 | 2904105 |
| C6 | 2940351 | 2941496 |
| C6 | 2952402 | 2953528 |
| C6 | 2952634 | 2953763 |
| C6 | 3012382 | 3013791 |
| C6 | 3091606 | 3092873 |
| C6 | 3771171 | 3772564 |
| C6 | 3825364 | 3826486 |
| C6 | 3848735 | 3850131 |
| C6 | 3849653 | 3850816 |
| C6 | 4078850 | 4079948 |
| C6 | 5010162 | 5011218 |
| C6 | 5134824 | 5136099 |
| C6 | 5827000 | 5828383 |
| C6 | 5836162 | 5837346 |
| C6 | 5891702 | 5892899 |
| C6 | 5995072 | 5996266 |
| C6 | 6264790 | 6265823 |
| C6 | 6319807 | 6320890 |
| C6 | 6702302 | 6703441 |
| C6 | 6724793 | 6725976 |
| C6 | 6748318 | 6749602 |
| C6 | 6774288 | 6775326 |
| C6 | 7050799 | 7052005 |
| C6 | 7314987 | 7316050 |
| C6 | 7343806 | 7344972 |
| C6 | 8998831 | 8999909 |
| C6 | 9024934 | 9026608 |
| C6 | 9722313 | 9723361 |
| C6 | 10381264 | 10382407 |
| C6 | 10452594 | 10453675 |
| C6 | 10659704 | 10660858 |
| C6 | 10719898 | 10721561 |
| C6 | 11199432 | 11200942 |
| C6 | 12011158 | 12012233 |
| C6 | 12537932 | 12538972 |
| C6 | 12653466 | 12654516 |
| C6 | 13278962 | 13280007 |
| C6 | 13284309 | 13285918 |
| C6 | 13427869 | 13429188 |
| C6 | 13487340 | 13488417 |
| C6 | 13742975 | 13744375 |
| C6 | 13859581 | 13860754 |
| C6 | 13888192 | 13889234 |
| C6 | 14117816 | 14119005 |
| C6 | 14479739 | 14480876 |
| C6 | 14933509 | 14934574 |
| C6 | 15189477 | 15190777 |
| C6 | 16179888 | 16181160 |
| C6 | 16197963 | 16199058 |
| C6 | 16479738 | 16480798 |
| C6 | 17015726 | 17016864 |
| C6 | 17015960 | 17017093 |
| C6 | 17580487 | 17581588 |
| C6 | 17599976 | 17601063 |
| C6 | 17703805 | 17704895 |
| C6 | 17749730 | 17751054 |
| C6 | 18019503 | 18020572 |
| C6 | 18019674 | 18020790 |
| C6 | 18310054 | 18311064 |
| C6 | 18866885 | 18867969 |
| C6 | 19149008 | 19150237 |
| C6 | 19673434 | 19674665 |
| C6 | 19676084 | 19677762 |
| C6 | 20023571 | 20024606 |
| C6 | 20032035 | 20033200 |
| C6 | 20319518 | 20321083 |
| C6 | 20405116 | 20406305 |
| C6 | 20765032 | 20766130 |
| C6 | 21479124 | 21480293 |
| C6 | 21667075 | 21668408 |
| C6 | 22062897 | 22064084 |
| C6 | 23052807 | 23053936 |
| C6 | 23279801 | 23280834 |
| C6 | 25041817 | 25043151 |
| C6 | 25179645 | 25180944 |
| C6 | 25820077 | 25821519 |
| C6 | 25881873 | 25883295 |
| C6 | 26042228 | 26043653 |
| C6 | 26171708 | 26172780 |
| C6 | 26230289 | 26231637 |
| C6 | 26232690 | 26234162 |
| C6 | 26239969 | 26241662 |
| C6 | 26253898 | 26255105 |
| C6 | 26550298 | 26551364 |
| C6 | 26723508 | 26724642 |
| C6 | 26743090 | 26744185 |
| C6 | 26758066 | 26759292 |
| C6 | 26777570 | 26778657 |
| C6 | 27099929 | 27101056 |
| C6 | 27113814 | 27114880 |
| C6 | 27185092 | 27186702 |
| C6 | 27451982 | 27453101 |
| C6 | 27482546 | 27483661 |
| C6 | 27510528 | 27511680 |
| C6 | 27620837 | 27622066 |
| C6 | 27705681 | 27707088 |
| C6 | 27724605 | 27725768 |
| C6 | 27729442 | 27730753 |
| C6 | 27731245 | 27732399 |
| C6 | 27770359 | 27771397 |
| C6 | 27777511 | 27778626 |
| C6 | 27783444 | 27784591 |
| C6 | 27793984 | 27795078 |
| C6 | 27794133 | 27795345 |
| C6 | 27794372 | 27795616 |
| C6 | 27794652 | 27795748 |
| C6 | 27795042 | 27796158 |
| C6 | 27840123 | 27841913 |
| C6 | 27857795 | 27858932 |
| C6 | 28058215 | 28059430 |
| C6 | 28175010 | 28176105 |
| C6 | 28366021 | 28367205 |
| C6 | 28367075 | 28368150 |
| C6 | 28414285 | 28415606 |
| C6 | 28445955 | 28447086 |
| C6 | 28633467 | 28634768 |
| C6 | 28723615 | 28724826 |
| C6 | 28748960 | 28750163 |
| C6 | 28805177 | 28806314 |
| C6 | 28851740 | 28853584 |
| C6 | 28917781 | 28918973 |
| C6 | 29229637 | 29230699 |
| C6 | 29648313 | 29649462 |
| C6 | 29686596 | 29687694 |
| C6 | 29689715 | 29691267 |
| C6 | 29831414 | 29832525 |
| C6 | 29893695 | 29894966 |
| C6 | 29894205 | 29895331 |
| C6 | 30155262 | 30156413 |
| C6 | 30420230 | 30421347 |
| C6 | 30485889 | 30486956 |
| C6 | 30547389 | 30548611 |
| C6 | 30659237 | 30660412 |
| C6 | 31211972 | 31213067 |
| C6 | 31238441 | 31239524 |
| C6 | 31238548 | 31239642 |
| C6 | 31525394 | 31526536 |
| C6 | 31650109 | 31651673 |
| C6 | 31690569 | 31691727 |
| C6 | 31867217 | 31868258 |
| C6 | 31900128 | 31901285 |
| C6 | 32063799 | 32064877 |
| C6 | 32116038 | 32117283 |
| C6 | 32117807 | 32118876 |
| C6 | 32312682 | 32314353 |
| C6 | 32449273 | 32450298 |
| C6 | 32489053 | 32490302 |
| C6 | 32489522 | 32490693 |
| C6 | 32514609 | 32515824 |
| C6 | 32551505 | 32552607 |
| C6 | 32561681 | 32563079 |
| C6 | 32562568 | 32563671 |
| C6 | 32563024 | 32564128 |
| C6 | 32666905 | 32668142 |
| C6 | 32671260 | 32672420 |
| C6 | 32728856 | 32729942 |
| C6 | 33026894 | 33028201 |
| C6 | 33093122 | 33094208 |
| C6 | 33104233 | 33105369 |
| C6 | 33395306 | 33396400 |
| C6 | 33408041 | 33413989 |
| C6 | 33452916 | 33454007 |
| C6 | 33492839 | 33494016 |
| C6 | 33600328 | 33601502 |
| C6 | 33655719 | 33656738 |
| C6 | 33660628 | 33661804 |
| C6 | 34033334 | 34034691 |
| C6 | 34202917 | 34204080 |
| C6 | 34365354 | 34366715 |
| C6 | 34481898 | 34482994 |
| C6 | 34723992 | 34725048 |
| C6 | 34856490 | 34857529 |
| C6 | 35031626 | 35032700 |
| C6 | 35149636 | 35150714 |
| C6 | 35285721 | 35286821 |
| C6 | 35285942 | 35287018 |
| C6 | 35307686 | 35308810 |
| C6 | 35307953 | 35309080 |
| C6 | 35338467 | 35340062 |
| C6 | 35453419 | 35454593 |
| C6 | 35529259 | 35530727 |
| C6 | 35733258 | 35734311 |
| C6 | 35754235 | 35755359 |
| C6 | 36391166 | 36392462 |
| C6 | 36538333 | 36539343 |
| C6 | 36551015 | 36552246 |
| C6 | 36560183 | 36561416 |
| C6 | 37313612 | 37314692 |
| C6 | 37617414 | 37618509 |
| C6 | 37721158 | 37722293 |
| C6 | 38127191 | 38128266 |
| C6 | 38921776 | 38922974 |
| C6 | 39200646 | 39201675 |
| C6 | 39280199 | 39281360 |
| C6 | 41068053 | 41069222 |
| C6 | 41068392 | 41069433 |
| C6 | 41338951 | 41340076 |
| C6 | 41340489 | 41341617 |
| C6 | 41374439 | 41375500 |
| C6 | 41376057 | 41377208 |
| C6 | 41376259 | 41377495 |
| C6 | 41527698 | 41528844 |
| C6 | 41702644 | 41703735 |
| C6 | 42072127 | 42073200 |
| C6 | 42419707 | 42420760 |
| C6 | 42521516 | 42522539 |
| C6 | 42581377 | 42582678 |
| C6 | 42694530 | 42695699 |
| C6 | 42927160 | 42928394 |
| C6 | 43083476 | 43084777 |
| C6 | 43251288 | 43252441 |
| C6 | 43252558 | 43253672 |
| C6 | 43476997 | 43478132 |
| C6 | 43547797 | 43548955 |
| C6 | 43548032 | 43549221 |
| C6 | 43612405 | 43613544 |
| C6 | 43696499 | 43697516 |
| C6 | 43737698 | 43738838 |
| C6 | 44242871 | 44244076 |
| C6 | 44243568 | 44244690 |
| C6 | 44587400 | 44588499 |
| C6 | 45361366 | 45362375 |
| C6 | 45630529 | 45631593 |
| C6 | 45795496 | 45796560 |
| C6 | 45832241 | 45833262 |
| C6 | 45966998 | 45968108 |
| C6 | 46272399 | 46273403 |
| C6 | 47923814 | 47924968 |
| C6 | 49346953 | 49349100 |
| C6 | 50673842 | 50674945 |
| C6 | 50790869 | 50792073 |
| C6 | 50808099 | 50809236 |
| C6 | 50812892 | 50813957 |
| C6 | 50813013 | 50814177 |
| C6 | 51318097 | 51319540 |
| C6 | 51739110 | 51740230 |
| C6 | 51739411 | 51740493 |
| C6 | 52519930 | 52521001 |
| C6 | 53169496 | 53170653 |
| C6 | 53344932 | 53345985 |
| C6 | 55005414 | 55006535 |
| C6 | 56297619 | 56298674 |
| C6 | 56758724 | 56759856 |
| C6 | 58148704 | 58149821 |
| C6 | 58287529 | 58288685 |
| C6 | 62053055 | 62054540 |
| C6 | 62053690 | 62055177 |
| C6 | 62995387 | 62996460 |
| C6 | 64281848 | 64282895 |
| C6 | 65184760 | 65185939 |
| C6 | 67158972 | 67160048 |
| C6 | 67771223 | 67772475 |
| C6 | 67771652 | 67772788 |
| C6 | 67865654 | 67866770 |
| C6 | 68706591 | 68707631 |
| C6 | 69285494 | 69286594 |
| C6 | 70141998 | 70143213 |
| C6 | 72297831 | 72299069 |
| C6 | 73262767 | 73263852 |
| C6 | 73328975 | 73331510 |
| C6 | 73883634 | 73884678 |
| C6 | 74008625 | 74009743 |
| C6 | 74018701 | 74020086 |
| C6 | 74019302 | 74020442 |
| C6 | 74019546 | 74020866 |
| C6 | 74023948 | 74025425 |
| C6 | 74058365 | 74059819 |
| C6 | 74104263 | 74105378 |
| C6 | 74107753 | 74108788 |
| C6 | 74159977 | 74161106 |
| C6 | 74289636 | 74291017 |
| C6 | 74405910 | 74407541 |
| C6 | 74422756 | 74424897 |
| C6 | 74434442 | 74435825 |
| C6 | 75139160 | 75140177 |
| C6 | 75793837 | 75795664 |
| C6 | 75802750 | 75803819 |
| C6 | 75915945 | 75918178 |
| C6 | 76058543 | 76060292 |
| C6 | 76927428 | 76928835 |
| C6 | 77752534 | 77755067 |
| C6 | 78171418 | 78174233 |
| C6 | 79338462 | 79339962 |
| C6 | 79619764 | 79620974 |
| C6 | 79620086 | 79621281 |
| C6 | 84954559 | 84955686 |
| C6 | 85472907 | 85473982 |
| C6 | 85771060 | 85772186 |
| C6 | 87718705 | 87719882 |
| C6 | 87831858 | 87832939 |
| C6 | 88757153 | 88758253 |
| C6 | 89657744 | 89659202 |
| C6 | 89827544 | 89828622 |
| C6 | 89855906 | 89857030 |
| C6 | 90552974 | 90554028 |
| C6 | 90611792 | 90612847 |
| C6 | 90660521 | 90661894 |
| C6 | 91295507 | 91296593 |
| C6 | 92807543 | 92808723 |
| C6 | 92910366 | 92911819 |
| C6 | 93353647 | 93354867 |
| C6 | 93432637 | 93433767 |
| C6 | 93432775 | 93433875 |
| C6 | 95637477 | 95638604 |
| C6 | 98690855 | 98691952 |
| C6 | 98693110 | 98694196 |
| C6 | 99449730 | 99450840 |
| C6 | 99536244 | 99537359 |
| C6 | 100895407 | 100896561 |
| C6 | 101850106 | 101851333 |
| C6 | 102435511 | 102436530 |
| C6 | 103779822 | 103781411 |
| C6 | 104936905 | 104938073 |
| C6 | 105387853 | 105388990 |
| C6 | 105400384 | 105401504 |
| C6 | 105567303 | 105568566 |
| C6 | 106433485 | 106434626 |
| C6 | 106433887 | 106435028 |
| C6 | 106474342 | 106475828 |
| C6 | 107778917 | 107780061 |
| C6 | 108123780 | 108124943 |
| C6 | 108201205 | 108202285 |
| C6 | 108274197 | 108275580 |
| C6 | 108321474 | 108322968 |
| C6 | 108434670 | 108435853 |
| C6 | 108478977 | 108480057 |
| C6 | 108488094 | 108489196 |
| C6 | 108492142 | 108493269 |
| C6 | 109119652 | 109120965 |
| C6 | 109571127 | 109572577 |
| C6 | 109611092 | 109612701 |
| C6 | 109696207 | 109697632 |
| C6 | 110199887 | 110201584 |
| C6 | 110677729 | 110678805 |
| C6 | 110720218 | 110721915 |
| C6 | 110797522 | 110798607 |
| C6 | 111510220 | 111511268 |
| C6 | 111547564 | 111548573 |
| C6 | 112070866 | 112071884 |
| C6 | 112687875 | 112688961 |
| C6 | 112688039 | 112689158 |
| C6 | 112829566 | 112831045 |
| C6 | 114180865 | 114181991 |
| C6 | 114181063 | 114182118 |
| C6 | 116782605 | 116783814 |
| C6 | 116783016 | 116784079 |
| C6 | 116885629 | 116886853 |
| C6 | 117729620 | 117731305 |
| C6 | 117803090 | 117804187 |
| C6 | 118847108 | 118849023 |
| C6 | 119666877 | 119668815 |
| C6 | 121318844 | 121319947 |
| C6 | 124843564 | 124844662 |
| C6 | 126069895 | 126071015 |
| C6 | 126090851 | 126092519 |
| C6 | 126166087 | 126167656 |
| C6 | 127522984 | 127524188 |
| C6 | 127795891 | 127797112 |
| C6 | 129450291 | 129451564 |
| C6 | 129493194 | 129494935 |
| C6 | 130839891 | 130840915 |
| C6 | 134195631 | 134196770 |
| C6 | 134436023 | 134437284 |
| C6 | 134588575 | 134590086 |
| C6 | 134953286 | 134954772 |
| C6 | 135078383 | 135079408 |
| C6 | 135203111 | 135204420 |
| C6 | 135228877 | 135230162 |
| C6 | 135376258 | 135377347 |
| C6 | 135583191 | 135584251 |
| C6 | 135589981 | 135592163 |
| C6 | 136235162 | 136236370 |
| C6 | 137114453 | 137115557 |
| C6 | 139012396 | 139013650 |
| C6 | 139222296 | 139223304 |
| C6 | 142290185 | 142291529 |
| C6 | 144077107 | 144078252 |
| C6 | 144199028 | 144200121 |
| C6 | 144328423 | 144329797 |
| C6 | 144328801 | 144330035 |
| C6 | 144535840 | 144537082 |
| C6 | 146303777 | 146304906 |
| C6 | 146474994 | 146476027 |
| C6 | 146587820 | 146589392 |
| C6 | 146626378 | 146627416 |
| C6 | 146864026 | 146865282 |
| C6 | 147980636 | 147981662 |
| C6 | 149867692 | 149868827 |
| C6 | 150039806 | 150041021 |
| C6 | 150106297 | 150107875 |
| C6 | 151170141 | 151171263 |
| C6 | 151411554 | 151412664 |
| C6 | 151533901 | 151534970 |
| C6 | 151655279 | 151656406 |
| C6 | 152219449 | 152220637 |
| C6 | 152426463 | 152427583 |
| C6 | 152864960 | 152865987 |
| C6 | 153033085 | 153034271 |
| C6 | 153154987 | 153156460 |
| C6 | 153196479 | 153197694 |
| C6 | 153494225 | 153495320 |
| C6 | 154171384 | 154172657 |
| C6 | 156650446 | 156652220 |
| C6 | 157469504 | 157470711 |
| C6 | 157957443 | 157958646 |
| C6 | 158066708 | 158067951 |
| C6 | 158401452 | 158402635 |
| C6 | 158506570 | 158508689 |
| C6 | 158630499 | 158631624 |
| C6 | 158660497 | 158661650 |
| C6 | 158719119 | 158720154 |
| C6 | 158727341 | 158728373 |
| C6 | 158752367 | 158753442 |
| C6 | 159549798 | 159550962 |
| C6 | 159926528 | 159927605 |
| C6 | 160192478 | 160193496 |
| C6 | 160425935 | 160427125 |
| C6 | 160426380 | 160427604 |
| C6 | 161051958 | 161053461 |
| C6 | 161099840 | 161100924 |
| C6 | 161105863 | 161107247 |
| C6 | 161187787 | 161188838 |
| C6 | 161226095 | 161227159 |
| C6 | 161446722 | 161447732 |
| C6 | 161796300 | 161797511 |
| C6 | 161865083 | 161866245 |
| C6 | 162236181 | 162237557 |
| C6 | 162759953 | 162761068 |
| C6 | 163070855 | 163071912 |
| C6 | 163264084 | 163265715 |
| C6 | 163693484 | 163694660 |
| C6 | 163843611 | 163844777 |
| C6 | 164254972 | 164256342 |
| C6 | 164287988 | 164289397 |
| C6 | 164406545 | 164407615 |
| C6 | 164460426 | 164461596 |
| C6 | 165540951 | 165542064 |
| C6 | 166487041 | 166488207 |
| C6 | 166510693 | 166511803 |
| C6 | 166535061 | 166536664 |
| C6 | 166574826 | 166575843 |
| C6 | 166806483 | 166807536 |
| C6 | 166822615 | 166823680 |
| C6 | 166825328 | 166826826 |
| C6 | 166889381 | 166890390 |
| C6 | 167040298 | 167041359 |
| C6 | 167047273 | 167048377 |
| C6 | 167161042 | 167162286 |
| C6 | 167368200 | 167369305 |
| C6 | 167679635 | 167680819 |
| C6 | 167708440 | 167709593 |
| C6 | 167789539 | 167790872 |
| C6 | 167835827 | 167836955 |
| C6 | 168052680 | 168053736 |
| C6 | 168139827 | 168141185 |
| C6 | 168365964 | 168367161 |
| C6 | 168390565 | 168392144 |
| C6 | 168499544 | 168500587 |
| C6 | 168513029 | 168514037 |
| C6 | 168646099 | 168647415 |
| C6 | 168652992 | 168654134 |
| C6 | 168684290 | 168685434 |
| C6 | 168745864 | 168747098 |
| C6 | 168757369 | 168758667 |
| C6 | 168758056 | 168759194 |
| C6 | 168759049 | 168760166 |
| C6 | 168773737 | 168774814 |
| C6 | 168972375 | 168973485 |
| C6 | 168993013 | 168994320 |
| C6 | 168993931 | 168995757 |
| C6 | 169222833 | 169224039 |
| C6 | 169330709 | 169331717 |
| C6 | 169574605 | 169575734 |
| C6 | 169740101 | 169741292 |
| C6 | 169824955 | 169826088 |
| C6 | 169851828 | 169852985 |
| C6 | 169852065 | 169853179 |
| C6 | 169976856 | 169977954 |
| C6 | 169977312 | 169978385 |
| C6 | 170054230 | 170056259 |
| C6 | 170337732 | 170338812 |
| C6 | 170337946 | 170339114 |
| C6 | 170402692 | 170403782 |
| C6 | 170410171 | 170411342 |
| C6 | 170451536 | 170452722 |
| C6 | 170452159 | 170453245 |
| C6 | 170491435 | 170492570 |
| C6 | 170546273 | 170547388 |
| C6 | 170554295 | 170555912 |
| C6 | 170555197 | 170556645 |
| C6 | 170557263 | 170558564 |
| C6 | 170581279 | 170582482 |
| C6 | 170605892 | 170607021 |
| C6 | 170731834 | 170732939 |
| C6 | 171025876 | 171027191 |
| C7 | 30748 | 31859 |
| C7 | 120129 | 121483 |
| C7 | 143833 | 145134 |
| C7 | 144274 | 145309 |
| C7 | 166649 | 167967 |
| C7 | 178385 | 179626 |
| C7 | 180679 | 181901 |
| C7 | 202320 | 203770 |
| C7 | 209440 | 210600 |
| C7 | 213196 | 214255 |
| C7 | 302751 | 303929 |
| C7 | 303190 | 304281 |
| C7 | 376051 | 377145 |
| C7 | 386196 | 387268 |
| C7 | 388921 | 390056 |
| C7 | 408577 | 409644 |
| C7 | 409189 | 410296 |
| C7 | 413995 | 415106 |
| C7 | 415737 | 416918 |
| C7 | 419139 | 420670 |
| C7 | 425574 | 426757 |
| C7 | 456930 | 457978 |
| C7 | 458830 | 460507 |
| C7 | 475954 | 477060 |
| C7 | 494899 | 496112 |
| C7 | 501788 | 502849 |
| C7 | 502806 | 504052 |
| C7 | 514522 | 515860 |
| C7 | 516218 | 517414 |
| C7 | 533093 | 534878 |
| C7 | 631280 | 632709 |
| C7 | 632426 | 633885 |
| C7 | 640591 | 641891 |
| C7 | 641957 | 643113 |
| C7 | 642961 | 644950 |
| C7 | 675176 | 676241 |
| C7 | 675282 | 676409 |
| C7 | 808044 | 809224 |
| C7 | 813486 | 814576 |
| C7 | 813771 | 814851 |
| C7 | 917205 | 918381 |
| C7 | 921665 | 922750 |
| C7 | 947982 | 949293 |
| C7 | 948311 | 949828 |
| C7 | 950737 | 951974 |
| C7 | 956046 | 957396 |
| C7 | 963197 | 964275 |
| C7 | 1022435 | 1023696 |
| C7 | 1024255 | 1025394 |
| C7 | 1029629 | 1030769 |
| C7 | 1045534 | 1046677 |
| C7 | 1051976 | 1053013 |
| C7 | 1078173 | 1079344 |
| C7 | 1080058 | 1081428 |
| C7 | 1093579 | 1094730 |
| C7 | 1094448 | 1095656 |
| C7 | 1098859 | 1100117 |
| C7 | 1099324 | 1101113 |
| C7 | 1131901 | 1132964 |
| C7 | 1147079 | 1148348 |
| C7 | 1186654 | 1187775 |
| C7 | 1198141 | 1199342 |
| C7 | 1208943 | 1210063 |
| C7 | 1239261 | 1240357 |
| C7 | 1250327 | 1251443 |
| C7 | 1274319 | 1275525 |
| C7 | 1307874 | 1309100 |
| C7 | 1446325 | 1447334 |
| C7 | 1552393 | 1553485 |
| C7 | 1625653 | 1627071 |
| C7 | 1626231 | 1627315 |
| C7 | 1687238 | 1688415 |
| C7 | 1730738 | 1732699 |
| C7 | 1765424 | 1766450 |
| C7 | 1785996 | 1787171 |
| C7 | 1792930 | 1793943 |
| C7 | 1798543 | 1799848 |
| C7 | 1858249 | 1860622 |
| C7 | 1951993 | 1953062 |
| C7 | 1959294 | 1960457 |
| C7 | 1961241 | 1962385 |
| C7 | 1980315 | 1981457 |
| C7 | 2113965 | 2115152 |
| C7 | 2115985 | 2117163 |
| C7 | 2121947 | 2122966 |
| C7 | 2132374 | 2133897 |
| C7 | 2143339 | 2144759 |
| C7 | 2150197 | 2151240 |
| C7 | 2151547 | 2152633 |
| C7 | 2154924 | 2156109 |
| C7 | 2211779 | 2213041 |
| C7 | 2286787 | 2288010 |
| C7 | 2306504 | 2307980 |
| C7 | 2307266 | 2308409 |
| C7 | 2423604 | 2424787 |
| C7 | 2444763 | 2445909 |
| C7 | 2446022 | 2447295 |
| C7 | 2533425 | 2534512 |
| C7 | 2563480 | 2564555 |
| C7 | 2570526 | 2572108 |
| C7 | 2573579 | 2574708 |
| C7 | 2609315 | 2610376 |
| C7 | 2647774 | 2649285 |
| C7 | 2649302 | 2650389 |
| C7 | 2653714 | 2655360 |
| C7 | 2676630 | 2677953 |
| C7 | 2680695 | 2681880 |
| C7 | 2704251 | 2705720 |
| C7 | 2718262 | 2719989 |
| C7 | 2773914 | 2775075 |
| C7 | 2775749 | 2776940 |
| C7 | 2801748 | 2802954 |
| C7 | 2832417 | 2833551 |
| C7 | 2967759 | 2968946 |
| C7 | 2967991 | 2969886 |
| C7 | 3133881 | 3135655 |
| C7 | 3220507 | 3221693 |
| C7 | 3919428 | 3921035 |
| C7 | 3982647 | 3983776 |
| C7 | 4243539 | 4245379 |
| C7 | 4347431 | 4348559 |
| C7 | 4352204 | 4353341 |
| C7 | 4450701 | 4451867 |
| C7 | 4455811 | 4456940 |
| C7 | 4744550 | 4745903 |
| C7 | 4754266 | 4755371 |
| C7 | 4764136 | 4765430 |
| C7 | 4765407 | 4766549 |
| C7 | 4831249 | 4832930 |
| C7 | 4849755 | 4850862 |
| C7 | 4853518 | 4854558 |
| C7 | 4910492 | 4911585 |
| C7 | 5170238 | 5172270 |
| C7 | 5228904 | 5230086 |
| C7 | 5270971 | 5272249 |
| C7 | 5336004 | 5337256 |
| C7 | 5488134 | 5489546 |
| C7 | 5516791 | 5518935 |
| C7 | 5532799 | 5533925 |
| C7 | 5734524 | 5735761 |
| C7 | 5735074 | 5736316 |
| C7 | 6071133 | 6072260 |
| C7 | 6388471 | 6389638 |
| C7 | 6463793 | 6464878 |
| C7 | 6473169 | 6474475 |
| C7 | 6583934 | 6585174 |
| C7 | 6617180 | 6618252 |
| C7 | 6693112 | 6694236 |
| C7 | 6758030 | 6759041 |
| C7 | 6802984 | 6804113 |
| C7 | 6806016 | 6807295 |
| C7 | 6884086 | 6885160 |
| C7 | 6910551 | 6911767 |
| C7 | 7679347 | 7680469 |
| C7 | 8475109 | 8476137 |
| C7 | 12058376 | 12059507 |
| C7 | 12442565 | 12443864 |
| C7 | 12723866 | 12725117 |
| C7 | 14910043 | 14911165 |
| C7 | 15586684 | 15587769 |
| C7 | 16890311 | 16891948 |
| C7 | 19185533 | 19186550 |
| C7 | 19397363 | 19398410 |
| C7 | 21250331 | 21251381 |
| C7 | 22372825 | 22374307 |
| C7 | 22539776 | 22540956 |
| C7 | 22823697 | 22825495 |
| C7 | 22875271 | 22876484 |
| C7 | 23065044 | 23066188 |
| C7 | 23085193 | 23086325 |
| C7 | 23167154 | 23168399 |
| C7 | 23220311 | 23221411 |
| C7 | 23245057 | 23246167 |
| C7 | 23511223 | 23512260 |
| C7 | 23529698 | 23530717 |
| C7 | 23943942 | 23945156 |
| C7 | 24037942 | 24040423 |
| C7 | 24768973 | 24770016 |
| C7 | 25019297 | 25020334 |
| C7 | 25044116 | 25045326 |
| C7 | 26118150 | 26119392 |
| C7 | 26283106 | 26284904 |
| C7 | 26301449 | 26302554 |
| C7 | 26344805 | 26346212 |
| C7 | 26555069 | 26556174 |
| C7 | 27018118 | 27019939 |
| C7 | 27135184 | 27136778 |
| C7 | 27147481 | 27148595 |
| C7 | 27147641 | 27148770 |
| C7 | 27152475 | 27153731 |
| C7 | 27152906 | 27154037 |
| C7 | 27153430 | 27155890 |
| C7 | 27161671 | 27162801 |
| C7 | 27162699 | 27163832 |
| C7 | 27163346 | 27164430 |
| C7 | 27169331 | 27170592 |
| C7 | 27169666 | 27171033 |
| C7 | 27170050 | 27171329 |
| C7 | 27178721 | 27179813 |
| C7 | 27181434 | 27182492 |
| C7 | 27182330 | 27183427 |
| C7 | 27182558 | 27183767 |
| C7 | 27182936 | 27184202 |
| C7 | 27184063 | 27185212 |
| C7 | 27184946 | 27186139 |
| C7 | 27206393 | 27207500 |
| C7 | 27207701 | 27208847 |
| C7 | 27231286 | 27232394 |
| C7 | 27278524 | 27279637 |
| C7 | 27282518 | 27283635 |
| C7 | 27702668 | 27703736 |
| C7 | 28498169 | 28499233 |
| C7 | 28580078 | 28581135 |
| C7 | 28736787 | 28737838 |
| C7 | 28815207 | 28816403 |
| C7 | 29417994 | 29419036 |
| C7 | 29881697 | 29883079 |
| C7 | 29911828 | 29912994 |
| C7 | 30478728 | 30480074 |
| C7 | 31051272 | 31052294 |
| C7 | 32137258 | 32138753 |
| C7 | 32357538 | 32359208 |
| C7 | 32426241 | 32427610 |
| C7 | 32801286 | 32802369 |
| C7 | 32801977 | 32803195 |
| C7 | 32944953 | 32946032 |
| C7 | 33533227 | 33534283 |
| C7 | 33566565 | 33567616 |
| C7 | 34918013 | 34919047 |
| C7 | 36006809 | 36008042 |
| C7 | 36010568 | 36011656 |
| C7 | 36010801 | 36011901 |
| C7 | 36022265 | 36023322 |
| C7 | 36275478 | 36276527 |
| C7 | 36320160 | 36321318 |
| C7 | 36776627 | 36777972 |
| C7 | 37381746 | 37382973 |
| C7 | 37392351 | 37393767 |
| C7 | 37456137 | 37457524 |
| C7 | 37686709 | 37687835 |
| C7 | 37687818 | 37689411 |
| C7 | 37702210 | 37703397 |
| C7 | 38392979 | 38395123 |
| C7 | 38507982 | 38509123 |
| C7 | 39893964 | 39895489 |
| C7 | 42230685 | 42231745 |
| C7 | 42895687 | 42897458 |
| C7 | 43337975 | 43339007 |
| C7 | 43350902 | 43352155 |
| C7 | 43354843 | 43355885 |
| C7 | 43454496 | 43456104 |
| C7 | 44151896 | 44153004 |
| C7 | 44184545 | 44185667 |
| C7 | 44260588 | 44261697 |
| C7 | 44307066 | 44308114 |
| C7 | 44312473 | 44313497 |
| C7 | 44641075 | 44642130 |
| C7 | 45002135 | 45003367 |
| C7 | 45065890 | 45069025 |
| C7 | 45570013 | 45571229 |
| C7 | 45979736 | 45980954 |
| C7 | 47091641 | 47092987 |
| C7 | 47576651 | 47577662 |
| C7 | 47734152 | 47735285 |
| C7 | 47847337 | 47848386 |
| C7 | 48240555 | 48241590 |
| C7 | 48325762 | 48326889 |
| C7 | 48333789 | 48335613 |
| C7 | 48494181 | 48495232 |
| C7 | 48646500 | 48647542 |
| C7 | 48887172 | 48888391 |
| C7 | 49551603 | 49552880 |
| C7 | 49812585 | 49813661 |
| C7 | 50015963 | 50017154 |
| C7 | 50305713 | 50307163 |
| C7 | 50343450 | 50344884 |
| C7 | 50345767 | 50347987 |
| C7 | 50467247 | 50468343 |
| C7 | 50664471 | 50665790 |
| C7 | 50679589 | 50680599 |
| C7 | 50811379 | 50812478 |
| C7 | 50849330 | 50850536 |
| C7 | 50849566 | 50850595 |
| C7 | 50849727 | 50850997 |
| C7 | 52094477 | 52095582 |
| C7 | 53195503 | 53196863 |
| C7 | 55072575 | 55073768 |
| C7 | 55086214 | 55087558 |
| C7 | 55111026 | 55112242 |
| C7 | 55176916 | 55178151 |
| C7 | 55186249 | 55187594 |
| C7 | 55209479 | 55210630 |
| C7 | 55210498 | 55211681 |
| C7 | 55213799 | 55214933 |
| C7 | 55218487 | 55219555 |
| C7 | 55219739 | 55220857 |
| C7 | 55221204 | 55222345 |
| C7 | 55223023 | 55224139 |
| C7 | 55223726 | 55224852 |
| C7 | 55223952 | 55225144 |
| C7 | 55224856 | 55225946 |
| C7 | 55227332 | 55228531 |
| C7 | 55228692 | 55229824 |
| C7 | 55230926 | 55232016 |
| C7 | 55232473 | 55233630 |
| C7 | 55235716 | 55236828 |
| C7 | 55237500 | 55239238 |
| C7 | 55238368 | 55239406 |
| C7 | 55240039 | 55241121 |
| C7 | 55240176 | 55241317 |
| C7 | 55241114 | 55242236 |
| C7 | 55241915 | 55243013 |
| C7 | 55248486 | 55249671 |
| C7 | 55258912 | 55260067 |
| C7 | 55259959 | 55261034 |
| C7 | 55265910 | 55267056 |
| C7 | 55267509 | 55268995 |
| C7 | 55268381 | 55269548 |
| C7 | 55268928 | 55269975 |
| C7 | 55269710 | 55271269 |
| C7 | 55272449 | 55279821 |
| C7 | 55287147 | 55288473 |
| C7 | 55300470 | 55301547 |
| C7 | 55323447 | 55324813 |
| C7 | 55376297 | 55377404 |
| C7 | 55409304 | 55410686 |
| C7 | 55516465 | 55517556 |
| C7 | 55695349 | 55697212 |
| C7 | 56039717 | 56040729 |
| C7 | 56218987 | 56220151 |
| C7 | 56296982 | 56298141 |
| C7 | 56866011 | 56867116 |
| C7 | 56884046 | 56885161 |
| C7 | 56885950 | 56887078 |
| C7 | 56886537 | 56887747 |
| C7 | 56887536 | 56888698 |
| C7 | 56888451 | 56889666 |
| C7 | 56974170 | 56977226 |
| C7 | 57016420 | 57017461 |
| C7 | 57075013 | 57076322 |
| C7 | 57484163 | 57485239 |
| C7 | 57568487 | 57569596 |
| C7 | 57706298 | 57707356 |
| C7 | 57786019 | 57787140 |
| C7 | 57802632 | 57803695 |
| C7 | 57878601 | 57880235 |
| C7 | 57881120 | 57882594 |
| C7 | 57927344 | 57928542 |
| C7 | 57927808 | 57929019 |
| C7 | 57928191 | 57929323 |
| C7 | 57967118 | 57970008 |
| C7 | 57970622 | 57972739 |
| C7 | 61116799 | 61118537 |
| C7 | 61126754 | 61129110 |
| C7 | 61232080 | 61234024 |
| C7 | 61744047 | 61745342 |
| C7 | 62776063 | 62778311 |
| C7 | 62873536 | 62874766 |
| C7 | 62901241 | 62903066 |
| C7 | 62952984 | 62954338 |
| C7 | 63063007 | 63064317 |
| C7 | 63073608 | 63075162 |
| C7 | 63085098 | 63086375 |
| C7 | 63087652 | 63088748 |
| C7 | 63087750 | 63088850 |
| C7 | 63105171 | 63107322 |
| C7 | 63217386 | 63219412 |
| C7 | 63391042 | 63392129 |
| C7 | 63923685 | 63924780 |
| C7 | 63923915 | 63925241 |
| C7 | 64269820 | 64271008 |
| C7 | 64722471 | 64723634 |
| C7 | 64973030 | 64974154 |
| C7 | 65005159 | 65006361 |
| C7 | 65969223 | 65970844 |
| C7 | 66432633 | 66433776 |
| C7 | 66829201 | 66830447 |
| C7 | 67999563 | 68000732 |
| C7 | 68461587 | 68462737 |
| C7 | 69111354 | 69112435 |
| C7 | 69675856 | 69676874 |
| C7 | 71723307 | 71724336 |
| C7 | 72381177 | 72382664 |
| C7 | 72419013 | 72420154 |
| C7 | 72439726 | 72441061 |
| C7 | 72473249 | 72474553 |
| C7 | 72626390 | 72627681 |
| C7 | 72698502 | 72699611 |
| C7 | 72711506 | 72712604 |
| C7 | 72715585 | 72717018 |
| C7 | 73098327 | 73099465 |
| C7 | 73182466 | 73183598 |
| C7 | 73211777 | 73212924 |
| C7 | 73245518 | 73246530 |
| C7 | 73250213 | 73251555 |
| C7 | 73304078 | 73305441 |
| C7 | 73507922 | 73509688 |
| C7 | 73624019 | 73625501 |
| C7 | 73632822 | 73633992 |
| C7 | 73735276 | 73736393 |
| C7 | 73752339 | 73753424 |
| C7 | 73994067 | 73995265 |
| C7 | 74020937 | 74022152 |
| C7 | 74181927 | 74183423 |
| C7 | 74203883 | 74205367 |
| C7 | 74283568 | 74284783 |
| C7 | 74394254 | 74396180 |
| C7 | 74566146 | 74568074 |
| C7 | 74571512 | 74572587 |
| C7 | 74572301 | 74573419 |
| C7 | 74988202 | 74989352 |
| C7 | 75017774 | 75018871 |
| C7 | 75457266 | 75459123 |
| C7 | 75580898 | 75582099 |
| C7 | 75585612 | 75586824 |
| C7 | 75601319 | 75602542 |
| C7 | 75617819 | 75619077 |
| C7 | 75674685 | 75676187 |
| C7 | 75779024 | 75780190 |
| C7 | 75779279 | 75780452 |
| C7 | 75795154 | 75796327 |
| C7 | 76085638 | 76087393 |
| C7 | 76128643 | 76129964 |
| C7 | 76129075 | 76130262 |
| C7 | 76228263 | 76229444 |
| C7 | 76628769 | 76629832 |
| C7 | 77162143 | 77163348 |
| C7 | 77474921 | 77476728 |
| C7 | 78230396 | 78231474 |
| C7 | 82072410 | 82073457 |
| C7 | 82946034 | 82947175 |
| C7 | 83947093 | 83948284 |
| C7 | 85956065 | 85957205 |
| C7 | 86069215 | 86070782 |
| C7 | 86273756 | 86275132 |
| C7 | 86836182 | 86837329 |
| C7 | 88499115 | 88500251 |
| C7 | 90583661 | 90585556 |
| C7 | 90895129 | 90896204 |
| C7 | 90896036 | 90897135 |
| C7 | 93553702 | 93554758 |
| C7 | 94022657 | 94024019 |
| C7 | 94285335 | 94286863 |
| C7 | 94286686 | 94287804 |
| C7 | 95292769 | 95294082 |
| C7 | 98288418 | 98289476 |
| C7 | 98836415 | 98837434 |
| C7 | 98990255 | 98991405 |
| C7 | 99195288 | 99196443 |
| C7 | 99768969 | 99770103 |
| C7 | 99774909 | 99776032 |
| C7 | 99915181 | 99916716 |
| C7 | 99978998 | 99980186 |
| C7 | 100090755 | 100091901 |
| C7 | 100120370 | 100121572 |
| C7 | 100166698 | 100167782 |
| C7 | 100171432 | 100172569 |
| C7 | 100180909 | 100182486 |
| C7 | 100230685 | 100231816 |
| C7 | 100304104 | 100305232 |
| C7 | 100318064 | 100319183 |
| C7 | 100339511 | 100340718 |
| C7 | 100342249 | 100343731 |
| C7 | 100411138 | 100412503 |
| C7 | 100433937 | 100435400 |
| C7 | 100635882 | 100637240 |
| C7 | 100778364 | 100779563 |
| C7 | 100845121 | 100846213 |
| C7 | 100875169 | 100876543 |
| C7 | 100884469 | 100885751 |
| C7 | 100908793 | 100909979 |
| C7 | 101294138 | 101295566 |
| C7 | 101333739 | 101335244 |
| C7 | 101459734 | 101460792 |
| C7 | 101556322 | 101558119 |
| C7 | 101943450 | 101944695 |
| C7 | 101943833 | 101944963 |
| C7 | 101961324 | 101962375 |
| C7 | 101961384 | 101962439 |
| C7 | 101961471 | 101962568 |
| C7 | 102133453 | 102134521 |
| C7 | 102232872 | 102233991 |
| C7 | 102249644 | 102250882 |
| C7 | 102313457 | 102315278 |
| C7 | 102329451 | 102330587 |
| C7 | 102716407 | 102717415 |
| C7 | 103722388 | 103723474 |
| C7 | 103969180 | 103970297 |
| C7 | 104349844 | 104351089 |
| C7 | 104580774 | 104582400 |
| C7 | 105219063 | 105220214 |
| C7 | 105279089 | 105280382 |
| C7 | 105595810 | 105597115 |
| C7 | 105705845 | 105706973 |
| C7 | 107641749 | 107642953 |
| C7 | 109390467 | 109391477 |
| C7 | 110352240 | 110353341 |
| C7 | 111202508 | 111203746 |
| C7 | 112084652 | 112085783 |
| C7 | 113420502 | 113422226 |
| C7 | 113953168 | 113954295 |
| C7 | 115116924 | 115118088 |
| C7 | 116483029 | 116484829 |
| C7 | 117119101 | 117120279 |
| C7 | 117119321 | 117120495 |
| C7 | 117167473 | 117169158 |
| C7 | 117312824 | 117314347 |
| C7 | 117545137 | 117546175 |
| C7 | 118792087 | 118793196 |
| C7 | 120880693 | 120881858 |
| C7 | 124698847 | 124700021 |
| C7 | 125018894 | 125019957 |
| C7 | 125264668 | 125265777 |
| C7 | 125788638 | 125789773 |
| C7 | 127671669 | 127672792 |
| C7 | 127765988 | 127767120 |
| C7 | 127841060 | 127842595 |
| C7 | 127856583 | 127857951 |
| C7 | 127880383 | 127881481 |
| C7 | 127910453 | 127911535 |
| C7 | 127910673 | 127911737 |
| C7 | 127990687 | 127991722 |
| C7 | 127991549 | 127992635 |
| C7 | 128336839 | 128338559 |
| C7 | 128470516 | 128471624 |
| C7 | 128555497 | 128556811 |
| C7 | 128594719 | 128595831 |
| C7 | 128808178 | 128809364 |
| C7 | 128808801 | 128809893 |
| C7 | 128864178 | 128865248 |
| C7 | 128910429 | 128911688 |
| C7 | 129422353 | 129423837 |
| C7 | 129434879 | 129436385 |
| C7 | 129713161 | 129714196 |
| C7 | 130013236 | 130014372 |
| C7 | 130130221 | 130131445 |
| C7 | 130131421 | 130132861 |
| C7 | 130132005 | 130133461 |
| C7 | 130282674 | 130283716 |
| C7 | 130417413 | 130418489 |
| C7 | 130517315 | 130518391 |
| C7 | 130605986 | 130607153 |
| C7 | 131190942 | 131192375 |
| C7 | 132185936 | 132186977 |
| C7 | 132212391 | 132213407 |
| C7 | 132339478 | 132340879 |
| C7 | 133915799 | 133917032 |
| C7 | 134088188 | 134089273 |
| C7 | 134142621 | 134143782 |
| C7 | 134143284 | 134144562 |
| C7 | 134233862 | 134234881 |
| C7 | 134850882 | 134853206 |
| C7 | 134917820 | 134919294 |
| C7 | 135398786 | 135400181 |
| C7 | 135513205 | 135514403 |
| C7 | 136834121 | 136835251 |
| C7 | 136851081 | 136852203 |
| C7 | 137368727 | 137369772 |
| C7 | 137684336 | 137685413 |
| C7 | 137831588 | 137832635 |
| C7 | 138249179 | 138250347 |
| C7 | 138348342 | 138349994 |
| C7 | 138349049 | 138350340 |
| C7 | 138402642 | 138403800 |
| C7 | 138874639 | 138875653 |
| C7 | 139010159 | 139011175 |
| C7 | 139043680 | 139044749 |
| C7 | 139184726 | 139185886 |
| C7 | 139649986 | 139651096 |
| C7 | 140134779 | 140136212 |
| C7 | 140226500 | 140227844 |
| C7 | 140915750 | 140917158 |
| C7 | 141044978 | 141046437 |
| C7 | 141260371 | 141261389 |
| C7 | 141966911 | 141968034 |
| C7 | 142273998 | 142275119 |
| C7 | 142428501 | 142429719 |
| C7 | 142582664 | 142584055 |
| C7 | 143235891 | 143237095 |
| C7 | 143581646 | 143582759 |
| C7 | 145351744 | 145352845 |
| C7 | 145394064 | 145395487 |
| C7 | 146027667 | 146028789 |
| C7 | 146969043 | 146970216 |
| C7 | 147510333 | 147511339 |
| C7 | 148761448 | 148762609 |
| C7 | 148768553 | 148769629 |
| C7 | 148869038 | 148870814 |
| C7 | 148887646 | 148889048 |
| C7 | 148897881 | 148898889 |
| C7 | 148902476 | 148903728 |
| C7 | 149111381 | 149112828 |
| C7 | 149120169 | 149121268 |
| C7 | 149484288 | 149485381 |
| C7 | 149844862 | 149845927 |
| C7 | 149965842 | 149967283 |
| C7 | 150037714 | 150038826 |
| C7 | 150038151 | 150039363 |
| C7 | 150548434 | 150549676 |
| C7 | 150644156 | 150645216 |
| C7 | 150759744 | 150760951 |
| C7 | 150777840 | 150778936 |
| C7 | 150778291 | 150779474 |
| C7 | 150811393 | 150812441 |
| C7 | 150811770 | 150812935 |
| C7 | 150822476 | 150823542 |
| C7 | 150822656 | 150823958 |
| C7 | 151086921 | 151088255 |
| C7 | 151148746 | 151149827 |
| C7 | 151329634 | 151330765 |
| C7 | 152282780 | 152283940 |
| C7 | 152282947 | 152284149 |
| C7 | 152283319 | 152284788 |
| C7 | 152558164 | 152559382 |
| C7 | 152848482 | 152849814 |
| C7 | 153012107 | 153013246 |
| C7 | 153417522 | 153418778 |
| C7 | 153446011 | 153447063 |
| C7 | 153782456 | 153783486 |
| C7 | 153824311 | 153826112 |
| C7 | 153899739 | 153900769 |
| C7 | 154002700 | 154003813 |
| C7 | 154101595 | 154102634 |
| C7 | 154399423 | 154400468 |
| C7 | 154414774 | 154415863 |
| C7 | 154875402 | 154876535 |
| C7 | 154997260 | 154998623 |
| C7 | 155006045 | 155007112 |
| C7 | 155030700 | 155031978 |
| C7 | 155142626 | 155143739 |
| C7 | 155150143 | 155151884 |
| C7 | 155241157 | 155242312 |
| C7 | 155302184 | 155303658 |
| C7 | 155365869 | 155367133 |
| C7 | 155385589 | 155386846 |
| C7 | 155556247 | 155557510 |
| C7 | 155595252 | 155596329 |
| C7 | 155595681 | 155596853 |
| C7 | 155826255 | 155827277 |
| C7 | 155896885 | 155897983 |
| C7 | 156028715 | 156029938 |
| C7 | 156261029 | 156262102 |
| C7 | 156399979 | 156401078 |
| C7 | 156400426 | 156401481 |
| C7 | 156408592 | 156409834 |
| C7 | 156408887 | 156410024 |
| C7 | 156685467 | 156686524 |
| C7 | 156739696 | 156740875 |
| C7 | 156740973 | 156742039 |
| C7 | 156741100 | 156742284 |
| C7 | 156870571 | 156871771 |
| C7 | 156930034 | 156931286 |
| C7 | 157071357 | 157072454 |
| C7 | 157094016 | 157095671 |
| C7 | 157262617 | 157263743 |
| C7 | 157346211 | 157347621 |
| C7 | 157351975 | 157353034 |
| C7 | 157400603 | 157401725 |
| C7 | 157405007 | 157406118 |
| C7 | 157436024 | 157437159 |
| C7 | 157476695 | 157477769 |
| C7 | 157480241 | 157481788 |
| C7 | 157481259 | 157482280 |
| C7 | 157485273 | 157486366 |
| C7 | 157502990 | 157504097 |
| C7 | 157527054 | 157528074 |
| C7 | 157579267 | 157580407 |
| C7 | 157591767 | 157592824 |
| C7 | 157618477 | 157619666 |
| C7 | 157626760 | 157627952 |
| C7 | 157630687 | 157631891 |
| C7 | 157640985 | 157642265 |
| C7 | 157642021 | 157643667 |
| C7 | 157659233 | 157660347 |
| C7 | 157665648 | 157666740 |
| C7 | 157684742 | 157685750 |
| C7 | 157702486 | 157703590 |
| C7 | 157821351 | 157822436 |
| C7 | 157889864 | 157890892 |
| C7 | 157918647 | 157919756 |
| C7 | 157951027 | 157952300 |
| C7 | 157980420 | 157981489 |
| C7 | 157980583 | 157981665 |
| C7 | 157980779 | 157981927 |
| C7 | 158029276 | 158030585 |
| C7 | 158058222 | 158059547 |
| C7 | 158087580 | 158088803 |
| C7 | 158130111 | 158131439 |
| C7 | 158130852 | 158132425 |
| C7 | 158141561 | 158143100 |
| C7 | 158180436 | 158181833 |
| C7 | 158218238 | 158219310 |
| C7 | 158248163 | 158249860 |
| C7 | 158361880 | 158362940 |
| C7 | 158362727 | 158363854 |
| C7 | 158379009 | 158380067 |
| C7 | 158379908 | 158380999 |
| C7 | 158496611 | 158497760 |
| C7 | 158621874 | 158622967 |
| C7 | 158765633 | 158766726 |
| C7 | 158765736 | 158766881 |
| C7 | 158766281 | 158767514 |
| C7 | 158768206 | 158769491 |
| C7 | 158789329 | 158790368 |
| C7 | 158798287 | 158799402 |
| C7 | 158798777 | 158799896 |
| C7 | 158808023 | 158809114 |
| C7 | 158882087 | 158883579 |
| C7 | 158893840 | 158894939 |
| C7 | 158904447 | 158905563 |
| C8 | 99192 | 100273 |
| C8 | 99713 | 101128 |
| C8 | 215902 | 216957 |
| C8 | 215986 | 217641 |
| C8 | 245690 | 247046 |
| C8 | 327421 | 328970 |
| C8 | 522080 | 523222 |
| C8 | 528176 | 529299 |
| C8 | 599087 | 600898 |
| C8 | 623072 | 624148 |
| C8 | 637139 | 638168 |
| C8 | 673653 | 675031 |
| C8 | 816498 | 817946 |
| C8 | 832020 | 833094 |
| C8 | 832109 | 833418 |
| C8 | 849658 | 850727 |
| C8 | 852183 | 853285 |
| C8 | 876334 | 877506 |
| C8 | 922769 | 923832 |
| C8 | 940965 | 942290 |
| C8 | 941613 | 943262 |
| C8 | 963770 | 965092 |
| C8 | 977033 | 978806 |
| C8 | 991166 | 992304 |
| C8 | 1048200 | 1049290 |
| C8 | 1112811 | 1113932 |
| C8 | 1185839 | 1187450 |
| C8 | 1263344 | 1264500 |
| C8 | 1264682 | 1265843 |
| C8 | 1320643 | 1322166 |
| C8 | 1347782 | 1349221 |
| C8 | 1354524 | 1355733 |
| C8 | 1364410 | 1365974 |
| C8 | 1409593 | 1410941 |
| C8 | 1411885 | 1413236 |
| C8 | 1496310 | 1497404 |
| C8 | 1509909 | 1511029 |
| C8 | 1555398 | 1556513 |
| C8 | 1555611 | 1556961 |
| C8 | 1590910 | 1592037 |
| C8 | 1617880 | 1619129 |
| C8 | 1639557 | 1641046 |
| C8 | 1684892 | 1685949 |
| C8 | 1763944 | 1765021 |
| C8 | 1764288 | 1765339 |
| C8 | 1789344 | 1790500 |
| C8 | 1789588 | 1790882 |
| C8 | 1821144 | 1822235 |
| C8 | 1950289 | 1951414 |
| C8 | 1971598 | 1972731 |
| C8 | 2133002 | 2134254 |
| C8 | 2178631 | 2179717 |
| C8 | 2222371 | 2223502 |
| C8 | 2480519 | 2481732 |
| C8 | 2590707 | 2591749 |
| C8 | 2669735 | 2670960 |
| C8 | 4212050 | 4213145 |
| C8 | 4644130 | 4645254 |
| C8 | 4711723 | 4712751 |
| C8 | 4853403 | 4854588 |
| C8 | 5581974 | 5583166 |
| C8 | 6156451 | 6158897 |
| C8 | 6397814 | 6398893 |
| C8 | 6932499 | 6933518 |
| C8 | 7024108 | 7025299 |
| C8 | 7024850 | 7026626 |
| C8 | 7126962 | 7128157 |
| C8 | 7131298 | 7132570 |
| C8 | 7190612 | 7192027 |
| C8 | 7430555 | 7432066 |
| C8 | 7571341 | 7572636 |
| C8 | 7825557 | 7826718 |
| C8 | 7873497 | 7875143 |
| C8 | 7876598 | 7877797 |
| C8 | 7894598 | 7895789 |
| C8 | 7936265 | 7939201 |
| C8 | 8267169 | 8268218 |
| C8 | 8559499 | 8560637 |
| C8 | 9005724 | 9007152 |
| C8 | 9106421 | 9107697 |
| C8 | 9741275 | 9742487 |
| C8 | 9991585 | 9992664 |
| C8 | 10250652 | 10251721 |
| C8 | 10404745 | 10405916 |
| C8 | 10708944 | 10710051 |
| C8 | 10792152 | 10794771 |
| C8 | 10795012 | 10796119 |
| C8 | 11773943 | 11775075 |
| C8 | 12069733 | 12071169 |
| C8 | 12089749 | 12091408 |
| C8 | 12274039 | 12275207 |
| C8 | 12383520 | 12384773 |
| C8 | 12455818 | 12457548 |
| C8 | 12788481 | 12789600 |
| C8 | 13169230 | 13170365 |
| C8 | 13797417 | 13798506 |
| C8 | 13958923 | 13959941 |
| C8 | 15807233 | 15808292 |
| C8 | 16564586 | 16566135 |
| C8 | 16961730 | 16962832 |
| C8 | 17103594 | 17104739 |
| C8 | 17198301 | 17199494 |
| C8 | 20812216 | 20813236 |
| C8 | 21242621 | 21243710 |
| C8 | 21766739 | 21767776 |
| C8 | 21882232 | 21883308 |
| C8 | 21923952 | 21925013 |
| C8 | 21943958 | 21945013 |
| C8 | 21973837 | 21974927 |
| C8 | 21995683 | 21996777 |
| C8 | 22237964 | 22239132 |
| C8 | 22446237 | 22447618 |
| C8 | 22856420 | 22857527 |
| C8 | 23559848 | 23560922 |
| C8 | 25066139 | 25067236 |
| C8 | 25653539 | 25654574 |
| C8 | 27166505 | 27167738 |
| C8 | 27194545 | 27195709 |
| C8 | 27612239 | 27613265 |
| C8 | 28196172 | 28197263 |
| C8 | 28358011 | 28359069 |
| C8 | 28630475 | 28631765 |
| C8 | 28852884 | 28853891 |
| C8 | 30391483 | 30392910 |
| C8 | 30441102 | 30442510 |
| C8 | 30588574 | 30589733 |
| C8 | 30698202 | 30699226 |
| C8 | 30769323 | 30770406 |
| C8 | 30904643 | 30905696 |
| C8 | 31570842 | 31571955 |
| C8 | 31804087 | 31805233 |
| C8 | 33410906 | 33413114 |
| C8 | 35092180 | 35093299 |
| C8 | 35092967 | 35094098 |
| C8 | 35141635 | 35143886 |
| C8 | 35161562 | 35162602 |
| C8 | 35170363 | 35172289 |
| C8 | 35172760 | 35174723 |
| C8 | 35276910 | 35281586 |
| C8 | 35286894 | 35290829 |
| C8 | 35389131 | 35390858 |
| C8 | 35396526 | 35398368 |
| C8 | 35475230 | 35476300 |
| C8 | 35544182 | 35545200 |
| C8 | 35571887 | 35574121 |
| C8 | 35723767 | 35724835 |
| C8 | 35756291 | 35758473 |
| C8 | 35772791 | 35773870 |
| C8 | 35977603 | 35979376 |
| C8 | 36049118 | 36050199 |
| C8 | 36091921 | 36095321 |
| C8 | 36130660 | 36135061 |
| C8 | 36176474 | 36178933 |
| C8 | 36178358 | 36179596 |
| C8 | 36183483 | 36184565 |
| C8 | 36208425 | 36210288 |
| C8 | 36216037 | 36218040 |
| C8 | 36255130 | 36259261 |
| C8 | 36294096 | 36296663 |
| C8 | 36296626 | 36298811 |
| C8 | 36299276 | 36301872 |
| C8 | 36306961 | 36308017 |
| C8 | 36347174 | 36348370 |
| C8 | 36463140 | 36469355 |
| C8 | 36504029 | 36505254 |
| C8 | 36531763 | 36532843 |
| C8 | 36946383 | 36947427 |
| C8 | 37031588 | 37033005 |
| C8 | 37081897 | 37083126 |
| C8 | 37118787 | 37120362 |
| C8 | 37182442 | 37184472 |
| C8 | 37430880 | 37433058 |
| C8 | 37494808 | 37496485 |
| C8 | 37554476 | 37555543 |
| C8 | 37555004 | 37556096 |
| C8 | 37555134 | 37556333 |
| C8 | 37555434 | 37556485 |
| C8 | 37555634 | 37556827 |
| C8 | 37560060 | 37561615 |
| C8 | 37560620 | 37561916 |
| C8 | 37561156 | 37562540 |
| C8 | 37654737 | 37655981 |
| C8 | 37655198 | 37656747 |
| C8 | 37682862 | 37684091 |
| C8 | 37698483 | 37699640 |
| C8 | 37698901 | 37700211 |
| C8 | 37755221 | 37756455 |
| C8 | 37757512 | 37759116 |
| C8 | 37821995 | 37823071 |
| C8 | 37822287 | 37824092 |
| C8 | 37823176 | 37824269 |
| C8 | 37823479 | 37824840 |
| C8 | 37938614 | 37940742 |
| C8 | 37996311 | 37998051 |
| C8 | 38007450 | 38009172 |
| C8 | 38289657 | 38290714 |
| C8 | 38440475 | 38441964 |
| C8 | 38507856 | 38509247 |
| C8 | 38523306 | 38524777 |
| C8 | 38585265 | 38586746 |
| C8 | 38613780 | 38615140 |
| C8 | 38614193 | 38615272 |
| C8 | 38614425 | 38615859 |
| C8 | 38627566 | 38628788 |
| C8 | 38706894 | 38709251 |
| C8 | 38756836 | 38758779 |
| C8 | 38839817 | 38840865 |
| C8 | 38856575 | 38857628 |
| C8 | 38964288 | 38965969 |
| C8 | 39171520 | 39172676 |
| C8 | 39327941 | 39329447 |
| C8 | 40022067 | 40023865 |
| C8 | 40601626 | 40603655 |
| C8 | 40841211 | 40842795 |
| C8 | 40957271 | 40958610 |
| C8 | 41165353 | 41167209 |
| C8 | 41166759 | 41167799 |
| C8 | 41217905 | 41219968 |
| C8 | 41304440 | 41306015 |
| C8 | 41400089 | 41401451 |
| C8 | 41401862 | 41403438 |
| C8 | 41416822 | 41417864 |
| C8 | 41423842 | 41425799 |
| C8 | 41483969 | 41485178 |
| C8 | 41508033 | 41509070 |
| C8 | 41510424 | 41511639 |
| C8 | 41511286 | 41512789 |
| C8 | 41520827 | 41522199 |
| C8 | 41540921 | 41542405 |
| C8 | 41560719 | 41562107 |
| C8 | 41569647 | 41571527 |
| C8 | 41573149 | 41574677 |
| C8 | 41654408 | 41656394 |
| C8 | 41679495 | 41681334 |
| C8 | 41693055 | 41694265 |
| C8 | 41732861 | 41734205 |
| C8 | 41733292 | 41734431 |
| C8 | 41753212 | 41754508 |
| C8 | 41753610 | 41754674 |
| C8 | 41754270 | 41756165 |
| C8 | 41786611 | 41788323 |
| C8 | 42009184 | 42010537 |
| C8 | 42035897 | 42037065 |
| C8 | 42036677 | 42038180 |
| C8 | 42039862 | 42041124 |
| C8 | 42122560 | 42124097 |
| C8 | 42188227 | 42189706 |
| C8 | 42257286 | 42258334 |
| C8 | 42262794 | 42264991 |
| C8 | 42355944 | 42357576 |
| C8 | 42426639 | 42427649 |
| C8 | 42627760 | 42628792 |
| C8 | 42650728 | 42652461 |
| C8 | 42751111 | 42752165 |
| C8 | 42946480 | 42947813 |
| C8 | 42995076 | 42996136 |
| C8 | 43101952 | 43103236 |
| C8 | 43131744 | 43132826 |
| C8 | 43131902 | 43133065 |
| C8 | 43401066 | 43402097 |
| C8 | 43811410 | 43813147 |
| C8 | 47528598 | 47529676 |
| C8 | 47573368 | 47574404 |
| C8 | 48571617 | 48572747 |
| C8 | 48571852 | 48573133 |
| C8 | 48675411 | 48676500 |
| C8 | 49426616 | 49428174 |
| C8 | 50219425 | 50220529 |
| C8 | 50821817 | 50822846 |
| C8 | 52321406 | 52322550 |
| C8 | 52367138 | 52368246 |
| C8 | 52411251 | 52412283 |
| C8 | 52866249 | 52867750 |
| C8 | 53477602 | 53478769 |
| C8 | 53478188 | 53479753 |
| C8 | 53488235 | 53489283 |
| C8 | 53573273 | 53574280 |
| C8 | 53853832 | 53854945 |
| C8 | 54624485 | 54625547 |
| C8 | 54934937 | 54936114 |
| C8 | 55037572 | 55039372 |
| C8 | 55086473 | 55087661 |
| C8 | 55285276 | 55286535 |
| C8 | 55365869 | 55366964 |
| C8 | 55366657 | 55367966 |
| C8 | 55367110 | 55368128 |
| C8 | 55367497 | 55368625 |
| C8 | 55370341 | 55371695 |
| C8 | 55371661 | 55373070 |
| C8 | 55378484 | 55379663 |
| C8 | 55381902 | 55383191 |
| C8 | 55827831 | 55829068 |
| C8 | 56362106 | 56363235 |
| C8 | 56851648 | 56852767 |
| C8 | 57025539 | 57026743 |
| C8 | 57029999 | 57031040 |
| C8 | 57357636 | 57358920 |
| C8 | 57802122 | 57803362 |
| C8 | 57819937 | 57822745 |
| C8 | 58054701 | 58056593 |
| C8 | 58117970 | 58119160 |
| C8 | 58119164 | 58120386 |
| C8 | 58124005 | 58125194 |
| C8 | 58192052 | 58193747 |
| C8 | 59147068 | 59148506 |
| C8 | 59308939 | 59309985 |
| C8 | 59826801 | 59827920 |
| C8 | 59879078 | 59881379 |
| C8 | 59952106 | 59953184 |
| C8 | 59959344 | 59960562 |
| C8 | 60114080 | 60115155 |
| C8 | 61325212 | 61326400 |
| C8 | 61626296 | 61627356 |
| C8 | 61626452 | 61627679 |
| C8 | 61777091 | 61778265 |
| C8 | 61834856 | 61835985 |
| C8 | 62664601 | 62665702 |
| C8 | 62929801 | 62930903 |
| C8 | 63776150 | 63777210 |
| C8 | 65292739 | 65293919 |
| C8 | 65492584 | 65493678 |
| C8 | 65497937 | 65499208 |
| C8 | 65499483 | 65500634 |
| C8 | 66086746 | 66087756 |
| C8 | 66933484 | 66935213 |
| C8 | 67089832 | 67090990 |
| C8 | 67344225 | 67345764 |
| C8 | 67433408 | 67435022 |
| C8 | 67454318 | 67455343 |
| C8 | 67742520 | 67743573 |
| C8 | 67838229 | 67839439 |
| C8 | 67874972 | 67876146 |
| C8 | 68223278 | 68225076 |
| C8 | 68262326 | 68263788 |
| C8 | 68915427 | 68916738 |
| C8 | 69116541 | 69117711 |
| C8 | 69152609 | 69153651 |
| C8 | 69242205 | 69243440 |
| C8 | 70157563 | 70158731 |
| C8 | 70268661 | 70270133 |
| C8 | 70601781 | 70603109 |
| C8 | 70744681 | 70745764 |
| C8 | 70767512 | 70768646 |
| C8 | 70906496 | 70907640 |
| C8 | 70946853 | 70948094 |
| C8 | 70983631 | 70984794 |
| C8 | 70984059 | 70985243 |
| C8 | 71520134 | 71521222 |
| C8 | 71629470 | 71630478 |
| C8 | 72273482 | 72274614 |
| C8 | 72467756 | 72469262 |
| C8 | 72470003 | 72471267 |
| C8 | 72695819 | 72696971 |
| C8 | 72753436 | 72754496 |
| C8 | 72753910 | 72755078 |
| C8 | 72754140 | 72755254 |
| C8 | 72755254 | 72756445 |
| C8 | 72819827 | 72820971 |
| C8 | 74282711 | 74283778 |
| C8 | 74332022 | 74333974 |
| C8 | 74483072 | 74484092 |
| C8 | 74485243 | 74486270 |
| C8 | 74806739 | 74807869 |
| C8 | 75896207 | 75897318 |
| C8 | 76126603 | 76127709 |
| C8 | 77584861 | 77586248 |
| C8 | 77585859 | 77586932 |
| C8 | 77617391 | 77618902 |
| C8 | 78371692 | 78372710 |
| C8 | 81113247 | 81114456 |
| C8 | 81212400 | 81214091 |
| C8 | 81417465 | 81418629 |
| C8 | 81477594 | 81478846 |
| C8 | 81543143 | 81544540 |
| C8 | 81919812 | 81920883 |
| C8 | 82077244 | 82078255 |
| C8 | 82192019 | 82193069 |
| C8 | 83353082 | 83354225 |
| C8 | 84060106 | 84061151 |
| C8 | 85178466 | 85179626 |
| C8 | 86051291 | 86052308 |
| C8 | 86349780 | 86351207 |
| C8 | 86557776 | 86558975 |
| C8 | 86574636 | 86575748 |
| C8 | 86774428 | 86775551 |
| C8 | 86827780 | 86828986 |
| C8 | 90912456 | 90914140 |
| C8 | 91696908 | 91698317 |
| C8 | 91997053 | 91998160 |
| C8 | 91997296 | 91998433 |
| C8 | 92539200 | 92540489 |
| C8 | 93381615 | 93382680 |
| C8 | 94507553 | 94508672 |
| C8 | 94923623 | 94924697 |
| C8 | 94967557 | 94968792 |
| C8 | 95131492 | 95133228 |
| C8 | 95329268 | 95330393 |
| C8 | 96085158 | 96086300 |
| C8 | 96207949 | 96209562 |
| C8 | 97097598 | 97098666 |
| C8 | 97161251 | 97162890 |
| C8 | 97165223 | 97166621 |
| C8 | 97166208 | 97167462 |
| C8 | 97166772 | 97167994 |
| C8 | 97169690 | 97170753 |
| C8 | 97171306 | 97172845 |
| C8 | 97430826 | 97431976 |
| C8 | 98441961 | 98443037 |
| C8 | 98795474 | 98796987 |
| C8 | 98879777 | 98881063 |
| C8 | 99076288 | 99077365 |
| C8 | 99176112 | 99178194 |
| C8 | 99394464 | 99395822 |
| C8 | 99439409 | 99440862 |
| C8 | 99711942 | 99713108 |
| C8 | 99953932 | 99955126 |
| C8 | 99959795 | 99961196 |
| C8 | 99960491 | 99964552 |
| C8 | 99985484 | 99987226 |
| C8 | 99986241 | 99987501 |
| C8 | 101117565 | 101118862 |
| C8 | 101156377 | 101157456 |
| C8 | 101177154 | 101178401 |
| C8 | 101327830 | 101329102 |
| C8 | 101347859 | 101349024 |
| C8 | 101521531 | 101523322 |
| C8 | 101803340 | 101804722 |
| C8 | 101871928 | 101873188 |
| C8 | 101962856 | 101963956 |
| C8 | 102217381 | 102218444 |
| C8 | 102235927 | 102237117 |
| C8 | 102450052 | 102451796 |
| C8 | 103394766 | 103396461 |
| C8 | 103628712 | 103630369 |
| C8 | 103727992 | 103729686 |
| C8 | 103750499 | 103751553 |
| C8 | 103807257 | 103808593 |
| C8 | 104382466 | 104383690 |
| C8 | 104382735 | 104383914 |
| C8 | 104620367 | 104621608 |
| C8 | 104800579 | 104802005 |
| C8 | 105341469 | 105343746 |
| C8 | 105379015 | 105380364 |
| C8 | 105457495 | 105458727 |
| C8 | 105478338 | 105479644 |
| C8 | 105561169 | 105562599 |
| C8 | 105644559 | 105646139 |
| C8 | 105757109 | 105758296 |
| C8 | 106330697 | 106331730 |
| C8 | 106399009 | 106400568 |
| C8 | 107069463 | 107070637 |
| C8 | 107104703 | 107105730 |
| C8 | 107227311 | 107228597 |
| C8 | 107358538 | 107359930 |
| C8 | 107459754 | 107460833 |
| C8 | 107589717 | 107590736 |
| C8 | 108582734 | 108583768 |
| C8 | 109143482 | 109144795 |
| C8 | 110400864 | 110402549 |
| C8 | 110509871 | 110511081 |
| C8 | 110766491 | 110767767 |
| C8 | 110985910 | 110987098 |
| C8 | 111214531 | 111215604 |
| C8 | 111502482 | 111503669 |
| C8 | 112291730 | 112292900 |
| C8 | 112346868 | 112348078 |
| C8 | 113055729 | 113057257 |
| C8 | 113094786 | 113095967 |
| C8 | 113144253 | 113145902 |
| C8 | 113969213 | 113970328 |
| C8 | 114325073 | 114326241 |
| C8 | 114340027 | 114341162 |
| C8 | 114388391 | 114389980 |
| C8 | 114444409 | 114445526 |
| C8 | 114444561 | 114446179 |
| C8 | 114448312 | 114449622 |
| C8 | 114472023 | 114473152 |
| C8 | 114690398 | 114691688 |
| C8 | 114973099 | 114974271 |
| C8 | 115322912 | 115324407 |
| C8 | 115597760 | 115598964 |
| C8 | 116659933 | 116661310 |
| C8 | 117691929 | 117693073 |
| C8 | 118203332 | 118204602 |
| C8 | 118438595 | 118440226 |
| C8 | 118917333 | 118918506 |
| C8 | 118958117 | 118959735 |
| C8 | 119446456 | 119447821 |
| C8 | 119513530 | 119514753 |
| C8 | 119560296 | 119561647 |
| C8 | 119570422 | 119571595 |
| C8 | 120044364 | 120045597 |
| C8 | 120176785 | 120178129 |
| C8 | 121163996 | 121165195 |
| C8 | 121681337 | 121682492 |
| C8 | 121880483 | 121881855 |
| C8 | 121982105 | 121983150 |
| C8 | 122168603 | 122169861 |
| C8 | 122231019 | 122232030 |
| C8 | 122385232 | 122386499 |
| C8 | 122493609 | 122494769 |
| C8 | 122595832 | 122597033 |
| C8 | 122602381 | 122603661 |
| C8 | 122668817 | 122670064 |
| C8 | 122679139 | 122680679 |
| C8 | 122745744 | 122746999 |
| C8 | 122764330 | 122765598 |
| C8 | 122785156 | 122786437 |
| C8 | 122790197 | 122791417 |
| C8 | 122806902 | 122808358 |
| C8 | 122846737 | 122847754 |
| C8 | 122874264 | 122875293 |
| C8 | 122944987 | 122946352 |
| C8 | 122959733 | 122961170 |
| C8 | 122960984 | 122962274 |
| C8 | 123256155 | 123257641 |
| C8 | 123694626 | 123695757 |
| C8 | 123707861 | 123709128 |
| C8 | 123716260 | 123717540 |
| C8 | 124027340 | 124028459 |
| C8 | 124054752 | 124056066 |
| C8 | 124055943 | 124057299 |
| C8 | 124173009 | 124174170 |
| C8 | 124219030 | 124220199 |
| C8 | 124253372 | 124254821 |
| C8 | 124288116 | 124289391 |
| C8 | 124327498 | 124328689 |
| C8 | 124877301 | 124878687 |
| C8 | 125312391 | 125313618 |
| C8 | 125313073 | 125314597 |
| C8 | 125616724 | 125617762 |
| C8 | 125701739 | 125703297 |
| C8 | 125769675 | 125770743 |
| C8 | 125846352 | 125847583 |
| C8 | 125984473 | 125985606 |
| C8 | 126425190 | 126426224 |
| C8 | 126445218 | 126446277 |
| C8 | 126463913 | 126464975 |
| C8 | 126588016 | 126589188 |
| C8 | 127304412 | 127305723 |
| C8 | 127330581 | 127331871 |
| C8 | 127353348 | 127354630 |
| C8 | 127366062 | 127367290 |
| C8 | 127370052 | 127371328 |
| C8 | 127437499 | 127438572 |
| C8 | 127464274 | 127465476 |
| C8 | 127772623 | 127773939 |
| C8 | 127816908 | 127818254 |
| C8 | 127963579 | 127964655 |
| C8 | 128865591 | 128867283 |
| C8 | 128919226 | 128920327 |
| C8 | 128961426 | 128962953 |
| C8 | 128963630 | 128965426 |
| C8 | 129039718 | 129041084 |
| C8 | 129084351 | 129085518 |
| C8 | 129164932 | 129166142 |
| C8 | 129710180 | 129711499 |
| C8 | 129756386 | 129757890 |
| C8 | 129837153 | 129838402 |
| C8 | 129838456 | 129839836 |
| C8 | 129908974 | 129910130 |
| C8 | 129996084 | 129997349 |
| C8 | 130052797 | 130053974 |
| C8 | 130076077 | 130077402 |
| C8 | 130393804 | 130394946 |
| C8 | 130508158 | 130509557 |
| C8 | 130995456 | 130996517 |
| C8 | 130999915 | 131001062 |
| C8 | 131086713 | 131087946 |
| C8 | 131589454 | 131590810 |
| C8 | 131688234 | 131689433 |
| C8 | 131716706 | 131717836 |
| C8 | 131734408 | 131735761 |
| C8 | 131819449 | 131820725 |
| C8 | 131874186 | 131875497 |
| C8 | 132052279 | 132053387 |
| C8 | 132053191 | 132054273 |
| C8 | 132053960 | 132055060 |
| C8 | 132055638 | 132057080 |
| C8 | 132167738 | 132169094 |
| C8 | 132236341 | 132237621 |
| C8 | 132283676 | 132284966 |
| C8 | 132409443 | 132410684 |
| C8 | 132415455 | 132416692 |
| C8 | 132476633 | 132477912 |
| C8 | 132498448 | 132500203 |
| C8 | 132521357 | 132522898 |
| C8 | 132588504 | 132589970 |
| C8 | 132645340 | 132646699 |
| C8 | 132646934 | 132648356 |
| C8 | 132782702 | 132784012 |
| C8 | 133140889 | 133142390 |
| C8 | 133259255 | 133260585 |
| C8 | 133261464 | 133263129 |
| C8 | 133448886 | 133450020 |
| C8 | 133641116 | 133642388 |
| C8 | 133686346 | 133687565 |
| C8 | 133863901 | 133865201 |
| C8 | 133894582 | 133895797 |
| C8 | 133935104 | 133936143 |
| C8 | 134041470 | 134042809 |
| C8 | 134142577 | 134143771 |
| C8 | 134223871 | 134225315 |
| C8 | 134257633 | 134258765 |
| C8 | 134509950 | 134513201 |
| C8 | 134582837 | 134584041 |
| C8 | 134768649 | 134770111 |
| C8 | 135475669 | 135476722 |
| C8 | 135476076 | 135477611 |
| C8 | 136002522 | 136003696 |
| C8 | 136261328 | 136262346 |
| C8 | 136270980 | 136272195 |
| C8 | 136472837 | 136474135 |
| C8 | 136526120 | 136528104 |
| C8 | 136548425 | 136549697 |
| C8 | 136674998 | 136676274 |
| C8 | 136683575 | 136684943 |
| C8 | 136702185 | 136703908 |
| C8 | 136704421 | 136705657 |
| C8 | 136887708 | 136889097 |
| C8 | 136888986 | 136890732 |
| C8 | 137069825 | 137071061 |
| C8 | 137085158 | 137086471 |
| C8 | 137126389 | 137127615 |
| C8 | 137184636 | 137185897 |
| C8 | 137238158 | 137239389 |
| C8 | 137284847 | 137286711 |
| C8 | 137542425 | 137543703 |
| C8 | 137805535 | 137806900 |
| C8 | 138037159 | 138038221 |
| C8 | 138120193 | 138121463 |
| C8 | 138124053 | 138125298 |
| C8 | 138131310 | 138132658 |
| C8 | 138142542 | 138144257 |
| C8 | 138199705 | 138200936 |
| C8 | 138207300 | 138208641 |
| C8 | 138246046 | 138247311 |
| C8 | 138264947 | 138266382 |
| C8 | 138270222 | 138271272 |
| C8 | 138323652 | 138324912 |
| C8 | 138335079 | 138336344 |
| C8 | 138394687 | 138395864 |
| C8 | 138549412 | 138550674 |
| C8 | 138575990 | 138577431 |
| C8 | 138615505 | 138616743 |
| C8 | 138640956 | 138642221 |
| C8 | 138747226 | 138748844 |
| C8 | 138766053 | 138767411 |
| C8 | 138766847 | 138768320 |
| C8 | 138808298 | 138809477 |
| C8 | 138860069 | 138861171 |
| C8 | 138908643 | 138909889 |
| C8 | 138909489 | 138910867 |
| C8 | 138990776 | 138992011 |
| C8 | 139059845 | 139061266 |
| C8 | 139075799 | 139078243 |
| C8 | 139095945 | 139097316 |
| C8 | 139109720 | 139111010 |
| C8 | 139110356 | 139111672 |
| C8 | 139127966 | 139129399 |
| C8 | 139139309 | 139140585 |
| C8 | 139154894 | 139156312 |
| C8 | 139170875 | 139172000 |
| C8 | 139262488 | 139263870 |
| C8 | 139318418 | 139320785 |
| C8 | 139328020 | 139329506 |
| C8 | 139356205 | 139357443 |
| C8 | 139382225 | 139384420 |
| C8 | 139502538 | 139503872 |
| C8 | 139508923 | 139509993 |
| C8 | 139610901 | 139612200 |
| C8 | 139736347 | 139737620 |
| C8 | 139766630 | 139768062 |
| C8 | 139786184 | 139787453 |
| C8 | 139805586 | 139806857 |
| C8 | 139856286 | 139857649 |
| C8 | 139889568 | 139890914 |
| C8 | 139893194 | 139894474 |
| C8 | 139940765 | 139942215 |
| C8 | 139955571 | 139956987 |
| C8 | 139971217 | 139972706 |
| C8 | 139980773 | 139981994 |
| C8 | 140257244 | 140258484 |
| C8 | 140304744 | 140305995 |
| C8 | 140305958 | 140307517 |
| C8 | 140313079 | 140314266 |
| C8 | 140317519 | 140318559 |
| C8 | 140420036 | 140421417 |
| C8 | 140428304 | 140429843 |
| C8 | 140467625 | 140468936 |
| C8 | 140470640 | 140471689 |
| C8 | 140474612 | 140475823 |
| C8 | 140603648 | 140604690 |
| C8 | 140612716 | 140614333 |
| C8 | 140623107 | 140625389 |
| C8 | 140630006 | 140631832 |
| C8 | 140635673 | 140637137 |
| C8 | 140638537 | 140639881 |
| C8 | 140645449 | 140646807 |
| C8 | 140652337 | 140653816 |
| C8 | 140769721 | 140771065 |
| C8 | 141045194 | 141046394 |
| C8 | 141108727 | 141109879 |
| C8 | 141110006 | 141111112 |
| C8 | 141160399 | 141161860 |
| C8 | 141358717 | 141360175 |
| C8 | 141402159 | 141403203 |
| C8 | 141493850 | 141494948 |
| C8 | 141554694 | 141555804 |
| C8 | 141598850 | 141600105 |
| C8 | 141608726 | 141609972 |
| C8 | 141691092 | 141692370 |
| C8 | 142045499 | 142047093 |
| C8 | 142105080 | 142106505 |
| C8 | 142138024 | 142139188 |
| C8 | 142147688 | 142148847 |
| C8 | 142159700 | 142160967 |
| C8 | 142202536 | 142203742 |
| C8 | 142218636 | 142219720 |
| C8 | 142218935 | 142220106 |
| C8 | 142244451 | 142245625 |
| C8 | 142275717 | 142277482 |
| C8 | 142367139 | 142368669 |
| C8 | 142384293 | 142385530 |
| C8 | 142387543 | 142388763 |
| C8 | 142412632 | 142413969 |
| C8 | 142413631 | 142414821 |
| C8 | 142426782 | 142428371 |
| C8 | 142427574 | 142428997 |
| C8 | 142455135 | 142456340 |
| C8 | 142492549 | 142493923 |
| C8 | 142509381 | 142510608 |
| C8 | 142522010 | 142523053 |
| C8 | 142527686 | 142528791 |
| C8 | 142528254 | 142529376 |
| C8 | 142554673 | 142555896 |
| C8 | 142623352 | 142624705 |
| C8 | 142628452 | 142629756 |
| C8 | 142629581 | 142631144 |
| C8 | 142661634 | 142662941 |
| C8 | 142669420 | 142670789 |
| C8 | 142694464 | 142696150 |
| C8 | 142745153 | 142746632 |
| C8 | 142747156 | 142748846 |
| C8 | 142816480 | 142817708 |
| C8 | 142818773 | 142819919 |
| C8 | 142818932 | 142820152 |
| C8 | 142821426 | 142822676 |
| C8 | 142832602 | 142834125 |
| C8 | 142835789 | 142837171 |
| C8 | 142839329 | 142840679 |
| C8 | 142841327 | 142842707 |
| C8 | 142852211 | 142853761 |
| C8 | 142857268 | 142858458 |
| C8 | 142887346 | 142888714 |
| C8 | 142891367 | 142892403 |
| C8 | 142895262 | 142896521 |
| C8 | 142983517 | 142984764 |
| C8 | 142983863 | 142985333 |
| C8 | 143007905 | 143008923 |
| C8 | 143008525 | 143009751 |
| C8 | 143013355 | 143014968 |
| C8 | 143049937 | 143051330 |
| C8 | 143054170 | 143055639 |
| C8 | 143096844 | 143098198 |
| C8 | 143104506 | 143105682 |
| C8 | 143113589 | 143114888 |
| C8 | 143125774 | 143127043 |
| C8 | 143149919 | 143151430 |
| C8 | 143151935 | 143153694 |
| C8 | 143164401 | 143165729 |
| C8 | 143181768 | 143183577 |
| C8 | 143186074 | 143187487 |
| C8 | 143200436 | 143201944 |
| C8 | 143204234 | 143205682 |
| C8 | 143206814 | 143208923 |
| C8 | 143216569 | 143218536 |
| C8 | 143234136 | 143235270 |
| C8 | 143249481 | 143250847 |
| C8 | 143264688 | 143266060 |
| C8 | 143274191 | 143275541 |
| C8 | 143307063 | 143308335 |
| C8 | 143313151 | 143314477 |
| C8 | 143316041 | 143317325 |
| C8 | 143318548 | 143319970 |
| C8 | 143324770 | 143326041 |
| C8 | 143325384 | 143326768 |
| C8 | 143331168 | 143332444 |
| C8 | 143332548 | 143334049 |
| C8 | 143340393 | 143341657 |
| C8 | 143386314 | 143387825 |
| C8 | 143508443 | 143510198 |
| C8 | 143521807 | 143522848 |
| C8 | 143545135 | 143546294 |
| C8 | 143557215 | 143558496 |
| C8 | 143557841 | 143559324 |
| C8 | 143558902 | 143560381 |
| C8 | 143572332 | 143573653 |
| C8 | 143580999 | 143582088 |
| C8 | 143581151 | 143582222 |
| C8 | 143596883 | 143598394 |
| C8 | 143597930 | 143599494 |
| C8 | 143606929 | 143608567 |
| C8 | 143614047 | 143615592 |
| C8 | 143617749 | 143618994 |
| C8 | 143618381 | 143619790 |
| C8 | 143622612 | 143624162 |
| C8 | 143624977 | 143626686 |
| C8 | 143630925 | 143632081 |
| C8 | 143644043 | 143645576 |
| C8 | 143655686 | 143656882 |
| C8 | 143656719 | 143657931 |
| C8 | 143659928 | 143661174 |
| C8 | 143662236 | 143663674 |
| C8 | 143665966 | 143667069 |
| C8 | 143678369 | 143679616 |
| C8 | 143693364 | 143694507 |
| C8 | 143694129 | 143695784 |
| C8 | 143781028 | 143782181 |
| C8 | 143809880 | 143811087 |
| C8 | 143823234 | 143824501 |
| C8 | 143857686 | 143858853 |
| C8 | 143915636 | 143917106 |
| C8 | 143993381 | 143994773 |
| C8 | 144003926 | 144005426 |
| C8 | 144008955 | 144010402 |
| C8 | 144019538 | 144021405 |
| C8 | 144040252 | 144041827 |
| C8 | 144061298 | 144062545 |
| C8 | 144119606 | 144121350 |
| C8 | 144122735 | 144123899 |
| C8 | 144150748 | 144151948 |
| C8 | 144153296 | 144154466 |
| C8 | 144153559 | 144155613 |
| C8 | 144166021 | 144167172 |
| C8 | 144177884 | 144179537 |
| C8 | 144187814 | 144189506 |
| C8 | 144189275 | 144190487 |
| C8 | 144189607 | 144191066 |
| C8 | 144203021 | 144204645 |
| C8 | 144206333 | 144207589 |
| C8 | 144211533 | 144212766 |
| C8 | 144214046 | 144215487 |
| C8 | 144221893 | 144222980 |
| C8 | 144222213 | 144223658 |
| C8 | 144238173 | 144239576 |
| C8 | 144253259 | 144254430 |
| C8 | 144262201 | 144263519 |
| C8 | 144266417 | 144267911 |
| C8 | 144278010 | 144279306 |
| C8 | 144292263 | 144293896 |
| C8 | 144300197 | 144301476 |
| C8 | 144303009 | 144304548 |
| C8 | 144311209 | 144312395 |
| C8 | 144317147 | 144318647 |
| C8 | 144328002 | 144329074 |
| C8 | 144343550 | 144344646 |
| C8 | 144360341 | 144362004 |
| C8 | 144366765 | 144368025 |
| C8 | 144371245 | 144372374 |
| C8 | 144404348 | 144405438 |
| C8 | 144436296 | 144439023 |
| C8 | 144551239 | 144552318 |
| C8 | 144597771 | 144599078 |
| C8 | 144626332 | 144627626 |
| C8 | 144629440 | 144630646 |
| C8 | 144631276 | 144632453 |
| C8 | 144635786 | 144637036 |
| C8 | 144642110 | 144643651 |
| C8 | 144648958 | 144650179 |
| C8 | 144650292 | 144651385 |
| C8 | 144652430 | 144653645 |
| C8 | 144659488 | 144661075 |
| C8 | 144759949 | 144761281 |
| C8 | 144765941 | 144767493 |
| C8 | 144778747 | 144779756 |
| C8 | 144787266 | 144788652 |
| C8 | 144787765 | 144789055 |
| C8 | 144788801 | 144789906 |
| C8 | 144788977 | 144790209 |
| C8 | 144789246 | 144790323 |
| C8 | 144789378 | 144790824 |
| C8 | 144808572 | 144809658 |
| C8 | 144809083 | 144810189 |
| C8 | 144809261 | 144810492 |
| C8 | 144809595 | 144811201 |
| C8 | 144819971 | 144821458 |
| C8 | 144854008 | 144855151 |
| C8 | 144882373 | 144883734 |
| C8 | 144894582 | 144895870 |
| C8 | 144923126 | 144924367 |
| C8 | 144935113 | 144936572 |
| C8 | 144951564 | 144952717 |
| C8 | 144977093 | 144979092 |
| C8 | 145002433 | 145003509 |
| C8 | 145003113 | 145004268 |
| C8 | 145007786 | 145009033 |
| C8 | 145011218 | 145012870 |
| C8 | 145013110 | 145014274 |
| C8 | 145024062 | 145025208 |
| C8 | 145027113 | 145028524 |
| C8 | 145051575 | 145052670 |
| C8 | 145052416 | 145053704 |
| C8 | 145096759 | 145098222 |
| C8 | 145098763 | 145100222 |
| C8 | 145099336 | 145100834 |
| C8 | 145100956 | 145102844 |
| C8 | 145102984 | 145104694 |
| C8 | 145104141 | 145105238 |
| C8 | 145105101 | 145106203 |
| C8 | 145106141 | 145107783 |
| C8 | 145107130 | 145108246 |
| C8 | 145113889 | 145115763 |
| C8 | 145134233 | 145135569 |
| C8 | 145138102 | 145139367 |
| C8 | 145145688 | 145146895 |
| C8 | 145163741 | 145164927 |
| C8 | 145163973 | 145165352 |
| C8 | 145179294 | 145180637 |
| C8 | 145239699 | 145241021 |
| C8 | 145256845 | 145258041 |
| C8 | 145257708 | 145259083 |
| C8 | 145325214 | 145326385 |
| C8 | 145325752 | 145326929 |
| C8 | 145327493 | 145328690 |
| C8 | 145490708 | 145491840 |
| C8 | 145494200 | 145495470 |
| C8 | 145496546 | 145497611 |
| C8 | 145496636 | 145497706 |
| C8 | 145501253 | 145502436 |
| C8 | 145536939 | 145538036 |
| C8 | 145537078 | 145538115 |
| C8 | 145559223 | 145560292 |
| C8 | 145577108 | 145578270 |
| C8 | 145577480 | 145578539 |
| C8 | 145592975 | 145594416 |
| C8 | 145607750 | 145609246 |
| C8 | 145615696 | 145617129 |
| C8 | 145634228 | 145635292 |
| C8 | 145638434 | 145639544 |
| C8 | 145647144 | 145648551 |
| C8 | 145653967 | 145655088 |
| C8 | 145658826 | 145660752 |
| C8 | 145690729 | 145691843 |
| C8 | 145725169 | 145726301 |
| C8 | 145725534 | 145726738 |
| C8 | 145725811 | 145726945 |
| C8 | 145730155 | 145731376 |
| C8 | 145730425 | 145731748 |
| C8 | 145731431 | 145732552 |
| C8 | 145747010 | 145748305 |
| C8 | 145747883 | 145748993 |
| C8 | 145749469 | 145750577 |
| C8 | 145749734 | 145750802 |
| C8 | 145749829 | 145750910 |
| C8 | 145751456 | 145752615 |
| C8 | 145755397 | 145756487 |
| C8 | 145755533 | 145756754 |
| C8 | 145900186 | 145901922 |
| C8 | 145911957 | 145913240 |
| C8 | 145923999 | 145925196 |
| C8 | 145955350 | 145956409 |
| C9 | 20679 | 22152 |
| C9 | 30071 | 33208 |
| C9 | 468763 | 470213 |
| C9 | 686958 | 688005 |
| C9 | 763647 | 764904 |
| C9 | 826083 | 827188 |
| C9 | 4739153 | 4740292 |
| C9 | 5042456 | 5043595 |
| C9 | 5482139 | 5483588 |
| C9 | 5599756 | 5600936 |
| C9 | 6756085 | 6757243 |
| C9 | 8140317 | 8141404 |
| C9 | 8858135 | 8859464 |
| C9 | 10207730 | 10208811 |
| C9 | 10360372 | 10361399 |
| C9 | 10785803 | 10786830 |
| C9 | 11862593 | 11864246 |
| C9 | 12627696 | 12628727 |
| C9 | 12728025 | 12729044 |
| C9 | 14345056 | 14346455 |
| C9 | 14977690 | 14978774 |
| C9 | 14992680 | 14993823 |
| C9 | 15232431 | 15233688 |
| C9 | 16310413 | 16311497 |
| C9 | 16743495 | 16744658 |
| C9 | 16855104 | 16856237 |
| C9 | 17214863 | 17216596 |
| C9 | 17353440 | 17354467 |
| C9 | 17367171 | 17368251 |
| C9 | 17401015 | 17402025 |
| C9 | 18253953 | 18255113 |
| C9 | 18949332 | 18950418 |
| C9 | 19535945 | 19538817 |
| C9 | 20657802 | 20659167 |
| C9 | 20804166 | 20805607 |
| C9 | 21696021 | 21697103 |
| C9 | 22352981 | 22354409 |
| C9 | 23363091 | 23364214 |
| C9 | 25033271 | 25034342 |
| C9 | 25593775 | 25595051 |
| C9 | 27070939 | 27071943 |
| C9 | 27690434 | 27691731 |
| C9 | 28117010 | 28118142 |
| C9 | 29950449 | 29951576 |
| C9 | 30757868 | 30758919 |
| C9 | 31210770 | 31212372 |
| C9 | 31627487 | 31628555 |
| C9 | 32693448 | 32694596 |
| C9 | 33314592 | 33316206 |
| C9 | 33318139 | 33319639 |
| C9 | 33676846 | 33677902 |
| C9 | 33751093 | 33752312 |
| C9 | 34370465 | 34371574 |
| C9 | 34371046 | 34372120 |
| C9 | 34651514 | 34652749 |
| C9 | 34719810 | 34720935 |
| C9 | 34987987 | 34989081 |
| C9 | 35035718 | 35037068 |
| C9 | 35080299 | 35081447 |
| C9 | 35675259 | 35676387 |
| C9 | 35689320 | 35690502 |
| C9 | 35815579 | 35816793 |
| C9 | 35828670 | 35829733 |
| C9 | 35961864 | 35963741 |
| C9 | 36739167 | 36740311 |
| C9 | 37002327 | 37003476 |
| C9 | 37118740 | 37119953 |
| C9 | 38240848 | 38241943 |
| C9 | 38242252 | 38243396 |
| C9 | 38487144 | 38488717 |
| C9 | 38832575 | 38833749 |
| C9 | 38906028 | 38907240 |
| C9 | 39012041 | 39013231 |
| C9 | 39132535 | 39133669 |
| C9 | 39368565 | 39369703 |
| C9 | 39590414 | 39591502 |
| C9 | 39636234 | 39637401 |
| C9 | 39814520 | 39815924 |
| C9 | 40053137 | 40054374 |
| C9 | 40073407 | 40074873 |
| C9 | 40094384 | 40095552 |
| C9 | 40355562 | 40356899 |
| C9 | 40408227 | 40409493 |
| C9 | 40496185 | 40497906 |
| C9 | 40506174 | 40508449 |
| C9 | 40899407 | 40900471 |
| C9 | 40902679 | 40904250 |
| C9 | 40909360 | 40910627 |
| C9 | 41037983 | 41039076 |
| C9 | 41219810 | 41220941 |
| C9 | 41221480 | 41223672 |
| C9 | 41228459 | 41229731 |
| C9 | 41291234 | 41293433 |
| C9 | 41737039 | 41739198 |
| C9 | 41781230 | 41782497 |
| C9 | 41861735 | 41863482 |
| C9 | 41889565 | 41890749 |
| C9 | 41910413 | 41911554 |
| C9 | 41995431 | 41997407 |
| C9 | 42178097 | 42180129 |
| C9 | 42251198 | 42253007 |
| C9 | 42252076 | 42253183 |
| C9 | 42252406 | 42253546 |
| C9 | 42255003 | 42256061 |
| C9 | 42255776 | 42257011 |
| C9 | 42320513 | 42322142 |
| C9 | 42321420 | 42322949 |
| C9 | 42341674 | 42343105 |
| C9 | 42342667 | 42343789 |
| C9 | 42491332 | 42492521 |
| C9 | 42491971 | 42493127 |
| C9 | 42503136 | 42504435 |
| C9 | 42988397 | 42989535 |
| C9 | 43139136 | 43140932 |
| C9 | 43351858 | 43353294 |
| C9 | 43384874 | 43386086 |
| C9 | 43388925 | 43390097 |
| C9 | 43453226 | 43454301 |
| C9 | 43453315 | 43454493 |
| C9 | 43475829 | 43477985 |
| C9 | 43481213 | 43482474 |
| C9 | 43524842 | 43526154 |
| C9 | 43525197 | 43526815 |
| C9 | 43525827 | 43527091 |
| C9 | 43554951 | 43556075 |
| C9 | 43914794 | 43915963 |
| C9 | 44117761 | 44118977 |
| C9 | 44118127 | 44119230 |
| C9 | 44118357 | 44120350 |
| C9 | 44245070 | 44246215 |
| C9 | 44245252 | 44246441 |
| C9 | 44507242 | 44508565 |
| C9 | 44607321 | 44609355 |
| C9 | 44728528 | 44729683 |
| C9 | 44729330 | 44730559 |
| C9 | 44743449 | 44744658 |
| C9 | 44759186 | 44760288 |
| C9 | 44827722 | 44829725 |
| C9 | 44841991 | 44843169 |
| C9 | 44842361 | 44843575 |
| C9 | 44858990 | 44860870 |
| C9 | 44989612 | 44990691 |
| C9 | 44992169 | 44993219 |
| C9 | 45004885 | 45005988 |
| C9 | 45047043 | 45048402 |
| C9 | 45170636 | 45172334 |
| C9 | 45385328 | 45386378 |
| C9 | 45436537 | 45437881 |
| C9 | 45437291 | 45438605 |
| C9 | 45438726 | 45439939 |
| C9 | 45439487 | 45440759 |
| C9 | 45440264 | 45441448 |
| C9 | 45727134 | 45728444 |
| C9 | 45731523 | 45732542 |
| C9 | 45733502 | 45734925 |
| C9 | 45737194 | 45738347 |
| C9 | 45740127 | 45741249 |
| C9 | 45742004 | 45743347 |
| C9 | 45762422 | 45763684 |
| C9 | 45781168 | 45782642 |
| C9 | 45783145 | 45784232 |
| C9 | 46112715 | 46113850 |
| C9 | 46117433 | 46118539 |
| C9 | 46132502 | 46133576 |
| C9 | 46149156 | 46150283 |
| C9 | 46354735 | 46355801 |
| C9 | 46379088 | 46380339 |
| C9 | 46385940 | 46387098 |
| C9 | 46401114 | 46403223 |
| C9 | 46761419 | 46763072 |
| C9 | 46844002 | 46845365 |
| C9 | 46861698 | 46862770 |
| C9 | 46907849 | 46909259 |
| C9 | 46934123 | 46935324 |
| C9 | 46981457 | 46982572 |
| C9 | 47048940 | 47050601 |
| C9 | 65602149 | 65603189 |
| C9 | 65744130 | 65745456 |
| C9 | 65783644 | 65784828 |
| C9 | 65874035 | 65875169 |
| C9 | 65879610 | 65880850 |
| C9 | 65897515 | 65899480 |
| C9 | 65914424 | 65915576 |
| C9 | 65968666 | 65969891 |
| C9 | 66343736 | 66345307 |
| C9 | 66486873 | 66488067 |
| C9 | 66494182 | 66495445 |
| C9 | 66500212 | 66501306 |
| C9 | 66553012 | 66554144 |
| C9 | 66757895 | 66759407 |
| C9 | 67272244 | 67273693 |
| C9 | 67339600 | 67340640 |
| C9 | 67339642 | 67340886 |
| C9 | 67339903 | 67341814 |
| C9 | 67605840 | 67607544 |
| C9 | 67664402 | 67665640 |
| C9 | 67673518 | 67675135 |
| C9 | 67695874 | 67697665 |
| C9 | 67784915 | 67786030 |
| C9 | 67790625 | 67791916 |
| C9 | 67791806 | 67792936 |
| C9 | 67793437 | 67794660 |
| C9 | 67900062 | 67901501 |
| C9 | 67924689 | 67925875 |
| C9 | 67972149 | 67974715 |
| C9 | 68302411 | 68303580 |
| C9 | 68408064 | 68409176 |
| C9 | 68409079 | 68410160 |
| C9 | 68455158 | 68456219 |
| C9 | 68707742 | 68709421 |
| C9 | 68709165 | 68710375 |
| C9 | 68726208 | 68727284 |
| C9 | 68809066 | 68810210 |
| C9 | 69200387 | 69201555 |
| C9 | 69225908 | 69228429 |
| C9 | 69333779 | 69334902 |
| C9 | 69356106 | 69357405 |
| C9 | 69379878 | 69381073 |
| C9 | 69381321 | 69382412 |
| C9 | 69480577 | 69483622 |
| C9 | 69497390 | 69498887 |
| C9 | 69498681 | 69500052 |
| C9 | 69934727 | 69936288 |
| C9 | 70089154 | 70090238 |
| C9 | 70089249 | 70091008 |
| C9 | 70176593 | 70177679 |
| C9 | 70177087 | 70178297 |
| C9 | 70177346 | 70178519 |
| C9 | 70625324 | 70626479 |
| C9 | 70649255 | 70650491 |
| C9 | 70649701 | 70650988 |
| C9 | 70650960 | 70652297 |
| C9 | 70717368 | 70718809 |
| C9 | 70917849 | 70919311 |
| C9 | 70921971 | 70923105 |
| C9 | 70938185 | 70939368 |
| C9 | 71654051 | 71655148 |
| C9 | 71911724 | 71912753 |
| C9 | 72131155 | 72132237 |
| C9 | 72131325 | 72132463 |
| C9 | 72375574 | 72376729 |
| C9 | 72989725 | 72990911 |
| C9 | 73402990 | 73403995 |
| C9 | 73449740 | 73450809 |
| C9 | 74268904 | 74270031 |
| C9 | 74383425 | 74384480 |
| C9 | 74526882 | 74528617 |
| C9 | 75451479 | 75452529 |
| C9 | 75695708 | 75696854 |
| C9 | 75738659 | 75739829 |
| C9 | 76380289 | 76381372 |
| C9 | 77482334 | 77483390 |
| C9 | 77642697 | 77643753 |
| C9 | 77703243 | 77704299 |
| C9 | 78248686 | 78249753 |
| C9 | 78657533 | 78658700 |
| C9 | 79186172 | 79187215 |
| C9 | 79626451 | 79627558 |
| C9 | 80262412 | 80263489 |
| C9 | 81056298 | 81057450 |
| C9 | 83088104 | 83089203 |
| C9 | 83158108 | 83159246 |
| C9 | 83934958 | 83936689 |
| C9 | 86419199 | 86420312 |
| C9 | 86594217 | 86595304 |
| C9 | 86716846 | 86717877 |
| C9 | 87151339 | 87152346 |
| C9 | 87627777 | 87628821 |
| C9 | 87691733 | 87692950 |
| C9 | 90132098 | 90133672 |
| C9 | 90559072 | 90560181 |
| C9 | 90859109 | 90860180 |
| C9 | 91048372 | 91049426 |
| C9 | 91538141 | 91539502 |
| C9 | 91562379 | 91564365 |
| C9 | 91896990 | 91898045 |
| C9 | 91925195 | 91926328 |
| C9 | 92002484 | 92003946 |
| C9 | 92036975 | 92038004 |
| C9 | 92754735 | 92755795 |
| C9 | 92912411 | 92914897 |
| C9 | 92989118 | 92990605 |
| C9 | 93295476 | 93296558 |
| C9 | 93602154 | 93603328 |
| C9 | 94648080 | 94649437 |
| C9 | 94765378 | 94766938 |
| C9 | 95591991 | 95593323 |
| C9 | 95857404 | 95858471 |
| C9 | 95857700 | 95858787 |
| C9 | 96053410 | 96054428 |
| C9 | 96058752 | 96059924 |
| C9 | 96060166 | 96061232 |
| C9 | 96114542 | 96116000 |
| C9 | 96197001 | 96198176 |
| C9 | 96328897 | 96330125 |
| C9 | 96338234 | 96339329 |
| C9 | 96729881 | 96731094 |
| C9 | 96792446 | 96793482 |
| C9 | 97069054 | 97070957 |
| C9 | 97866017 | 97867048 |
| C9 | 98075138 | 98076241 |
| C9 | 98810893 | 98812054 |
| C9 | 98811260 | 98812590 |
| C9 | 98980799 | 98982053 |
| C9 | 99686857 | 99687994 |
| C9 | 99838735 | 99839855 |
| C9 | 99999357 | 100000479 |
| C9 | 100069391 | 100070541 |
| C9 | 100069631 | 100070700 |
| C9 | 100076445 | 100077695 |
| C9 | 100392923 | 100394023 |
| C9 | 100610895 | 100612016 |
| C9 | 100746496 | 100747543 |
| C9 | 100849416 | 100850892 |
| C9 | 101642936 | 101643952 |
| C9 | 101867046 | 101868106 |
| C9 | 102021218 | 102022588 |
| C9 | 102581832 | 102582882 |
| C9 | 102586196 | 102587275 |
| C9 | 103172156 | 103173293 |
| C9 | 103174845 | 103175997 |
| C9 | 103869587 | 103870706 |
| C9 | 104425957 | 104428082 |
| C9 | 105248990 | 105250061 |
| C9 | 107051399 | 107052432 |
| C9 | 107120264 | 107121342 |
| C9 | 108456294 | 108457357 |
| C9 | 108543331 | 108544792 |
| C9 | 110032905 | 110034365 |
| C9 | 110044810 | 110045904 |
| C9 | 110249440 | 110250522 |
| C9 | 110478381 | 110479580 |
| C9 | 110538064 | 110539757 |
| C9 | 111616561 | 111618198 |
| C9 | 111661027 | 111662037 |
| C9 | 112429739 | 112430917 |
| C9 | 112934692 | 112935834 |
| C9 | 112955335 | 112956478 |
| C9 | 113228457 | 113229824 |
| C9 | 114657165 | 114658308 |
| C9 | 115697497 | 115698524 |
| C9 | 115821760 | 115823028 |
| C9 | 115822752 | 115823861 |
| C9 | 115823197 | 115824800 |
| C9 | 115823904 | 115825046 |
| C9 | 115824377 | 115826337 |
| C9 | 115847234 | 115848634 |
| C9 | 115849847 | 115851341 |
| C9 | 115850454 | 115851923 |
| C9 | 115850991 | 115852105 |
| C9 | 115851343 | 115852555 |
| C9 | 115851706 | 115852872 |
| C9 | 116225264 | 116226891 |
| C9 | 116263079 | 116264179 |
| C9 | 116764233 | 116765365 |
| C9 | 116987691 | 116988699 |
| C9 | 117086938 | 117088372 |
| C9 | 117848338 | 117850050 |
| C9 | 119396225 | 119397326 |
| C9 | 119833391 | 119834532 |
| C9 | 120404492 | 120405695 |
| C9 | 121482507 | 121483646 |
| C9 | 121571075 | 121572170 |
| C9 | 121771531 | 121772767 |
| C9 | 123476325 | 123477423 |
| C9 | 124088316 | 124089428 |
| C9 | 124460614 | 124461717 |
| C9 | 124658625 | 124659661 |
| C9 | 124733010 | 124734263 |
| C9 | 124890396 | 124891522 |
| C9 | 124981374 | 124982506 |
| C9 | 124981587 | 124982926 |
| C9 | 124983128 | 124984255 |
| C9 | 124987642 | 124988710 |
| C9 | 124988124 | 124989256 |
| C9 | 124988344 | 124990164 |
| C9 | 124989179 | 124990416 |
| C9 | 125167786 | 125169442 |
| C9 | 125706271 | 125707475 |
| C9 | 125735713 | 125736779 |
| C9 | 126030596 | 126031651 |
| C9 | 126127972 | 126128995 |
| C9 | 126149540 | 126150732 |
| C9 | 126940465 | 126941603 |
| C9 | 126945549 | 126946604 |
| C9 | 126986083 | 126987156 |
| C9 | 127261685 | 127263438 |
| C9 | 127267460 | 127268583 |
| C9 | 127510361 | 127511536 |
| C9 | 127600497 | 127601558 |
| C9 | 127600568 | 127601611 |
| C9 | 127961766 | 127962852 |
| C9 | 128090633 | 128091794 |
| C9 | 128385564 | 128386651 |
| C9 | 128386423 | 128387620 |
| C9 | 128984524 | 128985608 |
| C9 | 129233124 | 129234204 |
| C9 | 129244539 | 129245747 |
| C9 | 129276378 | 129277458 |
| C9 | 129311307 | 129312521 |
| C9 | 129616658 | 129617751 |
| C9 | 130324738 | 130325931 |
| C9 | 130487170 | 130488675 |
| C9 | 130503710 | 130504767 |
| C9 | 130515908 | 130516969 |
| C9 | 130515992 | 130517085 |
| C9 | 130516666 | 130517801 |
| C9 | 130516842 | 130518033 |
| C9 | 130571533 | 130572847 |
| C9 | 130587396 | 130588589 |
| C9 | 130590131 | 130591603 |
| C9 | 130815342 | 130816655 |
| C9 | 130904951 | 130906550 |
| C9 | 130954384 | 130955636 |
| C9 | 130954936 | 130956036 |
| C9 | 131072550 | 131073702 |
| C9 | 131154921 | 131155997 |
| C9 | 131199607 | 131200822 |
| C9 | 131308846 | 131309877 |
| C9 | 131359679 | 131360820 |
| C9 | 131417001 | 131418417 |
| C9 | 131548955 | 131550034 |
| C9 | 131579554 | 131580756 |
| C9 | 131642649 | 131643836 |
| C9 | 131709398 | 131710495 |
| C9 | 131830796 | 131832559 |
| C9 | 131832074 | 131833181 |
| C9 | 131960416 | 131961506 |
| C9 | 131961246 | 131962297 |
| C9 | 132108527 | 132109781 |
| C9 | 132173780 | 132174855 |
| C9 | 132198912 | 132200035 |
| C9 | 132256894 | 132258353 |
| C9 | 132387203 | 132388474 |
| C9 | 132564181 | 132565324 |
| C9 | 132624872 | 132626176 |
| C9 | 132628352 | 132629491 |
| C9 | 132650264 | 132651421 |
| C9 | 132652040 | 132653194 |
| C9 | 132799929 | 132801133 |
| C9 | 133252615 | 133253832 |
| C9 | 133295239 | 133296262 |
| C9 | 133757283 | 133758287 |
| C9 | 133911114 | 133912254 |
| C9 | 133942638 | 133943682 |
| C9 | 133999241 | 134000420 |
| C9 | 134127075 | 134128184 |
| C9 | 134127602 | 134129062 |
| C9 | 134391128 | 134392281 |
| C9 | 134473072 | 134474327 |
| C9 | 134763168 | 134764221 |
| C9 | 134954538 | 134955664 |
| C9 | 135073238 | 135074345 |
| C9 | 135075780 | 135077319 |
| C9 | 135083819 | 135085158 |
| C9 | 135307481 | 135308726 |
| C9 | 135401265 | 135402414 |
| C9 | 135472027 | 135473188 |
| C9 | 135764754 | 135765762 |
| C9 | 135946318 | 135947507 |
| C9 | 135959431 | 135960526 |
| C9 | 135959557 | 135960790 |
| C9 | 135994728 | 135995876 |
| C9 | 136060264 | 136061529 |
| C9 | 136097470 | 136098508 |
| C9 | 136103717 | 136104911 |
| C9 | 136149479 | 136150624 |
| C9 | 136181112 | 136182304 |
| C9 | 136281723 | 136282777 |
| C9 | 136370974 | 136372498 |
| C9 | 136395577 | 136396595 |
| C9 | 136413646 | 136414948 |
| C9 | 136426865 | 136428106 |
| C9 | 136427225 | 136428274 |
| C9 | 136427300 | 136428419 |
| C9 | 136465006 | 136466015 |
| C9 | 136507483 | 136508853 |
| C9 | 136566158 | 136567283 |
| C9 | 136708700 | 136709942 |
| C9 | 136782826 | 136784015 |
| C9 | 136844090 | 136845229 |
| C9 | 136869741 | 136870781 |
| C9 | 136877502 | 136878623 |
| C9 | 136878278 | 136879342 |
| C9 | 136983221 | 136984348 |
| C9 | 137030033 | 137031161 |
| C9 | 137110086 | 137111330 |
| C9 | 137224854 | 137226137 |
| C9 | 137251890 | 137252973 |
| C9 | 137330230 | 137331297 |
| C9 | 137331500 | 137332938 |
| C9 | 137356009 | 137357648 |
| C9 | 137357877 | 137358992 |
| C9 | 137429230 | 137430493 |
| C9 | 137457665 | 137458808 |
| C9 | 137519550 | 137520641 |
| C9 | 137535543 | 137536833 |
| C9 | 137646035 | 137647061 |
| C9 | 137675215 | 137676371 |
| C9 | 137684150 | 137685390 |
| C9 | 137714180 | 137715430 |
| C9 | 137730685 | 137731925 |
| C9 | 137746791 | 137748242 |
| C9 | 137806636 | 137807680 |
| C9 | 138145107 | 138146186 |
| C9 | 138149828 | 138151012 |
| C9 | 138177796 | 138178823 |
| C9 | 138230735 | 138231763 |
| C9 | 138288964 | 138290084 |
| C9 | 138332231 | 138333273 |
| C9 | 138332807 | 138334188 |
| C9 | 138333579 | 138334736 |
| C9 | 138347264 | 138348349 |
| C9 | 138361790 | 138363020 |
| C9 | 138552891 | 138554137 |
| C9 | 138642352 | 138643420 |
| C9 | 138677582 | 138678684 |
| C9 | 138677789 | 138678899 |
| C9 | 138679531 | 138680673 |
| C9 | 138683316 | 138684492 |
| C9 | 138873224 | 138874369 |
| C9 | 138881298 | 138882416 |
| C9 | 138898671 | 138899915 |
| C9 | 138936756 | 138938353 |
| C9 | 138942117 | 138943256 |
| C9 | 138943780 | 138945231 |
| C9 | 138947083 | 138948257 |
| C9 | 138974921 | 138976164 |
| C9 | 139084852 | 139085883 |
| C9 | 139143506 | 139144798 |
| C9 | 139304531 | 139305641 |
| C9 | 139365944 | 139367039 |
| C9 | 139366820 | 139367905 |
| C9 | 139404680 | 139405791 |
| C9 | 139406081 | 139407137 |
| C9 | 139425648 | 139426734 |
| C9 | 139504640 | 139506029 |
| C9 | 139508252 | 139509449 |
| C9 | 139513520 | 139515179 |
| C9 | 139515163 | 139516308 |
| C9 | 139520509 | 139521602 |
| C9 | 139553226 | 139554415 |
| C9 | 139556750 | 139557898 |
| C9 | 139564548 | 139565708 |
| C9 | 139581314 | 139582373 |
| C9 | 139584821 | 139585999 |
| C9 | 139633035 | 139634271 |
| C9 | 139664223 | 139665285 |
| C9 | 139664315 | 139665441 |
| C9 | 139680549 | 139681593 |
| C9 | 139715363 | 139716435 |
| C9 | 139715594 | 139716666 |
| C9 | 139739638 | 139740706 |
| C9 | 139760766 | 139761877 |
| C9 | 139834000 | 139835053 |
| C9 | 139837342 | 139838519 |
| C9 | 139879495 | 139880566 |
| C9 | 139888070 | 139889550 |
| C9 | 139888789 | 139889882 |
| C9 | 139888935 | 139889982 |
| C9 | 139889438 | 139890509 |
| C9 | 139907191 | 139908274 |
| C9 | 140040958 | 140042200 |
| C9 | 140067854 | 140068995 |
| C9 | 140068812 | 140069887 |
| C9 | 140069245 | 140070495 |
| C9 | 140171995 | 140173198 |
| C9 | 140173811 | 140174875 |
| C9 | 140174807 | 140176196 |
| C9 | 140182509 | 140183971 |
| C9 | 140188846 | 140189909 |
| C9 | 140197196 | 140198391 |
| C9 | 140201009 | 140202173 |
| C9 | 140223104 | 140224186 |
| C9 | 140247867 | 140249065 |
| C9 | 140310798 | 140311915 |
| C9 | 140311042 | 140312536 |
| C9 | 140311660 | 140312805 |
| C9 | 140311865 | 140313025 |
| C9 | 140344796 | 140346176 |
| C9 | 140347661 | 140348790 |
| C9 | 140372951 | 140374183 |
| C9 | 140473202 | 140474323 |
| C9 | 140501957 | 140503095 |
| C9 | 140541370 | 140542871 |
| C9 | 140704248 | 140705264 |
| C9 | 140712473 | 140713577 |
| C9 | 140849633 | 140850799 |
| C9 | 141005790 | 141007101 |
| C9 | 141070172 | 141071494 |
| C9 | 141110896 | 141112014 |
| C10 | 73603 | 75004 |
| C10 | 323149 | 324198 |
| C10 | 369448 | 370478 |
| C10 | 530279 | 531870 |
| C10 | 678671 | 680015 |
| C10 | 680847 | 682144 |
| C10 | 742750 | 743866 |
| C10 | 782507 | 783765 |
| C10 | 823092 | 824289 |
| C10 | 834981 | 836149 |
| C10 | 977388 | 978425 |
| C10 | 1206085 | 1207227 |
| C10 | 1227857 | 1228992 |
| C10 | 1251770 | 1253004 |
| C10 | 1283652 | 1285083 |
| C10 | 1319808 | 1320814 |
| C10 | 1404449 | 1405543 |
| C10 | 1404686 | 1406104 |
| C10 | 1405413 | 1406651 |
| C10 | 1416276 | 1417435 |
| C10 | 1520314 | 1521464 |
| C10 | 1584675 | 1585920 |
| C10 | 1595204 | 1596449 |
| C10 | 1609458 | 1610496 |
| C10 | 1677651 | 1678986 |
| C10 | 1739120 | 1740638 |
| C10 | 1743060 | 1744187 |
| C10 | 1879277 | 1880313 |
| C10 | 1956406 | 1957442 |
| C10 | 1994409 | 1995595 |
| C10 | 2006197 | 2007309 |
| C10 | 2061233 | 2062743 |
| C10 | 2073425 | 2074492 |
| C10 | 2136315 | 2138435 |
| C10 | 2141687 | 2143487 |
| C10 | 2156412 | 2157465 |
| C10 | 2358688 | 2359921 |
| C10 | 2543116 | 2544173 |
| C10 | 2543515 | 2544661 |
| C10 | 2564366 | 2565718 |
| C10 | 2600623 | 2601679 |
| C10 | 2619586 | 2621107 |
| C10 | 2671696 | 2673560 |
| C10 | 2693386 | 2695211 |
| C10 | 2698653 | 2699907 |
| C10 | 2733740 | 2736465 |
| C10 | 2814503 | 2815520 |
| C10 | 2848071 | 2849419 |
| C10 | 3148512 | 3150165 |
| C10 | 3150533 | 3151932 |
| C10 | 3329320 | 3330343 |
| C10 | 3329980 | 3331104 |
| C10 | 3524062 | 3525254 |
| C10 | 3598146 | 3599218 |
| C10 | 3677902 | 3679086 |
| C10 | 3694677 | 3695834 |
| C10 | 3814273 | 3815885 |
| C10 | 3828009 | 3829669 |
| C10 | 4375128 | 4376250 |
| C10 | 5061670 | 5062685 |
| C10 | 5144031 | 5145394 |
| C10 | 5344330 | 5345366 |
| C10 | 5412257 | 5413384 |
| C10 | 5521324 | 5522451 |
| C10 | 5540406 | 5541563 |
| C10 | 5540782 | 5542218 |
| C10 | 5625142 | 5626334 |
| C10 | 5657881 | 5659022 |
| C10 | 5734245 | 5735714 |
| C10 | 5928994 | 5930243 |
| C10 | 6019829 | 6020906 |
| C10 | 6041242 | 6042269 |
| C10 | 6177768 | 6178889 |
| C10 | 6182937 | 6183987 |
| C10 | 6186399 | 6187460 |
| C10 | 6223981 | 6225097 |
| C10 | 6247891 | 6249157 |
| C10 | 6416414 | 6417760 |
| C10 | 6527472 | 6528525 |
| C10 | 6556501 | 6557603 |
| C10 | 7213505 | 7214659 |
| C10 | 7255017 | 7256273 |
| C10 | 7310955 | 7312177 |
| C10 | 7317301 | 7318682 |
| C10 | 7367475 | 7369854 |
| C10 | 7409024 | 7410277 |
| C10 | 7465934 | 7467050 |
| C10 | 7567325 | 7568545 |
| C10 | 8075768 | 8076832 |
| C10 | 8084986 | 8086365 |
| C10 | 8093268 | 8094526 |
| C10 | 8093625 | 8094847 |
| C10 | 8120293 | 8121311 |
| C10 | 8178976 | 8180310 |
| C10 | 9314649 | 9315738 |
| C10 | 9314882 | 9316084 |
| C10 | 9448381 | 9450108 |
| C10 | 9897077 | 9898200 |
| C10 | 9998083 | 9999342 |
| C10 | 10307693 | 10309014 |
| C10 | 10692605 | 10693803 |
| C10 | 10708141 | 10709319 |
| C10 | 10732738 | 10735958 |
| C10 | 10952963 | 10954125 |
| C10 | 11183873 | 11185138 |
| C10 | 11219842 | 11221403 |
| C10 | 11343092 | 11344119 |
| C10 | 11362676 | 11364133 |
| C10 | 11412447 | 11413787 |
| C10 | 11428485 | 11429733 |
| C10 | 11492216 | 11493221 |
| C10 | 11722689 | 11723733 |
| C10 | 11733010 | 11734641 |
| C10 | 11778199 | 11779991 |
| C10 | 11834321 | 11835584 |
| C10 | 11911493 | 11912617 |
| C10 | 12027544 | 12028679 |
| C10 | 12095204 | 12096431 |
| C10 | 12170412 | 12171563 |
| C10 | 12235438 | 12236620 |
| C10 | 12334565 | 12336257 |
| C10 | 12391479 | 12392560 |
| C10 | 12394713 | 12395752 |
| C10 | 12493308 | 12494658 |
| C10 | 12503482 | 12505253 |
| C10 | 12520820 | 12522135 |
| C10 | 12548036 | 12549410 |
| C10 | 12548733 | 12550282 |
| C10 | 12564325 | 12565509 |
| C10 | 12573968 | 12575073 |
| C10 | 12594736 | 12596189 |
| C10 | 12614168 | 12615192 |
| C10 | 12619898 | 12621423 |
| C10 | 12723635 | 12725523 |
| C10 | 12776693 | 12777803 |
| C10 | 12827198 | 12828462 |
| C10 | 13199969 | 13201152 |
| C10 | 13273638 | 13274672 |
| C10 | 13313357 | 13314659 |
| C10 | 13446998 | 13448186 |
| C10 | 13700825 | 13702093 |
| C10 | 13784800 | 13786158 |
| C10 | 14194714 | 14196324 |
| C10 | 14850535 | 14851797 |
| C10 | 15032050 | 15033777 |
| C10 | 15035143 | 15036285 |
| C10 | 15035482 | 15036894 |
| C10 | 15037554 | 15038713 |
| C10 | 15209257 | 15210900 |
| C10 | 15254627 | 15255984 |
| C10 | 15255275 | 15256461 |
| C10 | 15276252 | 15277450 |
| C10 | 15470726 | 15471849 |
| C10 | 15576583 | 15577764 |
| C10 | 16026496 | 16027768 |
| C10 | 16918423 | 16919618 |
| C10 | 17042632 | 17043651 |
| C10 | 17266118 | 17267388 |
| C10 | 17552939 | 17554015 |
| C10 | 17669996 | 17671468 |
| C10 | 18094873 | 18096624 |
| C10 | 18103137 | 18104431 |
| C10 | 18119484 | 18120757 |
| C10 | 18121422 | 18123778 |
| C10 | 18208780 | 18210460 |
| C10 | 18217177 | 18219226 |
| C10 | 18223425 | 18224556 |
| C10 | 18742413 | 18743463 |
| C10 | 19396859 | 19397945 |
| C10 | 19777369 | 19778491 |
| C10 | 19793127 | 19794237 |
| C10 | 19871559 | 19873490 |
| C10 | 20060910 | 20062019 |
| C10 | 20414761 | 20416151 |
| C10 | 20632523 | 20633707 |
| C10 | 20979111 | 20980633 |
| C10 | 21718834 | 21720022 |
| C10 | 21788549 | 21789624 |
| C10 | 21788815 | 21790028 |
| C10 | 21798137 | 21799775 |
| C10 | 21798876 | 21799946 |
| C10 | 22170491 | 22171508 |
| C10 | 22523040 | 22524079 |
| C10 | 22542082 | 22543238 |
| C10 | 22566345 | 22567895 |
| C10 | 22623205 | 22624410 |
| C10 | 22634295 | 22635441 |
| C10 | 22765081 | 22766287 |
| C10 | 22765563 | 22766709 |
| C10 | 22766122 | 22767296 |
| C10 | 22843041 | 22844284 |
| C10 | 23983434 | 23984629 |
| C10 | 23984252 | 23985388 |
| C10 | 24831149 | 24833041 |
| C10 | 24945876 | 24947081 |
| C10 | 25034695 | 25035756 |
| C10 | 25754476 | 25756115 |
| C10 | 25809456 | 25810588 |
| C10 | 26051336 | 26052411 |
| C10 | 26855271 | 26856968 |
| C10 | 26986254 | 26987312 |
| C10 | 27182192 | 27183508 |
| C10 | 27547772 | 27548970 |
| C10 | 27584485 | 27585614 |
| C10 | 27584816 | 27585965 |
| C10 | 27608119 | 27609255 |
| C10 | 27608284 | 27609392 |
| C10 | 27702127 | 27703618 |
| C10 | 27895264 | 27896276 |
| C10 | 28029917 | 28031019 |
| C10 | 28112429 | 28113555 |
| C10 | 28155716 | 28156745 |
| C10 | 28790706 | 28792442 |
| C10 | 29096357 | 29097780 |
| C10 | 29431304 | 29432340 |
| C10 | 29533585 | 29535288 |
| C10 | 29970587 | 29971935 |
| C10 | 30343243 | 30344387 |
| C10 | 30497187 | 30498587 |
| C10 | 30675687 | 30676782 |
| C10 | 30724020 | 30725143 |
| C10 | 30931323 | 30932562 |
| C10 | 31039731 | 31040790 |
| C10 | 31371498 | 31372543 |
| C10 | 31445968 | 31447658 |
| C10 | 31757913 | 31759074 |
| C10 | 31986203 | 31987247 |
| C10 | 32156556 | 32157583 |
| C10 | 33294229 | 33295266 |
| C10 | 34251089 | 34252133 |
| C10 | 35034181 | 35035247 |
| C10 | 35624949 | 35625991 |
| C10 | 35927811 | 35928878 |
| C10 | 36564988 | 36566015 |
| C10 | 36684640 | 36685757 |
| C10 | 36812778 | 36813852 |
| C10 | 36899008 | 36900141 |
| C10 | 37524228 | 37525490 |
| C10 | 38644455 | 38645704 |
| C10 | 38691021 | 38692220 |
| C10 | 38691524 | 38692560 |
| C10 | 42540662 | 42542280 |
| C10 | 42607056 | 42608190 |
| C10 | 42644951 | 42646031 |
| C10 | 42645286 | 42646425 |
| C10 | 42672658 | 42673927 |
| C10 | 42737017 | 42738121 |
| C10 | 42737340 | 42738419 |
| C10 | 42737493 | 42738569 |
| C10 | 42738015 | 42739078 |
| C10 | 42738161 | 42739270 |
| C10 | 42738472 | 42739522 |
| C10 | 43277350 | 43278588 |
| C10 | 43446276 | 43447438 |
| C10 | 43446840 | 43447964 |
| C10 | 43698334 | 43699432 |
| C10 | 43832290 | 43833344 |
| C10 | 43845832 | 43846855 |
| C10 | 43890839 | 43891945 |
| C10 | 43891047 | 43892198 |
| C10 | 43930810 | 43931945 |
| C10 | 43995490 | 43996527 |
| C10 | 44197272 | 44198475 |
| C10 | 45157655 | 45159006 |
| C10 | 45825443 | 45826750 |
| C10 | 45922583 | 45924055 |
| C10 | 46095657 | 46096906 |
| C10 | 46874866 | 46875996 |
| C10 | 47087995 | 47089125 |
| C10 | 47254943 | 47256099 |
| C10 | 47271140 | 47272414 |
| C10 | 47424276 | 47425895 |
| C10 | 48827390 | 48828626 |
| C10 | 48964033 | 48965200 |
| C10 | 48998113 | 48999355 |
| C10 | 49199134 | 49200434 |
| C10 | 49347935 | 49349070 |
| C10 | 49667335 | 49668419 |
| C10 | 49673956 | 49675083 |
| C10 | 49674125 | 49675183 |
| C10 | 49674221 | 49675374 |
| C10 | 50155937 | 50156964 |
| C10 | 50243497 | 50245059 |
| C10 | 50284891 | 50285928 |
| C10 | 50649242 | 50650339 |
| C10 | 50954261 | 50955397 |
| C10 | 51784144 | 51785469 |
| C10 | 53827319 | 53828458 |
| C10 | 53992725 | 53994007 |
| C10 | 53994426 | 53996847 |
| C10 | 54860415 | 54861633 |
| C10 | 55928468 | 55929591 |
| C10 | 57023184 | 57024289 |
| C10 | 57701635 | 57702757 |
| C10 | 60352061 | 60353397 |
| C10 | 63626912 | 63628329 |
| C10 | 64683670 | 64684883 |
| C10 | 65710347 | 65711364 |
| C10 | 65801059 | 65802120 |
| C10 | 66812865 | 66814171 |
| C10 | 68304518 | 68305528 |
| C10 | 70461796 | 70462927 |
| C10 | 70852871 | 70853980 |
| C10 | 70856577 | 70857904 |
| C10 | 71077554 | 71078621 |
| C10 | 71214195 | 71215312 |
| C10 | 72015057 | 72016196 |
| C10 | 72254329 | 72255465 |
| C10 | 72359951 | 72361104 |
| C10 | 72446465 | 72447630 |
| C10 | 72677814 | 72679056 |
| C10 | 72726046 | 72727126 |
| C10 | 73398371 | 73399434 |
| C10 | 73628331 | 73629528 |
| C10 | 76874864 | 76876025 |
| C10 | 77351772 | 77353302 |
| C10 | 77924509 | 77925518 |
| C10 | 78800453 | 78801857 |
| C10 | 79054810 | 79055956 |
| C10 | 79231982 | 79233065 |
| C10 | 79595029 | 79596123 |
| C10 | 80236450 | 80237526 |
| C10 | 81157218 | 81158535 |
| C10 | 81318754 | 81319780 |
| C10 | 81466062 | 81467389 |
| C10 | 81598081 | 81599202 |
| C10 | 81966337 | 81967432 |
| C10 | 81966868 | 81967944 |
| C10 | 82293670 | 82294784 |
| C10 | 82295107 | 82296658 |
| C10 | 83294923 | 83295936 |
| C10 | 83722534 | 83723632 |
| C10 | 84810982 | 84812117 |
| C10 | 85625795 | 85626943 |
| C10 | 87957933 | 87958991 |
| C10 | 88022439 | 88023685 |
| C10 | 88142953 | 88143980 |
| C10 | 88281289 | 88282388 |
| C10 | 88515797 | 88516858 |
| C10 | 88840551 | 88842068 |
| C10 | 89166915 | 89168510 |
| C10 | 91010361 | 91011407 |
| C10 | 93786329 | 93787409 |
| C10 | 94135023 | 94136112 |
| C10 | 94135378 | 94136595 |
| C10 | 94179957 | 94181090 |
| C10 | 94433619 | 94434754 |
| C10 | 94433985 | 94435067 |
| C10 | 94449162 | 94450220 |
| C10 | 94449905 | 94451191 |
| C10 | 94455289 | 94456381 |
| C10 | 94607890 | 94608979 |
| C10 | 95379955 | 95380961 |
| C10 | 95691601 | 95692652 |
| C10 | 96163762 | 96164796 |
| C10 | 96800900 | 96802008 |
| C10 | 96989825 | 96991501 |
| C10 | 97103952 | 97104997 |
| C10 | 98207240 | 98208553 |
| C10 | 98413878 | 98414924 |
| C10 | 99072117 | 99073728 |
| C10 | 99167565 | 99168730 |
| C10 | 99209214 | 99210319 |
| C10 | 99320251 | 99321558 |
| C10 | 99525584 | 99526602 |
| C10 | 99531362 | 99532515 |
| C10 | 99695478 | 99696562 |
| C10 | 99803375 | 99804724 |
| C10 | 100845589 | 100846755 |
| C10 | 101245986 | 101247067 |
| C10 | 101280722 | 101282042 |
| C10 | 101281256 | 101282812 |
| C10 | 101286850 | 101287986 |
| C10 | 101287056 | 101288173 |
| C10 | 102288937 | 102290091 |
| C10 | 102455435 | 102456441 |
| C10 | 102507016 | 102508109 |
| C10 | 102770683 | 102771992 |
| C10 | 102995549 | 102996772 |
| C10 | 103469275 | 103470404 |
| C10 | 103529704 | 103530762 |
| C10 | 104192327 | 104193474 |
| C10 | 104402641 | 104403864 |
| C10 | 104648971 | 104650273 |
| C10 | 105343993 | 105345174 |
| C10 | 105420499 | 105421714 |
| C10 | 105427801 | 105429152 |
| C10 | 105725523 | 105726623 |
| C10 | 105881138 | 105882202 |
| C10 | 106034471 | 106035711 |
| C10 | 106065314 | 106066335 |
| C10 | 106073818 | 106074979 |
| C10 | 106248206 | 106249311 |
| C10 | 107250224 | 107251280 |
| C10 | 110075212 | 110076316 |
| C10 | 111292938 | 111294458 |
| C10 | 111766341 | 111767435 |
| C10 | 111965261 | 111966396 |
| C10 | 112097169 | 112098257 |
| C10 | 112388058 | 112389070 |
| C10 | 114073376 | 114074675 |
| C10 | 115614360 | 115615419 |
| C10 | 115804202 | 115805243 |
| C10 | 116306886 | 116308028 |
| C10 | 118989958 | 118991016 |
| C10 | 119294514 | 119295566 |
| C10 | 119304647 | 119305779 |
| C10 | 119806390 | 119807617 |
| C10 | 120353392 | 120354521 |
| C10 | 120786125 | 120787131 |
| C10 | 120834826 | 120835928 |
| C10 | 120940743 | 120941929 |
| C10 | 120966719 | 120967795 |
| C10 | 121020315 | 121021369 |
| C10 | 121252315 | 121253442 |
| C10 | 121770446 | 121771634 |
| C10 | 121770763 | 121771896 |
| C10 | 121822416 | 121823422 |
| C10 | 122216569 | 122217644 |
| C10 | 123054376 | 123055429 |
| C10 | 123339446 | 123340773 |
| C10 | 123354520 | 123356083 |
| C10 | 124546481 | 124547596 |
| C10 | 124577737 | 124578784 |
| C10 | 124804242 | 124805443 |
| C10 | 125729515 | 125730768 |
| C10 | 126223017 | 126224284 |
| C10 | 126359076 | 126360221 |
| C10 | 126388368 | 126389606 |
| C10 | 126529537 | 126530561 |
| C10 | 126682579 | 126683667 |
| C10 | 126715138 | 126716193 |
| C10 | 126850694 | 126851826 |
| C10 | 126916039 | 126917496 |
| C10 | 127461924 | 127463167 |
| C10 | 127462250 | 127463329 |
| C10 | 127574164 | 127576201 |
| C10 | 127577060 | 127578679 |
| C10 | 127583765 | 127584887 |
| C10 | 127584206 | 127585344 |
| C10 | 127584537 | 127586030 |
| C10 | 127586187 | 127587754 |
| C10 | 127589848 | 127591083 |
| C10 | 127590738 | 127592708 |
| C10 | 127592960 | 127593997 |
| C10 | 127606294 | 127607556 |
| C10 | 128164710 | 128165779 |
| C10 | 128594415 | 128595519 |
| C10 | 128917924 | 128918984 |
| C10 | 129536510 | 129537577 |
| C10 | 129948504 | 129949543 |
| C10 | 130362353 | 130363387 |
| C10 | 131186196 | 131187210 |
| C10 | 131668899 | 131669963 |
| C10 | 131717725 | 131718880 |
| C10 | 131986879 | 131988033 |
| C10 | 132001558 | 132002677 |
| C10 | 132098605 | 132100596 |
| C10 | 132402643 | 132403709 |
| C10 | 132557381 | 132558723 |
| C10 | 132558063 | 132559647 |
| C10 | 132583325 | 132584436 |
| C10 | 132818218 | 132819584 |
| C10 | 132892929 | 132894025 |
| C10 | 133120462 | 133121663 |
| C10 | 133792909 | 133794032 |
| C10 | 133877253 | 133878452 |
| C10 | 133937286 | 133938393 |
| C10 | 133993562 | 133994631 |
| C10 | 133993742 | 133994791 |
| C10 | 134044989 | 134046567 |
| C10 | 134062004 | 134063218 |
| C10 | 134067549 | 134068701 |
| C10 | 134095201 | 134096293 |
| C10 | 134319285 | 134320447 |
| C10 | 134440808 | 134441956 |
| C10 | 134612486 | 134613595 |
| C10 | 134699287 | 134700528 |
| C10 | 134726578 | 134727659 |
| C10 | 134727068 | 134728229 |
| C10 | 134728339 | 134729756 |
| C10 | 134737798 | 134738894 |
| C10 | 134745432 | 134746907 |
| C10 | 134751470 | 134752579 |
| C10 | 134756327 | 134757352 |
| C10 | 134777676 | 134779639 |
| C10 | 134790894 | 134792141 |
| C10 | 134795986 | 134797139 |
| C10 | 134796190 | 134797346 |
| C10 | 134817603 | 134819022 |
| C10 | 134830563 | 134831651 |
| C10 | 134834412 | 134835455 |
| C10 | 134835189 | 134836234 |
| C10 | 134840019 | 134841229 |
| C10 | 134849788 | 134850946 |
| C10 | 134854627 | 134855743 |
| C10 | 134876537 | 134877744 |
| C10 | 134877074 | 134878222 |
| C10 | 134937731 | 134938838 |
| C10 | 134937903 | 134938980 |
| C10 | 134940749 | 134941881 |
| C10 | 134953366 | 134954562 |
| C10 | 135001662 | 135002766 |
| C10 | 135014304 | 135015450 |
| C10 | 135014758 | 135015902 |
| C10 | 135016106 | 135017214 |
| C10 | 135033300 | 135034367 |
| C10 | 135033650 | 135034812 |
| C10 | 135035372 | 135037149 |
| C10 | 135037693 | 135038982 |
| C10 | 135051441 | 135052547 |
| C10 | 135054572 | 135055751 |
| C10 | 135104299 | 135105412 |
| C10 | 135143387 | 135144489 |
| C10 | 135237300 | 135238437 |
| C10 | 135237688 | 135239054 |
| C10 | 135259640 | 135260691 |
| C10 | 135293216 | 135294417 |
| C10 | 135341443 | 135342593 |
| C10 | 135349367 | 135350419 |
| C10 | 135356916 | 135357982 |
| C10 | 135381638 | 135382907 |
| C10 | 135382188 | 135383796 |
| C10 | 135436173 | 135437246 |
| C10 | 135453622 | 135454742 |
| C10 | 135478638 | 135479733 |
| C10 | 135481807 | 135482999 |
| C10 | 135490289 | 135491370 |
| C10 | 135490378 | 135491506 |
| C11 | 209228 | 210302 |
| C11 | 267558 | 268835 |
| C11 | 289427 | 290556 |
| C11 | 311186 | 312473 |
| C11 | 314307 | 315455 |
| C11 | 315737 | 316867 |
| C11 | 316025 | 317123 |
| C11 | 355690 | 356763 |
| C11 | 396093 | 398035 |
| C11 | 399117 | 401195 |
| C11 | 400223 | 401297 |
| C11 | 402112 | 403332 |
| C11 | 402347 | 403748 |
| C11 | 406351 | 407426 |
| C11 | 424489 | 425545 |
| C11 | 424570 | 425720 |
| C11 | 502962 | 504058 |
| C11 | 503736 | 504802 |
| C11 | 518065 | 519235 |
| C11 | 518320 | 519360 |
| C11 | 611302 | 612353 |
| C11 | 613072 | 614341 |
| C11 | 614380 | 615454 |
| C11 | 654966 | 656057 |
| C11 | 691335 | 692467 |
| C11 | 695373 | 696470 |
| C11 | 725213 | 726501 |
| C11 | 782361 | 783587 |
| C11 | 804599 | 805675 |
| C11 | 809494 | 810581 |
| C11 | 850194 | 851374 |
| C11 | 858850 | 859959 |
| C11 | 859170 | 860222 |
| C11 | 918784 | 919961 |
| C11 | 924969 | 926040 |
| C11 | 960845 | 962539 |
| C11 | 967558 | 968702 |
| C11 | 969820 | 970990 |
| C11 | 970124 | 971210 |
| C11 | 976463 | 977537 |
| C11 | 1036223 | 1037289 |
| C11 | 1077943 | 1079093 |
| C11 | 1102008 | 1103112 |
| C11 | 1251647 | 1252731 |
| C11 | 1315869 | 1316993 |
| C11 | 1331084 | 1332294 |
| C11 | 1451213 | 1452413 |
| C11 | 1474331 | 1475549 |
| C11 | 1477279 | 1478536 |
| C11 | 1510131 | 1511186 |
| C11 | 1767275 | 1768348 |
| C11 | 1768640 | 1770010 |
| C11 | 1777316 | 1778584 |
| C11 | 1802677 | 1804065 |
| C11 | 1884588 | 1885987 |
| C11 | 1902704 | 1903825 |
| C11 | 1911649 | 1913031 |
| C11 | 1926076 | 1927255 |
| C11 | 1948433 | 1949915 |
| C11 | 2017251 | 2018360 |
| C11 | 2019155 | 2020233 |
| C11 | 2020648 | 2022515 |
| C11 | 2023249 | 2024499 |
| C11 | 2143091 | 2144184 |
| C11 | 2157367 | 2158607 |
| C11 | 2211673 | 2212761 |
| C11 | 2373166 | 2374170 |
| C11 | 2405019 | 2406359 |
| C11 | 2435964 | 2437089 |
| C11 | 2720035 | 2721189 |
| C11 | 2720279 | 2721493 |
| C11 | 2720505 | 2721852 |
| C11 | 2721538 | 2722620 |
| C11 | 2828278 | 2829383 |
| C11 | 2904955 | 2905997 |
| C11 | 2911503 | 2912616 |
| C11 | 2930051 | 2931171 |
| C11 | 2930299 | 2931456 |
| C11 | 3174507 | 3175546 |
| C11 | 3476418 | 3477642 |
| C11 | 3497414 | 3498609 |
| C11 | 3604918 | 3606597 |
| C11 | 3818244 | 3819322 |
| C11 | 3986461 | 3987630 |
| C11 | 4158073 | 4159171 |
| C11 | 4987136 | 4988170 |
| C11 | 5291986 | 5293411 |
| C11 | 5445496 | 5446661 |
| C11 | 5636723 | 5637883 |
| C11 | 6164681 | 6165823 |
| C11 | 6291493 | 6292991 |
| C11 | 6591745 | 6592865 |
| C11 | 6592001 | 6593162 |
| C11 | 6592173 | 6593246 |
| C11 | 6650951 | 6652108 |
| C11 | 6804441 | 6805490 |
| C11 | 7526485 | 7527513 |
| C11 | 7686558 | 7687825 |
| C11 | 7819460 | 7821193 |
| C11 | 8190115 | 8191166 |
| C11 | 8227369 | 8228731 |
| C11 | 8385208 | 8386268 |
| C11 | 8641699 | 8642728 |
| C11 | 8704981 | 8706121 |
| C11 | 8797182 | 8798280 |
| C11 | 8999068 | 9000278 |
| C11 | 8999975 | 9001111 |
| C11 | 9284761 | 9285875 |
| C11 | 9595115 | 9596177 |
| C11 | 10463537 | 10464693 |
| C11 | 10463803 | 10464858 |
| C11 | 10647102 | 10648396 |
| C11 | 11203440 | 11204512 |
| C11 | 12156906 | 12157947 |
| C11 | 12693727 | 12694781 |
| C11 | 13689473 | 13690542 |
| C11 | 14112276 | 14113747 |
| C11 | 14911653 | 14912845 |
| C11 | 14926004 | 14927102 |
| C11 | 15962313 | 15963433 |
| C11 | 16928105 | 16929188 |
| C11 | 17481549 | 17482693 |
| C11 | 17496981 | 17498065 |
| C11 | 18210299 | 18211824 |
| C11 | 18476653 | 18478016 |
| C11 | 18656442 | 18657573 |
| C11 | 18878008 | 18879037 |
| C11 | 19282148 | 19283269 |
| C11 | 19328486 | 19330214 |
| C11 | 19984842 | 19986141 |
| C11 | 20364487 | 20366436 |
| C11 | 20569056 | 20570236 |
| C11 | 20569526 | 20570700 |
| C11 | 20617708 | 20619063 |
| C11 | 20884247 | 20885758 |
| C11 | 20973182 | 20974261 |
| C11 | 21695689 | 21696978 |
| C11 | 21878991 | 21880007 |
| C11 | 22404464 | 22405665 |
| C11 | 22548773 | 22550308 |
| C11 | 23324747 | 23326008 |
| C11 | 23674194 | 23675730 |
| C11 | 24958713 | 24959925 |
| C11 | 25071370 | 25072592 |
| C11 | 26472244 | 26473589 |
| C11 | 27307416 | 27308744 |
| C11 | 27668235 | 27669463 |
| C11 | 29026028 | 29027288 |
| C11 | 29290638 | 29291786 |
| C11 | 30075088 | 30076902 |
| C11 | 31036025 | 31037285 |
| C11 | 31151995 | 31153185 |
| C11 | 31820564 | 31822019 |
| C11 | 31825212 | 31826683 |
| C11 | 31916184 | 31917539 |
| C11 | 32354923 | 32356046 |
| C11 | 33173223 | 33174228 |
| C11 | 33264342 | 33265940 |
| C11 | 33452826 | 33454186 |
| C11 | 33480803 | 33481963 |
| C11 | 33537220 | 33538536 |
| C11 | 34517011 | 34518346 |
| C11 | 34713583 | 34714637 |
| C11 | 35191405 | 35192700 |
| C11 | 35574519 | 35575605 |
| C11 | 36012110 | 36014296 |
| C11 | 36308122 | 36309188 |
| C11 | 36397636 | 36398710 |
| C11 | 36537432 | 36538623 |
| C11 | 36561477 | 36562691 |
| C11 | 36578309 | 36579481 |
| C11 | 36960187 | 36961378 |
| C11 | 37961711 | 37962822 |
| C11 | 39854004 | 39855405 |
| C11 | 40276129 | 40277147 |
| C11 | 42632736 | 42633829 |
| C11 | 43372816 | 43373987 |
| C11 | 44613372 | 44614530 |
| C11 | 44978315 | 44979387 |
| C11 | 45671400 | 45672641 |
| C11 | 45943177 | 45944207 |
| C11 | 46368670 | 46369797 |
| C11 | 46368807 | 46369855 |
| C11 | 47118741 | 47119879 |
| C11 | 47282315 | 47283499 |
| C11 | 47376057 | 47377328 |
| C11 | 47376398 | 47377462 |
| C11 | 47380882 | 47382062 |
| C11 | 47415503 | 47416621 |
| C11 | 47574217 | 47575287 |
| C11 | 47611220 | 47612344 |
| C11 | 47914174 | 47915225 |
| C11 | 48921255 | 48922321 |
| C11 | 49069927 | 49071071 |
| C11 | 49071220 | 49072404 |
| C11 | 49211513 | 49213307 |
| C11 | 49873225 | 49874313 |
| C11 | 49914764 | 49915859 |
| C11 | 54715599 | 54717897 |
| C11 | 54786924 | 54789056 |
| C11 | 57143581 | 57144646 |
| C11 | 57424692 | 57425762 |
| C11 | 58067464 | 58068608 |
| C11 | 58597701 | 58598862 |
| C11 | 59241751 | 59242950 |
| C11 | 59788675 | 59789693 |
| C11 | 60251905 | 60253007 |
| C11 | 60254703 | 60255751 |
| C11 | 60300425 | 60302030 |
| C11 | 60414167 | 60415376 |
| C11 | 60619514 | 60620758 |
| C11 | 60718723 | 60720030 |
| C11 | 61048720 | 61049824 |
| C11 | 61158718 | 61159884 |
| C11 | 61283556 | 61284726 |
| C11 | 61544316 | 61545368 |
| C11 | 61636997 | 61638100 |
| C11 | 61671333 | 61672759 |
| C11 | 61736186 | 61737457 |
| C11 | 61879561 | 61880642 |
| C11 | 62137969 | 62139623 |
| C11 | 62349837 | 62350883 |
| C11 | 62369570 | 62370911 |
| C11 | 62420933 | 62422067 |
| C11 | 62445747 | 62446841 |
| C11 | 62483975 | 62485302 |
| C11 | 63529758 | 63530933 |
| C11 | 63531003 | 63532143 |
| C11 | 63938138 | 63939648 |
| C11 | 63974272 | 63975568 |
| C11 | 63998217 | 63999471 |
| C11 | 64107109 | 64108524 |
| C11 | 64108911 | 64110121 |
| C11 | 64206041 | 64207237 |
| C11 | 64227100 | 64228140 |
| C11 | 64374815 | 64375906 |
| C11 | 64468809 | 64469963 |
| C11 | 64578107 | 64579230 |
| C11 | 64626172 | 64627441 |
| C11 | 64637659 | 64639009 |
| C11 | 64641152 | 64642463 |
| C11 | 64903978 | 64905076 |
| C11 | 65006470 | 65007590 |
| C11 | 65178118 | 65179125 |
| C11 | 65246001 | 65247074 |
| C11 | 65275815 | 65277026 |
| C11 | 65306694 | 65307744 |
| C11 | 65313435 | 65314534 |
| C11 | 65318170 | 65319267 |
| C11 | 65320672 | 65321828 |
| C11 | 65320912 | 65321990 |
| C11 | 65352366 | 65353434 |
| C11 | 65358801 | 65360114 |
| C11 | 65359136 | 65360344 |
| C11 | 65359368 | 65360421 |
| C11 | 65359449 | 65360569 |
| C11 | 65382253 | 65383363 |
| C11 | 65413913 | 65415248 |
| C11 | 65414372 | 65415761 |
| C11 | 65544821 | 65545957 |
| C11 | 65600801 | 65601901 |
| C11 | 65687512 | 65688687 |
| C11 | 66207880 | 66209054 |
| C11 | 66310792 | 66311922 |
| C11 | 66323627 | 66325031 |
| C11 | 66326230 | 66327339 |
| C11 | 66609992 | 66611093 |
| C11 | 66650036 | 66651177 |
| C11 | 66944048 | 66945111 |
| C11 | 67036114 | 67037454 |
| C11 | 67050791 | 67052354 |
| C11 | 67052129 | 67053610 |
| C11 | 67053344 | 67054449 |
| C11 | 67157405 | 67158549 |
| C11 | 67176534 | 67177648 |
| C11 | 67219164 | 67220326 |
| C11 | 67219382 | 67220763 |
| C11 | 67349923 | 67351200 |
| C11 | 67413920 | 67414956 |
| C11 | 67499035 | 67500124 |
| C11 | 67634853 | 67636601 |
| C11 | 67758129 | 67759267 |
| C11 | 67758480 | 67759637 |
| C11 | 67844074 | 67845122 |
| C11 | 67983866 | 67984912 |
| C11 | 68204790 | 68205898 |
| C11 | 68517148 | 68518497 |
| C11 | 68517572 | 68518844 |
| C11 | 68522292 | 68523409 |
| C11 | 68554771 | 68555781 |
| C11 | 68637271 | 68638839 |
| C11 | 68651890 | 68652900 |
| C11 | 68658215 | 68659356 |
| C11 | 68673116 | 68674175 |
| C11 | 68772860 | 68773919 |
| C11 | 68773146 | 68774173 |
| C11 | 68823085 | 68824221 |
| C11 | 68873432 | 68875103 |
| C11 | 68882799 | 68883949 |
| C11 | 68924208 | 68925457 |
| C11 | 69191622 | 69193098 |
| C11 | 69285643 | 69286782 |
| C11 | 69414406 | 69415493 |
| C11 | 69457971 | 69459599 |
| C11 | 69472431 | 69473670 |
| C11 | 69517549 | 69518725 |
| C11 | 69519235 | 69520519 |
| C11 | 69519869 | 69521052 |
| C11 | 69933609 | 69934709 |
| C11 | 70116213 | 70117278 |
| C11 | 70210710 | 70212149 |
| C11 | 70269605 | 70270884 |
| C11 | 70289392 | 70290500 |
| C11 | 70368500 | 70369626 |
| C11 | 70487015 | 70488312 |
| C11 | 70507709 | 70509003 |
| C11 | 70649984 | 70651084 |
| C11 | 71081557 | 71082586 |
| C11 | 71144792 | 71145829 |
| C11 | 71191357 | 71193316 |
| C11 | 71310347 | 71311892 |
| C11 | 71541403 | 71542797 |
| C11 | 71542162 | 71543231 |
| C11 | 71789809 | 71790959 |
| C11 | 71791787 | 71793314 |
| C11 | 71823196 | 71824271 |
| C11 | 71954899 | 71956022 |
| C11 | 72212202 | 72213816 |
| C11 | 72531542 | 72533448 |
| C11 | 72532665 | 72533718 |
| C11 | 72532788 | 72533857 |
| C11 | 72532975 | 72534070 |
| C11 | 73053224 | 73054298 |
| C11 | 73053317 | 73054802 |
| C11 | 73098917 | 73100328 |
| C11 | 73539205 | 73540682 |
| C11 | 73667825 | 73669053 |
| C11 | 74177830 | 74179046 |
| C11 | 75111489 | 75112757 |
| C11 | 75138629 | 75140206 |
| C11 | 75140540 | 75141640 |
| C11 | 75587869 | 75589036 |
| C11 | 75588301 | 75589428 |
| C11 | 75896766 | 75897914 |
| C11 | 76292930 | 76294063 |
| C11 | 76293424 | 76294744 |
| C11 | 76429640 | 76430840 |
| C11 | 76750688 | 76751782 |
| C11 | 76858267 | 76859458 |
| C11 | 76902613 | 76903674 |
| C11 | 77596976 | 77598206 |
| C11 | 77906750 | 77908089 |
| C11 | 78393697 | 78394907 |
| C11 | 78394117 | 78395717 |
| C11 | 79968408 | 79969500 |
| C11 | 81555140 | 81556901 |
| C11 | 82681172 | 82682382 |
| C11 | 83026366 | 83027724 |
| C11 | 85194515 | 85195730 |
| C11 | 85393330 | 85394468 |
| C11 | 85645297 | 85646417 |
| C11 | 85822023 | 85823106 |
| C11 | 85913219 | 85914346 |
| C11 | 86758128 | 86759290 |
| C11 | 89518654 | 89519822 |
| C11 | 89519999 | 89521081 |
| C11 | 89592721 | 89593862 |
| C11 | 89592924 | 89594082 |
| C11 | 89593206 | 89594300 |
| C11 | 89676153 | 89677341 |
| C11 | 89711459 | 89712882 |
| C11 | 89712398 | 89713608 |
| C11 | 89712611 | 89713701 |
| C11 | 89712958 | 89714091 |
| C11 | 90023029 | 90024039 |
| C11 | 90704202 | 90705643 |
| C11 | 90795714 | 90796870 |
| C11 | 92245506 | 92246767 |
| C11 | 92740610 | 92741712 |
| C11 | 93153320 | 93155121 |
| C11 | 93214029 | 93215109 |
| C11 | 93477556 | 93478997 |
| C11 | 93679359 | 93680459 |
| C11 | 93749523 | 93750608 |
| C11 | 94013751 | 94014828 |
| C11 | 94277595 | 94278710 |
| C11 | 94278038 | 94279315 |
| C11 | 94770390 | 94771524 |
| C11 | 94839914 | 94841711 |
| C11 | 95053594 | 95054904 |
| C11 | 96160117 | 96161392 |
| C11 | 96236589 | 96237594 |
| C11 | 96848467 | 96849600 |
| C11 | 99597429 | 99598531 |
| C11 | 101416318 | 101417599 |
| C11 | 101452614 | 101454176 |
| C11 | 101842513 | 101843652 |
| C11 | 102157725 | 102158988 |
| C11 | 102359252 | 102360282 |
| C11 | 102701715 | 102702889 |
| C11 | 104065132 | 104066220 |
| C11 | 107243149 | 107244326 |
| C11 | 107412739 | 107414336 |
| C11 | 107545976 | 107547053 |
| C11 | 107879529 | 107880555 |
| C11 | 108243979 | 108245133 |
| C11 | 110079353 | 110080619 |
| C11 | 111092172 | 111093281 |
| C11 | 111092293 | 111093352 |
| C11 | 111154498 | 111155557 |
| C11 | 111947857 | 111948904 |
| C11 | 111956963 | 111958067 |
| C11 | 111972402 | 111973429 |
| C11 | 112125670 | 112126759 |
| C11 | 113322342 | 113323706 |
| C11 | 114103258 | 114104492 |
| C11 | 114831256 | 114832378 |
| C11 | 115452326 | 115453362 |
| C11 | 116578289 | 116579638 |
| C11 | 117362885 | 117364193 |
| C11 | 117680585 | 117681670 |
| C11 | 118505281 | 118506343 |
| C11 | 118559424 | 118560668 |
| C11 | 118986215 | 118987383 |
| C11 | 119556777 | 119557983 |
| C11 | 122029067 | 122030810 |
| C11 | 122427002 | 122428407 |
| C11 | 122535091 | 122536262 |
| C11 | 123007963 | 123009268 |
| C11 | 123274951 | 123276021 |
| C11 | 124621547 | 124622687 |
| C11 | 124628845 | 124630311 |
| C11 | 124735810 | 124736897 |
| C11 | 124853186 | 124854204 |
| C11 | 125406066 | 125407260 |
| C11 | 125757929 | 125759088 |
| C11 | 126095510 | 126096845 |
| C11 | 126617747 | 126618912 |
| C11 | 127809444 | 127811239 |
| C11 | 127813359 | 127814420 |
| C11 | 128375853 | 128376957 |
| C11 | 128411982 | 128413707 |
| C11 | 128418747 | 128419934 |
| C11 | 128456150 | 128457290 |
| C11 | 128559252 | 128560448 |
| C11 | 128559926 | 128560952 |
| C11 | 128693597 | 128694804 |
| C11 | 129960364 | 129961516 |
| C11 | 129960783 | 129961883 |
| C11 | 130430989 | 130432086 |
| C11 | 131042806 | 131045089 |
| C11 | 131209102 | 131210150 |
| C11 | 131468465 | 131469874 |
| C11 | 131529098 | 131530118 |
| C11 | 131690995 | 131692013 |
| C11 | 132083273 | 132084375 |
| C11 | 132191179 | 132193412 |
| C11 | 132517320 | 132518379 |
| C11 | 132558261 | 132559347 |
| C11 | 132662057 | 132663528 |
| C11 | 132813496 | 132814584 |
| C11 | 132863594 | 132864858 |
| C11 | 132869036 | 132870297 |
| C11 | 132933844 | 132935165 |
| C11 | 132951346 | 132952512 |
| C11 | 132952390 | 132953488 |
| C11 | 133079017 | 133080585 |
| C11 | 133134427 | 133135437 |
| C11 | 133327684 | 133328970 |
| C11 | 133385318 | 133386688 |
| C11 | 133702086 | 133703225 |
| C11 | 133702282 | 133703726 |
| C11 | 133788773 | 133790559 |
| C11 | 133814840 | 133815930 |
| C11 | 133869551 | 133871154 |
| C11 | 134132134 | 134133337 |
| C11 | 134399562 | 134401247 |
| C11 | 134605023 | 134606276 |
| C11 | 134606402 | 134608016 |
| C11 | 134631628 | 134633033 |
| C11 | 134708822 | 134710161 |
| C11 | 134756778 | 134757992 |
| C12 | 93148 | 94675 |
| C12 | 247573 | 248878 |
| C12 | 247970 | 249057 |
| C12 | 283389 | 284556 |
| C12 | 297005 | 298160 |
| C12 | 297274 | 298518 |
| C12 | 312160 | 313339 |
| C12 | 312369 | 313511 |
| C12 | 501206 | 502237 |
| C12 | 717765 | 718807 |
| C12 | 738780 | 740487 |
| C12 | 739517 | 740980 |
| C12 | 864413 | 865627 |
| C12 | 866813 | 868215 |
| C12 | 1024820 | 1026273 |
| C12 | 1223308 | 1224371 |
| C12 | 1771441 | 1772505 |
| C12 | 1771641 | 1772733 |
| C12 | 1921463 | 1922559 |
| C12 | 1926403 | 1927606 |
| C12 | 1953044 | 1954368 |
| C12 | 1973433 | 1974507 |
| C12 | 2030762 | 2031884 |
| C12 | 2045537 | 2046730 |
| C12 | 2214558 | 2215720 |
| C12 | 2294476 | 2295704 |
| C12 | 2338947 | 2340043 |
| C12 | 2753645 | 2754699 |
| C12 | 3123261 | 3124837 |
| C12 | 3194052 | 3195103 |
| C12 | 3259753 | 3261241 |
| C12 | 3468833 | 3469908 |
| C12 | 3506795 | 3507840 |
| C12 | 3613712 | 3614874 |
| C12 | 3883100 | 3884108 |
| C12 | 3947472 | 3948873 |
| C12 | 4016286 | 4017363 |
| C12 | 4140435 | 4141552 |
| C12 | 4670810 | 4671960 |
| C12 | 5020068 | 5021158 |
| C12 | 5332694 | 5333699 |
| C12 | 5601427 | 5602480 |
| C12 | 6165328 | 6166679 |
| C12 | 6268208 | 6269267 |
| C12 | 6341314 | 6342636 |
| C12 | 6385501 | 6386513 |
| C12 | 6387401 | 6388499 |
| C12 | 6664496 | 6665564 |
| C12 | 6664635 | 6666046 |
| C12 | 6729218 | 6730405 |
| C12 | 6760292 | 6761301 |
| C12 | 6824965 | 6826098 |
| C12 | 6876323 | 6877423 |
| C12 | 6882577 | 6883679 |
| C12 | 7060324 | 7063298 |
| C12 | 7065935 | 7066975 |
| C12 | 7067810 | 7069230 |
| C12 | 7072082 | 7073164 |
| C12 | 7096288 | 7097671 |
| C12 | 7096994 | 7098179 |
| C12 | 7592180 | 7593644 |
| C12 | 7780482 | 7781747 |
| C12 | 7780788 | 7781956 |
| C12 | 7791964 | 7793256 |
| C12 | 8068100 | 8069227 |
| C12 | 8185026 | 8186160 |
| C12 | 8335995 | 8337166 |
| C12 | 8583603 | 8585303 |
| C12 | 8614329 | 8615738 |
| C12 | 8693615 | 8694760 |
| C12 | 8777556 | 8778654 |
| C12 | 8778033 | 8779144 |
| C12 | 8795344 | 8796563 |
| C12 | 8834728 | 8835824 |
| C12 | 8940501 | 8941636 |
| C12 | 9045317 | 9046373 |
| C12 | 9371178 | 9372223 |
| C12 | 9392276 | 9393535 |
| C12 | 9445288 | 9446375 |
| C12 | 9465104 | 9466366 |
| C12 | 9554939 | 9556121 |
| C12 | 9556621 | 9557788 |
| C12 | 9558059 | 9559222 |
| C12 | 9602490 | 9603856 |
| C12 | 9627677 | 9630011 |
| C12 | 9679342 | 9680511 |
| C12 | 9722782 | 9724372 |
| C12 | 9891695 | 9892765 |
| C12 | 9901166 | 9902243 |
| C12 | 9975975 | 9977021 |
| C12 | 10094962 | 10096850 |
| C12 | 10441701 | 10442720 |
| C12 | 10563447 | 10564606 |
| C12 | 11699457 | 11701346 |
| C12 | 11708550 | 11709616 |
| C12 | 12423504 | 12424980 |
| C12 | 12566349 | 12567471 |
| C12 | 14413033 | 14414166 |
| C12 | 14861261 | 14862641 |
| C12 | 16275261 | 16276333 |
| C12 | 19927792 | 19929294 |
| C12 | 19935997 | 19937249 |
| C12 | 20703859 | 20705012 |
| C12 | 22656105 | 22657160 |
| C12 | 23682929 | 23684652 |
| C12 | 25058920 | 25059951 |
| C12 | 25104243 | 25105844 |
| C12 | 25357223 | 25363345 |
| C12 | 25367871 | 25368994 |
| C12 | 25378048 | 25379207 |
| C12 | 25379668 | 25380846 |
| C12 | 25386253 | 25388660 |
| C12 | 25397708 | 25398829 |
| C12 | 25403185 | 25404370 |
| C12 | 25641803 | 25642940 |
| C12 | 26510533 | 26511751 |
| C12 | 27433541 | 27434666 |
| C12 | 28026453 | 28027579 |
| C12 | 29279807 | 29280948 |
| C12 | 29328048 | 29329062 |
| C12 | 29719465 | 29720481 |
| C12 | 30322290 | 30323847 |
| C12 | 30948000 | 30949574 |
| C12 | 31270539 | 31271699 |
| C12 | 31270964 | 31272099 |
| C12 | 31271169 | 31272306 |
| C12 | 31271694 | 31273071 |
| C12 | 31272384 | 31273649 |
| C12 | 32291967 | 32293050 |
| C12 | 32452024 | 32453071 |
| C12 | 32543557 | 32544677 |
| C12 | 32546978 | 32548016 |
| C12 | 33149261 | 33150340 |
| C12 | 33298614 | 33300182 |
| C12 | 33812543 | 33813632 |
| C12 | 34551678 | 34552866 |
| C12 | 38549456 | 38551385 |
| C12 | 38550478 | 38552435 |
| C12 | 38552893 | 38554783 |
| C12 | 38556148 | 38557299 |
| C12 | 38556447 | 38557959 |
| C12 | 39274294 | 39275489 |
| C12 | 39531496 | 39532695 |
| C12 | 40106382 | 40107854 |
| C12 | 40424919 | 40425957 |
| C12 | 40498914 | 40499952 |
| C12 | 43004339 | 43005454 |
| C12 | 43965353 | 43966486 |
| C12 | 44015842 | 44018234 |
| C12 | 44199224 | 44200318 |
| C12 | 44199377 | 44200485 |
| C12 | 45213613 | 45214886 |
| C12 | 46004617 | 46005839 |
| C12 | 47700663 | 47701763 |
| C12 | 48266712 | 48267846 |
| C12 | 48335596 | 48336737 |
| C12 | 48335849 | 48336990 |
| C12 | 48606012 | 48607400 |
| C12 | 48688721 | 48689868 |
| C12 | 48723173 | 48724278 |
| C12 | 49412051 | 49413144 |
| C12 | 49626301 | 49627511 |
| C12 | 49659960 | 49661025 |
| C12 | 49782613 | 49783927 |
| C12 | 49942779 | 49943840 |
| C12 | 50235519 | 50236642 |
| C12 | 50443777 | 50444927 |
| C12 | 51157216 | 51158390 |
| C12 | 51847998 | 51849586 |
| C12 | 52214370 | 52215591 |
| C12 | 52214971 | 52216047 |
| C12 | 52408042 | 52409421 |
| C12 | 52473183 | 52474333 |
| C12 | 52643529 | 52644630 |
| C12 | 52643688 | 52644774 |
| C12 | 52651989 | 52653081 |
| C12 | 52684536 | 52685597 |
| C12 | 52684632 | 52685762 |
| C12 | 53109642 | 53110671 |
| C12 | 53396831 | 53398004 |
| C12 | 54070455 | 54071508 |
| C12 | 54075798 | 54076927 |
| C12 | 54161302 | 54162429 |
| C12 | 54329422 | 54330436 |
| C12 | 54345576 | 54346735 |
| C12 | 54354896 | 54356120 |
| C12 | 54453845 | 54454947 |
| C12 | 54483693 | 54484702 |
| C12 | 54596462 | 54597480 |
| C12 | 54611355 | 54612488 |
| C12 | 54719259 | 54720463 |
| C12 | 54762933 | 54764107 |
| C12 | 54763468 | 54764808 |
| C12 | 54772226 | 54773314 |
| C12 | 54772338 | 54773427 |
| C12 | 54784401 | 54785490 |
| C12 | 54807646 | 54808958 |
| C12 | 55733738 | 55734750 |
| C12 | 55808299 | 55809345 |
| C12 | 56473141 | 56474666 |
| C12 | 56476597 | 56477832 |
| C12 | 56477035 | 56478186 |
| C12 | 56478279 | 56479900 |
| C12 | 56479803 | 56480956 |
| C12 | 56480861 | 56482446 |
| C12 | 56481827 | 56482940 |
| C12 | 56482032 | 56483280 |
| C12 | 56486031 | 56487104 |
| C12 | 56486270 | 56487360 |
| C12 | 56486629 | 56487834 |
| C12 | 56487048 | 56488276 |
| C12 | 56487383 | 56488840 |
| C12 | 56488541 | 56489811 |
| C12 | 56488949 | 56490090 |
| C12 | 56489232 | 56490906 |
| C12 | 56490032 | 56491130 |
| C12 | 56490329 | 56491514 |
| C12 | 56491069 | 56492224 |
| C12 | 56491535 | 56493264 |
| C12 | 56492932 | 56494029 |
| C12 | 56493122 | 56494313 |
| C12 | 56493458 | 56494584 |
| C12 | 56494345 | 56495645 |
| C12 | 56494813 | 56497789 |
| C12 | 57077018 | 57078193 |
| C12 | 57081571 | 57082624 |
| C12 | 57182563 | 57183832 |
| C12 | 57618270 | 57619406 |
| C12 | 57736495 | 57737575 |
| C12 | 57807878 | 57808917 |
| C12 | 57868588 | 57869790 |
| C12 | 58020073 | 58021140 |
| C12 | 58119685 | 58121211 |
| C12 | 58120362 | 58121532 |
| C12 | 58129206 | 58130521 |
| C12 | 58130777 | 58131927 |
| C12 | 58130987 | 58132124 |
| C12 | 58131164 | 58132306 |
| C12 | 58131356 | 58132452 |
| C12 | 58131517 | 58132681 |
| C12 | 58132056 | 58133327 |
| C12 | 58148340 | 58149390 |
| C12 | 58150151 | 58151290 |
| C12 | 58165350 | 58166462 |
| C12 | 62403879 | 62405233 |
| C12 | 63543361 | 63544476 |
| C12 | 64172918 | 64174043 |
| C12 | 64215000 | 64216138 |
| C12 | 64237595 | 64238738 |
| C12 | 64540683 | 64541800 |
| C12 | 64845426 | 64846479 |
| C12 | 64936220 | 64937804 |
| C12 | 65151554 | 65152705 |
| C12 | 65514715 | 65515835 |
| C12 | 65903916 | 65905257 |
| C12 | 66135117 | 66136146 |
| C12 | 69009477 | 69010689 |
| C12 | 70200893 | 70201971 |
| C12 | 70406403 | 70407514 |
| C12 | 71857934 | 71858965 |
| C12 | 72232537 | 72233645 |
| C12 | 76566809 | 76567856 |
| C12 | 77416978 | 77418261 |
| C12 | 77458701 | 77459822 |
| C12 | 79886986 | 79887998 |
| C12 | 80104476 | 80105565 |
| C12 | 80793939 | 80795224 |
| C12 | 80906285 | 80908032 |
| C12 | 81101993 | 81103473 |
| C12 | 83123181 | 83124377 |
| C12 | 83418110 | 83419828 |
| C12 | 84338279 | 84339429 |
| C12 | 86605993 | 86607127 |
| C12 | 88399631 | 88400694 |
| C12 | 89281152 | 89282205 |
| C12 | 89284690 | 89285728 |
| C12 | 90102123 | 90103179 |
| C12 | 91963135 | 91964197 |
| C12 | 92799143 | 92800248 |
| C12 | 93141576 | 93142626 |
| C12 | 94034513 | 94035750 |
| C12 | 95804003 | 95805113 |
| C12 | 97553996 | 97555401 |
| C12 | 98909573 | 98910596 |
| C12 | 98911316 | 98912410 |
| C12 | 99452160 | 99453236 |
| C12 | 104135707 | 104136853 |
| C12 | 104350471 | 104351553 |
| C12 | 104424169 | 104425325 |
| C12 | 104460123 | 104461152 |
| C12 | 104696812 | 104697955 |
| C12 | 104697324 | 104698484 |
| C12 | 106023665 | 106026178 |
| C12 | 106721752 | 106722801 |
| C12 | 107349237 | 107350271 |
| C12 | 107974522 | 107975627 |
| C12 | 108297602 | 108298729 |
| C12 | 109048996 | 109050345 |
| C12 | 109548697 | 109549900 |
| C12 | 109591927 | 109593010 |
| C12 | 109948523 | 109949559 |
| C12 | 110059256 | 110060398 |
| C12 | 110250443 | 110251499 |
| C12 | 111180906 | 111181970 |
| C12 | 111304504 | 111305614 |
| C12 | 111797188 | 111798286 |
| C12 | 112272265 | 112273360 |
| C12 | 112657992 | 112659016 |
| C12 | 113071378 | 113072631 |
| C12 | 113131848 | 113133167 |
| C12 | 113515153 | 113516256 |
| C12 | 113839817 | 113840951 |
| C12 | 114028226 | 114029352 |
| C12 | 114203421 | 114204570 |
| C12 | 114231961 | 114233269 |
| C12 | 115103355 | 115104578 |
| C12 | 115111922 | 115113027 |
| C12 | 115173551 | 115174720 |
| C12 | 115199801 | 115201068 |
| C12 | 115918072 | 115919162 |
| C12 | 116176686 | 116177722 |
| C12 | 116561136 | 116562178 |
| C12 | 116723524 | 116724625 |
| C12 | 117482302 | 117483680 |
| C12 | 118498430 | 118499488 |
| C12 | 119350362 | 119351403 |
| C12 | 119716154 | 119717203 |
| C12 | 119749018 | 119750066 |
| C12 | 119901374 | 119902402 |
| C12 | 120031931 | 120032941 |
| C12 | 121060278 | 121061312 |
| C12 | 121147701 | 121148769 |
| C12 | 121462312 | 121463576 |
| C12 | 121647678 | 121648716 |
| C12 | 121729628 | 121731069 |
| C12 | 121740172 | 121741692 |
| C12 | 122018580 | 122019664 |
| C12 | 122071846 | 122072938 |
| C12 | 122250115 | 122251540 |
| C12 | 122289330 | 122290425 |
| C12 | 122290759 | 122291810 |
| C12 | 122355440 | 122356556 |
| C12 | 122608217 | 122609506 |
| C12 | 122611979 | 122613112 |
| C12 | 123009315 | 123010522 |
| C12 | 123009722 | 123011253 |
| C12 | 123192908 | 123194106 |
| C12 | 123310035 | 123311279 |
| C12 | 123752743 | 123753805 |
| C12 | 124042518 | 124043639 |
| C12 | 124283065 | 124284084 |
| C12 | 124326635 | 124327678 |
| C12 | 124380960 | 124382082 |
| C12 | 124774174 | 124775226 |
| C12 | 124853487 | 124854606 |
| C12 | 124940927 | 124942802 |
| C12 | 124950227 | 124951267 |
| C12 | 125030781 | 125031878 |
| C12 | 125101822 | 125102928 |
| C12 | 125130377 | 125131825 |
| C12 | 125277693 | 125278934 |
| C12 | 125412477 | 125413534 |
| C12 | 125420222 | 125421372 |
| C12 | 126134744 | 126136006 |
| C12 | 126138154 | 126139204 |
| C12 | 126168042 | 126169101 |
| C12 | 126286453 | 126287665 |
| C12 | 126290328 | 126291479 |
| C12 | 126345478 | 126346628 |
| C12 | 126468836 | 126469887 |
| C12 | 126904802 | 126905883 |
| C12 | 127199520 | 127200638 |
| C12 | 127210921 | 127211991 |
| C12 | 127650013 | 127651178 |
| C12 | 127762841 | 127764438 |
| C12 | 127764838 | 127766015 |
| C12 | 128500541 | 128501641 |
| C12 | 128508445 | 128509469 |
| C12 | 128557884 | 128558894 |
| C12 | 128612289 | 128613362 |
| C12 | 128751254 | 128752379 |
| C12 | 128751417 | 128752596 |
| C12 | 128848980 | 128850056 |
| C12 | 128969877 | 128971217 |
| C12 | 129143899 | 129145187 |
| C12 | 129281557 | 129282742 |
| C12 | 129487464 | 129488648 |
| C12 | 129520237 | 129521251 |
| C12 | 129840423 | 129841520 |
| C12 | 130060007 | 130061341 |
| C12 | 130339157 | 130340395 |
| C12 | 130340936 | 130342251 |
| C12 | 130627264 | 130628305 |
| C12 | 130740687 | 130741843 |
| C12 | 130821786 | 130823042 |
| C12 | 130823240 | 130824342 |
| C12 | 130824283 | 130825516 |
| C12 | 130893448 | 130894591 |
| C12 | 131170876 | 131172001 |
| C12 | 131516958 | 131518112 |
| C12 | 131562241 | 131563306 |
| C12 | 131626960 | 131628283 |
| C12 | 131647367 | 131648527 |
| C12 | 131678775 | 131680484 |
| C12 | 131739983 | 131741113 |
| C12 | 131917851 | 131919264 |
| C12 | 131959013 | 131960133 |
| C12 | 132040728 | 132041790 |
| C12 | 132125991 | 132127058 |
| C12 | 132293075 | 132294213 |
| C12 | 132316323 | 132317567 |
| C12 | 132335389 | 132336619 |
| C12 | 132361508 | 132362524 |
| C12 | 132512777 | 132513815 |
| C12 | 132618745 | 132619887 |
| C12 | 132633961 | 132635058 |
| C12 | 132639195 | 132640243 |
| C12 | 132653084 | 132654292 |
| C12 | 132676207 | 132677345 |
| C12 | 132819870 | 132820945 |
| C12 | 132848112 | 132849231 |
| C12 | 132863117 | 132864258 |
| C12 | 132879628 | 132880688 |
| C12 | 132879756 | 132880960 |
| C12 | 132881336 | 132882500 |
| C12 | 132892074 | 132893230 |
| C12 | 132898027 | 132899109 |
| C12 | 132900348 | 132901580 |
| C12 | 132952085 | 132953255 |
| C12 | 132968898 | 132970092 |
| C12 | 132970161 | 132971302 |
| C12 | 132973027 | 132974318 |
| C12 | 132973897 | 132975092 |
| C12 | 133022150 | 133023288 |
| C12 | 133033425 | 133034547 |
| C12 | 133048863 | 133050055 |
| C12 | 133049338 | 133050493 |
| C12 | 133050253 | 133051326 |
| C12 | 133051529 | 133052679 |
| C12 | 133055246 | 133056308 |
| C12 | 133082927 | 133084211 |
| C12 | 133134390 | 133135533 |
| C12 | 133159497 | 133160518 |
| C12 | 133186324 | 133187569 |
| C12 | 133240790 | 133242132 |
| C12 | 133264429 | 133265600 |
| C12 | 133264780 | 133265906 |
| C12 | 133265302 | 133266378 |
| C12 | 133340313 | 133341466 |
| C12 | 133412455 | 133413600 |
| C12 | 133484632 | 133485886 |
| C12 | 133757577 | 133758649 |
| C12 | 133812049 | 133813145 |
| C13 | 19067538 | 19070021 |
| C13 | 19182833 | 19184002 |
| C13 | 19202713 | 19203817 |
| C13 | 19448838 | 19450046 |
| C13 | 19683156 | 19684203 |
| C13 | 19711768 | 19712937 |
| C13 | 20352583 | 20354181 |
| C13 | 20353261 | 20354408 |
| C13 | 20505509 | 20506545 |
| C13 | 20694520 | 20695602 |
| C13 | 20750776 | 20751991 |
| C13 | 20751000 | 20752053 |
| C13 | 20751113 | 20752246 |
| C13 | 20968255 | 20969585 |
| C13 | 21519312 | 21520456 |
| C13 | 21527472 | 21528499 |
| C13 | 21759500 | 21760979 |
| C13 | 21760027 | 21761139 |
| C13 | 21780523 | 21781611 |
| C13 | 21892716 | 21893981 |
| C13 | 22022217 | 22023458 |
| C13 | 22211187 | 22212241 |
| C13 | 23054924 | 23055993 |
| C13 | 23085380 | 23086519 |
| C13 | 23270233 | 23271360 |
| C13 | 23411885 | 23412947 |
| C13 | 23500490 | 23501967 |
| C13 | 23535045 | 23536379 |
| C13 | 23874266 | 23875282 |
| C13 | 24104597 | 24105635 |
| C13 | 24519429 | 24520934 |
| C13 | 24881623 | 24882794 |
| C13 | 24881926 | 24883030 |
| C13 | 24914070 | 24915231 |
| C13 | 25544106 | 25545578 |
| C13 | 25562805 | 25563913 |
| C13 | 25644698 | 25645811 |
| C13 | 25669563 | 25670865 |
| C13 | 26086521 | 26087641 |
| C13 | 26509907 | 26510932 |
| C13 | 26760773 | 26761994 |
| C13 | 27048298 | 27050063 |
| C13 | 27131422 | 27132624 |
| C13 | 27295354 | 27296511 |
| C13 | 27379899 | 27381004 |
| C13 | 27925928 | 27926962 |
| C13 | 28498255 | 28499333 |
| C13 | 28555113 | 28556834 |
| C13 | 29106100 | 29107134 |
| C13 | 29106147 | 29107258 |
| C13 | 29106648 | 29107872 |
| C13 | 29195784 | 29197212 |
| C13 | 29909302 | 29910785 |
| C13 | 30610685 | 30611689 |
| C13 | 30843632 | 30844763 |
| C13 | 31191371 | 31192453 |
| C13 | 31264190 | 31265642 |
| C13 | 31480473 | 31481531 |
| C13 | 32214622 | 32215773 |
| C13 | 32573777 | 32575351 |
| C13 | 33212556 | 33213647 |
| C13 | 33543276 | 33544313 |
| C13 | 34184418 | 34185513 |
| C13 | 34392549 | 34393673 |
| C13 | 35059610 | 35060765 |
| C13 | 35516801 | 35517875 |
| C13 | 36587563 | 36588852 |
| C13 | 37729243 | 37730352 |
| C13 | 38829114 | 38830157 |
| C13 | 38969811 | 38970938 |
| C13 | 38984046 | 38985168 |
| C13 | 41240809 | 41241900 |
| C13 | 41949456 | 41950462 |
| C13 | 42025831 | 42027008 |
| C13 | 43014639 | 43015765 |
| C13 | 44547340 | 44549557 |
| C13 | 44874133 | 44875153 |
| C13 | 44947159 | 44948653 |
| C13 | 45013930 | 45014943 |
| C13 | 45430755 | 45431772 |
| C13 | 46039703 | 46040992 |
| C13 | 46309630 | 46310664 |
| C13 | 46499577 | 46500616 |
| C13 | 46893728 | 46895012 |
| C13 | 47011618 | 47013044 |
| C13 | 47126778 | 47127822 |
| C13 | 48805984 | 48807038 |
| C13 | 48892258 | 48893328 |
| C13 | 48892521 | 48893927 |
| C13 | 48893403 | 48894487 |
| C13 | 48893939 | 48895016 |
| C13 | 48895013 | 48896054 |
| C13 | 49039283 | 49040437 |
| C13 | 49427298 | 49428782 |
| C13 | 49549809 | 49550924 |
| C13 | 49952636 | 49953933 |
| C13 | 50167117 | 50168461 |
| C13 | 50193571 | 50195337 |
| C13 | 50707086 | 50708215 |
| C13 | 50714535 | 50715958 |
| C13 | 50928253 | 50929259 |
| C13 | 51103143 | 51104204 |
| C13 | 51233407 | 51234717 |
| C13 | 51653507 | 51655132 |
| C13 | 51834769 | 51835800 |
| C13 | 53312835 | 53313958 |
| C13 | 53419665 | 53420745 |
| C13 | 55723660 | 55724761 |
| C13 | 60014939 | 60016143 |
| C13 | 63655867 | 63656970 |
| C13 | 64650465 | 64651636 |
| C13 | 64902875 | 64903972 |
| C13 | 64920663 | 64921801 |
| C13 | 66716853 | 66718265 |
| C13 | 68633703 | 68635069 |
| C13 | 70036106 | 70037243 |
| C13 | 70602418 | 70603430 |
| C13 | 71482988 | 71484093 |
| C13 | 73848962 | 73850060 |
| C13 | 75836972 | 75837999 |
| C13 | 76868858 | 76869963 |
| C13 | 79176058 | 79177169 |
| C13 | 80851436 | 80852735 |
| C13 | 81228399 | 81229915 |
| C13 | 86930865 | 86931884 |
| C13 | 88327223 | 88329073 |
| C13 | 90535911 | 90536954 |
| C13 | 91826954 | 91828033 |
| C13 | 95024224 | 95025401 |
| C13 | 95252929 | 95254171 |
| C13 | 95359218 | 95360289 |
| C13 | 95840823 | 95842045 |
| C13 | 95968896 | 95969963 |
| C13 | 96328682 | 96329754 |
| C13 | 98653929 | 98655048 |
| C13 | 98696099 | 98697444 |
| C13 | 99127376 | 99128548 |
| C13 | 99135016 | 99136247 |
| C13 | 100056061 | 100057287 |
| C13 | 100084473 | 100085827 |
| C13 | 100128386 | 100130075 |
| C13 | 100197914 | 100198950 |
| C13 | 100217636 | 100218736 |
| C13 | 100676844 | 100678023 |
| C13 | 101173080 | 101174140 |
| C13 | 101196678 | 101198139 |
| C13 | 101349990 | 101351155 |
| C13 | 101627651 | 101628782 |
| C13 | 103019742 | 103021059 |
| C13 | 103506618 | 103507699 |
| C13 | 103764826 | 103766152 |
| C13 | 104009460 | 104010621 |
| C13 | 105114422 | 105115474 |
| C13 | 105306407 | 105307912 |
| C13 | 106097069 | 106098148 |
| C13 | 107092178 | 107093195 |
| C13 | 107138205 | 107139703 |
| C13 | 107187815 | 107188844 |
| C13 | 107518736 | 107520036 |
| C13 | 107571127 | 107572563 |
| C13 | 109147688 | 109148824 |
| C13 | 109792382 | 109793465 |
| C13 | 109793060 | 109794186 |
| C13 | 110383812 | 110384883 |
| C13 | 110391496 | 110393271 |
| C13 | 110396344 | 110397526 |
| C13 | 110434271 | 110435863 |
| C13 | 110435069 | 110436125 |
| C13 | 110436212 | 110437329 |
| C13 | 110437423 | 110438532 |
| C13 | 110521501 | 110522586 |
| C13 | 110645839 | 110646846 |
| C13 | 110965324 | 110966401 |
| C13 | 111023529 | 111024697 |
| C13 | 111061663 | 111062767 |
| C13 | 111108816 | 111109938 |
| C13 | 111188320 | 111189860 |
| C13 | 111198783 | 111200620 |
| C13 | 111464528 | 111465683 |
| C13 | 111521441 | 111522612 |
| C13 | 111669696 | 111671266 |
| C13 | 112062291 | 112063444 |
| C13 | 112169759 | 112171082 |
| C13 | 112669292 | 112670412 |
| C13 | 112669592 | 112670717 |
| C13 | 112707315 | 112708405 |
| C13 | 112712106 | 112713295 |
| C13 | 112720584 | 112721660 |
| C13 | 112721958 | 112722998 |
| C13 | 112722098 | 112723231 |
| C13 | 112893612 | 112894771 |
| C13 | 112984784 | 112985852 |
| C13 | 113242393 | 113243497 |
| C13 | 113400071 | 113401220 |
| C13 | 113505031 | 113506146 |
| C13 | 113559920 | 113560931 |
| C13 | 113612577 | 113613862 |
| C13 | 113634269 | 113635553 |
| C13 | 113639152 | 113640232 |
| C13 | 113676443 | 113677613 |
| C13 | 113704396 | 113705535 |
| C13 | 113761356 | 113762595 |
| C13 | 113761781 | 113762918 |
| C13 | 113763831 | 113765072 |
| C13 | 113783810 | 113784858 |
| C13 | 113788771 | 113789783 |
| C13 | 113985150 | 113986409 |
| C13 | 114019635 | 114020775 |
| C13 | 114064916 | 114066529 |
| C13 | 114218696 | 114220116 |
| C13 | 114282655 | 114283848 |
| C13 | 114427954 | 114429056 |
| C13 | 114432196 | 114433362 |
| C13 | 114432687 | 114433839 |
| C13 | 114432938 | 114434080 |
| C13 | 114443615 | 114444836 |
| C13 | 114486049 | 114487087 |
| C13 | 114505038 | 114506480 |
| C13 | 114520568 | 114521677 |
| C13 | 114523384 | 114524659 |
| C13 | 114523803 | 114525218 |
| C13 | 114528902 | 114529981 |
| C13 | 114529077 | 114530167 |
| C13 | 114538066 | 114539146 |
| C13 | 114585368 | 114586447 |
| C13 | 114743050 | 114744468 |
| C13 | 114755996 | 114757219 |
| C13 | 114780040 | 114781570 |
| C13 | 114808807 | 114809952 |
| C13 | 114808988 | 114810159 |
| C13 | 114815972 | 114817093 |
| C13 | 114825473 | 114826832 |
| C13 | 114833041 | 114834344 |
| C13 | 114848625 | 114849728 |
| C13 | 114865116 | 114866250 |
| C13 | 114870922 | 114872103 |
| C13 | 114873157 | 114874396 |
| C13 | 114884729 | 114885867 |
| C13 | 114884968 | 114886146 |
| C13 | 114893979 | 114895135 |
| C13 | 114916819 | 114918196 |
| C13 | 114921011 | 114922443 |
| C13 | 114963601 | 114964679 |
| C13 | 114964540 | 114965628 |
| C13 | 114964717 | 114965804 |
| C13 | 114964904 | 114965952 |
| C13 | 114972417 | 114973489 |
| C14 | 19066370 | 19067465 |
| C14 | 19219672 | 19221605 |
| C14 | 19225283 | 19226557 |
| C14 | 19320103 | 19322208 |
| C14 | 19359697 | 19360976 |
| C14 | 19360039 | 19361233 |
| C14 | 19402814 | 19403909 |
| C14 | 19550641 | 19552461 |
| C14 | 19640901 | 19642610 |
| C14 | 19685768 | 19686845 |
| C14 | 19686393 | 19687594 |
| C14 | 19960009 | 19961109 |
| C14 | 19980262 | 19982103 |
| C14 | 20023722 | 20025374 |
| C14 | 20067445 | 20068531 |
| C14 | 20097169 | 20098410 |
| C14 | 20334279 | 20335362 |
| C14 | 20680403 | 20681515 |
| C14 | 21127777 | 21128918 |
| C14 | 21160655 | 21161708 |
| C14 | 21191280 | 21192360 |
| C14 | 21428814 | 21430084 |
| C14 | 21535126 | 21536784 |
| C14 | 21553268 | 21554427 |
| C14 | 21560275 | 21561476 |
| C14 | 21571286 | 21572380 |
| C14 | 21656086 | 21657145 |
| C14 | 22220340 | 22221479 |
| C14 | 23017644 | 23018902 |
| C14 | 23314689 | 23315759 |
| C14 | 23386338 | 23387399 |
| C14 | 23516743 | 23517963 |
| C14 | 23517022 | 23518090 |
| C14 | 23586296 | 23587420 |
| C14 | 24047378 | 24048620 |
| C14 | 24194824 | 24196271 |
| C14 | 24293699 | 24294743 |
| C14 | 24641099 | 24642459 |
| C14 | 24718013 | 24719079 |
| C14 | 24800430 | 24801525 |
| C14 | 24860357 | 24861598 |
| C14 | 24950605 | 24951789 |
| C14 | 26146878 | 26147958 |
| C14 | 26296887 | 26298066 |
| C14 | 28237643 | 28238724 |
| C14 | 29236320 | 29237399 |
| C14 | 29242904 | 29244254 |
| C14 | 30560677 | 30561702 |
| C14 | 31159097 | 31160540 |
| C14 | 31925983 | 31927043 |
| C14 | 34268462 | 34269581 |
| C14 | 34268744 | 34269962 |
| C14 | 34269166 | 34270279 |
| C14 | 34269638 | 34270919 |
| C14 | 34813839 | 34815028 |
| C14 | 35024000 | 35025097 |
| C14 | 35033940 | 35035030 |
| C14 | 35312772 | 35313891 |
| C14 | 35410104 | 35411152 |
| C14 | 35508348 | 35509836 |
| C14 | 35837754 | 35838846 |
| C14 | 35837913 | 35839051 |
| C14 | 35838648 | 35839741 |
| C14 | 36293731 | 36295254 |
| C14 | 36445550 | 36446682 |
| C14 | 36445891 | 36447019 |
| C14 | 36446316 | 36447419 |
| C14 | 36570923 | 36571963 |
| C14 | 36651459 | 36652612 |
| C14 | 36979171 | 36980203 |
| C14 | 36983071 | 36984237 |
| C14 | 37128049 | 37129843 |
| C14 | 38024292 | 38025373 |
| C14 | 38679923 | 38681185 |
| C14 | 38724495 | 38725548 |
| C14 | 38832508 | 38833659 |
| C14 | 39485934 | 39487140 |
| C14 | 39875245 | 39876305 |
| C14 | 40166453 | 40169378 |
| C14 | 40996149 | 40997345 |
| C14 | 42043684 | 42044872 |
| C14 | 42243816 | 42244954 |
| C14 | 43840455 | 43841928 |
| C14 | 43857887 | 43859192 |
| C14 | 44089318 | 44090450 |
| C14 | 45267255 | 45268341 |
| C14 | 45267623 | 45268699 |
| C14 | 45365830 | 45366945 |
| C14 | 48142968 | 48144043 |
| C14 | 48144159 | 48145731 |
| C14 | 48229278 | 48230536 |
| C14 | 48333891 | 48335030 |
| C14 | 49808824 | 49809888 |
| C14 | 50287211 | 50288785 |
| C14 | 51302834 | 51304386 |
| C14 | 51338304 | 51339385 |
| C14 | 51443935 | 51445009 |
| C14 | 52116636 | 52117865 |
| C14 | 52265779 | 52267369 |
| C14 | 52733746 | 52734856 |
| C14 | 52734157 | 52735274 |
| C14 | 52735043 | 52736069 |
| C14 | 52781108 | 52782219 |
| C14 | 52781436 | 52782917 |
| C14 | 53301395 | 53303275 |
| C14 | 54418115 | 54419352 |
| C14 | 55034146 | 55035231 |
| C14 | 55092091 | 55093292 |
| C14 | 56913837 | 56915010 |
| C14 | 57030463 | 57031837 |
| C14 | 59950243 | 59951318 |
| C14 | 60431969 | 60433096 |
| C14 | 60793615 | 60795092 |
| C14 | 60981980 | 60983108 |
| C14 | 61527134 | 61528229 |
| C14 | 61654635 | 61655746 |
| C14 | 62549663 | 62550710 |
| C14 | 63541777 | 63542821 |
| C14 | 64228013 | 64229151 |
| C14 | 64908619 | 64909752 |
| C14 | 65099155 | 65100257 |
| C14 | 65444951 | 65446633 |
| C14 | 65509956 | 65511125 |
| C14 | 66158614 | 66159849 |
| C14 | 67734567 | 67735868 |
| C14 | 68285286 | 68286473 |
| C14 | 69015866 | 69016930 |
| C14 | 69094594 | 69095657 |
| C14 | 69094815 | 69096188 |
| C14 | 69095199 | 69096439 |
| C14 | 69256064 | 69257508 |
| C14 | 69282221 | 69283356 |
| C14 | 70039092 | 70040193 |
| C14 | 70316520 | 70317802 |
| C14 | 70700354 | 70701427 |
| C14 | 71720519 | 71721669 |
| C14 | 73208059 | 73209349 |
| C14 | 73289617 | 73290745 |
| C14 | 73395241 | 73396335 |
| C14 | 73602580 | 73603673 |
| C14 | 74005824 | 74006993 |
| C14 | 74226433 | 74227606 |
| C14 | 74288056 | 74289199 |
| C14 | 75593729 | 75594938 |
| C14 | 75800171 | 75801302 |
| C14 | 75800461 | 75801523 |
| C14 | 76733765 | 76734869 |
| C14 | 76939400 | 76940489 |
| C14 | 77191940 | 77193071 |
| C14 | 77296144 | 77297232 |
| C14 | 77491334 | 77492647 |
| C14 | 77491700 | 77492890 |
| C14 | 77491908 | 77493610 |
| C14 | 77498837 | 77500020 |
| C14 | 77786648 | 77787705 |
| C14 | 78249423 | 78250573 |
| C14 | 78566763 | 78567779 |
| C14 | 78887604 | 78888659 |
| C14 | 80888928 | 80889965 |
| C14 | 81686880 | 81687916 |
| C1 | 81780832 | 81781905 |
| C14 | 83558235 | 83559295 |
| C14 | 83990224 | 83991300 |
| C14 | 86705257 | 86706321 |
| C14 | 86912668 | 86913677 |
| C14 | 87555551 | 87556810 |
| C14 | 88526712 | 88527735 |
| C14 | 89444515 | 89445696 |
| C14 | 89698588 | 89699928 |
| C14 | 89738418 | 89739467 |
| C14 | 90144756 | 90145773 |
| C14 | 90190606 | 90191710 |
| C14 | 90244951 | 90246678 |
| C14 | 90991334 | 90992395 |
| C14 | 91510444 | 91511489 |
| C14 | 91526424 | 91527486 |
| C14 | 91828487 | 91829512 |
| C14 | 92828761 | 92830149 |
| C14 | 92852640 | 92853769 |
| C14 | 93008973 | 93010177 |
| C14 | 93115760 | 93117075 |
| C14 | 93152736 | 93154003 |
| C14 | 93153342 | 93154544 |
| C14 | 93185074 | 93186179 |
| C14 | 93216467 | 93217728 |
| C14 | 93377225 | 93378357 |
| C14 | 93377758 | 93378957 |
| C14 | 93581155 | 93582189 |
| C14 | 94684160 | 94685581 |
| C14 | 94812082 | 94813271 |
| C14 | 94927968 | 94929000 |
| C14 | 95234902 | 95235989 |
| C14 | 95364290 | 95365305 |
| C14 | 96134183 | 96135293 |
| C14 | 96337821 | 96338949 |
| C14 | 96858648 | 96859755 |
| C14 | 97774771 | 97775812 |
| C14 | 97924822 | 97925939 |
| C14 | 99584148 | 99585231 |
| C14 | 99640411 | 99641729 |
| C14 | 99665365 | 99666716 |
| C14 | 100141298 | 100142410 |
| C14 | 100181607 | 100182817 |
| C14 | 100304769 | 100306502 |
| C14 | 100658084 | 100659219 |
| C14 | 100750231 | 100751310 |
| C14 | 100810807 | 100811888 |
| C14 | 101022885 | 101023955 |
| C14 | 101069217 | 101070768 |
| C14 | 101197820 | 101198967 |
| C14 | 101291849 | 101293102 |
| C14 | 101293135 | 101294253 |
| C14 | 101348933 | 101350089 |
| C14 | 101362515 | 101363698 |
| C14 | 101370356 | 101371647 |
| C14 | 101531708 | 101532737 |
| C14 | 101594696 | 101595827 |
| C14 | 101617625 | 101618794 |
| C14 | 101695349 | 101696793 |
| C14 | 101695907 | 101696987 |
| C14 | 101825546 | 101826609 |
| C14 | 102050187 | 102051267 |
| C14 | 102050435 | 102051522 |
| C14 | 102227415 | 102228458 |
| C14 | 102676576 | 102677816 |
| C14 | 102976281 | 102977421 |
| C14 | 102989711 | 102990841 |
| C14 | 103393472 | 103395230 |
| C14 | 103396080 | 103397207 |
| C14 | 103436448 | 103437548 |
| C14 | 103568303 | 103569344 |
| C14 | 103592764 | 103593932 |
| C14 | 103592964 | 103594252 |
| C14 | 103604372 | 103605485 |
| C14 | 103738832 | 103740080 |
| C14 | 103838070 | 103839136 |
| C14 | 104090802 | 104091871 |
| C14 | 104159612 | 104160881 |
| C14 | 104193438 | 104194454 |
| C14 | 104386114 | 104387434 |
| C14 | 104394444 | 104395553 |
| C14 | 104408349 | 104409500 |
| C14 | 104551604 | 104552659 |
| C14 | 104621451 | 104622649 |
| C14 | 104623608 | 104624725 |
| C14 | 104640864 | 104642182 |
| C14 | 105045472 | 105046653 |
| C14 | 105103118 | 105104229 |
| C14 | 105119454 | 105120541 |
| C14 | 105126643 | 105127776 |
| C14 | 105179069 | 105180168 |
| C14 | 105195492 | 105196654 |
| C14 | 105217995 | 105219113 |
| C14 | 105235186 | 105237257 |
| C14 | 105236582 | 105239289 |
| C14 | 105238715 | 105240216 |
| C14 | 105239292 | 105245408 |
| C14 | 105245925 | 105247053 |
| C14 | 105252128 | 105253204 |
| C14 | 105258435 | 105261365 |
| C14 | 105260848 | 105261881 |
| C14 | 105261321 | 105262588 |
| C14 | 105323184 | 105324248 |
| C14 | 105371043 | 105372158 |
| C14 | 105398675 | 105399773 |
| C14 | 105491948 | 105493133 |
| C14 | 105530607 | 105531832 |
| C14 | 105577221 | 105578288 |
| C14 | 105715068 | 105716181 |
| C14 | 105859791 | 105860994 |
| C14 | 105902376 | 105903495 |
| C14 | 105943596 | 105944791 |
| C14 | 105943979 | 105945104 |
| C14 | 105944334 | 105945803 |
| C14 | 105944883 | 105946125 |
| C14 | 105957281 | 105958336 |
| C14 | 105995495 | 105996956 |
| C14 | 106040328 | 106041423 |
| C14 | 106064516 | 106065770 |
| C14 | 106108873 | 106110029 |
| C14 | 106145412 | 106146610 |
| C14 | 106148335 | 106149573 |
| C14 | 106206577 | 106207859 |
| C14 | 106234341 | 106235504 |
| C14 | 106333201 | 106334329 |
| C14 | 106448869 | 106451348 |
| C14 | 106527999 | 106529798 |
| C14 | 106891548 | 106892645 |
| C14 | 106937393 | 106938698 |
| C14 | 106954039 | 106955140 |
| C14 | 107094629 | 107095903 |
| C14 | 107252278 | 107253422 |
| C15 | 20148287 | 20149369 |
| C15 | 20148632 | 20149715 |
| C15 | 20148773 | 20149865 |
| C15 | 20149282 | 20150400 |
| C15 | 20252441 | 20253856 |
| C15 | 20254154 | 20255561 |
| C15 | 20346204 | 20348083 |
| C15 | 20352971 | 20354019 |
| C15 | 20373281 | 20374343 |
| C15 | 20641073 | 20642727 |
| C15 | 20671839 | 20672963 |
| C15 | 20702902 | 20704163 |
| C15 | 20710756 | 20711837 |
| C15 | 20711224 | 20712323 |
| C15 | 20786464 | 20788100 |
| C15 | 20787468 | 20788622 |
| C15 | 20960895 | 20962014 |
| C15 | 20980772 | 20981842 |
| C15 | 20988063 | 20989329 |
| C15 | 20988733 | 20989891 |
| C15 | 21004693 | 21005948 |
| C15 | 21005568 | 21006823 |
| C15 | 21019449 | 21020664 |
| C15 | 21021211 | 21022821 |
| C15 | 21070444 | 21071820 |
| C15 | 21082721 | 21083828 |
| C15 | 21082991 | 21084053 |
| C15 | 21083664 | 21084847 |
| C15 | 21171016 | 21172152 |
| C15 | 21213782 | 21216103 |
| C15 | 21216421 | 21217857 |
| C15 | 21286322 | 21287370 |
| C15 | 21359366 | 21360430 |
| C15 | 21364593 | 21365724 |
| C15 | 21379899 | 21381012 |
| C15 | 21967594 | 21968674 |
| C15 | 21994769 | 21996025 |
| C15 | 21995641 | 21997112 |
| C15 | 21996849 | 21997982 |
| C15 | 22094009 | 22095222 |
| C15 | 22094660 | 22095952 |
| C15 | 22156874 | 22158105 |
| C15 | 22664594 | 22665721 |
| C15 | 22803021 | 22804306 |
| C15 | 22809443 | 22810510 |
| C15 | 23034951 | 23036023 |
| C15 | 23157477 | 23158625 |
| C15 | 23161447 | 23162577 |
| C15 | 23207844 | 23208894 |
| C15 | 23281126 | 23282269 |
| C15 | 23300686 | 23301863 |
| C15 | 23356061 | 23357345 |
| C15 | 23375613 | 23376755 |
| C15 | 23377401 | 23378597 |
| C15 | 23397875 | 23399286 |
| C15 | 23453644 | 23454861 |
| C15 | 23454492 | 23455703 |
| C15 | 23454780 | 23455965 |
| C15 | 23454990 | 23456138 |
| C15 | 23500889 | 23502055 |
| C15 | 23890731 | 23891819 |
| C15 | 23931929 | 23932963 |
| C15 | 23931998 | 23933034 |
| C15 | 24206646 | 24207806 |
| C15 | 24595842 | 24597148 |
| C15 | 25017679 | 25018743 |
| C15 | 25017858 | 25018959 |
| C15 | 25122937 | 25124157 |
| C15 | 25199624 | 25200695 |
| C15 | 25200348 | 25201468 |
| C15 | 25200929 | 25202092 |
| C15 | 26012790 | 26013809 |
| C15 | 26075867 | 26077613 |
| C15 | 26327309 | 26328396 |
| C15 | 26873679 | 26875006 |
| C15 | 26915191 | 26916215 |
| C15 | 27508445 | 27509552 |
| C15 | 27924894 | 27926030 |
| C15 | 28217579 | 28218637 |
| C15 | 28567091 | 28568144 |
| C15 | 28657702 | 28659013 |
| C15 | 28685839 | 28686974 |
| C15 | 28788258 | 28789506 |
| C15 | 28969884 | 28970963 |
| C15 | 29007825 | 29009212 |
| C15 | 29034138 | 29036108 |
| C15 | 29210422 | 29211522 |
| C15 | 29395326 | 29396440 |
| C15 | 29758891 | 29759932 |
| C15 | 29967365 | 29968429 |
| C15 | 30336222 | 30337338 |
| C15 | 30336571 | 30337636 |
| C15 | 30578955 | 30580213 |
| C15 | 30781192 | 30782386 |
| C15 | 31476820 | 31477886 |
| C15 | 31528031 | 31529424 |
| C15 | 31657668 | 31658824 |
| C15 | 31775179 | 31776274 |
| C15 | 31775930 | 31777030 |
| C15 | 32502173 | 32503320 |
| C15 | 32574673 | 32576471 |
| C15 | 32604281 | 32605408 |
| C15 | 32605027 | 32606127 |
| C15 | 32633954 | 32635039 |
| C15 | 32726183 | 32727282 |
| C15 | 32753686 | 32755356 |
| C15 | 32760880 | 32762210 |
| C15 | 32871318 | 32872588 |
| C15 | 32878686 | 32879913 |
| C15 | 32933335 | 32934515 |
| C15 | 34629775 | 34630852 |
| C15 | 34729317 | 34730368 |
| C15 | 34782204 | 34783261 |
| C15 | 36487124 | 36488341 |
| C15 | 36542292 | 36543438 |
| C15 | 36870691 | 36871751 |
| C15 | 37169843 | 37170921 |
| C15 | 39267406 | 39268432 |
| C15 | 39423506 | 39424579 |
| C15 | 40268201 | 40269354 |
| C15 | 40268495 | 40269561 |
| C15 | 40364145 | 40365206 |
| C15 | 40365617 | 40366703 |
| C15 | 40395232 | 40396567 |
| C15 | 40452684 | 40453733 |
| C15 | 40582564 | 40583713 |
| C15 | 40582975 | 40584341 |
| C15 | 40599383 | 40600785 |
| C15 | 40778148 | 40779266 |
| C15 | 40875501 | 40876726 |
| C15 | 40974765 | 40976008 |
| C15 | 41194578 | 41195649 |
| C15 | 41217227 | 41218361 |
| C15 | 41217711 | 41218856 |
| C15 | 41674291 | 41675390 |
| C15 | 41872525 | 41873550 |
| C15 | 42371166 | 42372241 |
| C15 | 43368746 | 43369911 |
| C15 | 44543952 | 44545091 |
| C15 | 45092214 | 45093259 |
| C15 | 45427436 | 45428617 |
| C15 | 46307855 | 46308992 |
| C15 | 47423966 | 47425061 |
| C15 | 47425204 | 47426505 |
| C15 | 48549914 | 48551037 |
| C15 | 49842008 | 49843200 |
| C15 | 51490111 | 51492043 |
| C15 | 52029099 | 52030138 |
| C15 | 52263973 | 52265053 |
| C15 | 53083018 | 53084175 |
| C15 | 53431211 | 53432684 |
| C15 | 53516911 | 53518033 |
| C15 | 55141859 | 55143721 |
| C15 | 55595067 | 55597032 |
| C15 | 55609956 | 55611174 |
| C15 | 55956357 | 55957370 |
| C15 | 56298825 | 56299944 |
| C15 | 56298982 | 56300137 |
| C15 | 59041035 | 59042093 |
| C15 | 59156926 | 59158024 |
| C15 | 59771648 | 59772662 |
| C15 | 59831355 | 59832526 |
| C15 | 60144697 | 60145707 |
| C15 | 61775822 | 61776924 |
| C15 | 62636720 | 62637802 |
| C15 | 65066049 | 65067305 |
| C15 | 65688712 | 65689766 |
| C15 | 66546337 | 66547449 |
| C15 | 66947431 | 66948647 |
| C15 | 66953149 | 66954734 |
| C15 | 66993278 | 66994347 |
| C15 | 67141966 | 67143048 |
| C15 | 67325487 | 67326534 |
| C15 | 68367659 | 68368713 |
| C15 | 68723170 | 68724300 |
| C15 | 68983190 | 68984281 |
| C15 | 69222139 | 69223342 |
| C15 | 69222395 | 69223588 |
| C15 | 69743888 | 69744963 |
| C15 | 69755674 | 69756708 |
| C15 | 70013506 | 70014661 |
| C15 | 70104859 | 70105960 |
| C15 | 70482046 | 70483064 |
| C15 | 71035801 | 71036956 |
| C15 | 72061159 | 72062626 |
| C15 | 72928992 | 72930551 |
| C15 | 72978791 | 72980105 |
| C15 | 74265602 | 74266673 |
| C15 | 74426230 | 74427264 |
| C15 | 74833222 | 74834326 |
| C15 | 75077607 | 75079114 |
| C15 | 75218115 | 75219133 |
| C15 | 75429917 | 75430972 |
| C15 | 75866900 | 75868005 |
| C15 | 76483831 | 76484877 |
| C15 | 76628031 | 76629294 |
| C15 | 77264651 | 77265680 |
| C15 | 77910524 | 77911659 |
| C15 | 78200159 | 78201339 |
| C15 | 78401602 | 78402628 |
| C15 | 78605423 | 78606456 |
| C15 | 78632823 | 78634106 |
| C15 | 79090462 | 79092292 |
| C15 | 79092333 | 79093355 |
| C15 | 79123351 | 79124425 |
| C15 | 80188982 | 80190105 |
| C15 | 80882767 | 80883956 |
| C15 | 81410216 | 81411585 |
| C15 | 81425677 | 81427111 |
| C15 | 81454816 | 81456007 |
| C15 | 81622983 | 81624079 |
| C15 | 81887420 | 81889446 |
| C15 | 82629703 | 82631271 |
| C15 | 82957696 | 82959943 |
| C15 | 83274863 | 83276023 |
| C15 | 83314702 | 83315813 |
| C15 | 83665708 | 83667066 |
| C15 | 83682556 | 83683625 |
| C15 | 85360146 | 85361262 |
| C15 | 85976713 | 85978052 |
| C15 | 86379021 | 86380069 |
| C15 | 87939689 | 87940750 |
| C15 | 89182158 | 89183340 |
| C15 | 89251440 | 89252596 |
| C15 | 89939516 | 89940559 |
| C15 | 89950858 | 89952041 |
| C15 | 90012869 | 90013954 |
| C15 | 90356885 | 90358014 |
| C15 | 90357021 | 90358161 |
| C15 | 90411232 | 90412332 |
| C15 | 90437371 | 90438505 |
| C15 | 90592742 | 90593916 |
| C15 | 90625225 | 90626462 |
| C15 | 90701711 | 90702767 |
| C15 | 90726992 | 90728358 |
| C15 | 91364905 | 91365964 |
| C15 | 91499643 | 91500764 |
| C15 | 91918830 | 91919840 |
| C15 | 92792780 | 92793885 |
| C15 | 92875966 | 92878313 |
| C15 | 93113266 | 93114775 |
| C15 | 93276598 | 93278168 |
| C15 | 93350453 | 93351608 |
| C15 | 93477956 | 93479017 |
| C15 | 93551262 | 93552300 |
| C15 | 93803273 | 93804478 |
| C15 | 93848805 | 93849838 |
| C15 | 93999756 | 94000805 |
| C15 | 94409425 | 94410860 |
| C15 | 94644538 | 94645624 |
| C15 | 95869974 | 95870993 |
| C15 | 96553318 | 96554457 |
| C15 | 96894829 | 96896229 |
| C15 | 96896180 | 96897736 |
| C15 | 96903343 | 96904507 |
| C15 | 96908454 | 96909871 |
| C15 | 97527082 | 97528170 |
| C15 | 98146788 | 98147820 |
| C15 | 98414307 | 98415532 |
| C15 | 98418356 | 98419536 |
| C15 | 98503737 | 98504798 |
| C15 | 98647225 | 98648286 |
| C15 | 99068467 | 99069744 |
| C15 | 99237505 | 99238553 |
| C15 | 99332024 | 99333243 |
| C15 | 99407906 | 99409233 |
| C15 | 99408773 | 99409879 |
| C15 | 99557415 | 99558526 |
| C15 | 99601276 | 99602367 |
| C15 | 99705900 | 99706930 |
| C15 | 100466614 | 100467708 |
| C15 | 100468058 | 100469254 |
| C15 | 100671763 | 100672851 |
| C15 | 101084065 | 101085230 |
| C15 | 101084283 | 101085445 |
| C15 | 101096794 | 101097907 |
| C15 | 101327161 | 101328318 |
| C15 | 101403642 | 101404669 |
| C15 | 101873801 | 101875161 |
| C15 | 101938080 | 101939265 |
| C15 | 101991100 | 101992396 |
| C15 | 102031755 | 102032857 |
| C15 | 102263783 | 102264913 |
| C15 | 102285848 | 102287004 |
| C15 | 102294401 | 102295555 |
| C15 | 102296101 | 102297264 |
| C15 | 102296718 | 102297944 |
| C15 | 102297742 | 102298810 |
| C15 | 102298392 | 102299545 |
| C15 | 102478985 | 102480060 |
| C16 | 60050 | 61227 |
| C16 | 60274 | 61345 |
| C16 | 60365 | 61616 |
| C16 | 60642 | 62142 |
| C16 | 65244 | 66307 |
| C16 | 72344 | 73519 |
| C16 | 77002 | 78401 |
| C16 | 214080 | 215247 |
| C16 | 222753 | 223935 |
| C16 | 231642 | 232807 |
| C16 | 318118 | 319341 |
| C16 | 374570 | 375988 |
| C16 | 393448 | 394727 |
| C16 | 402225 | 403287 |
| C16 | 411861 | 413024 |
| C16 | 437724 | 438787 |
| C16 | 450401 | 451522 |
| C16 | 506388 | 507639 |
| C16 | 597015 | 598081 |
| C16 | 628506 | 629610 |
| C16 | 645784 | 647047 |
| C16 | 688980 | 690201 |
| C16 | 698779 | 699968 |
| C16 | 710718 | 712046 |
| C16 | 734362 | 735695 |
| C16 | 743364 | 744469 |
| C16 | 771308 | 772535 |
| C16 | 817414 | 818640 |
| C16 | 818402 | 819681 |
| C16 | 853674 | 854828 |
| C16 | 857528 | 858589 |
| C16 | 866594 | 868455 |
| C16 | 928079 | 929150 |
| C16 | 932488 | 933534 |
| C16 | 962471 | 963601 |
| C16 | 1010121 | 1011262 |
| C16 | 1013207 | 1014519 |
| C16 | 1034651 | 1035782 |
| C16 | 1036560 | 1037663 |
| C16 | 1040261 | 1041865 |
| C16 | 1068802 | 1070338 |
| C16 | 1069528 | 1070699 |
| C16 | 1092742 | 1093937 |
| C16 | 1128463 | 1129567 |
| C16 | 1175105 | 1176179 |
| C16 | 1199036 | 1200081 |
| C16 | 1206543 | 1207647 |
| C16 | 1208104 | 1209309 |
| C16 | 1208336 | 1209505 |
| C16 | 1210017 | 1211445 |
| C16 | 1216698 | 1217905 |
| C16 | 1224175 | 1225397 |
| C16 | 1232849 | 1234081 |
| C16 | 1233168 | 1234494 |
| C16 | 1234270 | 1235346 |
| C16 | 1235297 | 1236423 |
| C16 | 1243003 | 1244163 |
| C16 | 1244566 | 1245862 |
| C16 | 1248793 | 1249894 |
| C16 | 1262117 | 1263227 |
| C16 | 1267459 | 1268611 |
| C16 | 1267823 | 1269089 |
| C16 | 1270671 | 1271788 |
| C16 | 1270949 | 1272123 |
| C16 | 1282847 | 1284019 |
| C16 | 1285187 | 1286669 |
| C16 | 1294776 | 1295875 |
| C16 | 1303284 | 1304341 |
| C16 | 1307431 | 1308582 |
| C16 | 1326270 | 1327462 |
| C16 | 1337794 | 1338954 |
| C16 | 1361400 | 1362670 |
| C16 | 1471170 | 1472390 |
| C16 | 1476808 | 1477889 |
| C16 | 1477824 | 1478950 |
| C16 | 1478148 | 1479200 |
| C16 | 1482300 | 1484261 |
| C16 | 1503232 | 1504456 |
| C16 | 1507006 | 1508107 |
| C16 | 1519360 | 1520564 |
| C16 | 1534017 | 1535424 |
| C16 | 1544817 | 1545928 |
| C16 | 1583647 | 1584725 |
| C16 | 1644551 | 1645562 |
| C16 | 1649296 | 1650475 |
| C16 | 1651630 | 1652983 |
| C16 | 1661793 | 1662827 |
| C16 | 1844596 | 1845735 |
| C16 | 1921582 | 1922656 |
| C16 | 2042123 | 2043250 |
| C16 | 2083984 | 2085328 |
| C16 | 2139262 | 2140395 |
| C16 | 2140636 | 2141716 |
| C16 | 2140788 | 2141853 |
| C16 | 2141001 | 2142072 |
| C16 | 2173012 | 2174115 |
| C16 | 2183670 | 2184848 |
| C16 | 2210632 | 2211974 |
| C16 | 2228563 | 2229627 |
| C16 | 2256164 | 2257471 |
| C16 | 2264404 | 2265466 |
| C16 | 2274018 | 2275278 |
| C16 | 2546793 | 2548075 |
| C16 | 2707111 | 2709022 |
| C16 | 2713849 | 2714871 |
| C16 | 2739671 | 2741261 |
| C16 | 2767242 | 2768351 |
| C16 | 2847991 | 2849058 |
| C16 | 2848438 | 2849518 |
| C16 | 2866727 | 2867799 |
| C16 | 2866876 | 2867975 |
| C16 | 2866993 | 2868209 |
| C16 | 2876193 | 2877436 |
| C16 | 2902453 | 2903628 |
| C16 | 2932547 | 2933650 |
| C16 | 2961288 | 2962364 |
| C16 | 2986592 | 2987742 |
| C16 | 3030048 | 3031065 |
| C16 | 3054898 | 3056221 |
| C16 | 3126668 | 3127755 |
| C16 | 3130444 | 3131770 |
| C16 | 3139101 | 3140212 |
| C16 | 3184502 | 3185589 |
| C16 | 3187929 | 3189001 |
| C16 | 3230055 | 3231443 |
| C16 | 3285435 | 3286607 |
| C16 | 3304070 | 3305195 |
| C16 | 3312959 | 3314110 |
| C16 | 3344646 | 3345725 |
| C16 | 3492928 | 3493966 |
| C16 | 3493577 | 3494676 |
| C16 | 3549587 | 3550706 |
| C16 | 3638593 | 3639773 |
| C16 | 3638953 | 3640083 |
| C16 | 3640484 | 3641703 |
| C16 | 3718411 | 3719509 |
| C16 | 4094402 | 4095455 |
| C16 | 4290262 | 4291334 |
| C16 | 4306596 | 4308318 |
| C16 | 4357264 | 4358350 |
| C16 | 4425732 | 4427067 |
| C16 | 4674159 | 4675265 |
| C16 | 4697805 | 4699163 |
| C16 | 4698211 | 4699318 |
| C16 | 4729763 | 4730851 |
| C16 | 4729901 | 4731102 |
| C16 | 4732509 | 4733579 |
| C16 | 4742830 | 4743878 |
| C16 | 4824457 | 4825600 |
| C16 | 4845430 | 4846481 |
| C16 | 4896798 | 4897908 |
| C16 | 4909594 | 4910755 |
| C16 | 5048111 | 5049273 |
| C16 | 5074031 | 5075071 |
| C16 | 5251703 | 5252981 |
| C16 | 5458004 | 5459060 |
| C16 | 6299311 | 6300386 |
| C16 | 6708860 | 6709910 |
| C16 | 6759268 | 6760900 |
| C16 | 6897151 | 6898183 |
| C16 | 7108095 | 7109109 |
| C16 | 8066260 | 8067374 |
| C16 | 8228654 | 8229995 |
| C16 | 8234934 | 8236013 |
| C16 | 8443296 | 8444401 |
| C16 | 8865315 | 8866319 |
| C16 | 9047326 | 9048444 |
| C16 | 9269124 | 9270158 |
| C16 | 9443572 | 9444715 |
| C16 | 9573071 | 9574245 |
| C16 | 9699399 | 9700455 |
| C16 | 9753803 | 9754856 |
| C16 | 9767080 | 9768988 |
| C16 | 9823126 | 9824162 |
| C16 | 10132960 | 10134052 |
| C16 | 10140637 | 10141691 |
| C16 | 10273570 | 10274700 |
| C16 | 10308786 | 10309798 |
| C16 | 10340075 | 10341109 |
| C16 | 10448948 | 10450113 |
| C16 | 10499530 | 10500548 |
| C16 | 10705447 | 10707239 |
| C16 | 10911715 | 10912899 |
| C16 | 11001556 | 11002614 |
| C16 | 11326317 | 11327886 |
| C16 | 11429416 | 11430635 |
| C16 | 11587291 | 11588392 |
| C16 | 11678156 | 11679286 |
| C16 | 12130395 | 12131892 |
| C16 | 12426271 | 12427333 |
| C16 | 12861219 | 12862825 |
| C16 | 13167143 | 13168168 |
| C16 | 13649783 | 13650977 |
| C16 | 13686796 | 13688139 |
| C16 | 13740399 | 13741505 |
| C16 | 14953904 | 14955037 |
| C16 | 15006541 | 15007888 |
| C16 | 15013358 | 15014446 |
| C16 | 15082842 | 15084843 |
| C16 | 15155202 | 15156945 |
| C16 | 15229789 | 15231000 |
| C16 | 15246915 | 15248155 |
| C16 | 15247731 | 15248793 |
| C16 | 15337806 | 15339680 |
| C16 | 15382500 | 15384090 |
| C16 | 15704492 | 15705598 |
| C16 | 15905465 | 15906717 |
| C16 | 15997030 | 15998054 |
| C16 | 16138915 | 16140449 |
| C16 | 16215673 | 16216867 |
| C16 | 16342853 | 16344871 |
| C16 | 16418102 | 16419517 |
| C16 | 16418865 | 16420091 |
| C16 | 16450971 | 16452445 |
| C16 | 16451665 | 16453131 |
| C16 | 16456444 | 16457627 |
| C16 | 16459337 | 16460551 |
| C16 | 16462983 | 16464071 |
| C16 | 16571900 | 16573930 |
| C16 | 16623093 | 16624282 |
| C16 | 16714009 | 16715701 |
| C16 | 16716335 | 16717544 |
| C16 | 16793432 | 16795102 |
| C16 | 17428915 | 17430033 |
| C16 | 17599030 | 17600203 |
| C16 | 17808927 | 17810050 |
| C16 | 17862978 | 17864012 |
| C16 | 18141999 | 18143034 |
| C16 | 18213791 | 18215000 |
| C16 | 18325510 | 18327466 |
| C16 | 18431458 | 18432633 |
| C16 | 18475700 | 18476912 |
| C16 | 18481592 | 18482698 |
| C16 | 18486998 | 18488502 |
| C16 | 18664796 | 18666600 |
| C16 | 18723888 | 18727257 |
| C16 | 18735585 | 18737031 |
| C16 | 18801577 | 18802766 |
| C16 | 18917296 | 18918311 |
| C16 | 18938331 | 18939544 |
| C16 | 19008676 | 19010002 |
| C16 | 19014199 | 19015375 |
| C16 | 19079388 | 19080551 |
| C16 | 20084399 | 20085428 |
| C16 | 20381909 | 20382945 |
| C16 | 21139342 | 21140533 |
| C16 | 21199902 | 21201093 |
| C16 | 21294841 | 21295911 |
| C16 | 21363551 | 21364589 |
| C16 | 21369196 | 21370291 |
| C16 | 21414487 | 21415658 |
| C16 | 21656039 | 21657257 |
| C16 | 21831129 | 21832623 |
| C16 | 21847047 | 21848202 |
| C16 | 21847217 | 21848458 |
| C16 | 22086327 | 22087476 |
| C16 | 22825063 | 22826090 |
| C16 | 23847007 | 23848075 |
| C16 | 23847086 | 23848266 |
| C16 | 23999976 | 24001866 |
| C16 | 24051995 | 24053620 |
| C16 | 24094092 | 24095131 |
| C16 | 24100519 | 24101529 |
| C16 | 24106839 | 24107845 |
| C16 | 24119318 | 24120797 |
| C16 | 24120655 | 24121783 |
| C16 | 24405090 | 24406503 |
| C16 | 24504877 | 24505893 |
| C16 | 26237083 | 26238088 |
| C16 | 26295083 | 26296259 |
| C16 | 26586188 | 26587213 |
| C16 | 26607594 | 26608708 |
| C16 | 26891959 | 26893126 |
| C16 | 26985142 | 26986215 |
| C16 | 27064408 | 27065637 |
| C16 | 27177802 | 27178813 |
| C16 | 27248716 | 27249881 |
| C16 | 28379375 | 28380474 |
| C16 | 28382525 | 28384057 |
| C16 | 28427740 | 28429509 |
| C16 | 28435935 | 28437515 |
| C16 | 28459545 | 28460802 |
| C16 | 28564779 | 28565863 |
| C16 | 28611745 | 28612867 |
| C16 | 28611929 | 28613131 |
| C16 | 28677030 | 28678105 |
| C16 | 28699631 | 28701010 |
| C16 | 28708612 | 28709859 |
| C16 | 28708936 | 28710049 |
| C16 | 28710143 | 28711270 |
| C16 | 28754210 | 28755592 |
| C16 | 28757619 | 28758775 |
| C16 | 28758119 | 28759222 |
| C16 | 28790832 | 28792037 |
| C16 | 29064475 | 29065646 |
| C16 | 29067689 | 29069256 |
| C16 | 29070756 | 29071961 |
| C16 | 29085876 | 29086970 |
| C16 | 29168135 | 29169450 |
| C16 | 29229158 | 29230399 |
| C16 | 29229453 | 29230782 |
| C16 | 29231270 | 29233003 |
| C16 | 29289435 | 29290477 |
| C16 | 29296074 | 29297194 |
| C16 | 29313403 | 29314646 |
| C16 | 29393562 | 29394733 |
| C16 | 29613087 | 29614476 |
| C16 | 29675515 | 29678389 |
| C16 | 29795947 | 29797121 |
| C16 | 29816449 | 29817497 |
| C16 | 29817097 | 29818243 |
| C16 | 29911887 | 29913161 |
| C16 | 30022756 | 30024036 |
| C16 | 30078104 | 30079336 |
| C16 | 30086065 | 30087221 |
| C16 | 30180824 | 30182540 |
| C16 | 30401666 | 30402856 |
| C16 | 30597441 | 30598752 |
| C16 | 30975600 | 30976729 |
| C16 | 31116121 | 31117487 |
| C16 | 31127180 | 31128298 |
| C16 | 31180533 | 31181714 |
| C16 | 31438843 | 31439997 |
| C16 | 31453040 | 31454116 |
| C16 | 31476919 | 31477987 |
| C16 | 31488212 | 31489278 |
| C16 | 31499824 | 31500884 |
| C16 | 31846891 | 31847951 |
| C16 | 32034836 | 32036230 |
| C16 | 32095369 | 32096495 |
| C16 | 32095596 | 32096650 |
| C16 | 32096274 | 32097369 |
| C16 | 32168843 | 32170511 |
| C16 | 32198902 | 32199952 |
| C16 | 32199121 | 32200175 |
| C16 | 32225408 | 32227679 |
| C16 | 32263591 | 32264822 |
| C16 | 32264032 | 32265398 |
| C16 | 32289652 | 32290781 |
| C16 | 32319066 | 32320828 |
| C16 | 32320521 | 32321895 |
| C16 | 32661580 | 32662990 |
| C16 | 32715699 | 32716940 |
| C16 | 32728228 | 32730211 |
| C16 | 32751893 | 32753204 |
| C16 | 32822468 | 32823519 |
| C16 | 32822721 | 32823792 |
| C16 | 32894069 | 32895669 |
| C16 | 32895381 | 32896653 |
| C16 | 32896576 | 32897638 |
| C16 | 32961510 | 32963285 |
| C16 | 32976483 | 32978519 |
| C16 | 33038365 | 33039870 |
| C16 | 33039120 | 33040271 |
| C16 | 33069680 | 33070746 |
| C16 | 33070249 | 33071349 |
| C16 | 33176160 | 33177312 |
| C16 | 33318678 | 33319733 |
| C16 | 33329921 | 33331755 |
| C16 | 33572592 | 33573647 |
| C16 | 33572832 | 33573885 |
| C16 | 33573095 | 33574153 |
| C16 | 33700871 | 33702479 |
| C16 | 33778116 | 33779595 |
| C16 | 33852083 | 33853190 |
| C16 | 33938450 | 33939532 |
| C16 | 33962348 | 33964121 |
| C16 | 33963384 | 33964562 |
| C16 | 33964124 | 33965223 |
| C16 | 33964525 | 33965631 |
| C16 | 34427063 | 34428103 |
| C16 | 46473514 | 46474576 |
| C16 | 46877798 | 46878881 |
| C16 | 46877970 | 46879040 |
| C16 | 46918632 | 46919902 |
| C16 | 47169926 | 47171064 |
| C16 | 48985418 | 48986717 |
| C16 | 49498778 | 49499897 |
| C16 | 49639062 | 49640100 |
| C16 | 49661043 | 49662049 |
| C16 | 49710488 | 49711500 |
| C16 | 49909674 | 49910748 |
| C16 | 50537613 | 50538635 |
| C16 | 50816392 | 50817439 |
| C16 | 51129454 | 51130648 |
| C16 | 51187846 | 51188890 |
| C16 | 52034229 | 52035488 |
| C16 | 52366507 | 52367518 |
| C16 | 52414482 | 52415573 |
| C16 | 53504982 | 53506006 |
| C16 | 53682015 | 53683038 |
| C16 | 53856512 | 53857854 |
| C16 | 54075666 | 54076834 |
| C16 | 54685080 | 54686362 |
| C16 | 55362238 | 55363361 |
| C16 | 55794023 | 55795491 |
| C16 | 56650683 | 56651739 |
| C16 | 56772582 | 56773747 |
| C16 | 56891884 | 56893055 |
| C16 | 57012385 | 57013436 |
| C16 | 57278843 | 57279929 |
| C16 | 57562076 | 57563247 |
| C16 | 57562434 | 57563485 |
| C16 | 57562567 | 57563825 |
| C16 | 57831173 | 57832341 |
| C16 | 57974589 | 57975982 |
| C16 | 58318877 | 58319939 |
| C16 | 59031836 | 59033010 |
| C16 | 60057949 | 60058968 |
| C16 | 60556495 | 60557648 |
| C16 | 60681058 | 60682227 |
| C16 | 62775880 | 62777458 |
| C16 | 63183858 | 63186263 |
| C16 | 63210589 | 63211846 |
| C16 | 63650638 | 63652163 |
| C16 | 65154762 | 65156056 |
| C16 | 66955622 | 66956741 |
| C16 | 66958829 | 66959995 |
| C16 | 67232468 | 67233624 |
| C16 | 67328629 | 67329649 |
| C16 | 67429670 | 67430859 |
| C16 | 67513315 | 67514559 |
| C16 | 67562202 | 67563253 |
| C16 | 67681636 | 67682740 |
| C16 | 67681808 | 67683167 |
| C16 | 67686172 | 67687255 |
| C16 | 67686264 | 67687395 |
| C16 | 67686458 | 67687513 |
| C16 | 67686840 | 67687925 |
| C16 | 67729851 | 67731119 |
| C16 | 67918125 | 67919394 |
| C16 | 67955490 | 67956742 |
| C16 | 68001678 | 68002766 |
| C16 | 68146048 | 68147130 |
| C16 | 68390032 | 68391373 |
| C16 | 68881128 | 68882358 |
| C16 | 69188279 | 69189293 |
| C16 | 69344549 | 69345720 |
| C16 | 69998538 | 69999639 |
| C16 | 70112333 | 70113854 |
| C16 | 70222172 | 70223427 |
| C16 | 70238701 | 70239918 |
| C16 | 70321356 | 70322676 |
| C16 | 70321713 | 70322811 |
| C16 | 70381196 | 70382372 |
| C16 | 70484258 | 70485657 |
| C16 | 71068385 | 71069457 |
| C16 | 71070197 | 71071789 |
| C16 | 71323549 | 71324759 |
| C16 | 72042673 | 72043819 |
| C16 | 72678773 | 72679783 |
| C16 | 72752939 | 72754093 |
| C16 | 72820937 | 72822133 |
| C16 | 72822021 | 72823274 |
| C16 | 73724850 | 73725923 |
| C16 | 75019551 | 75020686 |
| C16 | 75032526 | 75033579 |
| C16 | 75147649 | 75149136 |
| C16 | 75461886 | 75462971 |
| C16 | 75498035 | 75499085 |
| C16 | 75529721 | 75530969 |
| C16 | 75551356 | 75552582 |
| C16 | 75915103 | 75916189 |
| C16 | 77080640 | 77081994 |
| C16 | 77707373 | 77708759 |
| C16 | 77818147 | 77819292 |
| C16 | 77900635 | 77901663 |
| C16 | 78351139 | 78352793 |
| C16 | 78559597 | 78560634 |
| C16 | 78775974 | 78777148 |
| C16 | 78899931 | 78900955 |
| C16 | 79001503 | 79002575 |
| C16 | 79252406 | 79254114 |
| C16 | 79446765 | 79447910 |
| C16 | 79500897 | 79501963 |
| C16 | 79625139 | 79626167 |
| C16 | 79661276 | 79662297 |
| C16 | 80093784 | 80095766 |
| C16 | 80271225 | 80272450 |
| C16 | 81416193 | 81417200 |
| C16 | 81525522 | 81527319 |
| C16 | 81532899 | 81534258 |
| C16 | 81556743 | 81557770 |
| C16 | 81586630 | 81587827 |
| C16 | 81742884 | 81743978 |
| C16 | 81752310 | 81753524 |
| C16 | 82076645 | 82077745 |
| C16 | 82979685 | 82980732 |
| C16 | 83986930 | 83988119 |
| C16 | 84002187 | 84003315 |
| C16 | 84429368 | 84430692 |
| C16 | 84540525 | 84541775 |
| C16 | 84577595 | 84578658 |
| C16 | 84627698 | 84628789 |
| C16 | 84944528 | 84945606 |
| C16 | 84944637 | 84945821 |
| C16 | 85025765 | 85026800 |
| C16 | 85115812 | 85117534 |
| C16 | 85157313 | 85158386 |
| C16 | 85168008 | 85169115 |
| C16 | 85296147 | 85297204 |
| C16 | 85324674 | 85325751 |
| C16 | 85361030 | 85362621 |
| C16 | 85382515 | 85384120 |
| C16 | 85388101 | 85389260 |
| C16 | 85435088 | 85436275 |
| C16 | 85495894 | 85497045 |
| C16 | 85525124 | 85526311 |
| C16 | 85551001 | 85552264 |
| C16 | 85560705 | 85561764 |
| C16 | 85658412 | 85659474 |
| C16 | 85659066 | 85660486 |
| C16 | 85742986 | 85744150 |
| C16 | 85751828 | 85753039 |
| C16 | 85845262 | 85846371 |
| C16 | 85916924 | 85918299 |
| C16 | 85981347 | 85982448 |
| C16 | 86099997 | 86101062 |
| C16 | 86283841 | 86285190 |
| C16 | 86599766 | 86600841 |
| C16 | 86613021 | 86614097 |
| C16 | 86766182 | 86767328 |
| C16 | 86771627 | 86772721 |
| C16 | 86773635 | 86774690 |
| C16 | 86869418 | 86870758 |
| C16 | 86911959 | 86913038 |
| C16 | 87091814 | 87093147 |
| C16 | 87196075 | 87197304 |
| C16 | 87250106 | 87251482 |
| C16 | 87338705 | 87340460 |
| C16 | 87378324 | 87379643 |
| C16 | 87440888 | 87442116 |
| C16 | 87468689 | 87469847 |
| C16 | 87647942 | 87649040 |
| C16 | 87681465 | 87682634 |
| C16 | 87704762 | 87706173 |
| C16 | 87866124 | 87867309 |
| C16 | 87886036 | 87887158 |
| C16 | 87983108 | 87984317 |
| C16 | 88184471 | 88185600 |
| C16 | 88217330 | 88218595 |
| C16 | 88217753 | 88218854 |
| C16 | 88219775 | 88220815 |
| C16 | 88262674 | 88263763 |
| C16 | 88365373 | 88366576 |
| C16 | 88442228 | 88443668 |
| C16 | 88518817 | 88519904 |
| C16 | 88536460 | 88537673 |
| C16 | 88539534 | 88540664 |
| C16 | 88556000 | 88557421 |
| C16 | 88559261 | 88560442 |
| C16 | 88560349 | 88561450 |
| C16 | 88563612 | 88564786 |
| C16 | 88563823 | 88564893 |
| C16 | 88577549 | 88579010 |
| C16 | 88599392 | 88600485 |
| C16 | 88599655 | 88600799 |
| C16 | 88599946 | 88601122 |
| C16 | 88612800 | 88614423 |
| C16 | 88624307 | 88625516 |
| C16 | 88705861 | 88706927 |
| C16 | 88709107 | 88710237 |
| C16 | 88768000 | 88769067 |
| C16 | 88769241 | 88770532 |
| C16 | 88843927 | 88845226 |
| C16 | 88857423 | 88858661 |
| C16 | 88859907 | 88861365 |
| C16 | 88868288 | 88869359 |
| C16 | 88869112 | 88870243 |
| C16 | 88889298 | 88890363 |
| C16 | 88905804 | 88907963 |
| C16 | 88947256 | 88948438 |
| C16 | 88949136 | 88950264 |
| C16 | 88980641 | 88981913 |
| C16 | 89002973 | 89004196 |
| C16 | 89044168 | 89045964 |
| C16 | 89052752 | 89053950 |
| C16 | 89055075 | 89056644 |
| C16 | 89065419 | 89066815 |
| C16 | 89065912 | 89067016 |
| C16 | 89100912 | 89102135 |
| C16 | 89117690 | 89119449 |
| C16 | 89136609 | 89137906 |
| C16 | 89143719 | 89144852 |
| C16 | 89149431 | 89151331 |
| C16 | 89185140 | 89186409 |
| C16 | 89292789 | 89293858 |
| C16 | 89299574 | 89300750 |
| C16 | 89321023 | 89322260 |
| C16 | 89396318 | 89397531 |
| C16 | 89435340 | 89436402 |
| C16 | 89642633 | 89643720 |
| C16 | 89653785 | 89654933 |
| C16 | 89707041 | 89708343 |
| C16 | 89713740 | 89714804 |
| C16 | 89830551 | 89831763 |
| C16 | 89918601 | 89919681 |
| C16 | 90015550 | 90016656 |
| C16 | 90148496 | 90149633 |
| C16 | 90235622 | 90236778 |
| C16 | 90250977 | 90252433 |
| C17 | 56588 | 57732 |
| C17 | 83045 | 85284 |
| C17 | 136385 | 137804 |
| C17 | 152815 | 154520 |
| C17 | 153907 | 155075 |
| C17 | 184270 | 185396 |
| C17 | 210749 | 211975 |
| C17 | 213888 | 215145 |
| C17 | 216877 | 218051 |
| C17 | 224777 | 226019 |
| C17 | 405749 | 406858 |
| C17 | 438130 | 439205 |
| C17 | 623976 | 625013 |
| C17 | 649708 | 650810 |
| C17 | 655840 | 656934 |
| C17 | 656007 | 657114 |
| C17 | 714868 | 715961 |
| C17 | 738322 | 739505 |
| C17 | 803753 | 805009 |
| C17 | 824088 | 825245 |
| C17 | 830276 | 831572 |
| C17 | 847844 | 849010 |
| C17 | 851379 | 852471 |
| C17 | 874895 | 875901 |
| C17 | 909566 | 910852 |
| C17 | 913498 | 914560 |
| C17 | 934196 | 935501 |
| C17 | 934503 | 935588 |
| C17 | 950075 | 951197 |
| C17 | 998359 | 999448 |
| C17 | 1028090 | 1029345 |
| C17 | 1051583 | 1052684 |
| C17 | 1056136 | 1057400 |
| C17 | 1116420 | 1117493 |
| C17 | 1123132 | 1124259 |
| C17 | 1150109 | 1151249 |
| C17 | 1213981 | 1215174 |
| C17 | 1306090 | 1307234 |
| C17 | 1360101 | 1361314 |
| C17 | 1478492 | 1479849 |
| C17 | 1507659 | 1508789 |
| C17 | 1509424 | 1510506 |
| C17 | 1545850 | 1546987 |
| C17 | 1568288 | 1569366 |
| C17 | 1686096 | 1687135 |
| C17 | 1686171 | 1687292 |
| C17 | 1743280 | 1744360 |
| C17 | 1839643 | 1840693 |
| C17 | 1953864 | 1954945 |
| C17 | 1959322 | 1960711 |
| C17 | 1960258 | 1961360 |
| C17 | 1960749 | 1961867 |
| C17 | 1961122 | 1962216 |
| C17 | 2014990 | 2016195 |
| C17 | 2299478 | 2300665 |
| C17 | 2350711 | 2351903 |
| C17 | 2593054 | 2594101 |
| C17 | 2594749 | 2595844 |
| C17 | 2736300 | 2737479 |
| C17 | 3140052 | 3142068 |
| C17 | 3186738 | 3188069 |
| C17 | 3274659 | 3275782 |
| C17 | 3289053 | 3290115 |
| C17 | 3322969 | 3324639 |
| C17 | 3438314 | 3439425 |
| C17 | 3539146 | 3540255 |
| C17 | 3555790 | 3556834 |
| C17 | 3571512 | 3572568 |
| C17 | 3588565 | 3589671 |
| C17 | 3588678 | 3589839 |
| C17 | 3589269 | 3590603 |
| C17 | 3637013 | 3638119 |
| C17 | 3643817 | 3644997 |
| C17 | 3705375 | 3706637 |
| C17 | 3818630 | 3819947 |
| C17 | 3847799 | 3848998 |
| C17 | 3886798 | 3888250 |
| C17 | 3906813 | 3907925 |
| C17 | 3907150 | 3908195 |
| C17 | 4112374 | 4113427 |
| C17 | 4125024 | 4126097 |
| C17 | 4422239 | 4423515 |
| C17 | 4481557 | 4482791 |
| C17 | 4542703 | 4543761 |
| C17 | 4559568 | 4560654 |
| C17 | 4612021 | 4613318 |
| C17 | 4617447 | 4618972 |
| C17 | 4620485 | 4621725 |
| C17 | 4642837 | 4643954 |
| C17 | 4736109 | 4737159 |
| C17 | 4804411 | 4805591 |
| C17 | 4804618 | 4805710 |
| C17 | 4804727 | 4805867 |
| C17 | 5015309 | 5016441 |
| C17 | 5015493 | 5016620 |
| C17 | 5025893 | 5026956 |
| C17 | 5673632 | 5675238 |
| C17 | 5674382 | 5675469 |
| C17 | 5933326 | 5934420 |
| C17 | 6219836 | 6220868 |
| C17 | 6444175 | 6445235 |
| C17 | 6557700 | 6559609 |
| C17 | 6796952 | 6798157 |
| C17 | 6898914 | 6900069 |
| C17 | 7076919 | 7078570 |
| C17 | 7210536 | 7211599 |
| C17 | 7252748 | 7254022 |
| C17 | 7253076 | 7254256 |
| C17 | 7253619 | 7254773 |
| C17 | 7253880 | 7255085 |
| C17 | 7311161 | 7312525 |
| C17 | 7559718 | 7560912 |
| C17 | 7564597 | 7565832 |
| C17 | 7568904 | 7570062 |
| C17 | 7571220 | 7573508 |
| C17 | 7573427 | 7574533 |
| C17 | 7576025 | 7577157 |
| C17 | 7576353 | 7577426 |
| C17 | 7576519 | 7577655 |
| C17 | 7576999 | 7578108 |
| C17 | 7577344 | 7578789 |
| C17 | 7577871 | 7579311 |
| C17 | 7578812 | 7580090 |
| C17 | 7579200 | 7580440 |
| C17 | 7580143 | 7581252 |
| C17 | 7590195 | 7591356 |
| C17 | 7643198 | 7644407 |
| C17 | 7757863 | 7758948 |
| C17 | 7758349 | 7759452 |
| C17 | 7761344 | 7762516 |
| C17 | 7813672 | 7814745 |
| C17 | 7832180 | 7833326 |
| C17 | 8060309 | 8061820 |
| C17 | 8129452 | 8130756 |
| C17 | 8218271 | 8219392 |
| C17 | 8249959 | 8251485 |
| C17 | 8383754 | 8384983 |
| C17 | 8565141 | 8566161 |
| C17 | 8953567 | 8955238 |
| C17 | 9018311 | 9019689 |
| C17 | 9449296 | 9450501 |
| C17 | 9519221 | 9520294 |
| C17 | 9784358 | 9785477 |
| C17 | 9979072 | 9980643 |
| C17 | 10292354 | 10293556 |
| C17 | 10449821 | 10450863 |
| C17 | 11143786 | 11145250 |
| C17 | 11253019 | 11254122 |
| C17 | 11380806 | 11383012 |
| C17 | 11565633 | 11566824 |
| C17 | 11680863 | 11681867 |
| C17 | 13367491 | 13368532 |
| C17 | 13446137 | 13447206 |
| C17 | 13505583 | 13506774 |
| C17 | 13683960 | 13685050 |
| C17 | 14095912 | 14097325 |
| C17 | 14114734 | 14115899 |
| C17 | 14197865 | 14199052 |
| C17 | 14207094 | 14208290 |
| C17 | 14212725 | 14214019 |
| C17 | 14462041 | 14463578 |
| C17 | 14639136 | 14640748 |
| C17 | 15244538 | 15245625 |
| C17 | 15689280 | 15690547 |
| C17 | 16367589 | 16368709 |
| C17 | 16389335 | 16390458 |
| C17 | 16483548 | 16484720 |
| C17 | 16586273 | 16587359 |
| C17 | 16649585 | 16650658 |
| C17 | 16732465 | 16734237 |
| C17 | 17141426 | 17142677 |
| C17 | 17203518 | 17204678 |
| C17 | 17306782 | 17308162 |
| C17 | 17387357 | 17388426 |
| C17 | 17414903 | 17416235 |
| C17 | 17603485 | 17604679 |
| C17 | 17625177 | 17626383 |
| C17 | 17660825 | 17662038 |
| C17 | 17712813 | 17714383 |
| C17 | 17715462 | 17716669 |
| C17 | 18023376 | 18025023 |
| C17 | 18085388 | 18086542 |
| C17 | 18338999 | 18340270 |
| C17 | 18357421 | 18358544 |
| C17 | 18405264 | 18407131 |
| C17 | 18527984 | 18529089 |
| C17 | 18528138 | 18529374 |
| C17 | 18537710 | 18538841 |
| C17 | 18642772 | 18644542 |
| C17 | 18720753 | 18722323 |
| C17 | 18736994 | 18738192 |
| C17 | 18791585 | 18792641 |
| C17 | 19428754 | 19430026 |
| C17 | 19530081 | 19531519 |
| C17 | 19627345 | 19628532 |
| C17 | 19627680 | 19628797 |
| C17 | 19869093 | 19870113 |
| C17 | 19882928 | 19884193 |
| C17 | 20430632 | 20432276 |
| C17 | 20491904 | 20492963 |
| C17 | 20504983 | 20506223 |
| C17 | 20687070 | 20688203 |
| C17 | 20687261 | 20688348 |
| C17 | 20718672 | 20719740 |
| C17 | 20743589 | 20744804 |
| C17 | 20744143 | 20745261 |
| C17 | 20746335 | 20747393 |
| C17 | 20798970 | 20800044 |
| C17 | 20888304 | 20889409 |
| C17 | 21146577 | 21147970 |
| C17 | 21319266 | 21320292 |
| C17 | 21476984 | 21478434 |
| C17 | 21902935 | 21904014 |
| C17 | 22019979 | 22021305 |
| C17 | 25289158 | 25290295 |
| C17 | 25539114 | 25540344 |
| C17 | 25676012 | 25677176 |
| C17 | 25778901 | 25780026 |
| C17 | 25793264 | 25794515 |
| C17 | 26698696 | 26699798 |
| C17 | 26707749 | 26709237 |
| C17 | 27312739 | 27313851 |
| C17 | 27899012 | 27900062 |
| C17 | 27899140 | 27900342 |
| C17 | 27938945 | 27940400 |
| C17 | 27939461 | 27940626 |
| C17 | 27939721 | 27941192 |
| C17 | 27943282 | 27944396 |
| C17 | 28256834 | 28257906 |
| C17 | 28468435 | 28469479 |
| C17 | 29141375 | 29142729 |
| C17 | 29671957 | 29673084 |
| C17 | 30243700 | 30244824 |
| C17 | 31062987 | 31064551 |
| C17 | 31149074 | 31150313 |
| C17 | 31248670 | 31249762 |
| C17 | 31249374 | 31250468 |
| C17 | 31340110 | 31341332 |
| C17 | 31477228 | 31478347 |
| C17 | 31763414 | 31764541 |
| C17 | 32225079 | 32226101 |
| C17 | 32366609 | 32367744 |
| C17 | 32624614 | 32625737 |
| C17 | 33477616 | 33478861 |
| C17 | 33776273 | 33777387 |
| C17 | 33841698 | 33842756 |
| C17 | 34274041 | 34275103 |
| C17 | 34498703 | 34500296 |
| C17 | 34584436 | 34585774 |
| C17 | 34586804 | 34588340 |
| C17 | 34617476 | 34619359 |
| C17 | 34745810 | 34747522 |
| C17 | 34795377 | 34796687 |
| C17 | 34819848 | 34821474 |
| C17 | 34964841 | 34965950 |
| C17 | 34998090 | 34999286 |
| C17 | 35143079 | 35144214 |
| C17 | 35148763 | 35149945 |
| C17 | 35284953 | 35286201 |
| C17 | 35299544 | 35300598 |
| C17 | 35299877 | 35300956 |
| C17 | 35754511 | 35755891 |
| C17 | 35848798 | 35849839 |
| C17 | 35873249 | 35874373 |
| C17 | 36104835 | 36106214 |
| C17 | 36259306 | 36260423 |
| C17 | 36300492 | 36303988 |
| C17 | 36347891 | 36349214 |
| C17 | 36361290 | 36362372 |
| C17 | 36377094 | 36378143 |
| C17 | 36387353 | 36388387 |
| C17 | 36408791 | 36410047 |
| C17 | 36476129 | 36477164 |
| C17 | 36584675 | 36586290 |
| C17 | 36609206 | 36610281 |
| C17 | 36665582 | 36666684 |
| C17 | 36688613 | 36689662 |
| C17 | 36717302 | 36719961 |
| C17 | 36718987 | 36720437 |
| C17 | 36719844 | 36720981 |
| C17 | 36739548 | 36740784 |
| C17 | 36750974 | 36752063 |
| C17 | 36762146 | 36763284 |
| C17 | 36813142 | 36814215 |
| C17 | 36814383 | 36816173 |
| C17 | 36825478 | 36826926 |
| C17 | 36826658 | 36828277 |
| C17 | 36830804 | 36831865 |
| C17 | 36847652 | 36848664 |
| C17 | 36876143 | 36877345 |
| C17 | 36880229 | 36881744 |
| C17 | 36882680 | 36883733 |
| C17 | 36883888 | 36885345 |
| C17 | 36886687 | 36888019 |
| C17 | 36923885 | 36925695 |
| C17 | 36980161 | 36981353 |
| C17 | 36996354 | 36998070 |
| C17 | 37000205 | 37001739 |
| C17 | 37053861 | 37056564 |
| C17 | 37057541 | 37058680 |
| C17 | 37086411 | 37087539 |
| C17 | 37311244 | 37312373 |
| C17 | 37320875 | 37322569 |
| C17 | 37325074 | 37326171 |
| C17 | 37331506 | 37332625 |
| C17 | 37331697 | 37332856 |
| C17 | 37361338 | 37362447 |
| C17 | 37365009 | 37367383 |
| C17 | 37368675 | 37370008 |
| C17 | 37380407 | 37381461 |
| C17 | 37482758 | 37484615 |
| C17 | 37490535 | 37492831 |
| C17 | 37493732 | 37495263 |
| C17 | 37519488 | 37520804 |
| C17 | 37568577 | 37570101 |
| C17 | 37719330 | 37720722 |
| C17 | 37728789 | 37729976 |
| C17 | 37759964 | 37761047 |
| C17 | 37761076 | 37762197 |
| C17 | 37763593 | 37764658 |
| C17 | 37782105 | 37783365 |
| C17 | 37782988 | 37784379 |
| C17 | 37785895 | 37786937 |
| C17 | 37796553 | 37797631 |
| C17 | 37815473 | 37817006 |
| C17 | 37818626 | 37819843 |
| C17 | 37831109 | 37832696 |
| C17 | 37835200 | 37836744 |
| C17 | 37842764 | 37844488 |
| C17 | 37843667 | 37845031 |
| C17 | 37844449 | 37845553 |
| C17 | 37849014 | 37850078 |
| C17 | 37850434 | 37851934 |
| C17 | 37855290 | 37856340 |
| C17 | 37855729 | 37857064 |
| C17 | 37856753 | 37858335 |
| C17 | 37857364 | 37863372 |
| C17 | 37862502 | 37864130 |
| C17 | 37863157 | 37865287 |
| C17 | 37864997 | 37866205 |
| C17 | 37865566 | 37866634 |
| C17 | 37865839 | 37866954 |
| C17 | 37866093 | 37867234 |
| C17 | 37867681 | 37869201 |
| C17 | 37868906 | 37870022 |
| C17 | 37871039 | 37872112 |
| C17 | 37871199 | 37872289 |
| C17 | 37871493 | 37873358 |
| C17 | 37872727 | 37874410 |
| C17 | 37875540 | 37877050 |
| C17 | 37879072 | 37880210 |
| C17 | 37879291 | 37880413 |
| C17 | 37879665 | 37880763 |
| C17 | 37880479 | 37881664 |
| C17 | 37880802 | 37883104 |
| C17 | 37882315 | 37883412 |
| C17 | 37882568 | 37883756 |
| C17 | 37883048 | 37884300 |
| C17 | 37883442 | 37885415 |
| C17 | 37885001 | 37886008 |
| C17 | 37885776 | 37887179 |
| C17 | 37886708 | 37887969 |
| C17 | 37896014 | 37897357 |
| C17 | 38023790 | 38025093 |
| C17 | 38099807 | 38101091 |
| C17 | 38104734 | 38106329 |
| C17 | 38122884 | 38123898 |
| C17 | 38123213 | 38124587 |
| C17 | 38166245 | 38168120 |
| C17 | 38263107 | 38265493 |
| C17 | 38332323 | 38333394 |
| C17 | 38333777 | 38334904 |
| C17 | 38334032 | 38335343 |
| C17 | 38472374 | 38473590 |
| C17 | 38472601 | 38473697 |
| C17 | 38532790 | 38533964 |
| C17 | 38574281 | 38575459 |
| C17 | 38577081 | 38578191 |
| C17 | 38582002 | 38583212 |
| C17 | 38656621 | 38658434 |
| C17 | 38668191 | 38669458 |
| C17 | 38703948 | 38706756 |
| C17 | 38707965 | 38709214 |
| C17 | 38711393 | 38712743 |
| C17 | 38712870 | 38714371 |
| C17 | 38773718 | 38776477 |
| C17 | 38874775 | 38875839 |
| C17 | 38935247 | 38936685 |
| C17 | 39018888 | 39019986 |
| C17 | 39059282 | 39061112 |
| C17 | 39112287 | 39115397 |
| C17 | 39142497 | 39143982 |
| C17 | 39202517 | 39204002 |
| C17 | 39204211 | 39206525 |
| C17 | 39215455 | 39216797 |
| C17 | 39276113 | 39277636 |
| C17 | 39282963 | 39283998 |
| C17 | 39464138 | 39465831 |
| C17 | 39803145 | 39804317 |
| C17 | 39925742 | 39930058 |
| C17 | 40191521 | 40192803 |
| C17 | 40274366 | 40275525 |
| C17 | 40439190 | 40440591 |
| C17 | 40440323 | 40442288 |
| C17 | 40609737 | 40610963 |
| C17 | 40714523 | 40715602 |
| C17 | 40811490 | 40812697 |
| C17 | 40821667 | 40822929 |
| C17 | 40822069 | 40823410 |
| C17 | 40822531 | 40823777 |
| C17 | 40835322 | 40836575 |
| C17 | 40836573 | 40837623 |
| C17 | 41116563 | 41118170 |
| C17 | 41124738 | 41125925 |
| C17 | 41320756 | 41321964 |
| C17 | 41476718 | 41477878 |
| C17 | 41673343 | 41674389 |
| C17 | 42245788 | 42246867 |
| C17 | 42287388 | 42288471 |
| C17 | 42314436 | 42315567 |
| C17 | 42402963 | 42404135 |
| C17 | 42403195 | 42404340 |
| C17 | 42430546 | 42432281 |
| C17 | 42431359 | 42432612 |
| C17 | 42451824 | 42452973 |
| C17 | 42646050 | 42647297 |
| C17 | 42863844 | 42864942 |
| C17 | 42963576 | 42964658 |
| C17 | 43044222 | 43045585 |
| C17 | 43061330 | 43062655 |
| C17 | 43197829 | 43199272 |
| C17 | 43224369 | 43225465 |
| C17 | 43253470 | 43254903 |
| C17 | 43317856 | 43319012 |
| C17 | 43318609 | 43319646 |
| C17 | 43318692 | 43319994 |
| C17 | 43350451 | 43351520 |
| C17 | 43506759 | 43507827 |
| C17 | 43507076 | 43508167 |
| C17 | 43510697 | 43511822 |
| C17 | 43578122 | 43579155 |
| C17 | 43617169 | 43618431 |
| C17 | 43659813 | 43661640 |
| C17 | 43678956 | 43680058 |
| C17 | 44073333 | 44074450 |
| C17 | 44251175 | 44252599 |
| C17 | 44846685 | 44847889 |
| C17 | 44918675 | 44919824 |
| C17 | 45330452 | 45331605 |
| C17 | 45427878 | 45429189 |
| C17 | 45785666 | 45786898 |
| C17 | 45785974 | 45787047 |
| C17 | 45810810 | 45812036 |
| C17 | 45968089 | 45969167 |
| C17 | 46076243 | 46077257 |
| C17 | 46102224 | 46103341 |
| C17 | 46233055 | 46234325 |
| C17 | 46589594 | 46591325 |
| C17 | 46619294 | 46620482 |
| C17 | 46624832 | 46626313 |
| C17 | 46627403 | 46628543 |
| C17 | 46631592 | 46632683 |
| C17 | 46632668 | 46633968 |
| C17 | 46641239 | 46642270 |
| C17 | 46648007 | 46649116 |
| C17 | 46673895 | 46675040 |
| C17 | 46681808 | 46682925 |
| C17 | 46684792 | 46686082 |
| C17 | 46687552 | 46688761 |
| C17 | 46692761 | 46693913 |
| C17 | 46795797 | 46796829 |
| C17 | 46829551 | 46830690 |
| C17 | 47020717 | 47021961 |
| C17 | 47072493 | 47073664 |
| C17 | 47072718 | 47073881 |
| C17 | 47075153 | 47076258 |
| C17 | 47075511 | 47076722 |
| C17 | 47090718 | 47092307 |
| C17 | 47091545 | 47092678 |
| C17 | 47093455 | 47094980 |
| C17 | 47112682 | 47114132 |
| C17 | 47169718 | 47171724 |
| C17 | 47176924 | 47178057 |
| C17 | 47189007 | 47190088 |
| C17 | 47228167 | 47229563 |
| C17 | 47270252 | 47272161 |
| C17 | 47287021 | 47288685 |
| C17 | 47291486 | 47292861 |
| C17 | 47296048 | 47297413 |
| C17 | 47301658 | 47302942 |
| C17 | 47306762 | 47308074 |
| C17 | 47308006 | 47309311 |
| C17 | 47464003 | 47465303 |
| C17 | 47492605 | 47493653 |
| C17 | 47546853 | 47547975 |
| C17 | 47754400 | 47755498 |
| C17 | 47864563 | 47866042 |
| C17 | 47927587 | 47928815 |
| C17 | 47950429 | 47951453 |
| C17 | 47963686 | 47965164 |
| C17 | 47967468 | 47968591 |
| C17 | 47967785 | 47968884 |
| C17 | 47986332 | 47987850 |
| C17 | 48041555 | 48042853 |
| C17 | 48042000 | 48043195 |
| C17 | 48042336 | 48043563 |
| C17 | 48048454 | 48049545 |
| C17 | 48049579 | 48051505 |
| C17 | 48070273 | 48071532 |
| C17 | 48086925 | 48087982 |
| C17 | 48127700 | 48128732 |
| C17 | 48153516 | 48154706 |
| C17 | 48177849 | 48179181 |
| C17 | 48178256 | 48179500 |
| C17 | 48189785 | 48190973 |
| C17 | 48192744 | 48194212 |
| C17 | 48195033 | 48196167 |
| C17 | 48204357 | 48205922 |
| C17 | 48276383 | 48277472 |
| C17 | 48434538 | 48435564 |
| C17 | 48545569 | 48546717 |
| C17 | 48545771 | 48547539 |
| C17 | 48552943 | 48554232 |
| C17 | 48564264 | 48565922 |
| C17 | 48769840 | 48771686 |
| C17 | 48858136 | 48859438 |
| C17 | 49241720 | 49242793 |
| C17 | 49651299 | 49652360 |
| C17 | 50363708 | 50364901 |
| C17 | 51673620 | 51674688 |
| C17 | 53341206 | 53342733 |
| C17 | 53342857 | 53344231 |
| C17 | 53920341 | 53921448 |
| C17 | 54119418 | 54120479 |
| C17 | 54587494 | 54588653 |
| C17 | 54755465 | 54756535 |
| C17 | 55213220 | 55214469 |
| C17 | 56139212 | 56140257 |
| C17 | 56160032 | 56161185 |
| C17 | 56160290 | 56161421 |
| C17 | 56219262 | 56220423 |
| C17 | 56264363 | 56265412 |
| C17 | 56344177 | 56345864 |
| C17 | 56354708 | 56355971 |
| C17 | 56404954 | 56406535 |
| C17 | 56407805 | 56409086 |
| C17 | 56409462 | 56411208 |
| C17 | 56410297 | 56411964 |
| C17 | 56564522 | 56565875 |
| C17 | 56584057 | 56585219 |
| C17 | 56596173 | 56597634 |
| C17 | 57285899 | 57287439 |
| C17 | 57385864 | 57387016 |
| C17 | 57915847 | 57917843 |
| C17 | 58075346 | 58076370 |
| C17 | 58085454 | 58086774 |
| C17 | 58085887 | 58087022 |
| C17 | 58179532 | 58180612 |
| C17 | 58187047 | 58189304 |
| C17 | 58498212 | 58499334 |
| C17 | 58604426 | 58605468 |
| C17 | 58797288 | 58798297 |
| C17 | 58964062 | 58965184 |
| C17 | 59360602 | 59361614 |
| C17 | 59475275 | 59476430 |
| C17 | 59480690 | 59481875 |
| C17 | 59487740 | 59488868 |
| C17 | 59529207 | 59530377 |
| C17 | 59531844 | 59532972 |
| C17 | 59533319 | 59534458 |
| C17 | 59533823 | 59534977 |
| C17 | 59534998 | 59536023 |
| C17 | 60013301 | 60015127 |
| C17 | 60215393 | 60216860 |
| C17 | 60216019 | 60217161 |
| C17 | 60555753 | 60556837 |
| C17 | 60757347 | 60758787 |
| C17 | 61496890 | 61498076 |
| C17 | 61512852 | 61514056 |
| C17 | 61777701 | 61778899 |
| C17 | 61925733 | 61927273 |
| C17 | 62095604 | 62096731 |
| C17 | 62686728 | 62687734 |
| C17 | 62696167 | 62697770 |
| C17 | 62711831 | 62712923 |
| C17 | 62774285 | 62775467 |
| C17 | 62892920 | 62894239 |
| C17 | 63224415 | 63225893 |
| C17 | 63770085 | 63772036 |
| C17 | 64264396 | 64265652 |
| C17 | 64298954 | 64300209 |
| C17 | 64312052 | 64313062 |
| C17 | 64571455 | 64572463 |
| C17 | 64741534 | 64742585 |
| C17 | 64860717 | 64862548 |
| C17 | 65273195 | 65274432 |
| C17 | 65486824 | 65487968 |
| C17 | 65516074 | 65517370 |
| C17 | 65610689 | 65612096 |
| C17 | 66194054 | 66195195 |
| C17 | 66470302 | 66471478 |
| C17 | 66575843 | 66577049 |
| C17 | 66676139 | 66677568 |
| C17 | 66812695 | 66814201 |
| C17 | 66823142 | 66824361 |
| C17 | 66876495 | 66877524 |
| C17 | 66941463 | 66943006 |
| C17 | 66980324 | 66981757 |
| C17 | 66981256 | 66982848 |
| C17 | 67087194 | 67088325 |
| C17 | 67141772 | 67142922 |
| C17 | 67833122 | 67834192 |
| C17 | 68092800 | 68095477 |
| C17 | 68124593 | 68125643 |
| C17 | 68164704 | 68165798 |
| C17 | 68295272 | 68296536 |
| C17 | 68305249 | 68307136 |
| C17 | 68311687 | 68313248 |
| C17 | 68448802 | 68450237 |
| C17 | 68454917 | 68456043 |
| C17 | 68542504 | 68544707 |
| C17 | 70106015 | 70107082 |
| C17 | 70214904 | 70216247 |
| C17 | 71143619 | 71145126 |
| C17 | 71277204 | 71278229 |
| C17 | 71338861 | 71340046 |
| C17 | 71390410 | 71392152 |
| C17 | 71451520 | 71452646 |
| C17 | 71537375 | 71538442 |
| C17 | 71582110 | 71583230 |
| C17 | 71623060 | 71624484 |
| C17 | 71715397 | 71716549 |
| C17 | 71863780 | 71864834 |
| C17 | 72107020 | 72108213 |
| C17 | 72208353 | 72209580 |
| C17 | 72269894 | 72271197 |
| C17 | 72352468 | 72353530 |
| C17 | 72359929 | 72361093 |
| C17 | 72368530 | 72369743 |
| C17 | 72462430 | 72463887 |
| C17 | 72523135 | 72524648 |
| C17 | 72665390 | 72666407 |
| C17 | 72775184 | 72776589 |
| C17 | 72907228 | 72908232 |
| C17 | 72915124 | 72916299 |
| C17 | 72915749 | 72917092 |
| C17 | 72920322 | 72921537 |
| C17 | 73020907 | 73022550 |
| C17 | 73072945 | 73074095 |
| C17 | 73073313 | 73074467 |
| C17 | 73427238 | 73428377 |
| C17 | 73584198 | 73585369 |
| C17 | 73615350 | 73617328 |
| C17 | 73631076 | 73632462 |
| C17 | 73749087 | 73750678 |
| C17 | 73989303 | 73990449 |
| C17 | 74017212 | 74018347 |
| C17 | 74072203 | 74073457 |
| C17 | 74299160 | 74300268 |
| C17 | 74473318 | 74474376 |
| C17 | 74493710 | 74494889 |
| C17 | 74511087 | 74512602 |
| C17 | 74558025 | 74559165 |
| C17 | 74564417 | 74565589 |
| C17 | 74684377 | 74685813 |
| C17 | 74866054 | 74867154 |
| C17 | 74987165 | 74988343 |
| C17 | 75142632 | 75143695 |
| C17 | 75284086 | 75285477 |
| C17 | 75315856 | 75316959 |
| C17 | 75320486 | 75321691 |
| C17 | 75462640 | 75463716 |
| C17 | 75463272 | 75464342 |
| C17 | 75551387 | 75552577 |
| C17 | 75612640 | 75613790 |
| C17 | 75711260 | 75712611 |
| C17 | 75720949 | 75722087 |
| C17 | 75772344 | 75773455 |
| C17 | 75793587 | 75795197 |
| C17 | 75811795 | 75812933 |
| C17 | 76036509 | 76037576 |
| C17 | 76117099 | 76118347 |
| C17 | 76120899 | 76122140 |
| C17 | 76137537 | 76139221 |
| C17 | 76170242 | 76171323 |
| C17 | 76182746 | 76183784 |
| C17 | 76210755 | 76211827 |
| C17 | 76258835 | 76260036 |
| C17 | 76348294 | 76349425 |
| C17 | 76360615 | 76361812 |
| C17 | 76415647 | 76416801 |
| C17 | 76417463 | 76418564 |
| C17 | 76440046 | 76441222 |
| C17 | 76446147 | 76447278 |
| C17 | 76449113 | 76450742 |
| C17 | 76461030 | 76462375 |
| C17 | 76464384 | 76465631 |
| C17 | 76497937 | 76499199 |
| C17 | 76547271 | 76548760 |
| C17 | 76594173 | 76595314 |
| C17 | 76712674 | 76713936 |
| C17 | 76822822 | 76824203 |
| C17 | 76882605 | 76883889 |
| C17 | 76886202 | 76887441 |
| C17 | 76895943 | 76897787 |
| C17 | 76909405 | 76910892 |
| C17 | 76915182 | 76916373 |
| C17 | 76927000 | 76928267 |
| C17 | 76930212 | 76931269 |
| C17 | 77023614 | 77024765 |
| C17 | 77029518 | 77031109 |
| C17 | 77085522 | 77087061 |
| C17 | 77175666 | 77176710 |
| C17 | 77183017 | 77184040 |
| C17 | 77193068 | 77194096 |
| C17 | 77206784 | 77207873 |
| C17 | 77218970 | 77220006 |
| C17 | 77302701 | 77303888 |
| C17 | 77371340 | 77372413 |
| C17 | 77393491 | 77394521 |
| C17 | 77413219 | 77415076 |
| C17 | 77472998 | 77474139 |
| C17 | 77482907 | 77484257 |
| C17 | 77726060 | 77727216 |
| C17 | 77749331 | 77750344 |
| C17 | 77754644 | 77756346 |
| C17 | 77766480 | 77767580 |
| C17 | 77772508 | 77773672 |
| C17 | 77801330 | 77802407 |
| C17 | 77895044 | 77896228 |
| C17 | 77899109 | 77900208 |
| C17 | 77900566 | 77902025 |
| C17 | 77997366 | 77998451 |
| C17 | 78165315 | 78166383 |
| C17 | 78233175 | 78234407 |
| C17 | 78610142 | 78611283 |
| C17 | 78752818 | 78753871 |
| C17 | 78753854 | 78755943 |
| C17 | 78793517 | 78795065 |
| C17 | 78865423 | 78866602 |
| C17 | 78915678 | 78916926 |
| C17 | 78932712 | 78934389 |
| C17 | 78962876 | 78963966 |
| C17 | 78970861 | 78971915 |
| C17 | 78970956 | 78972142 |
| C17 | 78996872 | 78998020 |
| C17 | 79000176 | 79001695 |
| C17 | 79000789 | 79002030 |
| C17 | 79009476 | 79010999 |
| C17 | 79017403 | 79018859 |
| C17 | 79022444 | 79023643 |
| C17 | 79068481 | 79069619 |
| C17 | 79092535 | 79093622 |
| C17 | 79092759 | 79093825 |
| C17 | 79093457 | 79094581 |
| C17 | 79095594 | 79096673 |
| C17 | 79095697 | 79096867 |
| C17 | 79099514 | 79100838 |
| C17 | 79100924 | 79102411 |
| C17 | 79102315 | 79103443 |
| C17 | 79170344 | 79171505 |
| C17 | 79243570 | 79244855 |
| C17 | 79250823 | 79252063 |
| C17 | 79258181 | 79260483 |
| C17 | 79272452 | 79273518 |
| C17 | 79315999 | 79317082 |
| C17 | 79427642 | 79428694 |
| C17 | 79427756 | 79428795 |
| C17 | 79465679 | 79466817 |
| C17 | 79481248 | 79482350 |
| C17 | 79495065 | 79496400 |
| C17 | 79495473 | 79496790 |
| C17 | 79614452 | 79615560 |
| C17 | 79619551 | 79621311 |
| C17 | 79707254 | 79708390 |
| C17 | 79798899 | 79799958 |
| C17 | 79801220 | 79802360 |
| C17 | 79825192 | 79826451 |
| C17 | 79849266 | 79850318 |
| C17 | 79878792 | 79880036 |
| C17 | 79887106 | 79888520 |
| C17 | 79893624 | 79895011 |
| C17 | 79896767 | 79898111 |
| C17 | 79898928 | 79900129 |
| C17 | 79919078 | 79920488 |
| C17 | 79924116 | 79925284 |
| C17 | 79924469 | 79925735 |
| C17 | 79924776 | 79926177 |
| C17 | 79935762 | 79937126 |
| C17 | 79938943 | 79941315 |
| C17 | 79944120 | 79945126 |
| C17 | 79944227 | 79945768 |
| C17 | 79949034 | 79950740 |
| C17 | 79956895 | 79958471 |
| C17 | 79960810 | 79962687 |
| C17 | 79962687 | 79963857 |
| C17 | 79969716 | 79970894 |
| C17 | 79970824 | 79972022 |
| C17 | 79971819 | 79973147 |
| C17 | 79981512 | 79982743 |
| C17 | 79981790 | 79983155 |
| C17 | 79987484 | 79988645 |
| C17 | 79991253 | 79993045 |
| C17 | 79993926 | 79995218 |
| C17 | 80022168 | 80023267 |
| C17 | 80023641 | 80024881 |
| C17 | 80038951 | 80039955 |
| C17 | 80040474 | 80041687 |
| C17 | 80046315 | 80047599 |
| C17 | 80046655 | 80047854 |
| C17 | 80047754 | 80049290 |
| C17 | 80048368 | 80049506 |
| C17 | 80048631 | 80050072 |
| C17 | 80050125 | 80051147 |
| C17 | 80050201 | 80051361 |
| C17 | 80050376 | 80052415 |
| C17 | 80051439 | 80055448 |
| C17 | 80057320 | 80058553 |
| C17 | 80058166 | 80059494 |
| C17 | 80060826 | 80064140 |
| C17 | 80174970 | 80176325 |
| C17 | 80202256 | 80203470 |
| C17 | 80260866 | 80262109 |
| C17 | 80263183 | 80264330 |
| C17 | 80266462 | 80267603 |
| C17 | 80289331 | 80290633 |
| C17 | 80331876 | 80332989 |
| C17 | 80342160 | 80343331 |
| C17 | 80346017 | 80347214 |
| C17 | 80400248 | 80401266 |
| C17 | 80571505 | 80572620 |
| C17 | 80593229 | 80594293 |
| C17 | 80695683 | 80696784 |
| C17 | 80707846 | 80709252 |
| C17 | 80740066 | 80741175 |
| C17 | 80764033 | 80765082 |
| C17 | 80797133 | 80798832 |
| C17 | 80840252 | 80841345 |
| C17 | 80844546 | 80845608 |
| C17 | 80844841 | 80845972 |
| C17 | 80846263 | 80848047 |
| C17 | 80871847 | 80872972 |
| C17 | 80969393 | 80970555 |
| C17 | 81024195 | 81025326 |
| C17 | 81025477 | 81026589 |
| C17 | 81025805 | 81026941 |
| C17 | 81025975 | 81027120 |
| C17 | 81047242 | 81048377 |
| C17 | 81058337 | 81059592 |
| C17 | 81059743 | 81060977 |
| C17 | 81059998 | 81061511 |
| C17 | 81066587 | 81067801 |
| C17 | 81081910 | 81083125 |
| C17 | 81082170 | 81083455 |
| C17 | 81146888 | 81148487 |
| C17 | 81152823 | 81153949 |
| C17 | 81153057 | 81154125 |
| C17 | 81155698 | 81156739 |
| C17 | 81162203 | 81163210 |
| C18 | 72746 | 73816 |
| C18 | 111810 | 112915 |
| C18 | 513819 | 515130 |
| C18 | 514528 | 515622 |
| C18 | 712525 | 713734 |
| C18 | 836594 | 837721 |
| C18 | 908849 | 910073 |
| C18 | 2770326 | 2771339 |
| C18 | 2876080 | 2877376 |
| C18 | 3013647 | 3014746 |
| C18 | 3623529 | 3624847 |
| C18 | 3636034 | 3637060 |
| C18 | 4030955 | 4031989 |
| C18 | 4144469 | 4145531 |
| C18 | 4197492 | 4198676 |
| C18 | 4477245 | 4478595 |
| C18 | 5773403 | 5774442 |
| C18 | 5891122 | 5892182 |
| C18 | 5891451 | 5892565 |
| C18 | 6261820 | 6262894 |
| C18 | 6413915 | 6415103 |
| C18 | 6955455 | 6956491 |
| C18 | 7010717 | 7012199 |
| C18 | 7361727 | 7362873 |
| C18 | 7566098 | 7567167 |
| C18 | 7577760 | 7579055 |
| C18 | 8053113 | 8054143 |
| C18 | 8062536 | 8063713 |
| C18 | 8684333 | 8685365 |
| C18 | 8975374 | 8976413 |
| C18 | 9405152 | 9406201 |
| C18 | 9411949 | 9412986 |
| C18 | 9621547 | 9622686 |
| C18 | 9914039 | 9915077 |
| C18 | 10032354 | 10033483 |
| C18 | 10098503 | 10099589 |
| C18 | 10606807 | 10608242 |
| C18 | 10725719 | 10727150 |
| C18 | 10763674 | 10765625 |
| C18 | 10792491 | 10793713 |
| C18 | 10921830 | 10922878 |
| C18 | 11030547 | 11031581 |
| C18 | 11677581 | 11678618 |
| C18 | 11972106 | 11973181 |
| C18 | 12068001 | 12069059 |
| C18 | 12075935 | 12077299 |
| C18 | 12207688 | 12208798 |
| C18 | 12227475 | 12229208 |
| C18 | 12254039 | 12255133 |
| C18 | 12268957 | 12270020 |
| C18 | 12375107 | 12376667 |
| C18 | 12585593 | 12586607 |
| C18 | 12611268 | 12613061 |
| C18 | 12776594 | 12778197 |
| C18 | 12874063 | 12875670 |
| C18 | 12896307 | 12897591 |
| C18 | 12911402 | 12912822 |
| C18 | 13265619 | 13267434 |
| C18 | 13374411 | 13375588 |
| C18 | 13395114 | 13396241 |
| C18 | 13499736 | 13500930 |
| C18 | 13500916 | 13502234 |
| C18 | 13627836 | 13628971 |
| C18 | 13641694 | 13642848 |
| C18 | 14425042 | 14426056 |
| C18 | 14457832 | 14458993 |
| C18 | 14458444 | 14459814 |
| C18 | 14489039 | 14490452 |
| C18 | 14822521 | 14823607 |
| C18 | 14858377 | 14859579 |
| C18 | 14999216 | 15000418 |
| C18 | 14999600 | 15000763 |
| C18 | 15197732 | 15198808 |
| C18 | 18607367 | 18608474 |
| C18 | 19934488 | 19935540 |
| C18 | 20618769 | 20619802 |
| C18 | 20685346 | 20686689 |
| C18 | 20780229 | 20781294 |
| C18 | 20861838 | 20863290 |
| C18 | 20910557 | 20911860 |
| C18 | 21964487 | 21965670 |
| C18 | 23184659 | 23186242 |
| C18 | 23713269 | 23714429 |
| C18 | 24126871 | 24128146 |
| C18 | 28448445 | 28449572 |
| C18 | 29340380 | 29341587 |
| C18 | 29719159 | 29720629 |
| C18 | 31399498 | 31400817 |
| C18 | 31399992 | 31401047 |
| C18 | 31400067 | 31401181 |
| C18 | 31738743 | 31739941 |
| C18 | 32785499 | 32786607 |
| C18 | 34833063 | 34834303 |
| C18 | 37238931 | 37239941 |
| C18 | 37378821 | 37380103 |
| C18 | 40209081 | 40210141 |
| C18 | 40260895 | 40262078 |
| C18 | 41453140 | 41454275 |
| C18 | 42161084 | 42162453 |
| C18 | 42596388 | 42597408 |
| C18 | 43417071 | 43418118 |
| C18 | 43607558 | 43608676 |
| C18 | 44012719 | 44013771 |
| C18 | 44125282 | 44126383 |
| C18 | 44554152 | 44555211 |
| C18 | 45072097 | 45073118 |
| C18 | 46307067 | 46308637 |
| C18 | 46377098 | 46378351 |
| C18 | 46728937 | 46730245 |
| C18 | 47019323 | 47020575 |
| C18 | 47104676 | 47105775 |
| C18 | 47794188 | 47795370 |
| C18 | 47875413 | 47876590 |
| C18 | 48255366 | 48256441 |
| C18 | 48345931 | 48347008 |
| C18 | 49868048 | 49869241 |
| C18 | 50382352 | 50383616 |
| C18 | 54678413 | 54679487 |
| C18 | 54813761 | 54814875 |
| C18 | 54946608 | 54949284 |
| C18 | 54994450 | 54995552 |
| C18 | 55176871 | 55178337 |
| C18 | 56756157 | 56757753 |
| C18 | 57250418 | 57251675 |
| C18 | 57636221 | 57637344 |
| C18 | 57701671 | 57703146 |
| C18 | 59220952 | 59222021 |
| C18 | 59221029 | 59222112 |
| C18 | 59221272 | 59222452 |
| C18 | 60051511 | 60052689 |
| C18 | 60247366 | 60248559 |
| C18 | 60251770 | 60252843 |
| C18 | 60262881 | 60264084 |
| C18 | 60573231 | 60574347 |
| C18 | 61669370 | 61670472 |
| C18 | 62275947 | 62277086 |
| C18 | 62873389 | 62874603 |
| C18 | 64741033 | 64742086 |
| C18 | 64992398 | 64993458 |
| C18 | 65085276 | 65086431 |
| C18 | 66268778 | 66270938 |
| C18 | 67378792 | 67379890 |
| C18 | 67523670 | 67524720 |
| C18 | 67563062 | 67564118 |
| C18 | 68879208 | 68880352 |
| C18 | 69876167 | 69877209 |
| C18 | 70358892 | 70359991 |
| C18 | 70534523 | 70535539 |
| C18 | 70535715 | 70536873 |
| C18 | 70703973 | 70705027 |
| C18 | 70741425 | 70742492 |
| C18 | 70993830 | 70994932 |
| C18 | 71355450 | 71356617 |
| C18 | 71697560 | 71698809 |
| C18 | 72388017 | 72389125 |
| C18 | 72894875 | 72895952 |
| C18 | 72915644 | 72917359 |
| C18 | 74114218 | 74115287 |
| C18 | 74117558 | 74118611 |
| C18 | 74310491 | 74312043 |
| C18 | 74357022 | 74358124 |
| C18 | 74499348 | 74500545 |
| C18 | 74533783 | 74534821 |
| C18 | 74664571 | 74666508 |
| C18 | 74764797 | 74765849 |
| C18 | 74798728 | 74799895 |
| C18 | 74798995 | 74800141 |
| C18 | 74823780 | 74824914 |
| C18 | 75084618 | 75086048 |
| C18 | 75531866 | 75533554 |
| C18 | 75616722 | 75617781 |
| C18 | 75884016 | 75885037 |
| C18 | 76069099 | 76070171 |
| C18 | 76261979 | 76263262 |
| C18 | 76322370 | 76323456 |
| C18 | 76733706 | 76734758 |
| C18 | 76739648 | 76740856 |
| C18 | 76740183 | 76741244 |
| C18 | 76751784 | 76752858 |
| C18 | 76751905 | 76752971 |
| C18 | 77157315 | 77158452 |
| C18 | 77158874 | 77160114 |
| C18 | 77195635 | 77196757 |
| C18 | 77204263 | 77205289 |
| C18 | 77218191 | 77219363 |
| C18 | 77263696 | 77264735 |
| C18 | 77265686 | 77266770 |
| C18 | 77268627 | 77269819 |
| C18 | 77279671 | 77280980 |
| C18 | 77284376 | 77285446 |
| C18 | 77291729 | 77293056 |
| C18 | 77312308 | 77313454 |
| C18 | 77326416 | 77327614 |
| C18 | 77366501 | 77367527 |
| C18 | 77376202 | 77377322 |
| C18 | 77376431 | 77377527 |
| C18 | 77376810 | 77377974 |
| C18 | 77377108 | 77378263 |
| C18 | 77377288 | 77378387 |
| C18 | 77377531 | 77378673 |
| C18 | 77408011 | 77409262 |
| C18 | 77523968 | 77525657 |
| C18 | 77544692 | 77545847 |
| C18 | 77623425 | 77624498 |
| C18 | 77623819 | 77624979 |
| C18 | 77645381 | 77646609 |
| C18 | 77658535 | 77659648 |
| C18 | 77748700 | 77749906 |
| C18 | 77751387 | 77752398 |
| C18 | 77772474 | 77773552 |
| C18 | 77905681 | 77906918 |
| C18 | 77906095 | 77907323 |
| C18 | 77917168 | 77918223 |
| C18 | 77917327 | 77918602 |
| C18 | 77917663 | 77918874 |
| C18 | 77917961 | 77919081 |
| C19 | 403698 | 405050 |
| C19 | 406705 | 407901 |
| C19 | 407660 | 408793 |
| C19 | 422042 | 423092 |
| C19 | 426652 | 427887 |
| C19 | 430932 | 432052 |
| C19 | 438283 | 439447 |
| C19 | 501181 | 502297 |
| C19 | 512089 | 513796 |
| C19 | 518194 | 519762 |
| C19 | 518765 | 519851 |
| C19 | 522795 | 523895 |
| C19 | 523097 | 524209 |
| C19 | 548427 | 549555 |
| C19 | 565869 | 566983 |
| C19 | 640967 | 642149 |
| C19 | 667095 | 668419 |
| C19 | 720175 | 721452 |
| C19 | 780195 | 781242 |
| C19 | 795775 | 797008 |
| C19 | 827722 | 828770 |
| C19 | 835321 | 836602 |
| C19 | 845824 | 846932 |
| C19 | 852885 | 854016 |
| C19 | 853249 | 854635 |
| C19 | 866167 | 867250 |
| C19 | 888211 | 889399 |
| C19 | 911313 | 912465 |
| C19 | 947150 | 948399 |
| C19 | 947464 | 948659 |
| C19 | 1008638 | 1009763 |
| C19 | 1070651 | 1071734 |
| C19 | 1070761 | 1071855 |
| C19 | 1070874 | 1072280 |
| C19 | 1074489 | 1075568 |
| C19 | 1081099 | 1082201 |
| C19 | 1081353 | 1082651 |
| C19 | 1082593 | 1083721 |
| C19 | 1087777 | 1088874 |
| C19 | 1093427 | 1094565 |
| C19 | 1104841 | 1105935 |
| C19 | 1111892 | 1113064 |
| C19 | 1130560 | 1131726 |
| C19 | 1154399 | 1155552 |
| C19 | 1211563 | 1212822 |
| C19 | 1214649 | 1215895 |
| C19 | 1241940 | 1243033 |
| C19 | 1249832 | 1250895 |
| C19 | 1250292 | 1251438 |
| C19 | 1250909 | 1252030 |
| C19 | 1274077 | 1275171 |
| C19 | 1274400 | 1275492 |
| C19 | 1288737 | 1289860 |
| C19 | 1299336 | 1300479 |
| C19 | 1396565 | 1397931 |
| C19 | 1423055 | 1424331 |
| C19 | 1438950 | 1440185 |
| C19 | 1456061 | 1457194 |
| C19 | 1465493 | 1466599 |
| C19 | 1495756 | 1497243 |
| C19 | 1498641 | 1499781 |
| C19 | 1533242 | 1534399 |
| C19 | 1608269 | 1609510 |
| C19 | 1759338 | 1760376 |
| C19 | 1789204 | 1790352 |
| C19 | 1851195 | 1852250 |
| C19 | 1856728 | 1857900 |
| C19 | 1871921 | 1873096 |
| C19 | 1876643 | 1877967 |
| C19 | 1880872 | 1881988 |
| C19 | 1901553 | 1902641 |
| C19 | 1947976 | 1949175 |
| C19 | 1966797 | 1967836 |
| C19 | 1992267 | 1993625 |
| C19 | 1997924 | 1999169 |
| C19 | 1998667 | 1999829 |
| C19 | 2039237 | 2040325 |
| C19 | 2050454 | 2051524 |
| C19 | 2096385 | 2097441 |
| C19 | 2165290 | 2166361 |
| C19 | 2262875 | 2263964 |
| C19 | 2288802 | 2290101 |
| C19 | 2290953 | 2292168 |
| C19 | 2291193 | 2292302 |
| C19 | 2300117 | 2301279 |
| C19 | 2301985 | 2303120 |
| C19 | 2315560 | 2316699 |
| C19 | 2384851 | 2385934 |
| C19 | 2423569 | 2424699 |
| C19 | 2424944 | 2426034 |
| C19 | 2425089 | 2426253 |
| C19 | 2427778 | 2428972 |
| C19 | 2490706 | 2491813 |
| C19 | 2525330 | 2526511 |
| C19 | 2546098 | 2547483 |
| C19 | 2622172 | 2623304 |
| C19 | 2634402 | 2635467 |
| C19 | 2736983 | 2738225 |
| C19 | 2769482 | 2770638 |
| C19 | 2885497 | 2886764 |
| C19 | 2888176 | 2889381 |
| C19 | 2908847 | 2910022 |
| C19 | 3093029 | 3094274 |
| C19 | 3136066 | 3138048 |
| C19 | 3178496 | 3179526 |
| C19 | 3178596 | 3179632 |
| C19 | 3178854 | 3179932 |
| C19 | 3179178 | 3180273 |
| C19 | 3179308 | 3180434 |
| C19 | 3193293 | 3194382 |
| C19 | 3289885 | 3291005 |
| C19 | 3403045 | 3404212 |
| C19 | 3403456 | 3404569 |
| C19 | 3403811 | 3404974 |
| C19 | 3404472 | 3405574 |
| C19 | 3423146 | 3424271 |
| C19 | 3434479 | 3435689 |
| C19 | 3576494 | 3577895 |
| C19 | 3618033 | 3619152 |
| C19 | 3648804 | 3649887 |
| C19 | 3648936 | 3650054 |
| C19 | 3671435 | 3672525 |
| C19 | 3700022 | 3701054 |
| C19 | 3718638 | 3719747 |
| C19 | 3816101 | 3817251 |
| C19 | 3929245 | 3930295 |
| C19 | 3964364 | 3965474 |
| C19 | 3973651 | 3974726 |
| C19 | 3983665 | 3984771 |
| C19 | 4059396 | 4060458 |
| C19 | 4060799 | 4062003 |
| C19 | 4088733 | 4090190 |
| C19 | 4328187 | 4329469 |
| C19 | 4374067 | 4375102 |
| C19 | 4463727 | 4465031 |
| C19 | 4508352 | 4509543 |
| C19 | 4531229 | 4532421 |
| C19 | 4542546 | 4544129 |
| C19 | 4543371 | 4544653 |
| C19 | 4566562 | 4567814 |
| C19 | 4680943 | 4681962 |
| C19 | 4709799 | 4710935 |
| C19 | 4725861 | 4727366 |
| C19 | 4900321 | 4901592 |
| C19 | 4987476 | 4988593 |
| C19 | 5043044 | 5044275 |
| C19 | 5075704 | 5076762 |
| C19 | 5102817 | 5104023 |
| C19 | 5228970 | 5230205 |
| C19 | 5622497 | 5623577 |
| C19 | 5687319 | 5688711 |
| C19 | 5720489 | 5721611 |
| C19 | 5947187 | 5948294 |
| C19 | 6024293 | 6025381 |
| C19 | 6046939 | 6047979 |
| C19 | 6100004 | 6101231 |
| C19 | 6369707 | 6370760 |
| C19 | 6460586 | 6461734 |
| C19 | 6475018 | 6476342 |
| C19 | 6475999 | 6477434 |
| C19 | 6476495 | 6477619 |
| C19 | 6660675 | 6661793 |
| C19 | 6677607 | 6678686 |
| C19 | 6736931 | 6737991 |
| C19 | 6744280 | 6745515 |
| C19 | 6752018 | 6753374 |
| C19 | 6753253 | 6754440 |
| C19 | 6868769 | 6869948 |
| C19 | 7003874 | 7005222 |
| C19 | 7341003 | 7342287 |
| C19 | 7591868 | 7593121 |
| C19 | 7615395 | 7616539 |
| C19 | 7682180 | 7683274 |
| C19 | 7683840 | 7685236 |
| C19 | 7761255 | 7762360 |
| C19 | 7922635 | 7923698 |
| C19 | 7926284 | 7927425 |
| C19 | 7927663 | 7928729 |
| C19 | 7927741 | 7928858 |
| C19 | 7928040 | 7929103 |
| C19 | 7936894 | 7938135 |
| C19 | 8137459 | 8138610 |
| C19 | 8340015 | 8341771 |
| C19 | 8389448 | 8390507 |
| C19 | 8548778 | 8549935 |
| C19 | 8590331 | 8591540 |
| C19 | 8590945 | 8592332 |
| C19 | 8850902 | 8852207 |
| C19 | 8882391 | 8883652 |
| C19 | 8891487 | 8893074 |
| C19 | 9473290 | 9474416 |
| C19 | 9907525 | 9908784 |
| C19 | 10076815 | 10077882 |
| C19 | 10206929 | 10208050 |
| C19 | 10332818 | 10334106 |
| C19 | 10397356 | 10398520 |
| C19 | 10398013 | 10399298 |
| C19 | 10402903 | 10404095 |
| C19 | 10404619 | 10405914 |
| C19 | 10444442 | 10445722 |
| C19 | 10444784 | 10445874 |
| C19 | 10445465 | 10446689 |
| C19 | 10456729 | 10458019 |
| C19 | 10463202 | 10464380 |
| C19 | 10513491 | 10514605 |
| C19 | 10624077 | 10625242 |
| C19 | 10625053 | 10626121 |
| C19 | 10654392 | 10655524 |
| C19 | 10679256 | 10680308 |
| C19 | 10706787 | 10707954 |
| C19 | 10735575 | 10736875 |
| C19 | 10839726 | 10840846 |
| C19 | 10841776 | 10842964 |
| C19 | 10928008 | 10929196 |
| C19 | 11031939 | 11033038 |
| C19 | 11208165 | 11209171 |
| C19 | 11429546 | 11431361 |
| C19 | 11563052 | 11564218 |
| C19 | 11591239 | 11592290 |
| C19 | 11593978 | 11595289 |
| C19 | 11616477 | 11617602 |
| C19 | 11678009 | 11679178 |
| C19 | 11895500 | 11896670 |
| C19 | 11925845 | 11927089 |
| C19 | 12203230 | 12204330 |
| C19 | 12623802 | 12625096 |
| C19 | 12665767 | 12666981 |
| C19 | 12732052 | 12733209 |
| C19 | 12752309 | 12754013 |
| C19 | 12758422 | 12759498 |
| C19 | 12876162 | 12877520 |
| C19 | 12880181 | 12881289 |
| C19 | 12886381 | 12887621 |
| C19 | 12983977 | 12985214 |
| C19 | 13060749 | 13061872 |
| C19 | 13067898 | 13069021 |
| C19 | 13106958 | 13108049 |
| C19 | 13120334 | 13121613 |
| C19 | 13122002 | 13123106 |
| C19 | 13135377 | 13136527 |
| C19 | 13142155 | 13143564 |
| C19 | 13205748 | 13206894 |
| C19 | 13205991 | 13207144 |
| C19 | 13206932 | 13208154 |
| C19 | 13209212 | 13210322 |
| C19 | 13209624 | 13211130 |
| C19 | 13259554 | 13260827 |
| C19 | 13317419 | 13319171 |
| C19 | 13408788 | 13410072 |
| C19 | 13616439 | 13617531 |
| C19 | 13928522 | 13929610 |
| C19 | 13928641 | 13929762 |
| C19 | 13941147 | 13942201 |
| C19 | 13950333 | 13951695 |
| C19 | 14028421 | 14029561 |
| C19 | 14083322 | 14084415 |
| C19 | 14088764 | 14090086 |
| C19 | 14096698 | 14097935 |
| C19 | 14201303 | 14202385 |
| C19 | 14260082 | 14261201 |
| C19 | 14276313 | 14277548 |
| C19 | 14479979 | 14482064 |
| C19 | 14583904 | 14585427 |
| C19 | 14672347 | 14673615 |
| C19 | 15004348 | 15005384 |
| C19 | 15082975 | 15084417 |
| C19 | 15287680 | 15289368 |
| C19 | 15326554 | 15328083 |
| C19 | 15342167 | 15343580 |
| C19 | 15343546 | 15344674 |
| C19 | 15343751 | 15344905 |
| C19 | 15359376 | 15360680 |
| C19 | 15413235 | 15414785 |
| C19 | 15425335 | 15426939 |
| C19 | 15440904 | 15442173 |
| C19 | 15484882 | 15486348 |
| C19 | 15487832 | 15489070 |
| C19 | 15530646 | 15531896 |
| C19 | 15563008 | 15564224 |
| C19 | 15563397 | 15564722 |
| C19 | 15567269 | 15569101 |
| C19 | 15568239 | 15569435 |
| C19 | 15574297 | 15575479 |
| C19 | 15574849 | 15576255 |
| C19 | 15579766 | 15581457 |
| C19 | 15582284 | 15583372 |
| C19 | 15661772 | 15662915 |
| C19 | 15694879 | 15695959 |
| C19 | 15699272 | 15700351 |
| C19 | 15838466 | 15839602 |
| C19 | 15917934 | 15919111 |
| C19 | 16186937 | 16188025 |
| C19 | 16221100 | 16222341 |
| C19 | 16253231 | 16254540 |
| C19 | 16403420 | 16404649 |
| C19 | 16436057 | 16437259 |
| C19 | 16436446 | 16437697 |
| C19 | 16691504 | 16692667 |
| C19 | 17006171 | 17007283 |
| C19 | 17007556 | 17008694 |
| C19 | 17008097 | 17009489 |
| C19 | 17293743 | 17295975 |
| C19 | 17354323 | 17355573 |
| C19 | 17395054 | 17396081 |
| C19 | 17403507 | 17404701 |
| C19 | 17404662 | 17405758 |
| C19 | 17421835 | 17423686 |
| C19 | 17433681 | 17434897 |
| C19 | 17434053 | 17435268 |
| C19 | 17438030 | 17439256 |
| C19 | 17438457 | 17439987 |
| C19 | 17439202 | 17440268 |
| C19 | 17439281 | 17440931 |
| C19 | 17445600 | 17446857 |
| C19 | 17480236 | 17482034 |
| C19 | 17487564 | 17488743 |
| C19 | 17504087 | 17505820 |
| C19 | 17518138 | 17519507 |
| C19 | 17543578 | 17545049 |
| C19 | 17633316 | 17635291 |
| C19 | 17637296 | 17638532 |
| C19 | 17670511 | 17671724 |
| C19 | 17675578 | 17677471 |
| C19 | 17701590 | 17703065 |
| C19 | 17716294 | 17717680 |
| C19 | 17744895 | 17746203 |
| C19 | 17873950 | 17874970 |
| C19 | 17876969 | 17878129 |
| C19 | 17886291 | 17887387 |
| C19 | 17889014 | 17890119 |
| C19 | 17905051 | 17906127 |
| C19 | 17918238 | 17919427 |
| C19 | 17940768 | 17941919 |
| C19 | 17941250 | 17942979 |
| C19 | 17952126 | 17953492 |
| C19 | 17957449 | 17960376 |
| C19 | 17973198 | 17974317 |
| C19 | 17977498 | 17978829 |
| C19 | 17980566 | 17981703 |
| C19 | 17982931 | 17984242 |
| C19 | 18103107 | 18104850 |
| C19 | 18118787 | 18119889 |
| C19 | 18159703 | 18160824 |
| C19 | 18162392 | 18163499 |
| C19 | 18182397 | 18183619 |
| C19 | 18199627 | 18200860 |
| C19 | 18201675 | 18202844 |
| C19 | 18259660 | 18260753 |
| C19 | 18272043 | 18273429 |
| C19 | 18277888 | 18278935 |
| C19 | 18283380 | 18284620 |
| C19 | 18305137 | 18306372 |
| C19 | 18367999 | 18369142 |
| C19 | 18397613 | 18398781 |
| C19 | 18411420 | 18412603 |
| C19 | 18412645 | 18413786 |
| C19 | 18415378 | 18416501 |
| C19 | 18427952 | 18429565 |
| C19 | 18432224 | 18433267 |
| C19 | 18484645 | 18485891 |
| C19 | 18502926 | 18504436 |
| C19 | 18507782 | 18509141 |
| C19 | 18520585 | 18521758 |
| C19 | 18525092 | 18526548 |
| C19 | 18525551 | 18527095 |
| C19 | 18539164 | 18540699 |
| C19 | 18549138 | 18550181 |
| C19 | 18557071 | 18558088 |
| C19 | 18587463 | 18588489 |
| C19 | 18632551 | 18633694 |
| C19 | 18662089 | 18663767 |
| C19 | 18681187 | 18682412 |
| C19 | 18759833 | 18761019 |
| C19 | 18807188 | 18809193 |
| C19 | 18810277 | 18812789 |
| C19 | 18867215 | 18868479 |
| C19 | 18887996 | 18889091 |
| C19 | 18888154 | 18889316 |
| C19 | 18897740 | 18899134 |
| C19 | 18898500 | 18899582 |
| C19 | 18898903 | 18900177 |
| C19 | 18976967 | 18978192 |
| C19 | 18978949 | 18980031 |
| C19 | 18979244 | 18981509 |
| C19 | 19042697 | 19043857 |
| C19 | 19322383 | 19323416 |
| C19 | 19586739 | 19587908 |
| C19 | 19624632 | 19625819 |
| C19 | 19624857 | 19625952 |
| C19 | 19647853 | 19648964 |
| C19 | 19655136 | 19656264 |
| C19 | 19726018 | 19727147 |
| C19 | 19728951 | 19730036 |
| C19 | 19971091 | 19972240 |
| C19 | 20258907 | 20260578 |
| C19 | 20601331 | 20602340 |
| C19 | 20606235 | 20608995 |
| C19 | 21078383 | 21079425 |
| C19 | 21264396 | 21266080 |
| C19 | 21423662 | 21424724 |
| C19 | 21664663 | 21665940 |
| C19 | 21666344 | 21667428 |
| C19 | 21688881 | 21690232 |
| C19 | 21769323 | 21770802 |
| C19 | 21904010 | 21905420 |
| C19 | 22443942 | 22445040 |
| C19 | 22468622 | 22469945 |
| C19 | 22699778 | 22701567 |
| C19 | 22816008 | 22817251 |
| C19 | 23110285 | 23111760 |
| C19 | 23184917 | 23186502 |
| C19 | 23320445 | 23321572 |
| C19 | 23851111 | 23852169 |
| C19 | 24146339 | 24147357 |
| C19 | 28130876 | 28132036 |
| C19 | 28915355 | 28917027 |
| C19 | 29522680 | 29523913 |
| C19 | 29569987 | 29571021 |
| C19 | 29924964 | 29926018 |
| C19 | 29949834 | 29951003 |
| C19 | 30019138 | 30020241 |
| C19 | 30386117 | 30387337 |
| C19 | 30561675 | 30562886 |
| C19 | 30808441 | 30809497 |
| C19 | 30853614 | 30854641 |
| C19 | 30864283 | 30865368 |
| C19 | 30893492 | 30894619 |
| C19 | 31014425 | 31015475 |
| C19 | 31016853 | 31018157 |
| C19 | 31168775 | 31169797 |
| C19 | 31613384 | 31614400 |
| C19 | 31792314 | 31793361 |
| C19 | 31842287 | 31843395 |
| C19 | 31843434 | 31844554 |
| C19 | 32384784 | 32386890 |
| C19 | 32561887 | 32562945 |
| C19 | 33062024 | 33063113 |
| C19 | 33146725 | 33147798 |
| C19 | 33184265 | 33185680 |
| C19 | 33467554 | 33469138 |
| C19 | 33613023 | 33614029 |
| C19 | 33751076 | 33752783 |
| C19 | 33757645 | 33758753 |
| C19 | 33770119 | 33771458 |
| C19 | 33863146 | 33864317 |
| C19 | 33884618 | 33885783 |
| C19 | 34262465 | 34263765 |
| C19 | 34262932 | 34264406 |
| C19 | 34287101 | 34288139 |
| C19 | 34288809 | 34289886 |
| C19 | 34570318 | 34571407 |
| C19 | 35329435 | 35330518 |
| C19 | 35412665 | 35413903 |
| C19 | 35527822 | 35528900 |
| C19 | 35715524 | 35716726 |
| C19 | 35786052 | 35787237 |
| C19 | 35800086 | 35801244 |
| C19 | 35861776 | 35862941 |
| C19 | 35939925 | 35941777 |
| C19 | 36048473 | 36049932 |
| C19 | 36245906 | 36247003 |
| C19 | 36246034 | 36247076 |
| C19 | 36246287 | 36247383 |
| C19 | 36484644 | 36485779 |
| C19 | 36484868 | 36486052 |
| C19 | 36642771 | 36644433 |
| C19 | 36759407 | 36760591 |
| C19 | 36761442 | 36762573 |
| C19 | 36761807 | 36762934 |
| C19 | 36764355 | 36765429 |
| C19 | 36787290 | 36788944 |
| C19 | 36800245 | 36801338 |
| C19 | 36980993 | 36982112 |
| C19 | 36981120 | 36982527 |
| C19 | 37178980 | 37180249 |
| C19 | 37266105 | 37267711 |
| C19 | 37283585 | 37285065 |
| C19 | 37288090 | 37289154 |
| C19 | 37463023 | 37464130 |
| C19 | 37700194 | 37701413 |
| C19 | 37759150 | 37760587 |
| C19 | 37772086 | 37773348 |
| C19 | 37772824 | 37774312 |
| C19 | 37774699 | 37775769 |
| C19 | 37789067 | 37790122 |
| C19 | 37792946 | 37794040 |
| C19 | 37806832 | 37808024 |
| C19 | 37807157 | 37808274 |
| C19 | 37813799 | 37814805 |
| C19 | 38346099 | 38347266 |
| C19 | 38548052 | 38549098 |
| C19 | 38699834 | 38701043 |
| C19 | 38707399 | 38708667 |
| C19 | 38712742 | 38713797 |
| C19 | 38719677 | 38720760 |
| C19 | 38735467 | 38736804 |
| C19 | 38875908 | 38877721 |
| C19 | 38885125 | 38886551 |
| C19 | 38993057 | 38994244 |
| C19 | 38997000 | 38998159 |
| C19 | 39017644 | 39019231 |
| C19 | 39086440 | 39087542 |
| C19 | 39086575 | 39087686 |
| C19 | 39218448 | 39219711 |
| C19 | 39281704 | 39282781 |
| C19 | 39322658 | 39324294 |
| C19 | 39402189 | 39403330 |
| C19 | 39623949 | 39625054 |
| C19 | 39694141 | 39695512 |
| C19 | 39737949 | 39739220 |
| C19 | 39755061 | 39756991 |
| C19 | 39797335 | 39799006 |
| C19 | 39798568 | 39799634 |
| C19 | 39889391 | 39890567 |
| C19 | 39905647 | 39906840 |
| C19 | 39934117 | 39935620 |
| C19 | 39997223 | 39998285 |
| C19 | 40257093 | 40258220 |
| C19 | 40348855 | 40350275 |
| C19 | 40365672 | 40367090 |
| C19 | 40382196 | 40383321 |
| C19 | 40389080 | 40390331 |
| C19 | 40397493 | 40398872 |
| C19 | 40420867 | 40422132 |
| C19 | 40623968 | 40625374 |
| C19 | 40675733 | 40676853 |
| C19 | 40901652 | 40903221 |
| C19 | 40930874 | 40931995 |
| C19 | 41085688 | 41086906 |
| C19 | 41167977 | 41169132 |
| C19 | 41223629 | 41224814 |
| C19 | 41254647 | 41256045 |
| C19 | 41304549 | 41305587 |
| C19 | 41385638 | 41386861 |
| C19 | 41829158 | 41830382 |
| C19 | 41836268 | 41837481 |
| C19 | 41836515 | 41837582 |
| C19 | 42070943 | 42072106 |
| C19 | 42473826 | 42474985 |
| C19 | 42568547 | 42569976 |
| C19 | 42730034 | 42731070 |
| C19 | 42775408 | 42776690 |
| C19 | 43143247 | 43144337 |
| C19 | 43531755 | 43533057 |
| C19 | 43591845 | 43592892 |
| C19 | 43864276 | 43865833 |
| C19 | 43875765 | 43876850 |
| C19 | 43911727 | 43912905 |
| C19 | 43969148 | 43970340 |
| C19 | 44127542 | 44128643 |
| C19 | 44258648 | 44259728 |
| C19 | 44278010 | 44279078 |
| C19 | 44278101 | 44279209 |
| C19 | 45449823 | 45450888 |
| C19 | 45594309 | 45595562 |
| C19 | 45655046 | 45656119 |
| C19 | 45655370 | 45656461 |
| C19 | 45737189 | 45738643 |
| C19 | 45748293 | 45749420 |
| C19 | 45796864 | 45798181 |
| C19 | 45898382 | 45899618 |
| C19 | 45899307 | 45900421 |
| C19 | 45899459 | 45900815 |
| C19 | 45901052 | 45902441 |
| C19 | 45931568 | 45932602 |
| C19 | 46094071 | 46095217 |
| C19 | 46151043 | 46152138 |
| C19 | 46234223 | 46235373 |
| C19 | 46269769 | 46270912 |
| C19 | 46318066 | 46319161 |
| C19 | 46318626 | 46319912 |
| C19 | 46387397 | 46388639 |
| C19 | 46417100 | 46418245 |
| C19 | 46442939 | 46444030 |
| C19 | 46443056 | 46444407 |
| C19 | 46503475 | 46504947 |
| C19 | 46581635 | 46582686 |
| C19 | 46761937 | 46763101 |
| C19 | 46915051 | 46916264 |
| C19 | 46923962 | 46925390 |
| C19 | 46955809 | 46956879 |
| C19 | 46996890 | 46998037 |
| C19 | 46997535 | 46998655 |
| C19 | 46997732 | 46998936 |
| C19 | 47200418 | 47201612 |
| C19 | 47224975 | 47226209 |
| C19 | 47329215 | 47330801 |
| C19 | 47759470 | 47760560 |
| C19 | 47876917 | 47877978 |
| C19 | 48048280 | 48049401 |
| C19 | 48402850 | 48404373 |
| C19 | 48406973 | 48408233 |
| C19 | 48423197 | 48424488 |
| C19 | 48426416 | 48427673 |
| C19 | 48438762 | 48440083 |
| C19 | 48444307 | 48445476 |
| C19 | 48448531 | 48449869 |
| C19 | 48448898 | 48450625 |
| C19 | 48459872 | 48462011 |
| C19 | 48697518 | 48698737 |
| C19 | 48697830 | 48698926 |
| C19 | 48698153 | 48699420 |
| C19 | 48864033 | 48865072 |
| C19 | 48901117 | 48902162 |
| C19 | 48983083 | 48984197 |
| C19 | 49000062 | 49001562 |
| C19 | 49001325 | 49002447 |
| C19 | 49001515 | 49002573 |
| C19 | 49001761 | 49002983 |
| C19 | 49002226 | 49003499 |
| C19 | 49186076 | 49187336 |
| C19 | 49223652 | 49224831 |
| C19 | 49253614 | 49254805 |
| C19 | 49361753 | 49362828 |
| C19 | 49374383 | 49375824 |
| C19 | 49392897 | 49394215 |
| C19 | 49527158 | 49528448 |
| C19 | 49535377 | 49536670 |
| C19 | 49540171 | 49541350 |
| C19 | 49547893 | 49549186 |
| C19 | 49550139 | 49551278 |
| C19 | 49564356 | 49565389 |
| C19 | 49587373 | 49588538 |
| C19 | 49635827 | 49636963 |
| C19 | 49636083 | 49637119 |
| C19 | 49636871 | 49637953 |
| C19 | 49712996 | 49714145 |
| C19 | 49713298 | 49714739 |
| C19 | 49841519 | 49842574 |
| C19 | 49842235 | 49843317 |
| C19 | 49842507 | 49843538 |
| C19 | 49843010 | 49844118 |
| C19 | 49890858 | 49891905 |
| C19 | 49925411 | 49926495 |
| C19 | 49972976 | 49974152 |
| C19 | 49990201 | 49991294 |
| C19 | 50016900 | 50018071 |
| C19 | 50030204 | 50031351 |
| C19 | 50030623 | 50031706 |
| C19 | 50094270 | 50095356 |
| C19 | 50192570 | 50193726 |
| C19 | 50192899 | 50194108 |
| C19 | 50222329 | 50223441 |
| C19 | 50370824 | 50371966 |
| C19 | 50383918 | 50385015 |
| C19 | 50392407 | 50393452 |
| C19 | 50419672 | 50420998 |
| C19 | 50665755 | 50666909 |
| C19 | 50666449 | 50667905 |
| C19 | 50860715 | 50862434 |
| C19 | 50893931 | 50894992 |
| C19 | 50918476 | 50919551 |
| C19 | 50920381 | 50921469 |
| C19 | 51012617 | 51013758 |
| C19 | 51014915 | 51015951 |
| C19 | 51049549 | 51050767 |
| C19 | 51108932 | 51110020 |
| C19 | 51116075 | 51117122 |
| C19 | 51124171 | 51125370 |
| C19 | 51227901 | 51228994 |
| C19 | 51373241 | 51374631 |
| C19 | 51601384 | 51602422 |
| C19 | 51601559 | 51602934 |
| C19 | 51684969 | 51686075 |
| C19 | 51685335 | 51686465 |
| C19 | 51797186 | 51798460 |
| C19 | 51966816 | 51967920 |
| C19 | 52103948 | 52105114 |
| C19 | 52146727 | 52148469 |
| C19 | 52222334 | 52223416 |
| C19 | 52792669 | 52793709 |
| C19 | 52872564 | 52873714 |
| C19 | 52995327 | 52996509 |
| C19 | 52995541 | 52996584 |
| C19 | 53361025 | 53362168 |
| C19 | 53422645 | 53423804 |
| C19 | 53700390 | 53701606 |
| C19 | 53757988 | 53759109 |
| C19 | 53820391 | 53821600 |
| C19 | 53861038 | 53862145 |
| C19 | 54040727 | 54042318 |
| C19 | 54057538 | 54058745 |
| C19 | 54296973 | 54298326 |
| C19 | 54298273 | 54299423 |
| C19 | 54334481 | 54336028 |
| C19 | 54337718 | 54339443 |
| C19 | 54430506 | 54431579 |
| C19 | 54485176 | 54486231 |
| C19 | 54568182 | 54569462 |
| C19 | 54605653 | 54606718 |
| C19 | 54619177 | 54620376 |
| C19 | 54640769 | 54641833 |
| C19 | 54695611 | 54696638 |
| C19 | 54754490 | 54755787 |
| C19 | 54779877 | 54781865 |
| C19 | 54802017 | 54803245 |
| C19 | 54833755 | 54835113 |
| C19 | 54855694 | 54856740 |
| C19 | 54856212 | 54857429 |
| C19 | 54895585 | 54896691 |
| C19 | 54904606 | 54905714 |
| C19 | 54927535 | 54928577 |
| C19 | 54976039 | 54977092 |
| C19 | 55022883 | 55024057 |
| C19 | 55037687 | 55038874 |
| C19 | 55119211 | 55120706 |
| C19 | 55249391 | 55250592 |
| C19 | 55258792 | 55259864 |
| C19 | 55290913 | 55292136 |
| C19 | 55352493 | 55353594 |
| C19 | 55412081 | 55413456 |
| C19 | 55422077 | 55423357 |
| C19 | 55494554 | 55495804 |
| C19 | 55506763 | 55508167 |
| C19 | 55622468 | 55623531 |
| C19 | 55668090 | 55669311 |
| C19 | 55683084 | 55684158 |
| C19 | 55686016 | 55687228 |
| C19 | 55815441 | 55816533 |
| C19 | 55929538 | 55930707 |
| C19 | 55963481 | 55964584 |
| C19 | 56060934 | 56062010 |
| C19 | 56151043 | 56152201 |
| C19 | 56163753 | 56164863 |
| C19 | 56180437 | 56181469 |
| C19 | 56214740 | 56215977 |
| C19 | 56598761 | 56599866 |
| C19 | 56708339 | 56709389 |
| C19 | 56752999 | 56754455 |
| C19 | 56850108 | 56851551 |
| C19 | 56914862 | 56915904 |
| C19 | 57078527 | 57079577 |
| C19 | 57349308 | 57350370 |
| C19 | 57349748 | 57350886 |
| C19 | 57350830 | 57351952 |
| C19 | 57351024 | 57352196 |
| C19 | 57351431 | 57352488 |
| C19 | 57351507 | 57352643 |
| C19 | 57453812 | 57454968 |
| C19 | 57491757 | 57492928 |
| C19 | 57741589 | 57742950 |
| C19 | 58175160 | 58176282 |
| C19 | 58520374 | 58521648 |
| C19 | 58521577 | 58522634 |
| C19 | 58566143 | 58567274 |
| C19 | 58570914 | 58571996 |
| C19 | 58571254 | 58572364 |
| C19 | 58617385 | 58618520 |
| C19 | 58816932 | 58818071 |
| C19 | 58861035 | 58862590 |
| C19 | 58867304 | 58868372 |
| C19 | 58867464 | 58868665 |
| C19 | 58867715 | 58868792 |
| C19 | 58916009 | 58917393 |
| C19 | 58950916 | 58952402 |
| C19 | 58977850 | 58978916 |
| C20 | 259039 | 260304 |
| C20 | 259358 | 260706 |
| C20 | 290695 | 292170 |
| C20 | 677551 | 678569 |
| C20 | 798447 | 799734 |
| C20 | 2634045 | 2635198 |
| C20 | 2635961 | 2637021 |
| C20 | 2801301 | 2802388 |
| C20 | 2801464 | 2802604 |
| C20 | 2804103 | 2805316 |
| C20 | 2805039 | 2806166 |
| C20 | 3072470 | 3073548 |
| C20 | 3148029 | 3149061 |
| C20 | 3220264 | 3221427 |
| C20 | 3421476 | 3422582 |
| C20 | 3640723 | 3641799 |
| C20 | 3730129 | 3731254 |
| C20 | 3730506 | 3731727 |
| C20 | 3731606 | 3732723 |
| C20 | 3731797 | 3732865 |
| C20 | 3731944 | 3733792 |
| C20 | 3869471 | 3870526 |
| C20 | 4012722 | 4013866 |
| C20 | 4012981 | 4014182 |
| C20 | 4013582 | 4014709 |
| C20 | 4110058 | 4112013 |
| C20 | 4125494 | 4126557 |
| C20 | 4228166 | 4229236 |
| C20 | 5007948 | 5010421 |
| C20 | 5484467 | 5485848 |
| C20 | 5764741 | 5765797 |
| C20 | 6700610 | 6702100 |
| C20 | 7621556 | 7622703 |
| C20 | 7999388 | 8000471 |
| C20 | 10603162 | 10604362 |
| C20 | 10647335 | 10648443 |
| C20 | 11359586 | 11360826 |
| C20 | 11613155 | 11614294 |
| C20 | 11939208 | 11940241 |
| C20 | 12051759 | 12052893 |
| C20 | 12538763 | 12539931 |
| C20 | 12643825 | 12645545 |
| C20 | 13323657 | 13324741 |
| C20 | 14206445 | 14207713 |
| C20 | 14206888 | 14207995 |
| C20 | 15765237 | 15766923 |
| C20 | 16207256 | 16208342 |
| C20 | 17102997 | 17104255 |
| C20 | 17875575 | 17876705 |
| C20 | 18479761 | 18480903 |
| C20 | 18484705 | 18485851 |
| C20 | 19810075 | 19811096 |
| C20 | 19880922 | 19881963 |
| C20 | 20344725 | 20345815 |
| C20 | 20362801 | 20363811 |
| C20 | 20957826 | 20958838 |
| C20 | 21080078 | 21081344 |
| C20 | 21371753 | 21372845 |
| C20 | 21489626 | 21490768 |
| C20 | 21490430 | 21491543 |
| C20 | 21495915 | 21497055 |
| C20 | 21522779 | 21523794 |
| C20 | 21679343 | 21680523 |
| C20 | 22502223 | 22503258 |
| C20 | 22535196 | 22536219 |
| C20 | 22557733 | 22558853 |
| C20 | 23027926 | 23028943 |
| C20 | 23028207 | 23029313 |
| C20 | 23342099 | 23343154 |
| C20 | 23483878 | 23485053 |
| C20 | 24466942 | 24467985 |
| C20 | 24632353 | 24633425 |
| C20 | 24647417 | 24648442 |
| C20 | 24664142 | 24665185 |
| C20 | 25128503 | 25130063 |
| C20 | 25310511 | 25311779 |
| C20 | 25371897 | 25373026 |
| C20 | 25838553 | 25839581 |
| C20 | 25964979 | 25966411 |
| C20 | 25990213 | 25991292 |
| C20 | 26188257 | 26189419 |
| C20 | 26188457 | 26189574 |
| C20 | 26189470 | 26190734 |
| C20 | 26231290 | 26232920 |
| C20 | 26260521 | 26262277 |
| C20 | 29515893 | 29516975 |
| C20 | 29517862 | 29519432 |
| C20 | 29524054 | 29525185 |
| C20 | 29534631 | 29535698 |
| C20 | 29930722 | 29931955 |
| C20 | 30005285 | 30006379 |
| C20 | 30057325 | 30058412 |
| C20 | 30071469 | 30072578 |
| C20 | 30134549 | 30135659 |
| C20 | 30174167 | 30175337 |
| C20 | 30225090 | 30226207 |
| C20 | 30561119 | 30562395 |
| C20 | 30582480 | 30583598 |
| C20 | 30583847 | 30584986 |
| C20 | 30708931 | 30710117 |
| C20 | 30763233 | 30764935 |
| C20 | 30764155 | 30765319 |
| C20 | 30764408 | 30765540 |
| C20 | 30764580 | 30765667 |
| C20 | 31080642 | 31081676 |
| C20 | 31260157 | 31261759 |
| C20 | 32255357 | 32256417 |
| C20 | 32288917 | 32290062 |
| C20 | 32378894 | 32379926 |
| C20 | 32449775 | 32450881 |
| C20 | 32720258 | 32721709 |
| C20 | 32765855 | 32766897 |
| C20 | 32810731 | 32812126 |
| C20 | 33063601 | 33064849 |
| C20 | 33684739 | 33685941 |
| C20 | 33741343 | 33742448 |
| C20 | 33741606 | 33742912 |
| C20 | 34043365 | 34044713 |
| C20 | 34329724 | 34330819 |
| C20 | 34329864 | 34331009 |
| C20 | 34589038 | 34590111 |
| C20 | 35089169 | 35090207 |
| C20 | 35232919 | 35234011 |
| C20 | 35697542 | 35698738 |
| C20 | 35815221 | 35816637 |
| C20 | 35944161 | 35945524 |
| C20 | 36011673 | 36012849 |
| C20 | 36012096 | 36013219 |
| C20 | 36012265 | 36013664 |
| C20 | 36146775 | 36147843 |
| C20 | 36148172 | 36149304 |
| C20 | 36148374 | 36150207 |
| C20 | 36149347 | 36150806 |
| C20 | 36329662 | 36330879 |
| C20 | 36767353 | 36768552 |
| C20 | 36780346 | 36781858 |
| C20 | 37006521 | 37007754 |
| C20 | 37089329 | 37090456 |
| C20 | 37297159 | 37298239 |
| C20 | 37301918 | 37303182 |
| C20 | 37435015 | 37436346 |
| C20 | 39311018 | 39312124 |
| C20 | 39311817 | 39312929 |
| C20 | 39487446 | 39488991 |
| C20 | 40183399 | 40184487 |
| C20 | 40626993 | 40628010 |
| C20 | 40627273 | 40628360 |
| C20 | 40835413 | 40836951 |
| C20 | 42086052 | 42087183 |
| C20 | 42141994 | 42143123 |
| C20 | 42142858 | 42144064 |
| C20 | 42465061 | 42466096 |
| C20 | 42533849 | 42535522 |
| C20 | 42696520 | 42698053 |
| C20 | 43378274 | 43379890 |
| C20 | 43538968 | 43540180 |
| C20 | 43932352 | 43933741 |
| C20 | 43934706 | 43935892 |
| C20 | 43936353 | 43937462 |
| C20 | 43944847 | 43945931 |
| C20 | 43944975 | 43946033 |
| C20 | 44563086 | 44564349 |
| C20 | 44639250 | 44640424 |
| C20 | 44639804 | 44640883 |
| C20 | 44640527 | 44641635 |
| C20 | 44657298 | 44658350 |
| C20 | 44658441 | 44659498 |
| C20 | 44685424 | 44686643 |
| C20 | 44686121 | 44687248 |
| C20 | 44686502 | 44687775 |
| C20 | 44754083 | 44755097 |
| C20 | 44829087 | 44830228 |
| C20 | 44829253 | 44830309 |
| C20 | 44838222 | 44839378 |
| C20 | 44838388 | 44839462 |
| C20 | 44869201 | 44870312 |
| C20 | 44927724 | 44928742 |
| C20 | 44992512 | 44993581 |
| C20 | 45127088 | 45128186 |
| C20 | 45540880 | 45541989 |
| C20 | 46018532 | 46019839 |
| C20 | 46210707 | 46211749 |
| C20 | 46523340 | 46525044 |
| C20 | 46596969 | 46598042 |
| C20 | 46606528 | 46607987 |
| C20 | 46777783 | 46779244 |
| C20 | 46971435 | 46972587 |
| C20 | 47012801 | 47013861 |
| C20 | 47087281 | 47088886 |
| C20 | 47338279 | 47339987 |
| C20 | 47339636 | 47340899 |
| C20 | 47551556 | 47552742 |
| C20 | 47628046 | 47629125 |
| C20 | 47723923 | 47725323 |
| C20 | 47846463 | 47848088 |
| C20 | 48526013 | 48527205 |
| C20 | 48532279 | 48533489 |
| C20 | 48625973 | 48627259 |
| C20 | 48695545 | 48697107 |
| C20 | 48821138 | 48822145 |
| C20 | 48859256 | 48860354 |
| C20 | 48860509 | 48861518 |
| C20 | 48886602 | 48887935 |
| C20 | 48914911 | 48916357 |
| C20 | 49030969 | 49031989 |
| C20 | 49031790 | 49033041 |
| C20 | 49203718 | 49204935 |
| C20 | 49307166 | 49308425 |
| C20 | 49348700 | 49350490 |
| C20 | 49360220 | 49361998 |
| C20 | 49620705 | 49621879 |
| C20 | 49728679 | 49730186 |
| C20 | 49798100 | 49799165 |
| C20 | 49843713 | 49844766 |
| C20 | 49889941 | 49890993 |
| C20 | 49999012 | 50000624 |
| C20 | 50033235 | 50034602 |
| C20 | 50108524 | 50109975 |
| C20 | 50148674 | 50149869 |
| C20 | 50216526 | 50217760 |
| C20 | 50382443 | 50383874 |
| C20 | 50426787 | 50427866 |
| C20 | 50720508 | 50721650 |
| C20 | 51654015 | 51655218 |
| C20 | 51701137 | 51702360 |
| C20 | 51978152 | 51979421 |
| C20 | 51988256 | 51989940 |
| C20 | 52036129 | 52037718 |
| C20 | 52197515 | 52198664 |
| C20 | 52276273 | 52277551 |
| C20 | 53086699 | 53087954 |
| C20 | 53133624 | 53134720 |
| C20 | 53181541 | 53183013 |
| C20 | 53232887 | 53234108 |
| C20 | 53530121 | 53531279 |
| C20 | 54465753 | 54466809 |
| C20 | 54541822 | 54543131 |
| C20 | 54556566 | 54557625 |
| C20 | 54579372 | 54580457 |
| C20 | 54692179 | 54693869 |
| C20 | 54788373 | 54789379 |
| C20 | 54912195 | 54913244 |
| C20 | 54986519 | 54987577 |
| C20 | 55027313 | 55028351 |
| C20 | 55067214 | 55068561 |
| C20 | 55200088 | 55201212 |
| C20 | 55200390 | 55201578 |
| C20 | 55202511 | 55203532 |
| C20 | 55778771 | 55780145 |
| C20 | 55790768 | 55791805 |
| C20 | 55799247 | 55800458 |
| C20 | 55922876 | 55924056 |
| C20 | 55965807 | 55966872 |
| C20 | 55977739 | 55979116 |
| C20 | 56004571 | 56005772 |
| C20 | 56015567 | 56017383 |
| C20 | 56025415 | 56026755 |
| C20 | 56055225 | 56057760 |
| C20 | 56059646 | 56060678 |
| C20 | 56077872 | 56079162 |
| C20 | 56204139 | 56205858 |
| C20 | 56272289 | 56273363 |
| C20 | 56323460 | 56324530 |
| C20 | 56780039 | 56781266 |
| C20 | 57224298 | 57225562 |
| C20 | 57267461 | 57268556 |
| C20 | 57330752 | 57331914 |
| C20 | 57386048 | 57387304 |
| C20 | 57391893 | 57393072 |
| C20 | 57408582 | 57409928 |
| C20 | 57415310 | 57416408 |
| C20 | 57415721 | 57417563 |
| C20 | 57416591 | 57417734 |
| C20 | 57425868 | 57426896 |
| C20 | 57426426 | 57427458 |
| C20 | 57426926 | 57428037 |
| C20 | 57428810 | 57430254 |
| C20 | 57429434 | 57430925 |
| C20 | 57430480 | 57431726 |
| C20 | 57463077 | 57464201 |
| C20 | 57463283 | 57464312 |
| C20 | 57463689 | 57464926 |
| C20 | 57464273 | 57465375 |
| C20 | 57464466 | 57465701 |
| C20 | 57464939 | 57465966 |
| C20 | 57725366 | 57727212 |
| C20 | 57765897 | 57767158 |
| C20 | 57766674 | 57767794 |
| C20 | 57783655 | 57784700 |
| C20 | 57846135 | 57847153 |
| C20 | 57894947 | 57896216 |
| C20 | 57911962 | 57913204 |
| C20 | 57934181 | 57935544 |
| C20 | 57985937 | 57986958 |
| C20 | 58007200 | 58008406 |
| C20 | 58044324 | 58045422 |
| C20 | 58045233 | 58046333 |
| C20 | 58180188 | 58181286 |
| C20 | 58293377 | 58294475 |
| C20 | 58322502 | 58323582 |
| C20 | 58403077 | 58404156 |
| C20 | 58508078 | 58509267 |
| C20 | 58546655 | 58548330 |
| C20 | 58713219 | 58714285 |
| C20 | 58756298 | 58757340 |
| C20 | 58910176 | 58911693 |
| C20 | 58967486 | 58968982 |
| C20 | 59212591 | 59214977 |
| C20 | 59356107 | 59357334 |
| C20 | 59373338 | 59374444 |
| C20 | 59455162 | 59456237 |
| C20 | 59504625 | 59506120 |
| C20 | 59515620 | 59516693 |
| C20 | 59570300 | 59571586 |
| C20 | 59640778 | 59642238 |
| C20 | 59653714 | 59654738 |
| C20 | 59666081 | 59668252 |
| C20 | 59681522 | 59682681 |
| C20 | 59689354 | 59691148 |
| C20 | 59701447 | 59702480 |
| C20 | 59733347 | 59735261 |
| C20 | 59738305 | 59739705 |
| C20 | 59767350 | 59768921 |
| C20 | 59782752 | 59783784 |
| C20 | 59821350 | 59822896 |
| C20 | 59836418 | 59837673 |
| C20 | 59844729 | 59845758 |
| C20 | 59857682 | 59858763 |
| C20 | 59863222 | 59864345 |
| C20 | 59864289 | 59865507 |
| C20 | 59871757 | 59873167 |
| C20 | 59879843 | 59881285 |
| C20 | 59891662 | 59892867 |
| C20 | 59929062 | 59930468 |
| C20 | 59934675 | 59936148 |
| C20 | 59940941 | 59941966 |
| C20 | 59973729 | 59974935 |
| C20 | 60009024 | 60010110 |
| C20 | 60041888 | 60042976 |
| C20 | 60064288 | 60066420 |
| C20 | 60090428 | 60091490 |
| C20 | 60115640 | 60116919 |
| C20 | 60124125 | 60125160 |
| C20 | 60125637 | 60126663 |
| C20 | 60190465 | 60191604 |
| C20 | 60228807 | 60229873 |
| C20 | 60229338 | 60230571 |
| C20 | 60230822 | 60231924 |
| C20 | 60249911 | 60251157 |
| C20 | 60257872 | 60259575 |
| C20 | 60272636 | 60273861 |
| C20 | 60280507 | 60281711 |
| C20 | 60305845 | 60306905 |
| C20 | 60313288 | 60314559 |
| C20 | 60315696 | 60316955 |
| C20 | 60319417 | 60320762 |
| C20 | 60332887 | 60334163 |
| C20 | 60333950 | 60335137 |
| C20 | 60336090 | 60337653 |
| C20 | 60356049 | 60357108 |
| C20 | 60358765 | 60360078 |
| C20 | 60359326 | 60360366 |
| C20 | 60379977 | 60381631 |
| C20 | 60392050 | 60393382 |
| C20 | 60392981 | 60394478 |
| C20 | 60404826 | 60406596 |
| C20 | 60419270 | 60420421 |
| C20 | 60419898 | 60421092 |
| C20 | 60433647 | 60434937 |
| C20 | 60448120 | 60449312 |
| C20 | 60449941 | 60451526 |
| C20 | 60455491 | 60457032 |
| C20 | 60469373 | 60470781 |
| C20 | 60476195 | 60477321 |
| C20 | 60483007 | 60484286 |
| C20 | 60502364 | 60503504 |
| C20 | 60518303 | 60519403 |
| C20 | 60519461 | 60520578 |
| C20 | 60519605 | 60520681 |
| C20 | 60520624 | 60521693 |
| C20 | 60520705 | 60521812 |
| C20 | 60522494 | 60523644 |
| C20 | 60523075 | 60524194 |
| C20 | 60544444 | 60545533 |
| C20 | 60619638 | 60621067 |
| C20 | 60640631 | 60641752 |
| C20 | 60640974 | 60642032 |
| C20 | 60707853 | 60708974 |
| C20 | 60717920 | 60718957 |
| C20 | 60731510 | 60732573 |
| C20 | 60773639 | 60774782 |
| C20 | 60813169 | 60814272 |
| C20 | 60860241 | 60861366 |
| C20 | 60869214 | 60870298 |
| C20 | 60877403 | 60878516 |
| C20 | 60882166 | 60883362 |
| C20 | 60948052 | 60949123 |
| C20 | 60952382 | 60953552 |
| C20 | 61001275 | 61002362 |
| C20 | 61040922 | 61042051 |
| C20 | 61048890 | 61050053 |
| C20 | 61049516 | 61050649 |
| C20 | 61080862 | 61081904 |
| C20 | 61089775 | 61091031 |
| C20 | 61149393 | 61151146 |
| C20 | 61151965 | 61153233 |
| C20 | 61158030 | 61160054 |
| C20 | 61194442 | 61195473 |
| C20 | 61297160 | 61298476 |
| C20 | 61307213 | 61308701 |
| C20 | 61314776 | 61315967 |
| C20 | 61374925 | 61376272 |
| C20 | 61380429 | 61381863 |
| C20 | 61403040 | 61404233 |
| C20 | 61443251 | 61444379 |
| C20 | 61467005 | 61468268 |
| C20 | 61478345 | 61479660 |
| C20 | 61506057 | 61507322 |
| C20 | 61510094 | 61511532 |
| C20 | 61554325 | 61555446 |
| C20 | 61583393 | 61584411 |
| C20 | 61583598 | 61586766 |
| C20 | 61590029 | 61591174 |
| C20 | 61590251 | 61591550 |
| C20 | 61595706 | 61596836 |
| C20 | 61635917 | 61637092 |
| C20 | 61637971 | 61639139 |
| C20 | 61652900 | 61654038 |
| C20 | 61659806 | 61660952 |
| C20 | 61660060 | 61661122 |
| C20 | 61660153 | 61661633 |
| C20 | 61665815 | 61667054 |
| C20 | 61674666 | 61675889 |
| C20 | 61675007 | 61676121 |
| C20 | 61703256 | 61704390 |
| C20 | 61714695 | 61715882 |
| C20 | 61715094 | 61716282 |
| C20 | 61720277 | 61721561 |
| C20 | 61766179 | 61767340 |
| C20 | 61769731 | 61771140 |
| C20 | 61808114 | 61809335 |
| C20 | 61885512 | 61886584 |
| C20 | 61891021 | 61892213 |
| C20 | 61892935 | 61894339 |
| C20 | 61897563 | 61898712 |
| C20 | 61937079 | 61938209 |
| C20 | 61972684 | 61974416 |
| C20 | 61973498 | 61975513 |
| C20 | 61976866 | 61978314 |
| C20 | 61978838 | 61980072 |
| C20 | 61982024 | 61983134 |
| C20 | 62002281 | 62003532 |
| C20 | 62012495 | 62013669 |
| C20 | 62028184 | 62029401 |
| C20 | 62028607 | 62030215 |
| C20 | 62031878 | 62032912 |
| C20 | 62042329 | 62043602 |
| C20 | 62046157 | 62047461 |
| C20 | 62048733 | 62049874 |
| C20 | 62048978 | 62050156 |
| C20 | 62050156 | 62051373 |
| C20 | 62052030 | 62053164 |
| C20 | 62052748 | 62054033 |
| C20 | 62053574 | 62054923 |
| C20 | 62058174 | 62059300 |
| C20 | 62058392 | 62059410 |
| C20 | 62070537 | 62071956 |
| C20 | 62079257 | 62080396 |
| C20 | 62082725 | 62083883 |
| C20 | 62087542 | 62089024 |
| C20 | 62089572 | 62091167 |
| C20 | 62119135 | 62120270 |
| C20 | 62119476 | 62120617 |
| C20 | 62137330 | 62138338 |
| C20 | 62165755 | 62166886 |
| C20 | 62166802 | 62168071 |
| C20 | 62167654 | 62169024 |
| C20 | 62168075 | 62169183 |
| C20 | 62173045 | 62174282 |
| C20 | 62179983 | 62181037 |
| C20 | 62180066 | 62181149 |
| C20 | 62198307 | 62200438 |
| C20 | 62208186 | 62209551 |
| C20 | 62221113 | 62222223 |
| C20 | 62258016 | 62259072 |
| C20 | 62260999 | 62262150 |
| C20 | 62271933 | 62272994 |
| C20 | 62280459 | 62281605 |
| C20 | 62288461 | 62289600 |
| C20 | 62400318 | 62401549 |
| C20 | 62405339 | 62406717 |
| C20 | 62406149 | 62407249 |
| C20 | 62420941 | 62422104 |
| C20 | 62427080 | 62428269 |
| C20 | 62433438 | 62434543 |
| C20 | 62466779 | 62467893 |
| C20 | 62493892 | 62494969 |
| C20 | 62508741 | 62509791 |
| C20 | 62508796 | 62509869 |
| C20 | 62519978 | 62521018 |
| C20 | 62534722 | 62536172 |
| C20 | 62537274 | 62538465 |
| C20 | 62569389 | 62570496 |
| C20 | 62569747 | 62571023 |
| C20 | 62570129 | 62571231 |
| C20 | 62678925 | 62679990 |
| C20 | 62679321 | 62680397 |
| C20 | 62691664 | 62692933 |
| C20 | 62737400 | 62738570 |
| C20 | 62739024 | 62740245 |
| C20 | 62760565 | 62761870 |
| C20 | 62809236 | 62810396 |
| C20 | 62815092 | 62816107 |
| C20 | 62828659 | 62829954 |
| C20 | 62832751 | 62834004 |
| C21 | 9437895 | 9438955 |
| C21 | 9438106 | 9439169 |
| C21 | 9483137 | 9484186 |
| C21 | 9483376 | 9484460 |
| C21 | 9483693 | 9484761 |
| C21 | 9483775 | 9484881 |
| C21 | 9708482 | 9709633 |
| C21 | 9825170 | 9826915 |
| C21 | 9826006 | 9827031 |
| C21 | 9826162 | 9827578 |
| C21 | 9826913 | 9828048 |
| C21 | 9881647 | 9882823 |
| C21 | 9909284 | 9910729 |
| C21 | 9911440 | 9912632 |
| C21 | 9911816 | 9912911 |
| C21 | 9913024 | 9914350 |
| C21 | 9921651 | 9922840 |
| C21 | 9926028 | 9927133 |
| C21 | 9926307 | 9927387 |
| C21 | 9967852 | 9969001 |
| C21 | 10105056 | 10106443 |
| C21 | 10163577 | 10164811 |
| C21 | 10989687 | 10990768 |
| C21 | 10990082 | 10991144 |
| C21 | 10990618 | 10991689 |
| C21 | 11097574 | 11098779 |
| C21 | 11107810 | 11109402 |
| C21 | 11169229 | 11170288 |
| C21 | 14982274 | 14983456 |
| C21 | 15068848 | 15070006 |
| C21 | 15076593 | 15078311 |
| C21 | 15095262 | 15096317 |
| C21 | 15197393 | 15198494 |
| C21 | 15435475 | 15436977 |
| C21 | 15436227 | 15437328 |
| C21 | 15436391 | 15437462 |
| C21 | 15645833 | 15646953 |
| C21 | 15652951 | 15654024 |
| C21 | 16274128 | 16275196 |
| C21 | 17960407 | 17961494 |
| C21 | 18885049 | 18886092 |
| C21 | 19091064 | 19092299 |
| C21 | 19091828 | 19092947 |
| C21 | 19273895 | 19275597 |
| C21 | 19616939 | 19618085 |
| C21 | 21673713 | 21675546 |
| C21 | 21688016 | 21689113 |
| C21 | 22420171 | 22421422 |
| C21 | 22436945 | 22437977 |
| C21 | 23467130 | 23468671 |
| C21 | 23525367 | 23526371 |
| C21 | 23756033 | 23757172 |
| C21 | 23876037 | 23877816 |
| C21 | 23885544 | 23887621 |
| C21 | 25140847 | 25141879 |
| C21 | 25140909 | 25142122 |
| C21 | 27105068 | 27106235 |
| C21 | 27888806 | 27889882 |
| C21 | 27944511 | 27945766 |
| C21 | 28719542 | 28721032 |
| C21 | 30395577 | 30397004 |
| C21 | 30449004 | 30450336 |
| C21 | 30471764 | 30472883 |
| C21 | 31339709 | 31340759 |
| C21 | 33127339 | 33128357 |
| C21 | 33247337 | 33248720 |
| C21 | 33809574 | 33810810 |
| C21 | 33986854 | 33987921 |
| C21 | 34327284 | 34328389 |
| C21 | 34405675 | 34406745 |
| C21 | 34773937 | 34775036 |
| C21 | 35039501 | 35040668 |
| C21 | 35239564 | 35240736 |
| C21 | 35303088 | 35304251 |
| C21 | 35444544 | 35445662 |
| C21 | 36060094 | 36061296 |
| C21 | 36263027 | 36264272 |
| C21 | 36870613 | 36872144 |
| C21 | 37571804 | 37573055 |
| C21 | 37617875 | 37619312 |
| C21 | 38068579 | 38069662 |
| C21 | 38377795 | 38378874 |
| C21 | 38379246 | 38380285 |
| C21 | 38471959 | 38473825 |
| C21 | 39590217 | 39591284 |
| C21 | 39752961 | 39754615 |
| C21 | 39823301 | 39824308 |
| C21 | 39951567 | 39952599 |
| C21 | 40031997 | 40033047 |
| C21 | 40172965 | 40174104 |
| C21 | 40176514 | 40177690 |
| C21 | 40194586 | 40195954 |
| C21 | 40747078 | 40748163 |
| C21 | 40759055 | 40760187 |
| C21 | 40855717 | 40856788 |
| C21 | 42402400 | 42403486 |
| C21 | 42591231 | 42592465 |
| C21 | 43100203 | 43102038 |
| C21 | 43222404 | 43223548 |
| C21 | 43243212 | 43244396 |
| C21 | 43695845 | 43697113 |
| C21 | 43915852 | 43916979 |
| C21 | 43950787 | 43951867 |
| C21 | 43959799 | 43960823 |
| C21 | 44007373 | 44008515 |
| C21 | 44007560 | 44008654 |
| C21 | 44009146 | 44010337 |
| C21 | 44009693 | 44010810 |
| C21 | 44104738 | 44105837 |
| C21 | 44139829 | 44140902 |
| C21 | 44157922 | 44159210 |
| C21 | 44375034 | 44376466 |
| C21 | 44573471 | 44574523 |
| C21 | 44582972 | 44584073 |
| C21 | 44698398 | 44699623 |
| C21 | 44818912 | 44820585 |
| C21 | 44994084 | 44995436 |
| C21 | 45077354 | 45078401 |
| C21 | 45148379 | 45149485 |
| C21 | 45336089 | 45337734 |
| C21 | 45387360 | 45388503 |
| C21 | 45433661 | 45434931 |
| C21 | 45503425 | 45504628 |
| C21 | 45507075 | 45508421 |
| C21 | 45522045 | 45523164 |
| C21 | 45559033 | 45560778 |
| C21 | 45590680 | 45591841 |
| C21 | 45605860 | 45606869 |
| C21 | 45621748 | 45622908 |
| C21 | 45708329 | 45709396 |
| C21 | 45753796 | 45754940 |
| C21 | 45767805 | 45769067 |
| C21 | 45780106 | 45781240 |
| C21 | 45789549 | 45790631 |
| C21 | 45789995 | 45791206 |
| C21 | 45829852 | 45831255 |
| C21 | 45845066 | 45846120 |
| C21 | 45876321 | 45877421 |
| C21 | 45926142 | 45927319 |
| C21 | 45928352 | 45929902 |
| C21 | 45953160 | 45954267 |
| C21 | 46020054 | 46021183 |
| C21 | 46021936 | 46023152 |
| C21 | 46274560 | 46275610 |
| C21 | 46277741 | 46278825 |
| C21 | 46283391 | 46284633 |
| C21 | 46293601 | 46294673 |
| C21 | 46320875 | 46322144 |
| C21 | 46327251 | 46328593 |
| C21 | 46337902 | 46339570 |
| C21 | 46489338 | 46490505 |
| C21 | 46571310 | 46574217 |
| C21 | 46706195 | 46707259 |
| C21 | 46839722 | 46840931 |
| C21 | 46844222 | 46845385 |
| C21 | 46863531 | 46864727 |
| C21 | 46876158 | 46877252 |
| C21 | 46890558 | 46892004 |
| C21 | 46925270 | 46926497 |
| C21 | 46950317 | 46951384 |
| C21 | 46954371 | 46955459 |
| C21 | 46973109 | 46974430 |
| C21 | 46975446 | 46976777 |
| C21 | 47038435 | 47040005 |
| C21 | 47209422 | 47210548 |
| C21 | 47237818 | 47239048 |
| C21 | 47254352 | 47255451 |
| C21 | 47294496 | 47295579 |
| C21 | 47351811 | 47352970 |
| C21 | 47402254 | 47403315 |
| C21 | 47419998 | 47421085 |
| C21 | 47453823 | 47454945 |
| C21 | 47459903 | 47461126 |
| C21 | 47469595 | 47470740 |
| C21 | 47471850 | 47473035 |
| C21 | 47476497 | 47478191 |
| C21 | 47518101 | 47519183 |
| C21 | 47580459 | 47581543 |
| C21 | 47580580 | 47582465 |
| C21 | 47581498 | 47582564 |
| C21 | 47603791 | 47604887 |
| C21 | 47715620 | 47716894 |
| C21 | 47743407 | 47744498 |
| C21 | 47798657 | 47799752 |
| C21 | 47802639 | 47803709 |
| C21 | 47851638 | 47852835 |
| C21 | 47873788 | 47874832 |
| C21 | 47879391 | 47880444 |
| C21 | 47970080 | 47971229 |
| C21 | 48081110 | 48082372 |
| C21 | 48117799 | 48119130 |
| C21 | 48118378 | 48119414 |
| C21 | 48118558 | 48119746 |
| C22 | 16155449 | 16156545 |
| C22 | 16199662 | 16200831 |
| C22 | 16201697 | 16202992 |
| C22 | 16406526 | 16408761 |
| C22 | 16413921 | 16415225 |
| C22 | 16466076 | 16467213 |
| C22 | 16466491 | 16467613 |
| C22 | 16511676 | 16513617 |
| C22 | 16609987 | 16612211 |
| C22 | 16628062 | 16630121 |
| C22 | 16867269 | 16868370 |
| C22 | 17028066 | 17029809 |
| C22 | 17089443 | 17090495 |
| C22 | 17488387 | 17489434 |
| C22 | 17517110 | 17518180 |
| C22 | 17517612 | 17518681 |
| C22 | 17518271 | 17519497 |
| C22 | 17600460 | 17601579 |
| C22 | 17940663 | 17941757 |
| C22 | 18046797 | 18048044 |
| C22 | 18059445 | 18060600 |
| C22 | 18110011 | 18111282 |
| C22 | 18242902 | 18244103 |
| C22 | 18574404 | 18575714 |
| C22 | 18633087 | 18634178 |
| C22 | 18701900 | 18703341 |
| C22 | 18811629 | 18813054 |
| C22 | 18859241 | 18860365 |
| C22 | 18925980 | 18927614 |
| C22 | 19071534 | 19073033 |
| C22 | 19137416 | 19138526 |
| C22 | 19279549 | 19280580 |
| C22 | 19511616 | 19512699 |
| C22 | 19608483 | 19609534 |
| C22 | 19709279 | 19710396 |
| C22 | 19710601 | 19711774 |
| C22 | 19713001 | 19714498 |
| C22 | 19760011 | 19761118 |
| C22 | 19863113 | 19864129 |
| C22 | 20103235 | 20104395 |
| C22 | 20329240 | 20330584 |
| C22 | 20358964 | 20360779 |
| C22 | 20366348 | 20367617 |
| C22 | 20377973 | 20379033 |
| C22 | 20397715 | 20399188 |
| C22 | 20458775 | 20459851 |
| C22 | 20503584 | 20504767 |
| C22 | 20713385 | 20714449 |
| C22 | 20783302 | 20784371 |
| C22 | 21021473 | 21022614 |
| C22 | 21087837 | 21089183 |
| C22 | 21555688 | 21557530 |
| C22 | 21564814 | 21566040 |
| C22 | 21662729 | 21663799 |
| C22 | 21696399 | 21697478 |
| C22 | 21714779 | 21715837 |
| C22 | 21770511 | 21771587 |
| C22 | 21771177 | 21772228 |
| C22 | 21782251 | 21783388 |
| C22 | 21821496 | 21822649 |
| C22 | 21896601 | 21897863 |
| C22 | 21975357 | 21976571 |
| C22 | 21982650 | 21983812 |
| C22 | 22098757 | 22099790 |
| C22 | 22417237 | 22418401 |
| C22 | 22469018 | 22470088 |
| C22 | 22471460 | 22472589 |
| C22 | 22471660 | 22472860 |
| C22 | 22472059 | 22473164 |
| C22 | 22553692 | 22556181 |
| C22 | 22571095 | 22572181 |
| C22 | 22989290 | 22990703 |
| C22 | 23081891 | 23083197 |
| C22 | 23455580 | 23456954 |
| C22 | 23574908 | 23576159 |
| C22 | 23800859 | 23802135 |
| C22 | 23981168 | 23982209 |
| C22 | 24197248 | 24198385 |
| C22 | 24627561 | 24628580 |
| C22 | 24654305 | 24655948 |
| C22 | 24890589 | 24891694 |
| C22 | 24951322 | 24952503 |
| C22 | 25022919 | 25024040 |
| C22 | 25081437 | 25082462 |
| C22 | 25159570 | 25160709 |
| C22 | 25347945 | 25349052 |
| C22 | 25395850 | 25396956 |
| C22 | 25465662 | 25466873 |
| C22 | 25612682 | 25614118 |
| C22 | 26148329 | 26149379 |
| C22 | 27168213 | 27169331 |
| C22 | 27428050 | 27429209 |
| C22 | 27590763 | 27591846 |
| C22 | 27831461 | 27832617 |
| C22 | 28180654 | 28182592 |
| C22 | 28193671 | 28194801 |
| C22 | 28195192 | 28196457 |
| C22 | 28195471 | 28196645 |
| C22 | 28197955 | 28199039 |
| C22 | 28838412 | 28839853 |
| C22 | 29059041 | 29060150 |
| C22 | 29166493 | 29167656 |
| C22 | 29468613 | 29469686 |
| C22 | 29708939 | 29710066 |
| C22 | 29801454 | 29802575 |
| C22 | 29865781 | 29867061 |
| C22 | 29948082 | 29949251 |
| C22 | 29998183 | 29999434 |
| C22 | 30054394 | 30056680 |
| C22 | 30475528 | 30476622 |
| C22 | 30662472 | 30663576 |
| C22 | 30668513 | 30669898 |
| C22 | 30969961 | 30971028 |
| C22 | 30970229 | 30971370 |
| C22 | 31026905 | 31028182 |
| C22 | 31317311 | 31318972 |
| C22 | 32017160 | 32018206 |
| C22 | 32599314 | 32600468 |
| C22 | 32599881 | 32601003 |
| C22 | 33226271 | 33227377 |
| C22 | 33891661 | 33892690 |
| C22 | 34404534 | 34405588 |
| C22 | 35080369 | 35081550 |
| C22 | 36585677 | 36587038 |
| C22 | 36598195 | 36599204 |
| C22 | 36708921 | 36710309 |
| C22 | 36902564 | 36903688 |
| C22 | 37265878 | 37267078 |
| C22 | 37493332 | 37494534 |
| C22 | 37768923 | 37770056 |
| C22 | 37769607 | 37770750 |
| C22 | 37879547 | 37881254 |
| C22 | 37880653 | 37881881 |
| C22 | 38244099 | 38245247 |
| C22 | 38484607 | 38485664 |
| C22 | 38577953 | 38579078 |
| C22 | 38596641 | 38597694 |
| C22 | 38610104 | 38611172 |
| C22 | 38610276 | 38611351 |
| C22 | 38610396 | 38611795 |
| C22 | 38614051 | 38615264 |
| C22 | 38668627 | 38669761 |
| C22 | 39203164 | 39204359 |
| C22 | 39227625 | 39228812 |
| C22 | 39300698 | 39302030 |
| C22 | 39547307 | 39548604 |
| C22 | 39567250 | 39568293 |
| C22 | 39594588 | 39595707 |
| C22 | 39783897 | 39785052 |
| C22 | 39784475 | 39785630 |
| C22 | 39824881 | 39825933 |
| C22 | 39852381 | 39853460 |
| C22 | 39884094 | 39885218 |
| C22 | 39950651 | 39951709 |
| C22 | 40657340 | 40658822 |
| C22 | 40795751 | 40797048 |
| C22 | 40932727 | 40933794 |
| C22 | 41042117 | 41043291 |
| C22 | 41047806 | 41049533 |
| C22 | 41292560 | 41294247 |
| C22 | 41633806 | 41634948 |
| C22 | 41754510 | 41755594 |
| C22 | 41806271 | 41807309 |
| C22 | 41881044 | 41882106 |
| C22 | 42016751 | 42017861 |
| C22 | 42062789 | 42063926 |
| C22 | 42077818 | 42078909 |
| C22 | 42094847 | 42096004 |
| C22 | 42353236 | 42354350 |
| C22 | 42394302 | 42395354 |
| C22 | 42469536 | 42470624 |
| C22 | 42524642 | 42525673 |
| C22 | 42524913 | 42526046 |
| C22 | 42538315 | 42539406 |
| C22 | 42829248 | 42830646 |
| C22 | 43165381 | 43166536 |
| C22 | 43165639 | 43166705 |
| C22 | 43377745 | 43378982 |
| C22 | 43541935 | 43543068 |
| C22 | 43548873 | 43549959 |
| C22 | 43592120 | 43593313 |
| C22 | 43704049 | 43705257 |
| C22 | 44069263 | 44070342 |
| C22 | 44229737 | 44231464 |
| C22 | 44230519 | 44231723 |
| C22 | 44391509 | 44392731 |
| C22 | 44422096 | 44423453 |
| C22 | 44463682 | 44465735 |
| C22 | 44508022 | 44509944 |
| C22 | 44758644 | 44760169 |
| C22 | 44847599 | 44848604 |
| C22 | 45097262 | 45098335 |
| C22 | 45124803 | 45125980 |
| C22 | 45330320 | 45331344 |
| C22 | 45367714 | 45368909 |
| C22 | 45453120 | 45454331 |
| C22 | 45558272 | 45559483 |
| C22 | 45626048 | 45627250 |
| C22 | 45808328 | 45809706 |
| C22 | 45832430 | 45833540 |
| C22 | 45832834 | 45833935 |
| C22 | 46281374 | 46282566 |
| C22 | 46286670 | 46287754 |
| C22 | 46422996 | 46424013 |
| C22 | 46456665 | 46457853 |
| C22 | 46456910 | 46457992 |
| C22 | 46457011 | 46458089 |
| C22 | 46471951 | 46473108 |
| C22 | 46475297 | 46476601 |
| C22 | 46475758 | 46476957 |
| C22 | 46502505 | 46503790 |
| C22 | 46525133 | 46526309 |
| C22 | 46657988 | 46659075 |
| C22 | 46786799 | 46788407 |
| C22 | 46872667 | 46873998 |
| C22 | 46912374 | 46913773 |
| C22 | 46976061 | 46977194 |
| C22 | 46994633 | 46995839 |
| C22 | 47018038 | 47019202 |
| C22 | 47030963 | 47032037 |
| C22 | 47073504 | 47074780 |
| C22 | 47080540 | 47081657 |
| C22 | 47337809 | 47339575 |
| C22 | 47351248 | 47352287 |
| C22 | 47528906 | 47530123 |
| C22 | 47549907 | 47551329 |
| C22 | 47774324 | 47775478 |
| C22 | 48313949 | 48315058 |
| C22 | 48604576 | 48605852 |
| C22 | 48639945 | 48641216 |
| C22 | 48644718 | 48645955 |
| C22 | 48660782 | 48661919 |
| C22 | 48931669 | 48932785 |
| C22 | 49189460 | 49190524 |
| C22 | 49223400 | 49224466 |
| C22 | 49386740 | 49387858 |
| C22 | 49447330 | 49448955 |
| C22 | 49598828 | 49599979 |
| C22 | 49729426 | 49730513 |
| C22 | 49764573 | 49765639 |
| C22 | 49875286 | 49876452 |
| C22 | 49937206 | 49939140 |
| C22 | 50030691 | 50031833 |
| C22 | 50048334 | 50049680 |
| C22 | 50097733 | 50098765 |
| C22 | 50128562 | 50129672 |
| C22 | 50144053 | 50145135 |
| C22 | 50164736 | 50165897 |
| C22 | 50231803 | 50233051 |
| C22 | 50273509 | 50274600 |
| C22 | 50322087 | 50323271 |
| C22 | 50331215 | 50332504 |
| C22 | 50356123 | 50357228 |
| C22 | 50448109 | 50449121 |
| C22 | 50467516 | 50468615 |
| C22 | 50472253 | 50473331 |
| C22 | 50496103 | 50497177 |
| C22 | 50521747 | 50522961 |
| C22 | 50522183 | 50524204 |
| C22 | 50585173 | 50586564 |
| C22 | 50615992 | 50617172 |
| C22 | 50616290 | 50617479 |
| C22 | 50617985 | 50619126 |
| C22 | 50622863 | 50623936 |
| C22 | 50629507 | 50630600 |
| C22 | 50692061 | 50693466 |
| C22 | 50705641 | 50706742 |
| C22 | 50714274 | 50715431 |
| C22 | 50720620 | 50721795 |
| C22 | 50731147 | 50732343 |
| C22 | 50737247 | 50738572 |
| C22 | 50743494 | 50744927 |
| C22 | 50754843 | 50755964 |
| C22 | 50912426 | 50913505 |
| C22 | 50963475 | 50964555 |
| C22 | 50981874 | 50982988 |
| C22 | 50984825 | 50986048 |
| C22 | 50985230 | 50986451 |
| C22 | 50987054 | 50988214 |
| C22 | 51015771 | 51016977 |
| C22 | 51016040 | 51017204 |
| C22 | 51040637 | 51041714 |
| C22 | 51051585 | 51052833 |
| C22 | 51097455 | 51098953 |
| C22 | 51135099 | 51136721 |
| C22 | 51135750 | 51136960 |
| C22 | 51143387 | 51144520 |
| C22 | 51181024 | 51182165 |
| C22 | 51193460 | 51194508 |
| C22 | 51221612 | 51222719 |

In some embodiments, target genomic regions that are examined to determine the presence of MBC, MRD, or breast cancer recurrence in a subject may comprise about 700 to about 750 of the target regions listed in Table 1. In some embodiments, target genomic regions that are examined to, for example, determine the presence of MBC, the presence of or susceptibility to MRD, or the presence of or susceptibility of breast cancer recurrence in a subject comprise all the target regions listed in Table 1.

In some embodiments, the detection of cfDNA in the sample further comprises aligning the DNA sequences from the next-generation sequencing to a human reference genome. In a specific embodiment, the human reference genome GRCh37 (UCSC version hg19) is incorporated herein in its entirety. This genome assembly can be found, for example, at www.genome.ucsc.edu.

In some embodiments, the nucleotide sequences that are examined for nucleic acid methylation patterns include the target region sequences listed in Table 1 and also may include the immediately adjacent 1-100, 1-150, 1-200, 1-300, 1-400, 1-500, 500-1000, 1000-1500, 1500-2000, 2000-2500, 2500-3000, 3000-3500, or 3500-4000 nucleotides upstream or downstream of a target genomic region listed in Table 1.

In some embodiments, the methylation pattern of a target region of cfDNA is determined at a region within a selected gene or genes. Non-limiting examples include a region within an untranslated region (UTR) of the selected gene or genes, a region within 1.5 kb upstream of the transcription start site of the selected gene or genes, and a region within the first exon of the selected gene or genes. In other embodiments, the target regions of cfDNA are within non-gene regions of genomic DNA.

Embodiments of the methods described herein also may be used to determine the methylation pattern of certain target regions that are implicated in various cancers to predict, for example, malignancy or stages of malignancy, susceptibility of recurrence of a cancer, and/or the presence of or the susceptibility to MRD. Exemplary cancers include leukemias, including acute leukemias (such as 11q23-positive acute leukemia, acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia. Other tumors may include sarcomas and carcinomas, include fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, colon carcinoma, lymphoid malignancy, pancreatic cancer, breast cancer (including basal breast carcinoma, ductal carcinoma and lobular breast carcinoma), lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, and CNS tumors (such as a glioma, astrocytoma, medulloblastoma, craniopharyrgioma, ependymoma, pinealoma, hemangioblastoma, acoustic neuroma, oligodendroglioma, meningioma, melanoma, neuroblastoma and retinoblastoma). Any of the above listed cancers may result in MRD after treatment.

Using, for example, the target regions listed in Table 1, embodiments of the invention can have greater than 75% sensitivity in detecting breast cancer, breast cancer recurrence, MBC, or MRD; greater than 80% sensitivity in detecting breast cancer, breast cancer recurrence, MBC, or MRD; greater than 85% sensitivity in detecting breast cancer, breast cancer recurrence, MBC, or MRD; greater than 90% sensitivity in detecting breast cancer, breast cancer recurrence, MBC, or MRD; greater than 95% sensitivity in detecting breast cancer, breast cancer recurrence, MBC, or MRD; greater than 96% sensitivity in detecting breast cancer, breast cancer recurrence, MBC, or MRD; greater than 97% sensitivity in detecting breast cancer, breast cancer recurrence, MBC, or MRD; greater than 98% sensitivity in detecting breast cancer, breast cancer recurrence, MBC, or MRD; greater than 99% sensitivity in detecting breast cancer, breast cancer recurrence, MBC, or MRD; or 100% sensitivity in detecting breast cancer, breast cancer recurrence, MBC, or MRD.

In some embodiments, a subject maybe tested for the presence or absence of MRD using the methods described herein at any time during treatment of a cancer or after completion of a cancer treatment regimen.

Upon identifying a subject as likely to develop cancer (e.g., breast cancer), cancer recurrence (e.g., breast cancer), MBC, or MRD, a prophylactic procedure or therapy can be administered to the subject. For example, prophylactic measures include but are not limited to surgery, tamoxifen administration, and raloxifene administration. For solid tumors, surgical resection can be performed.

Upon identifying a subject as having breast cancer, breast cancer recurrence, MBC, or MRD, a clinical procedure or cancer therapy can be administered to the subject. Exemplary therapies or procedures include but are not limited to surgery, radiation therapy, chemotherapy, hormone therapy, targeted therapy, and/or administration of an effective mount of one or more therapeutic agents: angiogenesis inhibitors, such as angiostatin K1-3, DL-α-Difluoromethyl-ornithine, endostatin, fumagillin, genistein, minocycline, staurosporine, and (±)-thalidomide; DNA intercalator/cross-linkers, such as Bleomycin, Carboplatin, Carmustine, Chlorambucil, Cyclophosphamide, cis-Diammineplatinum(II) dichloride (Cisplatin), Melphalan, Mitoxantrone, and Oxaliplatin; DNA synthesis inhibitors, such as (±)-Amethopterin (Methotrexate), 3-Amino-1,2,4-benzotriazine 1,4-dioxide, Aminopterin, Cytosine β-D-arabinofuranoside, 5-Fluoro-5′-deoxyuridine, 5-Fluorouracil, Ganciclovir, Hydroxyurea, and Mitomycin C; DNA-RNA transcription regulators, such as Actinomycin D, Daunorubicin, Doxorubicin, Homoharringtonine, and Idarubicin; enzyme inhibitors, such as S(+)-Camptothecin, Curcumin, (−)-Deguelin, 5,6-Dichlorobenzimidazole 1-β-D-ribofuranoside, Etoposide, Formestane, Fostriecin, Hispidin, 2-Imino-1-imidazoli-dineacetic acid (Cyclocreatine), Mevinolin, Trichostatin A, Tyrphostin AG 34, and Tyrphostin AG 879; gene regulators, such as 5-Aza-2′-deoxycytidine, 5-Azacytidine, Cholecalciferol (Vitamin D3), 4-Hydroxytamoxifen, Melatonin, Mifepristone, Raloxifene, all trans-Retinal (Vitamin A aldehyde), Retinoic acid, all trans (Vitamin A acid), 9-cis-Retinoic Acid, 13-cis-Retinoic acid, Retinol (Vitamin A), Tamoxifen, and Troglitazone; microtubule inhibitors, such as Colchicine, Dolastatin 15, Nocodazole, Paclitaxel, Podophyllotoxin, Rhizoxin, Vinblastine, Vincristine, Vindesine, and Vinorelbine (Navelbine); and unclassified antitumor agents, such as 17-(Allylamino)-17-demethoxygeldanamycin, 4-Amino-1,8-naphthalimide, Apigenin, Brefeldin A, Cimetidine, Dichloromethylene-diphosphonic acid, Leuprolide (Leuprorelin), Luteinizing Hormone-Releasing Hormone, Pifithrin-α, Rapamycin, Sex hormone-binding globulin, Thapsigargin, and Urinary trypsin inhibitor fragment (Bikunin). The antitumor agent may be a neoantigen. Neoantigens are tumor-associated peptides that serve as active pharmaceutical ingredients of vaccine compositions which stimulate antitumor responses and are described in US Pub. No. 2011/0293637, which is incorporated by reference herein in its entirety. The antitumor agent may be a monoclonal antibody such as rituximab, alemtuzumab, Ipilimumab, Bevacizumab, Cetuximab, panitumumab, and trastuzumab, Vemurafenib imatinib mesylate, erlotinib, gefitinib, Vismodegib, ⁹⁰Y-ibritumomab tiuxetan, ¹³¹I-tositumomab, ado-trastuzumab emtansine, lapatinib, pertuzumab, ado-trastuzumab emtansine, regorafenib, sunitinib, Denosumab, sorafenib, pazopanib, axitinib, dasatinib, nilotinib, bosutinib, ofatumumab, obinutuzumab, ibrutinib, idelalisib, crizotinib, erlotinib (Tarceva®), afatinib dimaleate, ceritinib, Tositumomab and ¹³¹I-tositumomab, ibritumomab tiuxetan, brentuximab vedotin, bortezomib, siltuximab, trametinib, dabrafenib, pembrolizumab, carfilzomib, Ramucirumab, Cabozantinib, vandetanib, The antitumor agent may be a cytokine such as interferons (INFs), interleukins (ILs), or hematopoietic growth factors. The antitumor agent may be INF-α, IL-2, Aldesleukin, IL-2, Erythropoietin, Granulocyte-macrophage colony-stimulating factor (GM-CSF) or granulocyte colony-stimulating factor. The antitumor agent may be a targeted therapy such as toremifene, fulvestrant, anastrozole, exemestane, letrozole, ziv-aflibercept, Alitretinoin, temsirolimus, Tretinoin, denileukin diftitox, vorinostat, romidepsin, bexarotene, pralatrexate, lenaliomide, belinostat, pomalidomide, Cabazitaxel, enzalutamide, abiraterone acetate, ²²³radium chloride, or everolimus. The antitumor agent may be a checkpoint inhibitor such as an inhibitor of the programmed death-1 (PD-1) pathway, for example an anti-PD1 antibody (Nivolumab). The inhibitor may be an anti-cytotoxic T-lymphocyte-associated antigen (CTLA-4) antibody. The inhibitor may target another member of the CD28 CTLA4 Ig superfamily such as BTLA, LAG3, ICOS, PDL1 or KIR. A checkpoint inhibitor may target a member of the TNFR superfamily such as CD40, OX40, CD137, GITR, CD27 or TIM-3. Additionally, the antitumor agent may be an epigenetic targeted drug such as HDAC inhibitors, kinase inhibitors, DNA methyltransferase inhibitors, histone demethylase inhibitors, or histone methylation inhibitors. The epigenetic drugs may be Azacitidine, Decitabine, Vorinostat, Romidepsin, or Ruxolitinib.

In some embodiments, method of treatment of a cancer (e.g., breast cancer), cancer recurrence (e.g., breast cancer), MBC, or MRD may include administration of an effective amount of a suitable substance able to target intracellular proteins, small molecules, or nucleic acid molecules alone or in combination with an appropriate carrier or vehicle, including, but not limited to, an antibody or functional fragment thereof, (e.g., Fab′, F(ab′)2, Fab, Fv, rlgG, and scFv fragments and genetically engineered or otherwise modified forms of immunoglobulins such as intrabodies and chimeric antibodies), small molecule inhibitors of the protein, chimeric proteins or peptides, gene therapy for inhibition of transcription, or an RNA interference (RNAi)-related molecule or morpholino molecule able to inhibit gene expression and/or translation. In one embodiment the inhibitor is an RNAi-related molecule such as an siRNA or an shRNA for inhibition of translation. An RNA interference (RNAi) molecule is a small nucleic acid molecule, such as a short interfering RNA (siRNA), a double-stranded RNA (dsRNA), a micro-RNA (miRNA), or a short hairpin RNA (shRNA) molecule, that complementarily binds to a portion of a target gene or mRNA so as to provide for decreased levels of expression of the target.

Suitable pharmaceutical composition comprising one or more of the agents described herein is administered and dosed in accordance with good medical practice, taking into account the clinical condition of the individual patient, the site and method of administration, scheduling of administration, patient age, sex, body weight, and other factors known to medical practitioners. The therapeutically effective amount for purposes herein is thus determined by such considerations as are known in the art. For example, an effective amount of the pharmaceutical composition is that amount necessary to provide a therapeutically effective decrease in the expression of the targeted gene. The amount of the pharmaceutical composition should be effective to achieve improvement including but not limited to total prevention and to improved survival rate or more rapid recovery, or improvement or elimination of symptoms associated with the chronic inflammatory conditions being treated and other indicators as are selected as appropriate measures by those skilled in the art. In accordance with the present technology, a suitable single dose size is a dose that is capable of preventing or alleviating (reducing or eliminating) a symptom in a patient when administered one or more times over a suitable time period. One of skill in the art can readily determine appropriate single dose sizes for systemic administration based on the size of the patient and the route of administration.

The pharmaceutical compositions can be formulated according to known methods for preparing pharmaceutically useful compositions. Furthermore, as used herein, the phrase “pharmaceutically acceptable carrier” means any of the standard pharmaceutically acceptable carriers. The pharmaceutically acceptable carrier can include diluents, adjuvants, and vehicles, as well as implant carriers, and inert, non-toxic solid or liquid fillers, diluents, or encapsulating material that does not react with the active ingredients of the technology. Examples include, but are not limited to, phosphate buffered saline, physiological saline, water, and emulsions, such as oil/water emulsions. The carrier can be a solvent or dispersing medium containing, for example, ethanol, polyol (for example, glycerol, propylene glycol, liquid polyethylene glycol, and the like), suitable mixtures thereof, and vegetable oils.

Compositions containing pharmaceutically acceptable carriers are described in several resources which are well known and readily available to those skilled in the art. For example, Remington: The Science and Practice of Pharmacy (Gerbino, P. P. Philadelphia, Pa., Lippincott Williams & Wilkins, 21st ed.) describes formulations that can be used in connection with the subject technology. Formulations suitable for parenteral administration include, for example, aqueous sterile injection solutions, which may contain antioxidants, buffers, bacteriostats, and solutes which render the formulation isotonic with the blood of the intended recipient; and aqueous and nonaqueous sterile suspensions which may include suspending agents and thickening agents. The formulations may be presented in unit-dose or multi-dose containers, for example sealed ampoules and vials, and may be stored in a freeze dried (lyophilized) condition requiring only the condition of the sterile liquid carrier, for example, water for injections, prior to use. Extemporaneous injection solutions and suspensions may be prepared from sterile powder, granules, tablets, etc. In addition to the ingredients particularly mentioned above, the formulations of the subject technology can include other agents conventional in the art having regard to the type of formulation in question.

In some embodiments, the methods described herein also may be implemented by use of computer systems. For example, any of the steps described above for evaluating sequence reads to determine methylation status of a CpG site may be performed by means of software components loaded into a computer or other information appliance or digital device. When so enabled, the computer, appliance or device may then perform all or some of the above-described steps to assist the analysis of values associated with the methylation of a one or more CpG sites, or for comparing such associated values. The above features embodied in one or more computer programs may be performed by one or more computers running such programs.

Further, various aspects of the methods disclosed herein can be implemented using computer-based calculations, machine learning (e.g., support vector machine (SVM), Lasso, Generalized Linear Model (GLM), Gradient Boosted Model (GBM), Extreme Gradient Boosting (XGB), Elastic-Net Regularized Generalized Linear Models (Glmnet), Random Forest, Gradient boosting (on random forest), C5.0 decision trees), and other software tools, or combinations thereof. For example, a methylation status for a CpG site can be assigned by a computer based on an underlying sequence read of an amplicon from a sequencing assay. In another example, a methylation value for a DNA region or portion thereof can be compared by a computer to a threshold value, as described herein. The tools are advantageously provided in the form of computer programs that are executable by a general-purpose computer system of conventional design.

In some embodiments, the method used to analyze and/or determine methylation levels of a target polynucleotide region includes Metilene (Juhling et al., Genome Res., 2016; 26 (2): 256-262) or GenomeStudio Software available online from Illumina, Inc., or as described in Hovestadt et al., 2014; Nature, 510 (7506), 537-541.

In some embodiments, methods of identifying breast cancer, a severity of breast cancer, cancer recurrence, MBC, or MRD in a subject may comprise the use of a machine learning algorithm. The machine learning algorithm may be a trained algorithm. The machine learning algorithm may be trained on one or more features and trained be used to process a data set generated via assaying nucleic acid molecules in a sample (e.g., cell-free biological sample), which data set comprises a methylation profile of one or more genomic regions of the cell-free biological sample. Examples of machine algorithms use and training of said machine learning algorithm are described, for example in PCT Patent Publication No. WO/2022/178108 to Salhia et al.

In some embodiments, a computer comprising at least one processor may be configured to receive a plurality of sequencing results from the DNA methylation sequencing reactions that may comprise the methylation pattern of one or more target regions disclosed herein from a patient having, for example, a mass (e.g., breast mass) or other tumor, or suspected of having a cancer, or showing clinical signs of cancer. In some embodiments, the machine learning algorithm or program used to develop the MRD signature comprises analyzes methylation patterns of a plurality of target regions of cancerous samples as compared to methylation patterns of a plurality of target regions of non-cancerous samples. In some embodiments, the cancerous samples are from stage IV cancer samples, such as, for example, metastatic breast cancer. In some embodiments, the MRD signature is developed by determining and analyzing a methylation pattern of a plurality of target regions of both cancerous and non-cancerous samples wherein the plurality of target regions comprise at least 5%, at least 10%, at least 15%, at least 20%, at least 25%, at least 30%, at least 40%, at least 45%, at least 50%, at least 55%, at least 60%, at least 65%, at least 70%, at least 75%, at least 80%, a least 85%, at least 90%, at least 95%, at least 96%, at least 97%, at least 98%, at least 99%, or 100% of the target regions listed in Table 1. The methylation pattern of the cancerous samples may then be compared to the methylation pattern of the non-cancerous samples to develop the MRD signature as discussed in more detail below.

Any of the computer-readable media herein can be non-transitory (e.g., volatile memory such as DRAM or SRAM, nonvolatile memory such as magnetic storage, optical storage, or the like) and/or tangible. Any of the storing actions described herein can be implemented by storing in one or more computer-readable media (e.g., computer-readable storage media or other tangible media). Any of the things (e.g., data created and used during implementation) described as stored can be stored in one or more computer-readable media (e.g., computer-readable storage media or other tangible media). Computer-readable media can be limited to implementations not consisting of a signal.

### Results and Discussion

Breast cancer is the second leading cause of cancer deaths in women in the United States; metastatic breast cancer (MBC) accounts for 10-15% of breast cancer cases but has the worst outcome by far with a 5-year overall survival (OS) of less than 25%. MBC arises from disseminated cells from the primary tumor mass before treatment and/or minimal residual disease (MRD) remaining after therapy. Molecular based clinical tests have improved our ability to stratify patients based on recurrence risk using molecular profiles from primary tumor tissue. However, tumor tissue is not always available and offers only a snapshot of a tumor. Thus, there is a need for biomarkers that can be monitored noninvasively and repeatedly over time to predict recurrence risk. Cell-free (cf) DNA is a promising biomarker for MRD detection. Here, we use cfDNA methylation patterns as a marker of MRD. We have developed a bioinformatic pipeline to extract methylation information from each cfDNA fragment, as opposed to an average methylation (beta) value. Our results show that this approach may be more sensitive at detecting cancer specific methylation in a low disease burden setting, such as MRD. We can compare this ‘fragment-level’ approach with beta value in an in-silico spike in model to determine the theoretical limit of detection for each approach. This software can detect evidence for residual disease in a longitudinal cohort consisting of both women who recurred after primary treatment and disease-free survivors (DFS). This cohort consists of blood collections from four timepoints before, during, and after treatment. The test consists of 1564 differentially methylated regions (DMRs), some or all of which may be used to detect MRD, breast cancer recurrence, or MBC and indicate women at high risk of recurrence who may benefit from additional therapy. This represents a major step towards developing a blood test to monitor and predict distant recurrence in breast cancer.

Research to date has demonstrated that there are detectable differences in beta value between healthy individuals and patients with MBC. However, recent studies have found that fragment-level methylation is a powerful analytical tool to detect unique methylation states, particularly in cfDNA (Klein et al. Ann Oncol 32, 1167-1177, (2021); Liu et al. Mol Cancer 20, 36, (2021); Moss et al. Nat Commun 9, 5068, (2018); Kang et al. Genome Biol 18, 53, (2017); Guo et al. Nat Genet 49, 635-642, (2017)). To date, there are no publicly available tools to analyze fragment level methylation data, nor has it been directly compared against beta value-based analysis.

Beta value is the ratio of methylated CpGs at a given locus (β=ΣMethylated CpG/Total CpG), and can be evaluated per-CpG or averaged across a defined region. cfDNA is highly fragmented (˜160 bp fragments) and these fragments may derive from diverse sources: dying epithelial cells, leukocytes, necrotic tissue, or—most importantly—tumor tissue, each of which will contain unique methylation states. Thus, the beta value for a specific CpG, from a cfDNA sample, represents an average across multiple tissues of origin. Within a solid tumor sample, the measured beta will average across molecularly heterogeneous tumor cells, stromal cells, and adjacent normal tissue. For cfDNA, the challenge is to identify tumor-specific cfDNA with sufficient sensitivity and specificity in MRD, where tumor burden is expected to be especially low.

An emerging approach to methylation evaluation considers each DNA fragment (i.e., a sequencing read) to determine patterns of CpG methylation. This fragment-level approach offers several potential advantages over the beta value: 1) The context of adjacent CpGs is retained in fragment level data. When calculating regional methylation, beta values may be determined for individual CpGs, but these are averaged to provide a single metric for a differentially methylated region (DMR). In contrast, a fragment-level approach tabulates all methylation states for adjacent CpGs, thus retaining the context of surrounding CpGs. This improvement in data resolution may be expected to improve the performance of classifiers for early detection of MRD because it incorporates biological variability of all the possible DNA methylation states. For example, in FIG. 1 fragments IV and V, the mean beta value across the last 4 CpGs would be 0.5, yielding a Δβ value of 0. However, the fragments have completely opposite methylation patterns suggesting separate tissues of origin. 2) Fragment level methylation allows Boolean (binary) feature classification—that is, evaluating whether cancer specific fragments (CSFs) of DNA are present in a given sample. This approach may be more sensitive in low tumor burden situations, such as MRD.

We have developed a computer implemented package to evaluate fragment-level DNA methylation—Fragment Level Assessment and Methylation Extraction (FLAME). FLAME is comprised of 3 main functions: CpG clustering, methylation tabulation, and fragment analysis.

The CpG clustering subroutine of FLAME combines nearby CpGs into discrete blocks with n to m CpGs where n and m are user-specified. Crucially, these blocks must be less than the fragment length; to evaluate methylation patterns of CpGs within a block, a read must span these CpGs. The clustering algorithm functions in two stages: 1) Combine closely adjacent CpGs into contiguous regions. Any region with less than n CpGs is removed; regions containing between n and m CpGs and are less than the maximum length are retained. 2) All other regions are recursively split until the user set constraints are met or the region is found to be unsuitable. Sub-division of regions is performed using k-means clustering based on nearest adjacent CpGs. Regions passing filter are hereafter referred to as ‘fragment assessment regions (FAR)’.

After FARs have been selected, methylation tabulation may be performed to count all methylation states in each fragment. FLAME takes two files as input: a bedGraph listing the genomic coordinates and number of CpGs per FAR, and a bam file containing mapped reads. The program filters the bam file, retaining only reads that overlap a FAR to speed up runtime and reduce the memory footprint of subsequent steps. For each read, the genomic coordinates, mapping information, and the methylation states are recorded in a custom data structure. Methylation tabulation has the following steps (a) Identification of all possible methylation patterns, given the number of CpGs in the FAR, (b) Selection of all reads that overlap the FAR, (c) extract the methylation states of each CpG in the read that spans the FAR, and (d) Count each distinct methylation pattern in the fragment, returning a data structure like that in Table 2. If no reads overlap a FAR, all values are assigned as NA. Finally, FLAME outputs a table with each row detailing the FAR location, the methylation pattern, and the count.

TABLE 2
Example output from fragment level analysis methods
from one region. All methylation states are tabulated
from 3 CpGs. The count number is the number of times
a specific methylation pattern is observed.

| Methylation status | Count |
|---|---|
| UUU | 11 |
| UUM | 3 |
| UMU | 0 |
| UMM | 9 |
| MUU | 3 |
| MUM | 10 |
| MMU | 0 |
| MMM | 45 |

Finally, FLAME merges fragment counts from multiple samples, normalizes based on sequencing depth, and looks for fragments that are differentially expressed between groups (i.e., to distinguish methylation patterns observed in cancerous samples compared to methylation patterns found in healthy control sample). Additionally, FLAME supports data visualization functions, and export functions for further analysis in packages such as SAS, SPSS, and Microsoft Excel. As used herein, the term “sequencing depth” refers to the number of times a genomic locus is covered by a read (e.g., 10 reads overlapping one locus=10× sequencing depth). In some embodiments, FLAME comprises at least statements of the embodiments numbers 11-14.

### Method Testing

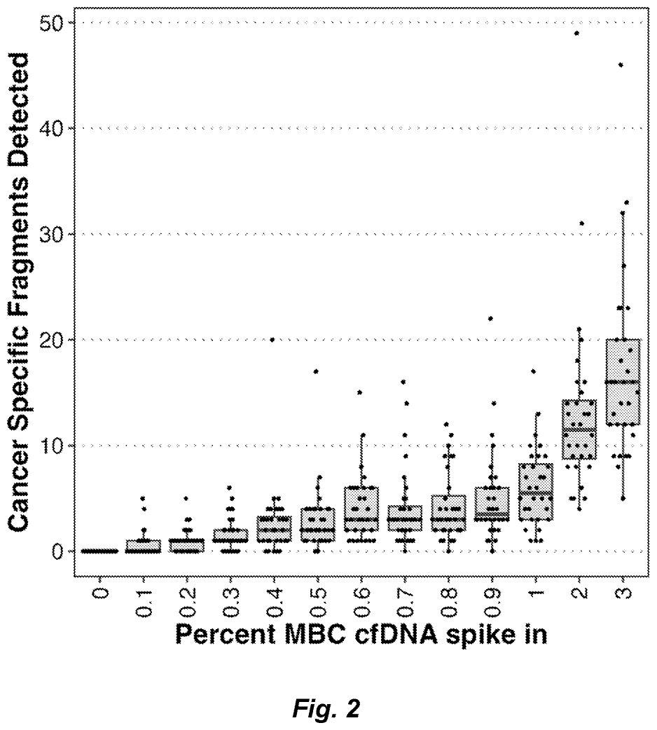

First, FLAME was beta tested to ensure it is functioning as intended. We compared region coverage as evaluated by FLAME to our standard methylation pipeline. We also compared beta values, which are trivial to calculate from fragment-level output. We observed a tight linear relationship between beta values calculated by FLAME and beta values calculated by bsseq, a commonly used R package to analyze bisulfite sequencing data (FIG. 2a). FLAME calculated lower total coverage than bsseq; this is because FLAME will only count reads that fully span FARs (FIG. 2b).

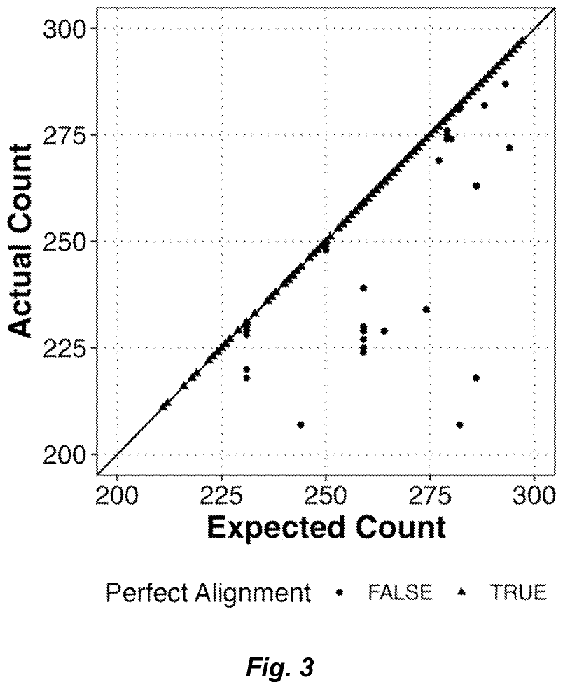

Since there is no publicly available software to provide similar comparisons for fragment analysis, we used a synthetic dataset with known fragment methylation patterns and counts. To generate these test data sets in silico, we focused on selected genomic (hg19) sequences and generated synthetic reads in which ‘methylation’ simulated by C to T substitutions with predefined patterns and counts. From these sequences we generated fastq files and ran alignment and fragment-level tabulation using the same software and parameters we would use for a biological sample. This provided a means to compare tabulation data against a ‘ground truth’. We observed that the methylation patterns counted by FLAME were identical to our expected counts in these synthetic samples (FIG. 3) except in cases of suboptimal alignment.

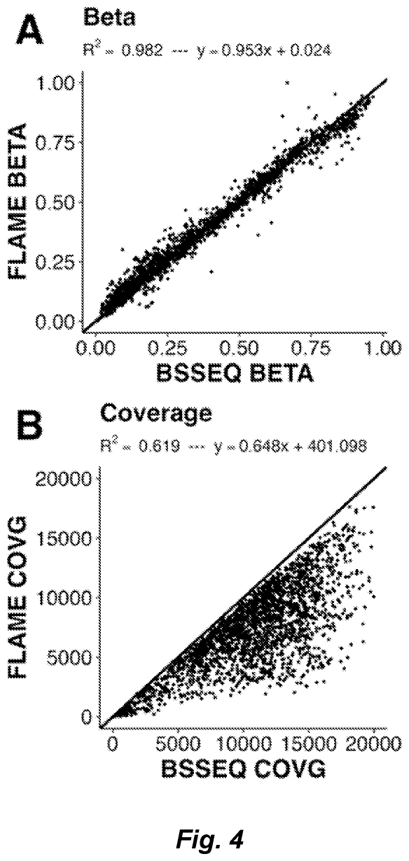

To compare BC-specific methylation signals in cfDNA, as evaluated FLAME, we performed an in-silico spike in of cfDNA from MBC patients into cfDNA from healthy individuals as a proxy for cfDNA tumor burden to determine a theoretical limit of detection. We found cancer specific fragments (CSFs) we had previously observed in MBC patients but not in healthy individuals. We observed CSFs at low simulated tumor fraction, indicating this method can detect evidence of MRD even in a low tumor burden scenario (FIG. 4).

To evaluate the ability of FLAME to identify DNA methylation changes genome-wide, we compare fragment-level data output genome wide to DMR results from Metilene. Since DMRs are predicated on differences in beta value, we find significant overlap between DMRs called by beta and regions called by fragment level analysis. We then compare the genomic loci of all significantly differentially methylated fragments to Metilene DMRs to assess what percent of regions overlap and which regions are gained. We then find regions with DNA methylation patterns that are significantly different but fail to be called by Metilene as the average Δβ across the region is low. These regions represent methylation changes that only a fragment-level approach can detect.

### Statistical Plan and Machine Learning

The output may be evaluated using two methods. First, comparing the sensitivity and specificity of machine learning (ML) models constructed with fragment level data to models built using beta in cfDNA from MBC patients. Briefly, MBC and healthy samples are split into 70/30 training/testing sets. Matrices containing fragment-level data and beta value data are used to train ML models to predict MBC versus healthy. These models are constructed using multiple algorithms including but not limited to Random Forest (RF), a support vector machine (SVM), a neural network, Generalized Linear Model (GLM), Gradient Boosted Model (GBM), Extreme Gradient Boosting (XGB), or a deep learning algorithm. The training may be repeatedly subdivided during the training process (repeated cross validation) as a precaution against overfitting the final model. The testing set is then evaluated by the final model, the sensitivity and specificity of the model are evaluated by receiver operating characteristic (ROC) analysis. We constructed a ROC curve and compute the area under the curve (AUC) to assess performance. We then directly compare the AUC of models built with fragment-level vs beta value data. This process may be repeated multiple times to get an average AUC for both input modalities.

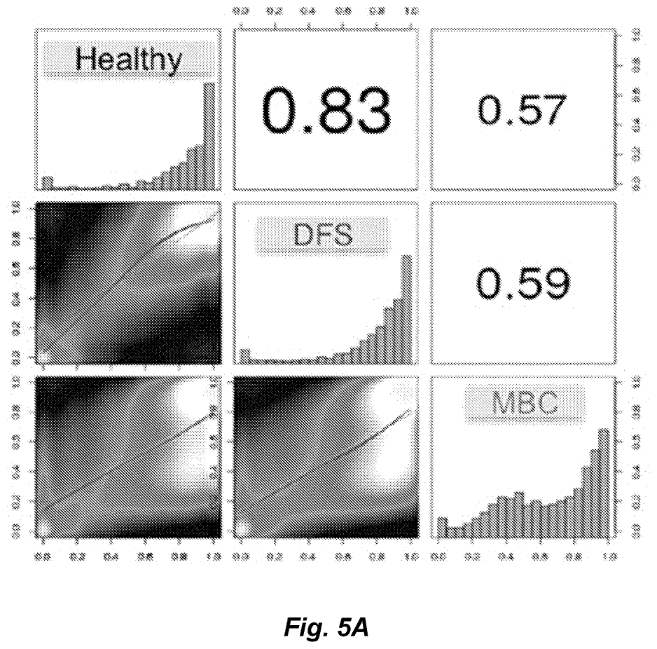

Previously published results from WGBS on cfDNA obtained from plasma samples in 3 cohorts of 40 individuals each: cohort 1 was from MBC to various organs; cohort 2 was from disease free survivors; cohort 3 was from healthy females with no history of cancer. Differential methylation analysis on WGBS data demonstrated that there were relatively few differences seen between healthy subjects and disease-free survivors as indicated by relatively few differentially methylated loci (n=87,935), a high Pearson correlation coefficient (0.83), hierarchical clustering and principal component analysis (FIG. 5). In contrast, approximately 5.0×10⁶ differentially methylated loci were detected between women with metastatic disease and those without. This suggests that methylation patterns in cfDNA may be useful to monitor treatment and detect the presence of minimal residual disease.

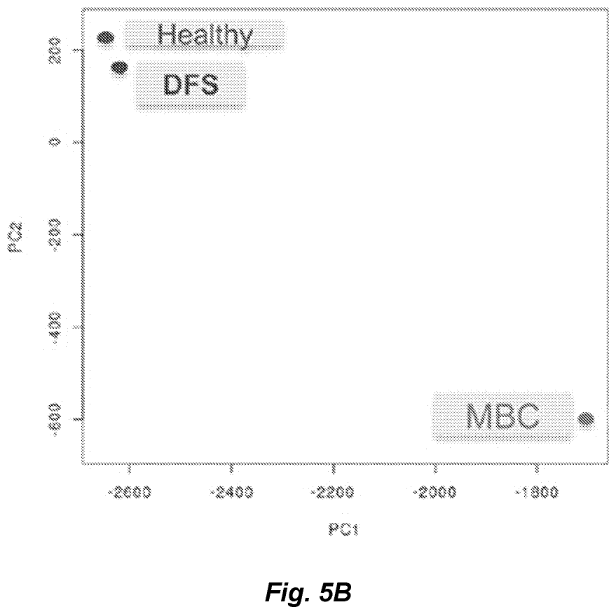

As a continuation of this work, an R01 study has expanded to include subtype specific pools: ER+/HER2− (n=13), ER−/HER2+ (n=8), ER+/HER2+ (n=9), TNBC (n=5), and TNBC-AA (an African American specific pool, n=20). Three additional pools from healthy individuals were created as controls: two from women of predominantly European descent (n=9,10) and one pool from African American women (n=20). All pools were profiled by WGBS. After sequencing, paired-end reads were aligned to hg19 (GRCh37) using Bismark Bisulfite Read Mapper (Krueger et al., Bioinformatics 27, 1571-1572, doi:10.1093/bioinformatics/btr167 (2011)) and DMRs were called using the open-source software Metilene (Juhling et al. Genome Res 26, 256-262, doi:10.1101/gr.196394.115 (2016).). DMRs were filtered based on |Δβ|, FDR corrected p-value, and sequencing depth resulting in a multi-subtype signature of 713 regions. In order to validate this MBC signature, we designed a targeted assay using hybrid probe capture, allowing us to cost-effectively sequence multiple samples. 96 samples (#MBC=64, #Healthy=32) were captured and sequenced to validate the 713 DMRs we previously discovered as a signature for MBC. A random forest model was constructed from mean beta values across all 713 regions to discriminate individuals with MBC from healthy individuals, with 30% of samples left out as a test set. These results show that these regions are highly capable of discriminating healthy samples from MBC (FIG. 6) with an AUC of 0.93, sensitivity of 0.8, specificity of 0.9, positive predictive value of 0.94 and negative predictive value of 0.69.

Next, the methylation profiles of patients with BC before and after treatment are evaluated to determine whether a BC signal from beta value or fragment level data could detect evidence of MRD. We obtained cfDNA from a cohort of 119 women with BC collected at 4 timepoints: pre neoadjuvant therapy, post neoadjuvant therapy, postoperative, and 1 year after treatment (Table 3) (hereafter referred to as ‘the Mayo cohort’).

TABLE 3
Description of all samples in the Mayo Cohort. Each subtype
is represented as a separate row. ‘Total collections’
represents the number of individual plasma samples obtained.

| | Total | | | | Total |
|---|---|---|---|---|---|
| Subtype | Patients | NDF | Recurrence | DFS¹ | Collections |
| ER−/HER2− | 5 | 1 | 0 | 4 | 79 |
| ER−/HER2+ | 10 | 0 | 3 | 7 | 175 |
| ER+/HER2− | 36 | 2 | 1 | 33 | 500 |
| ER+/HER2+ | 26 | 0 | 0 | 26 | 406 |
| TNBC | 42 | 1 | 7 | 34 | 583 |
| TOTAL | 119 | 4 | 11 | 104 | 1743 |

¹DFS as of March 2022.

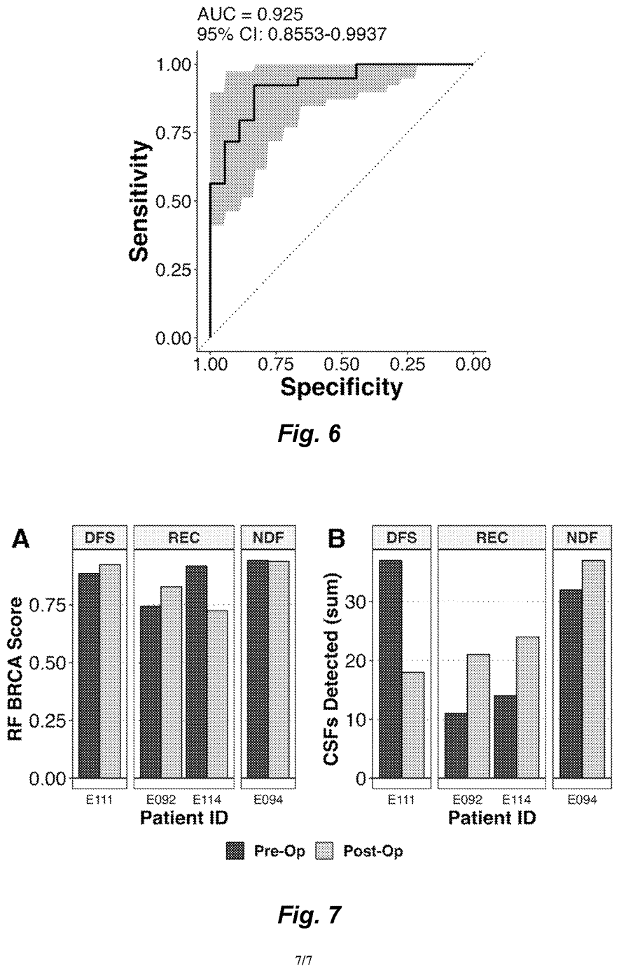

To generate pilot data from this cohort, we analyzed 16 cfDNA plasma samples from 4 patients from this dataset; 2 of these patients recurred, one was never disease-free (per physician's assessment), and one had not recurred at the time of analysis. To preliminarily assess the potential utility of using cfDNA methylation to detect evidence of recurrence, we attempted to predict recurrence by 1) predicting disease status using beta value and the RF model detailed above and 2) tabulating the number of CSFs present in each sample using FLAME (FIG. 7). Here a CSF is defined as a methylation pattern found in at least 5% of the 64 stage IV samples mentioned above and not found in any normal cfDNA samples; fragment counts were tabulated using the proof-of-concept version of software. Our results show that the RF model constructed using beta value has no significant change between timepoints, while CSF shows a clear decrease in signal in DFS, increase in signal in recurrent samples, and a modest increase in signal in never disease free.

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims.

All publications, patents, and patent documents are incorporated by reference herein, as though individually incorporated by reference including Legendre et al. Clin Epigenetics 2015 Sep. 16; 7 (1): 100. doi: 10.1186/s13148-015-0135-8; Buckley et al., Clin Cancer Res. 2023 Oct. 9. doi: 10.1158/1078-0432.CCR-23-1197. PMID: 37812492; U.S. Pat. No. 10,525,148 to Salhia et al.; U.S. Pat. No. 11,035,849 to Salhia et al.; U.S. Pat. Pub No. US20200340062 to Salhia et al; PCT Patent Publication No. WO/2020150258 to Olsen et al.; and PCT Patent Publication No. WO/2022/178108 to Salhia et al. No limitations inconsistent with this disclosure are to be understood therefrom. The invention has been described with reference to various specific and preferred embodiments and techniques. However, it should be understood that many variations and modifications may be made while remaining within the spirit and scope of the invention.

1. A method for determining whether a subject has Minimum Residual Disease (MRD) comprising steps:
a) training a machine learning model to develop an MRD signature, wherein the machine learning program is trained using target regions from cancerous samples and corresponding target regions from non-cancerous samples, wherein the MRD signature is based on a comparison of a methylation pattern of target regions of the cancerous samples compared to a methylation pattern of corresponding target regions of the non-cancerous samples;
b) determining a methylation pattern of target regions of a cell-free deoxyribonucleic acid (cfDNA) sample obtained from the subject;
c) applying the MRD signature to the methylation pattern of the target regions of the cfDNA obtained from the subject; and
d) determining that the subject has or does not have the MRD based on the MRD signature.

2. The method of claim 1 wherein the target regions in the cfDNA sample from the subject are identical to the target regions of both the cancerous sample and the non-cancerous samples used to develop the MRD signature.

3. The method of claim 2 wherein the methylation pattern of the target regions is determined using one or more of post whole genome library hybrid probe capture, enzymatic treatment, bisulfite amplicon sequencing (BSAS), bisulfite treatment of DNA, methylation sensitive polymerase chain reaction, and bisulfite conversion combined with bisulfite restriction analysis.

4. The method of claim 1 wherein the methylation pattern of each of the target regions is determined using a hybrid probe capture method.

5. The method of claim 4 wherein the hybrid probe capture method comprises using one or more hybrid capture probes comprising ribonucleic acid or deoxyribonucleic acid.

6. The method of claim 5 wherein each of the one or more hybrid capture probes further comprises an affinity tag selected from the group consisting of biotin and streptavidin

7. The method of claim 1 wherein the target regions from cancerous samples and from non-cancerous samples comprise about 60% to at about 70% of the target regions of Table 1.

8. The method of claim 7 wherein the target regions comprise about 70% to about 80% of the target regions of Table 1.

9. The method of claim 8 wherein the target regions comprise about 80% to about 90% of the target regions of Table 1.

10. The method of claim 9 wherein the target regions comprise greater than about 95% of the target regions of Table 1.

11. The method of claim 1 wherein the cfDNA sample is extracted from whole blood, plasma, serum, or urine.

12. The method of claim 1 further comprising steps:
e) combining adjacent CpGs of each of the target regions into contiguous n through m number of CpG blocks wherein n is at least 1 and m is less than a length of a corresponding target region;
f) removing any target region having less than the n number of CpG blocks and greater than the m number of CpG blocks; and
g) filtering the target regions remaining after step f) using a k-means clustering function based on adjacent CpGs to provide one or more fragment assessment regions (FAR).

13. The method of claim 12 further comprising tabulating a methylation state of each FAR according to the steps of:
h) identifying all or substantially all possible methylation patterns of CpGs in the FAR;
i) selecting all sequence reads that overlap the FAR;
j) extracting the methylation states of each of the CpGs in the sequence read that spans the FAR;
k) counting each distinct methylation pattern in the FAR to provide a count of methylation states; and
l) outputting a result of steps h)-k), wherein the output comprises one or more of the FAR locations, the methylation pattern of the FAR, and the count of the FAR.

14. The method of claim 13 further comprising:
merging each of the counts of the FAR;
normalizing the counts of the FAR based on sequence depth; and
identifying a FAR that is differentially methylated between the cfDNA sample of the subject and the cancerous samples and the non-cancerous samples.

15. The method of claim 1 comprising using the trained machine learning model to determine whether the subject is likely to have or develop metastatic breast cancer, breast cancer recurrence, or both metastatic breast cancer and breast cancer recurrence.

16. The method of claim 15 wherein the machine learning model comprises one or more of a RandomForest, a support vector machine (SVM), a neural network, Generalized Linear Model (GLM), Gradient Boosted Model (GBM), Extreme Gradient Boosting (XGB), and a deep learning algorithm.

17. The method of claim 1 wherein the cancerous samples and the non-cancerous samples comprise one or more of breast cancer samples, known metastatic breast cancer samples, breast cancer recurrence samples, samples from a subject that has completed a cancer treatment regimen, and samples from subjects with no evidence of disease using standard of care treatment.

18. The method of claim 1 further comprising treating the subject having the MRD, wherein the treatment comprises one or more of radiation therapy, surgery to remove the cancer, and administering a therapeutic agent to the patient, thereby treating the MRD.
